(19) United States
(12) Patent Application Publication
(10) Pub. No.: US 2016/0264593 A1
(43) Pub. Date: Sep. 15, 2016

(54) PROTEIN PHOSPHATASE INHIBITORS THAT CROSS THE BLOOD BRAIN BARRIER

(71) Applicants: John S. Kovach, East Setauket, NY (US); Francis Johnson, Setauket, NY (US); Ramakrishna Samudrala, Port Jefferson Station, NY (US); Ramesh C. Gupta, Stony Brook, NY (US)

(72) Inventors: John S. Kovach, East Setauket, NY (US); Francis Johnson, Setauket, NY (US); Ramakrishna Samudrala, Port Jefferson Station, NY (US); Ramesh C. Gupta, Stony Brook, NY (US)

(21) Appl. No.: 15/036,760

(22) PCT Filed: Nov. 14, 2014
(86) PCT No.: PCT/US14/65669

§ 371 Date: May 13, 2016

Related U.S. Application Data
(60) Provisional application No. 61/904,821, filed on Nov. 15, 2013.

(51) Int. Cl. C07D 493/08; C07F 9/6561

(52) U.S. Cl. CPC C07D 493/08; C07F 9/6561

(57) ABSTRACT

The present invention provides a method for in vivo delivery of endothal to a target cell in a subject, the method comprising administering to the subject a compound having the structure: Formula (I).

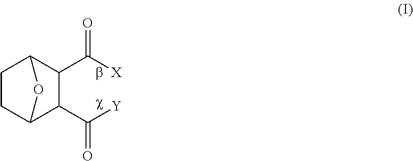

This application claims priority of U.S. Provisional Application No. 61/904,821, filed Nov. 15, 2013, the contents of which are hereby incorporated by reference.

Throughout this application various publications are referenced. The disclosures of these documents in their entireties are hereby incorporated by reference into this application in order to more fully describe the state of the art to which this invention pertains.

### BACKGROUND OF THE INVENTION

Retinoids, metabolites of vitamin A, have been examined therapeutically against a variety of tumors, including gliomas (Yung et al. 1996). Nuclear receptor co-repressor (N—CoR) is closely associated with the retinoid receptor and is released upon ligand binding to the receptor (Bastien et al. 2004). By preventing the action of protein phosphatase-1 and protein phosphatase-2A (PP2A), anti-phosphatases increase the phosphorylated form of N—CoR and promote its subsequent cytoplasmic translocation (Hermanson et al. 2002).

The phosphatase inhibitor, Cantharidin, has anti-tumor activity against human cancers of the liver (hepatomas) and of the upper gastrointestinal tract but is toxic to the urinary tract (Wang, 1989). Cantharidin acts as a protein phosphatase inhibitor, which prompted a more general interest in compounds with this type of chemical structure (Li and Casida 1992). Previously, it had been found that the simpler congener and its hydrolysis product (commercially available as the herbicide, Endothal) are hepatotoxic (Graziani and Casida, 1997). Binding studies have shown that the action of certain cantharidin homologs is direct on protein phosphatase-2A and indirect on protein phosphatase-1 (Honkanen et al., 1993; Li et al., 1993).

Of the known congeners of this type of compound, only the parent, cantharidin and its bis(normethyl)-derivative, norcantharidin, have seen any use as anti-cancer drug substances and only norcantharidin is used as an anti-neoplastic agent (Tsauer et al. 1997).

Despite these successes, few compounds of this type have been screened for anti-tumor or cytotoxic activity. Currently, there is a significant need to develop inhibitors of protein phosphatases that are more active, less toxic and more specific in action than the known substances mentioned above. In particular, the need is present for diseases such as high-grade malignant gliomas of children and adults.

Diffuse intrinsic pontine glioma (DIPG) is a non-operable cancer of the brainstem in children for which no treatment other than radiation has offered any extension of life, with survival with best care being about 12 months. Multiple trials of adjuvant chemotherapy have not significantly improved outcomes (Warren et al. 2011; Hawkins et al. 2011). There are about 300 new cases diagnosed annually in the United States. Glioblastoma multiforme (GBM) is an aggressive brain cancer occurring in about 20,000 adults annually in the US for which standard treatment (primary surgery, followed by 6-weeks of radiation plus temozolomide, followed by daily oral temozolomide) has only increased average lifespan from less than one year to about 18 months despite 50 years of testing experimental therapies (Stupp et al. 2009). There is an urgent need for new treatments of these gliomas.

Many chemotherapeutic agents used to treat cancer exhibit serious toxicity, resulting in unwanted side effects for patients and reducing efficacy by limiting the doses that can be safely administered. Prodrugs, which are converted to the active drug in vivo, can offer many advantages over parent drugs such as increased solubility, enhanced stability, improved bioavailability, reduced side effects, better selectivity and improved entry of the drug to certain tissues. Activation of prodrugs can involve many enzymes through a variety of mechanisms including hydrolytic activation (Yang, Y. et al. 2011). Enzymes involved in the hydrolytic activation of prodrugs include carboxylesterases and amidases.

Endothal is the common name for 7-oxabicyclo[2.2.1]heptane-2,3-dicarboxylic acid. It is an inhibitor of PP2A, an enzyme present in both plants and animals that is involved in the dephosphorylation of proteins. Endothal is structurally similar to cantharidin, a chemical compound secreted by many species of blister beetle. Endothal is known as an active defoliant and potent contact herbicide used in many agricultural situations. It is considered effective as a pre-harvest desiccant and as a selective pre-emergence herbicide. Endothal has been tested against a limited number of human cancer cell lines (Thiery J. P. et al. 1999).

### SUMMARY OF THE INVENTION

The present invention provides a method for in vivo delivery of endothal to a target cell in a subject, the method comprising administering to the subject a compound having the structure:

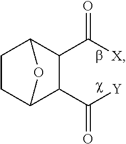

wherein
X is OR₃ or NR₄R₅,
   wherein each of R₃, R₄ and R₅ is H or an organic moiety, or
   R₄ and R₅ combine to form an organic moiety;
Y is OR₆ or NR₇R₈;
   wherein each of R₆, R₇ and R₈ is H or an organic moiety, or
   R₇ and R₈ combine to form an organic moiety;
wherein when one of X or Y is OH, then the other of X or Y is other than OH, NR₄R₅ or NR₇R₈ where R₄ and R₅ or R₇ and R₈ combine to form an N-methyl piperazine,
or a pharmaceutically acceptable salt or ester of the compound,
wherein one or both of bond β and bond χ is subject to in vivo hydrolytic cleavage in the subject,
so as to thereby deliver endothal to the target cell in the subject.

The present invention also provides a compound having the structure:

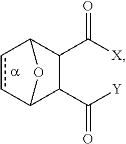

wherein
bond α is absent or present;
X is OR₁, OR₃ or NR₄R₅,
   wherein
   R₁ is C₁-C₂₀ alkyl, C₂-C₂₀ alkenyl, or C₂-C₂₀ alkynyl;
   R₃ is H, alkyl, alkylaryl, alkyl-P(O)(OR₉)₂, alkyl-OP(O)(OR₉)₂, alkyl-O(CO)—OR₁₀, alkyl-P(O)(O-alkyl-O(CO)—OR₁₀)₂, or alkyl-OP(O)(O-alkyl-O(CO)—OR₁₀)₂;
   R₄ and R₅ are each independently H, alkyl-P(O)(OR₉)₂, alkyl-OP(O)(OR₉)₂, alkyl-O(CO)—OR₁₀, alkyl-P(O)(O-alkyl-O(CO)—OR₁₀)₂, or alkyl-OP(O)(O-alkyl-O(CO)—OR₁₀)₂,
   or R₄ and R₅ combine to form an unsubstituted or substituted cycloalkyl, cycloalkenyl, cycloalkynyl or heterocycloalkyl,
      wherein R₉ and R₁₀ are each independently H, alkyl, alkenyl, or alkynyl; and
Y is OR₁, OR₆ or NR₇R₈,
   wherein
   R₁ is C₁-C₂₀ alkyl, C₂-C₂₀ alkenyl, or C₂-C₂₀ alkynyl;
   R₆ is alkyl-P(O)(OR₉)₂, alkyl-OP(O)(OR₉)₂, alkyl-O(CO)—OR₁₀, alkyl-P(O)(O-alkyl-O(CO)—OR₁₀)₂, or alkyl-OP(O)(O-alkyl-O(CO)—OR₁₀)₂; and
   R₇ and R₈ are each independently H, alkyl-P(O)(OR₉)₂, alkyl-OP(O)(OR₉)₂, alkyl-O(CO)—OR₁₀, alkyl-P(O)(O-alkyl-O(CO)OR₁₀)₂, or alkyl-OP(O)(O-alkyl-O(CO)—OR₁₀)₂,
   or R₇ and R₈ combine to form an unsubstituted or substituted cycloalkyl, cycloalkenyl, cycloalkynyl or heterocycloalkyl,
      wherein R₉ and R₁₀ are each independently H, alkyl, alkenyl, or alkynyl,
wherein one of X is OH, OCH₃ or O-alkylaryl, then Y is other than NR₇R₈ where R₇ and R₈ combine to form an unsubstituted or substituted piperazine, morpholine or thiomorpholine,
or a pharmaceutically acceptable salt or ester of the compound.

The present invention further provides a method of treating a subject afflicted with cancer comprising administering a therapeutically effective amount of a compound having the structure:

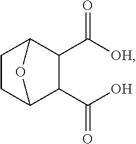 [SMILES: O=C(O)C1C2CCC(O2)C1C(=O)O]

or a pharmaceutically acceptable salt or ester thereof, so as to thereby treat the subject.

The present invention further provides a method of inhibiting proliferation or inducing apoptosis of a cancer cell in a human subject afflicted with cancer comprising administering a therapeutically effective amount of a compound having the structure:

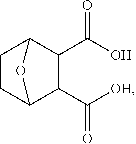 [SMILES: O=C(O)C1C2CCC(O2)C1C(=O)O]

or a pharmaceutically acceptable salt or ester thereof, so as to thereby inhibit proliferation or induce apoptosis of the cancer cell.

The present invention further provides a compound having the structure

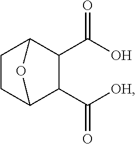 [SMILES: O=C(O)C1C2CCC(O2)C1C(=O)O]

or a pharmaceutically acceptable salt or ester thereof, for use in treating cancer in a subject.

### BRIEF DESCRIPTION OF THE FIGURES

FIG. 1: The inhibition effect of 100, 113, 151, 153 and 157 on PP2A in mouse livers. One way ANOVA was used in statistical analysis: vs vehicle 3 h, *p<0.05, **p<0.01, ***p<0.001; vs vehicle 6 h, #p<0.05, ##p<0.01, ###p<0.001.

FIG. 2: The inhibition effect of 100, 113, 151, 153 and 157 on PP2A in mouse brains. One way ANOVA was used in statistical analysis: vs vehicle 3 h, *p<0.05, **p<0.01, ***p<0.001; vs vehicle 6 h, #p<0.05, ##p<0.01, ###p<0.001.

FIG. 3: Cell viability effect of 100, 153, 157, 158, 159 against 2LMP cancer cells.

FIG. 4: Cell viability effect of 100, 153, 157, 158, 159 against U-87 cancer cells.

FIG. 5: Cell viability effect of 100, 153, 157, 158, 159 against A549 cancer cells.

FIG. 6A: Concentration versus time curves of 153 in plasma following iv or po administration, and in liver and brain following iv administration of 153 to SD rats.

FIG. 6B: Concentration versus time curves of Endothal in plasma following iv or po administration, and in liver following iv administration of 153 to SD rats.

FIG. 6C: Concentration versus time curves of 157 in plasma following iv or po administration, and in, liver and brain following iv administration of 157 to SD rats.

FIG. 6D: Concentration versus time curves of Endothal in plasma following iv or po administration, and in liver following iv administration of 157 to SD rats.

FIG. 7A: Mean plasma and liver concentration-time profiles of 105 after IV dose of 1 mg/kg in SD rats (N=2/time point).

FIG. 7B: Mean plasma and liver concentration-time profile of Endothal after IV dose of 1 mg/kg 105 in male SD rats (N=2/time point).

FIG. 7C: Mean plasma and liver concentration-time profile of 105 and Endothal after an IV dose of 1 mg/kg 105 in male SD rats (N=2/time point).

FIG. 8A: Mean plasma, brain and liver concentration-time profile of 113 after IV or PO dose of 1.4 mg/kg in male SD rats (N=2/time point).

FIG. 8B: Mean plasma and liver concentration-time profile of Endothal after IV dose of 1.4 mg/kg 113 in male SD rats (N=2/time point)

FIG. 8C: Mean plasma and liver concentration-time profile of 100 after IV dose of 1.4 mg/kg 113 in male SD rats (N=2/time point)

FIG. 8D: Mean plasma, brain and liver concentration-time profile of 113, 100 and Endothal after IV or PO dose of prodrug 113 at 1.4 mg/kg in male SD rats (N=2/time point)

FIG. 9A: Concentration versus time curves of 100 in plasma following iv administration of 100 to SD rats.

FIG. 9B: Concentration versus time curves of 100 in brain following iv administration of 100 to SD rats.

FIG. 9C: Concentration versus time curves of 100 in liver following iv administration of 100 to SD rats.

FIG. 9D: Concentration versus time curves of endothal in plasma following iv administration of 100 to SD rats.

FIG. 9E: Concentration versus time curves of endothal in liver following iv administration of 100 to SD rats.

### DETAILED DESCRIPTION OF THE INVENTION

The present invention provides a method for in vivo delivery of endothal to a target cell in a subject, the method comprising administering to the subject a compound having the structure:

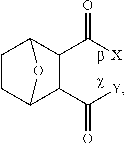

wherein
X is OR₃ or NR₄R₅,
   wherein each of R₃, R₄ and R₅ is H or an organic moiety, or R₄ and R₅ combine to form an organic moiety;
Y is OR₆ or NR₇R₈;
   wherein each of R₆, R₇ and R₈ is H or an organic moiety, or R₇ and R₈ combine to form an organic moiety;
wherein when one of X or Y is OH, then the other of X or Y is other than OH, NR₄R₅ or NR₇R₈ where R₄ and R₅ or R₇ and R₈ combine to form an N-methyl piperazine,
or a pharmaceutically acceptable salt or ester of the compound,
wherein one or both of bond β and bond χ is subject to in vivo hydrolytic cleavage in the subject,
so as to thereby deliver endothal to the target cell in the subject.

In some embodiments, the method wherein when one of X or Y is OH, then the other of X or Y is other than NR₄R₅ or NR₇R₈ where R₄ and R₅ or R₇ and R₈ combine to form an N-tert-butylcarboxylate piperazine.

In some embodiments, the method wherein when one of X or Y is OH, then the other of X or Y is other NR₄R₅ or NR₇R₈ where R₄ and R₅ or R₇ and R₈ combine to form an unsubstituted or substituted piperazine, morpholine or thiomorpholine.

In some embodiments, the method wherein when one of X or Y is NH₂, then the other of X or Y is other than OH or NH₂.

In some embodiments, the method wherein
X is OR₃ or NR₄R₅,
wherein
R₃ is H, alkyl, alkenyl, alkynyl, aryl, heteroaryl, alkylaryl, alkylheteroaryl, alkenylaryl, alkenylheteroaryl, alkynylaryl, alkynylheteroaryl, heteroalkyl, heteroalkenyl, heteroalkynyl, hydroxyalkyl, hydroxyalkenyl, hydroxyalkynyl, alkyl-P(O)(OR₉)₂, alkyl-OP(O)(OR₉)₂, alkyl-O(CO)—OR₁₀, alkyl-P(O)(O-alkyl-O(CO)—OR₁₀)₂, or alkyl-OP(O)(O-alkyl-O(CO)—OR₁₀)₂;
R₄ and R₅ are each independently H, alkyl, alkenyl, alkynyl, aryl, heteroaryl, alkylaryl, alkylheteroaryl, alkenylaryl, alkenylheteroaryl, alkynylaryl, alkynylheteroaryl, heteroalkyl, heteroalkenyl, heteroalkynyl, hydroxyalkyl, hydroxyalkenyl, hydroxyalkynyl, alkyl-P(O)(OR₉)₂, alkyl-OP(O)(OR₉)₂, alkyl-O(CO)—OR₁₀, alkyl-P(O)(O-alkyl-O(CO)—OR₁₀)₂, or alkyl-OP(O)(O-alkyl-O(CO)—OR₁₀)₂,
or R₄ and R₅ combine to form an unsubstituted or substituted cycloalkyl, cycloalkenyl, cycloalkynyl or heterocycloalkyl,
   wherein R₉ and R₁₀ are each independently H, alkyl, alkenyl, alkynyl, or aryl; and
      Y is OR₆ or NR₇R₅,
wherein
R₆ is H, alkyl, alkenyl, alkynyl, aryl, heteroaryl, alkylaryl, alkylheteroaryl, alkenylaryl, alkenylheteroaryl, alkynylaryl, alkynylheteroaryl, heteroalkyl, heteroalkenyl, heteroalkynyl, hydroxyalkyl, hydroxyalkenyl, hydroxyalkynyl, alkyl-P(O)(OR₉)₂, alkyl-OP(O)(OR₉)₂, alkyl-O(CO)—OR₁₀, alkyl-P(O)(O-alkyl-O(CO)—OR₁₀)₂, or alkyl-OP(O)(O-alkyl-O(CO)—OR₁₀)₂;
R₇ and R₈ are each independently H, alkyl, alkenyl, alkynyl, aryl, heteroaryl, alkylaryl, alkylheteroaryl, alkenylaryl, alkenylheteroaryl, alkynylaryl, alkynylheteroaryl, heteroalkyl, heteroalkenyl, heteroalkynyl, hydroxyalkyl, hydroxyalkenyl, hydroxyalkynyl, alkyl-P(O)(OR₉)₂, alkyl-OP(O)(OR₉)₂, alkyl-O(CO)—OR₁₀, alkyl-P(O)(O-alkyl-O(CO)—OR₁₀)₂, or alkyl-OP(O)(O-alkyl-O(CO)—OR₁₀)₂,
or R₇ and R₈ combine to form an unsubstituted or substituted cycloalkyl, cycloalkenyl, cycloalkynyl or heterocycloalkyl,
   wherein R₆ and R₁₀ are each independently H, alkyl, alkenyl, alkynyl, or aryl,
or a pharmaceutically acceptable salt or ester of the compound.

In some embodiments, the method wherein

X is OR₃ or NR₄R₅,
wherein
R₃ is H, alkyl, alkenyl, hydroxyalkyl, alkylaryl, alkyl-P(O)(OR₉)₂, alkyl-OP(O)(OR₉)₂, alkyl-O(CO)—OR₁₀, alkyl-P(O)(O-alkyl-O(CO)—OR₁₀)₂, or alkyl-OP(O)(O-alkyl-O(CO)—OR₁₀)₂;
R₄ and R₅ are each independently H, alkyl, alkenyl, hydroxyalkyl, alkyl-P(O)(OR₉)₂, alkyl-OP(O)(OR₉)₂, alkyl-O(CO)—OR₁₀, alkyl-P(O)(O-alkyl-O(CO)—OR₁₀)₂, or alkyl-OP(O)(O-alkyl-O(CO)—OR₁₀)₂, or R₄ and R₅ combine to form an unsubstituted or substituted heterocycloalkyl,
   wherein R₉ and R₁₀ are each independently H, alkyl, alkenyl, alkynyl, or aryl; and
Y is OR₆ or NR₇R₅,
wherein
R₆ is H, alkyl, alkenyl, hydroxyalkyl, alkylaryl, alkyl-P(O)(OR₉)₂, alkyl-OP(O)(OR₉)₂, alkyl-O(CO)—OR₁₀, alkyl-P(O)(O-alkyl-O(CO)—OR₁₀)₂, or alkyl-OP(O)(O-alkyl-O(CO)—OR₁₀)₂;
R₇ and R₈ are each independently H, alkyl, alkenyl, hydroxyalkyl, alkyl-P(O)(OR₉)₂, alkyl-OP(O)(OR₉)₂, alkyl-O(CO)—OR₁₀, alkyl-P(O)(O-alkyl-O(CO)—OR₁₀)₂, or alkyl-OP(O)(O-alkyl-O(CO)—OR₁₀)₂,
or R₇ and R₈ combine to form an unsubstituted or substituted heterocycloalkyl,
   wherein R₉ and R₁₀ are each independently H, alkyl, alkenyl, alkynyl, or aryl,
      or a pharmaceutically acceptable salt or ester of the compound.

In some embodiments, the method wherein
X is OR₃,

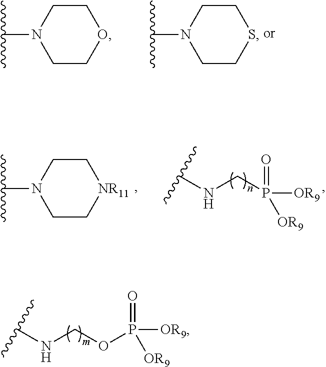

wherein R₃ is H, alkyl, alkenyl, hydroxyalkyl, alkylaryl, alkyl-P(O)(OR₉)₂, alkyl-OP(O)(OR₉)₂, alkyl-O(CO)—OR₁₀, alkyl-P(O)(O-alkyl-O(CO)—OR₁₀)₂, or alkyl-OP(O)(O-alkyl-O(CO)—OR₁₀)₂,
   wherein R₉ and R₁₀ are each independently H, alkyl, alkenyl, or alkynyl,
wherein R₁₁ is H, alkyl, hydroxyalkyl, alkenyl, alkenyl, alkynyl, aryl, alkylaryl, heteroaryl, alkylheteroaryl,

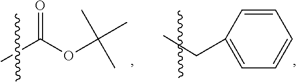

—CH₂CN, —CH₂CO₂R₁₂, —CH₂COR₁₂, —NHR₁₂, or —NH⁺(R₁₂)₂,
   where each R₁₂ is independently H, alkyl, alkenyl or alkynyl; and
Y is OR₆,

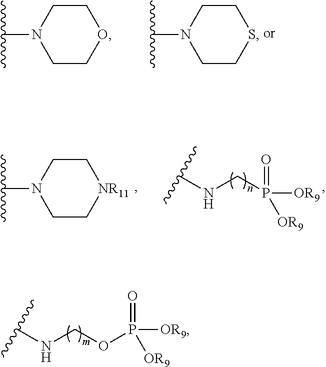

wherein R₆ is H, alkyl, alkenyl, hydroxyalkyl, alkylaryl, alkyl-P(O)(OR₉)₂, alkyl-OP(O)(OR₉)₂, alkyl-O(CO)—OR₁₀, alkyl-P(O)(O-alkyl-O(CO)—OR₁₀)₂, or alkyl-OP(O)(O-alkyl-O(CO)—OR₁₀)₂,
   wherein R₉ and R₁₀ are each independently H, alkyl, alkenyl, or alkynyl,
wherein R₁₁ is H, alkyl, hydroxyalkyl, alkenyl, alkenyl, alkynyl, aryl, alkylaryl, heteroaryl, alkylheteroaryl,

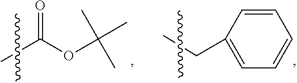

—CH₂CN, —CH₂CO₂R₁₂, —CH₂COR₁₂, —NHR₁₂, or —NH⁺(R₁₂)₂,
wherein R₉ and R₁₀ are each independently H, alkyl, alkenyl, or alkynyl,
   where each R₁₂ is independently H, alkyl, alkenyl or alkynyl.

In some embodiments, the method wherein the compound has the structure:

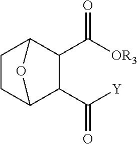

wherein
R₃ is H, alkyl, alkenyl, hydroxyalkyl, alkylaryl, alkyl-P(O)(OR₉)₂, alkyl-OP(O)(OR₉)₂, alkyl-O(CO)—OR₁₀, alkyl-P(O)(O-alkyl-O(CO)—OR₁₀)₂, or alkyl-OP(O)(O-alkyl-O(CO)—OR₁₀)₂,
   wherein R₉ and R₁₀ are each independently H, alkyl, alkenyl, or alkynyl; and
Y is OR₆,

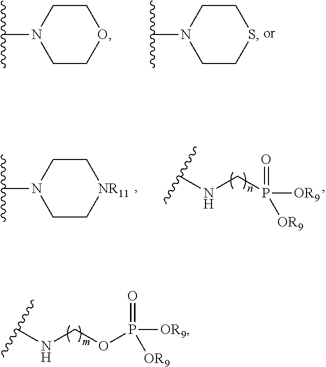

wherein R₆ is H, alkyl, alkenyl, hydroxyalkyl, alkyl-P(O)(OR₉)₂, alkyl-OP(O)(OR₉)₂, alkyl-O(CO)—OR₁₀, alkyl-P(O)(O-alkyl-O(CO)—OR₁₀)₂, or alkyl-OP(O)(O-alkyl-O(CO)—OR₁₀)₂,
   wherein R₉ and R₁₀ are each independently H, alkyl, alkenyl, or alkynyl,
wherein R₁₁ is H, alkyl, hydroxyalkyl, alkenyl, alkenyl, alkynyl, aryl, alkylaryl, heteroaryl, alkylheteroaryl,

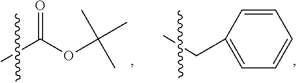

—CH₂CN, —CH₂CO₂R₁₂, —CH₂COR₁₂, —NHR₁₂, or —NH⁺(R₁₂)₂,
   where each R₁₂ is independently H, alkyl, alkenyl or alkynyl.

In some embodiments, the method wherein the compound has the structure:

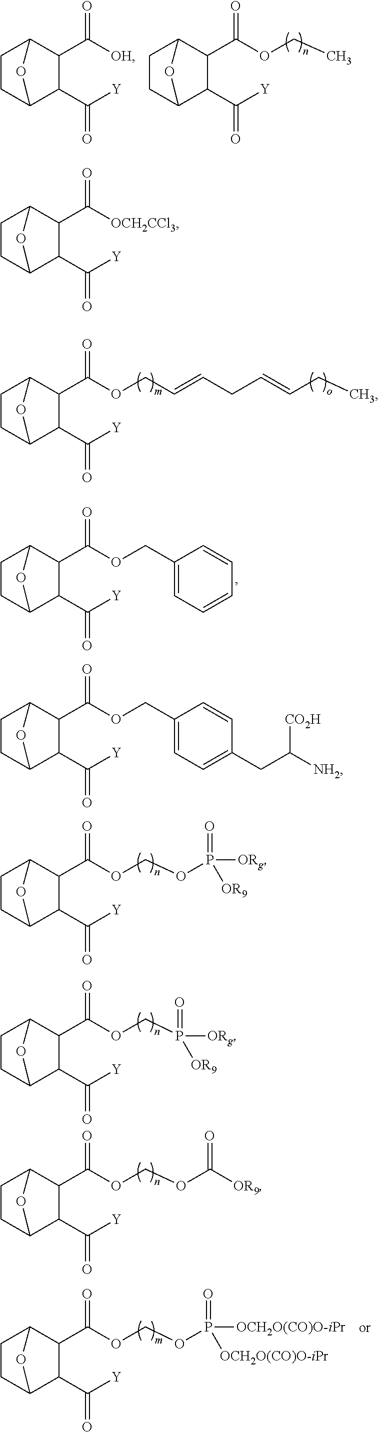

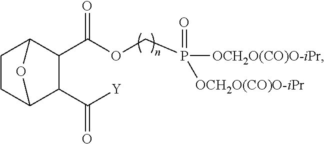

wherein
Each n=0-19, m=1-20, o=0-8 and o′=0-6,
R₉ are each independently H, alkyl, alkenyl, or alkynyl; and
Y is OR₆,

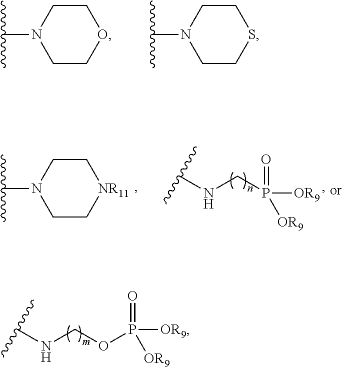

wherein R₆ is H, alkyl, alkenyl, hydroxyalkyl, alkyl-P(O)(OR₉)₂, alkyl-OP(O)(OR₉)₂, alkyl-O(CO)—OR₁₀, alkyl-P(O)(O-alkyl-O(CO)—OR₁₀)₂, or alkyl-OP(O)(O-alkyl-O(CO)—OR₁₀)₂,
   wherein R₉ and R₁₀ are each independently H, alkyl, alkenyl, or alkynyl,
wherein R₁₁ is H, alkyl, hydroxyalkyl, alkenyl, alkenyl, alkynyl, aryl, alkylaryl, heteroaryl, alkylheteroaryl,

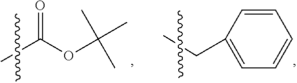

—CH₂CN, —CH₂CO₂R₁₂, —CH₂COR₁₂, —NHR₁₂, or —NH⁺(R₁₂)₂,
   where each R₁₂ is independently H, alkyl, alkenyl or alkynyl.

In some embodiments, the method wherein the compound has the structure:

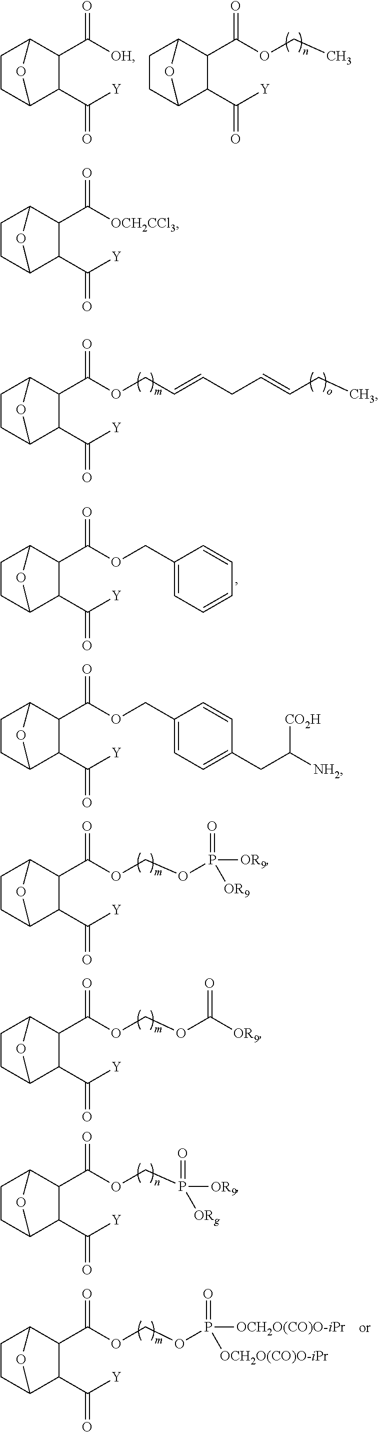

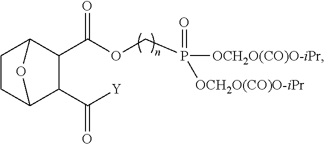

wherein
Each n=0-19, m=1-20, o=0-8 and o′=0-6,
Each R₉ is independently H, alkyl, alkenyl, or alkynyl; and
Y is

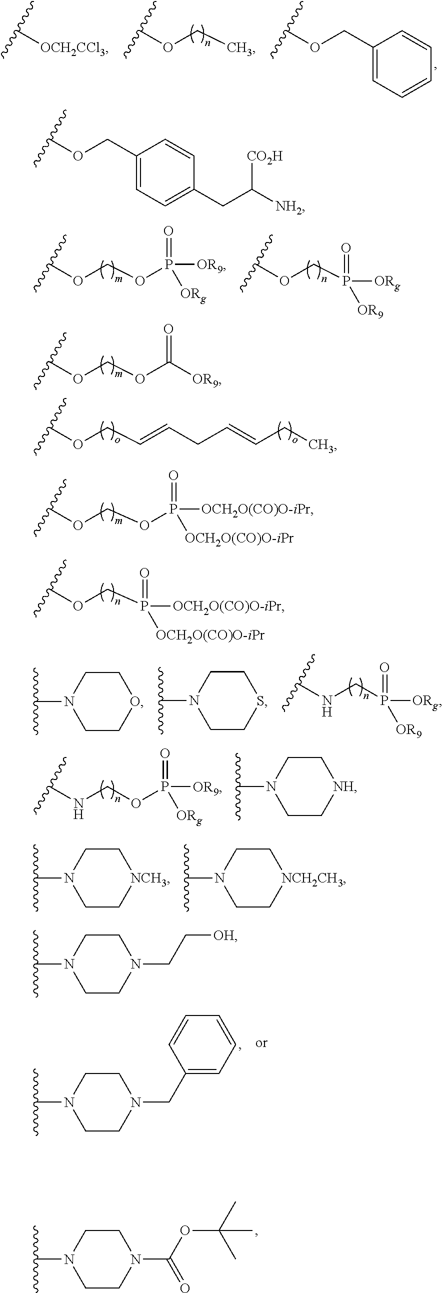

wherein
Each n=0-19, m=1-20, o=0-8 and o′=0-6,
Each R₉ is independently H, alkyl, alkenyl, or alkynyl.

In some embodiments, the method wherein the compound has the structure:

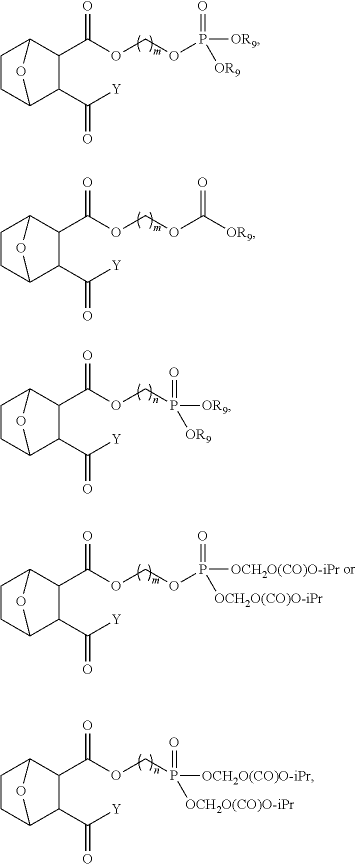

wherein
Each n=0-19, m=1-20, o=0-8 and o′=0-6,
Each R₉ is independently H, alkyl, alkenyl, or alkynyl; and
Y is

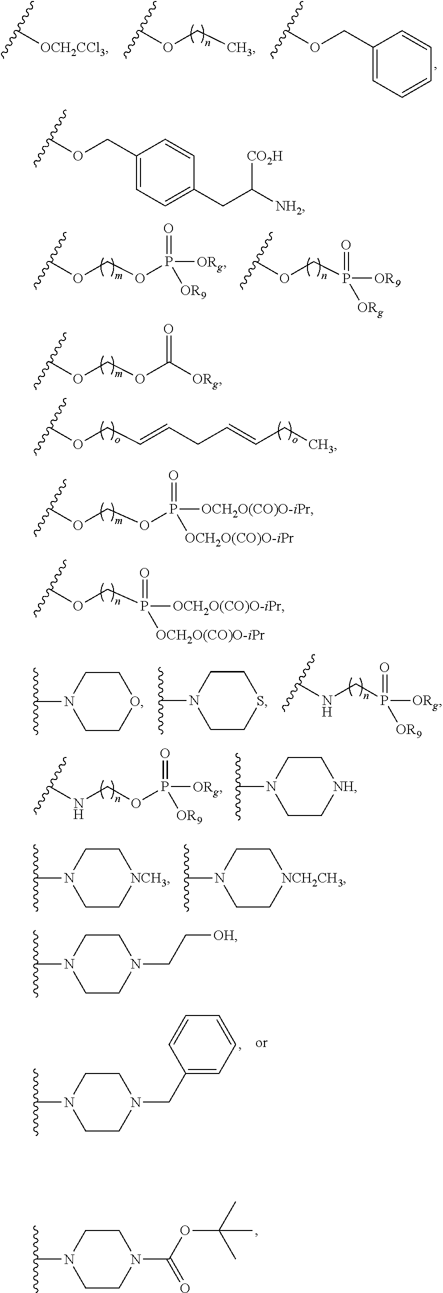

wherein
Each n=0-19, m=1-20, o=0-8 and o′=0-6,
Each R₉ is independently H, alkyl, alkenyl, or alkynyl.

In some embodiments, the method wherein the compound has the structure:

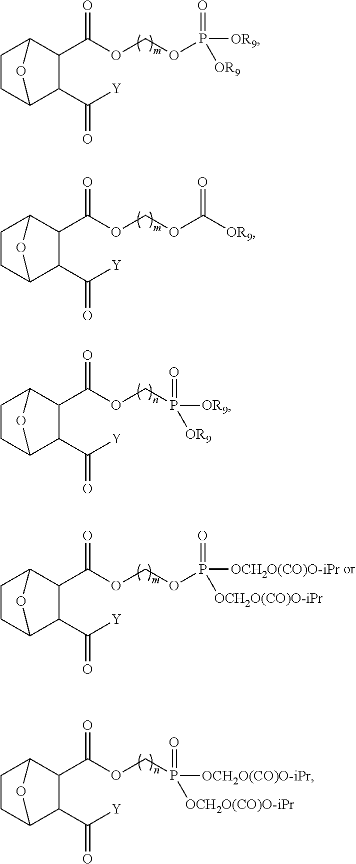

wherein
Each n=0-19, m=1-20,
Each R₉ is independently H, alkyl, alkenyl, or alkynyl; and
Y is

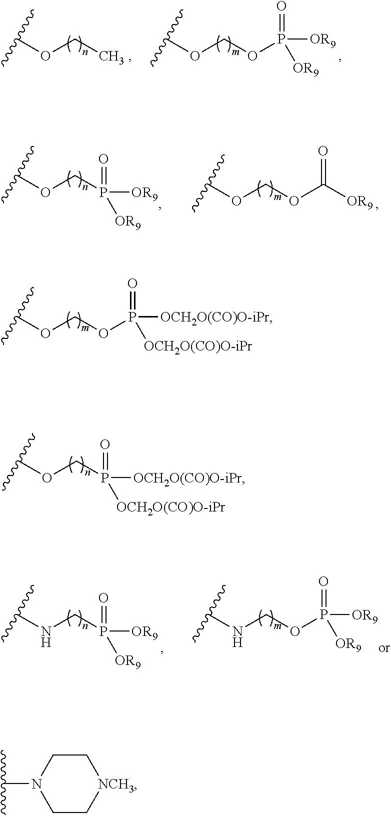

wherein
Each n=0-19, m=1-20,
Each R₉ is independently H, alkyl, alkenyl, or alkynyl.

In some embodiments, the method wherein the compound has the structure:

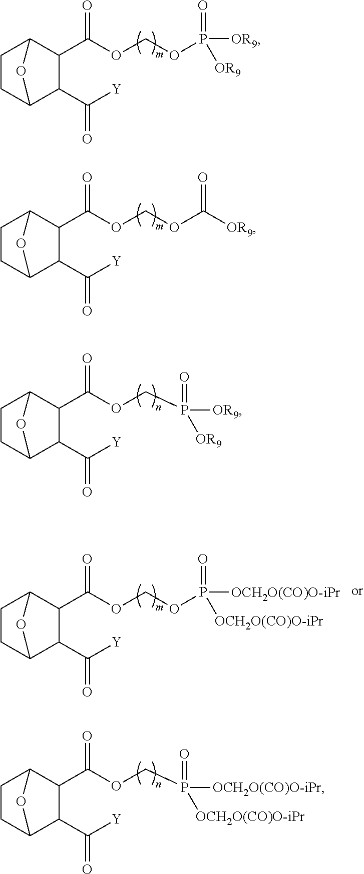

wherein
Each n=0-4 and m=2-4,
Each R₉ is independently H, CH₃, CH₂CH₃, or CH(CH₃)₂; and
Y is

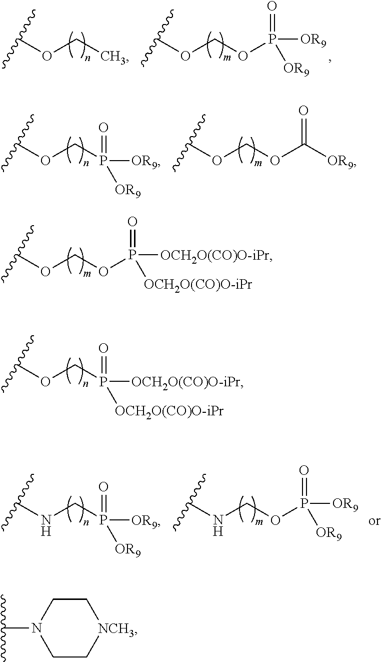

wherein
Each n=0-4 and m=2-4,
Each R₉ is independently H, CH₃, CH₂CH₃, or CH(CH₃)₂.

In some embodiments, the method wherein the compound has the structure:

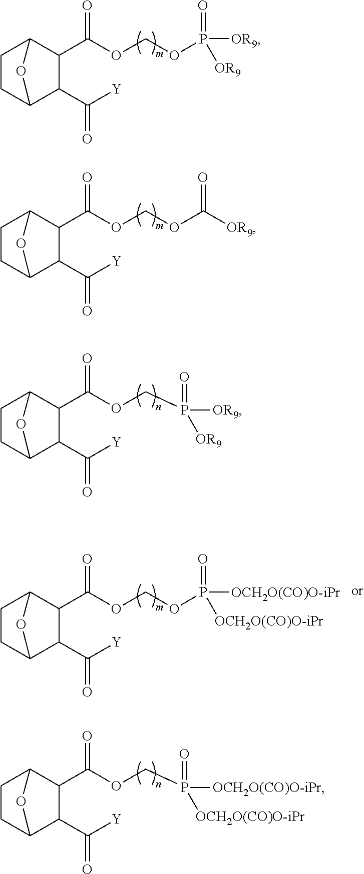

wherein
Each n=0-4 and m=2-4,
Each R₉ is independently H, CH₃, CH₂CH₃, or CH(CH₃)₂; and
Y is

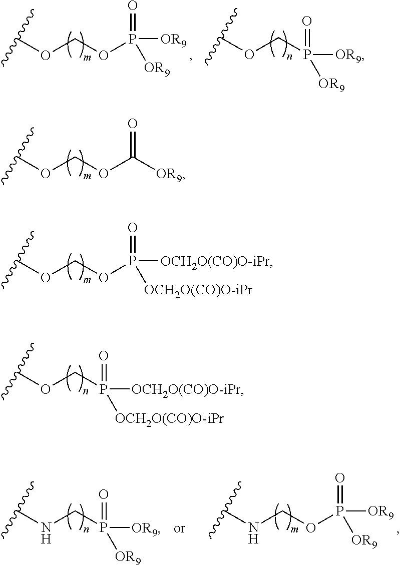

wherein
Each n=0-4 and m=2-4,
Each R₉ is independently H, CH₃, CH₂CH₃, or CH(CH₃)₂.

In some embodiments, the method wherein the compound has the structure:

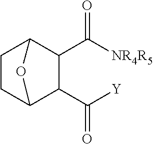

wherein
R₄ and R₅ are each H, alkyl, alkenyl, alkynyl, alkyl-P(O)(OR₉)₂, alkyl-OP(O)(OR₉)₂, alkyl-O(CO)—OR₁₀, alkyl-P(O)(O-alkyl-O(CO)—OR₁₀)₂, or alkyl-OP(O)(O-alkyl-O(CO)—OR₁₀)₂,
   wherein R₉ and R₁₀ are each independently H, alkyl, alkenyl, or alkynyl; and
Y is OR₆,

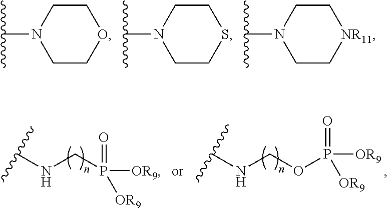

wherein R₆ is H, alkyl, alkenyl, hydroxyalkyl, alkylaryl, alkyl-P(O)(OR₉)₂, alkyl-OP(O)(OR₉)₂, alkyl-O(CO)—OR₁₀, alkyl-P(O)(O-alkyl-O(CO)—OR₁₀)₂, or alkyl-OP(O)(O-alkyl-O(CO)—OR₁₀)₂,
   wherein R₉ and R₁₀ are each independently H, alkyl, alkenyl, or alkynyl,
wherein R₁₁ is H, alkyl, hydroxyalkyl, alkenyl, alkenyl, alkynyl, aryl, alkylaryl, heteroaryl, alkylheteroaryl,

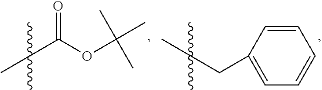

—CH₂CN, —CH₂CO₂R₁₂, —CH₂COR₁₂, —NHR₁₂, or —NH⁺(R₁₂)₂,
   where each R₁₂ is independently H, alkyl, alkenyl or alkynyl.

In some embodiments, the method wherein the compound has the structure:

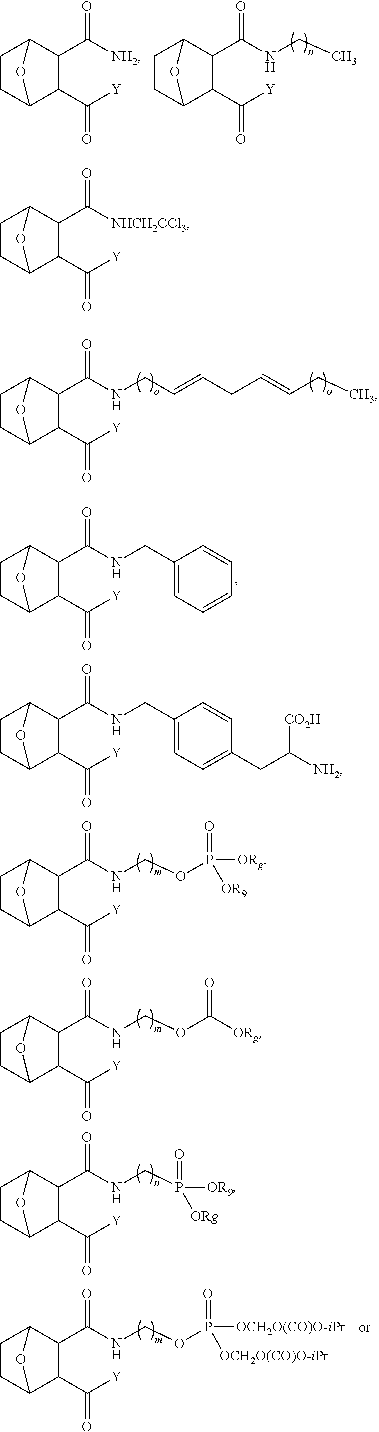

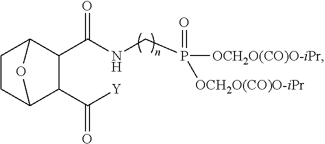

wherein
Each n=0-19, m=1-20, o=0-8 and o′=0-4,
Each R₉ is independently H, alkyl, alkenyl, or alkynyl; and
Y is ORF.

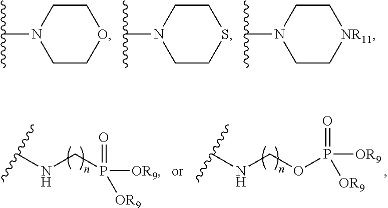

wherein R₆ is H, alkyl, alkenyl, hydroxyalkyl, alkyl-P(O)(OR₉)₂, alkyl-OP(O)(OR₉)₂, alkyl-O(CO)—OR₁₀, alkyl-P(O)(O-alkyl-O(CO)—OR₁₀)₂, or alkyl-OP(O)(O-alkyl-O(CO)—OR₁₀)₂,
   wherein R₉ and R₁₀ are each independently H, alkyl, alkenyl, or alkynyl,
wherein R₁₁ is H, alkyl, hydroxyalkyl, alkenyl, alkenyl, alkynyl, aryl, alkylaryl, heteroaryl, alkylheteroaryl,

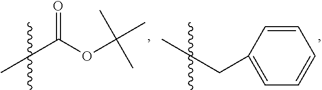

—CH₂CN, —CH₂CO₂R₁₂, —CH₂COR₁₂, —NHR₁₂, or —NH⁺(R₁₂)₂,
   where each R₁₂ is independently H, alkyl, alkenyl or alkynyl.

In some embodiments, the method wherein the compound has the structure:

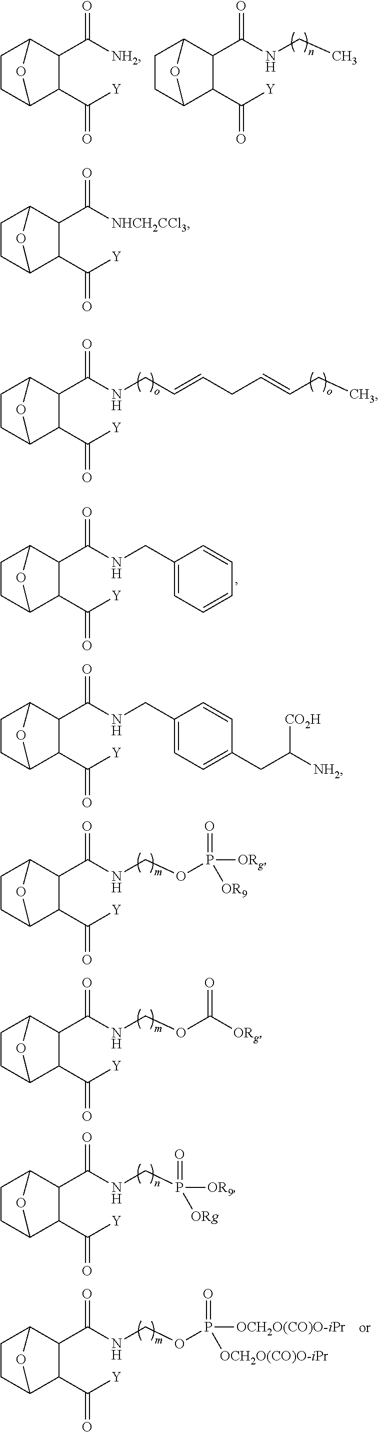

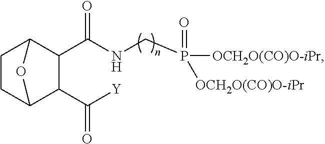

wherein
Each n=0-19, m=1-20, o=0-8 and o′=0-4,
Each R₉ is independently H, alkyl, alkenyl, or alkynyl; and
Y is

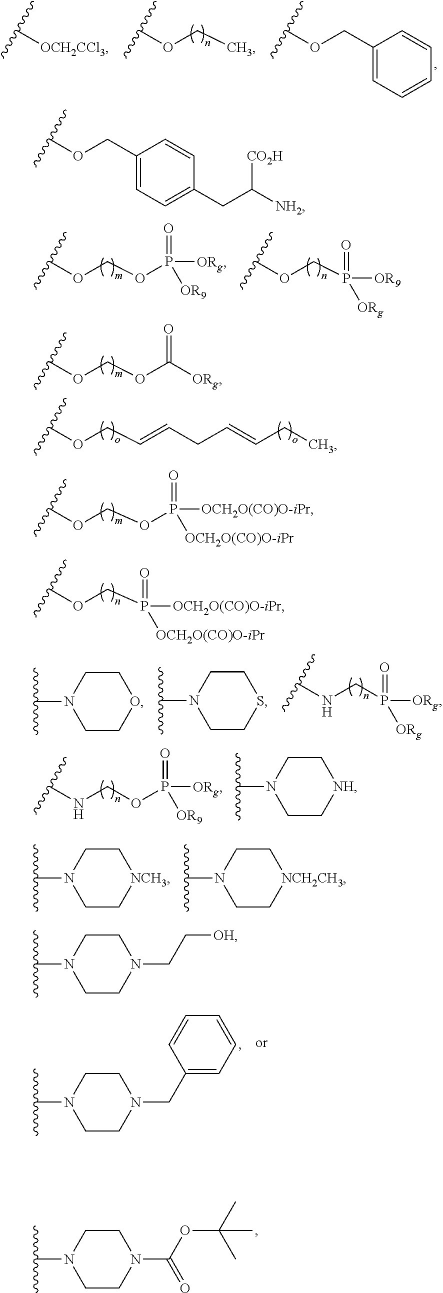

wherein
Each n=0-19, m=1-20, o=0-8 and o′=0-6,
Each R₉ is independently H, alkyl, alkenyl, or alkynyl.

In some embodiments, the method wherein the compound has the structure:

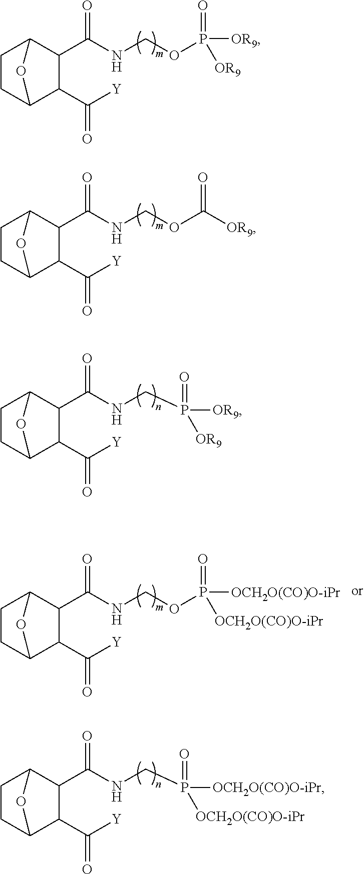

wherein
Each n=0-19, m=1-20, o=0-8 and o′=0-4,
Each R₉ is independently H, alkyl, alkenyl, or alkynyl; and
Y is

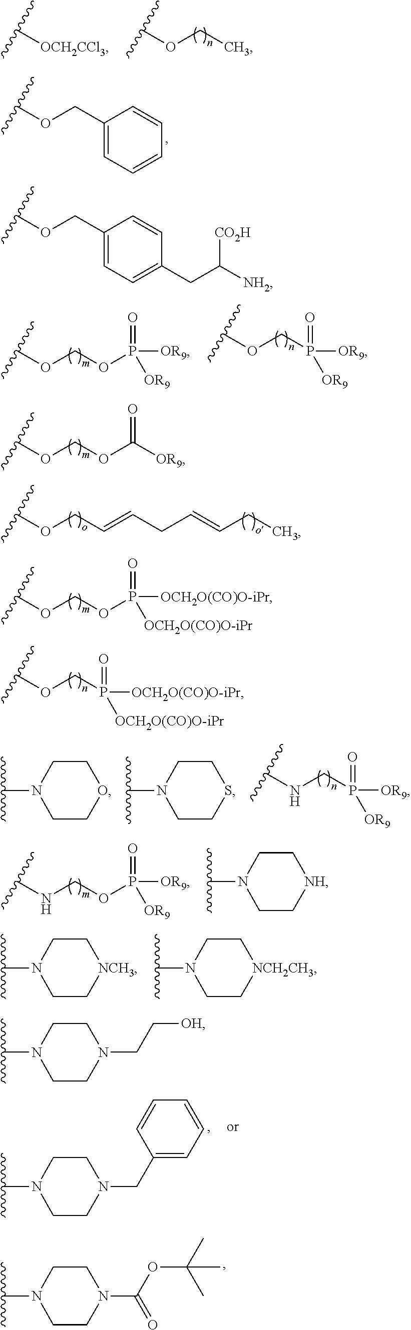

wherein
Each n=0-19, m=1-20, o=0-8 and o′=0-6,
Each 119 is independently H, alkyl, alkenyl, or alkynyl.

In some embodiments, the method wherein the compound has the structure:

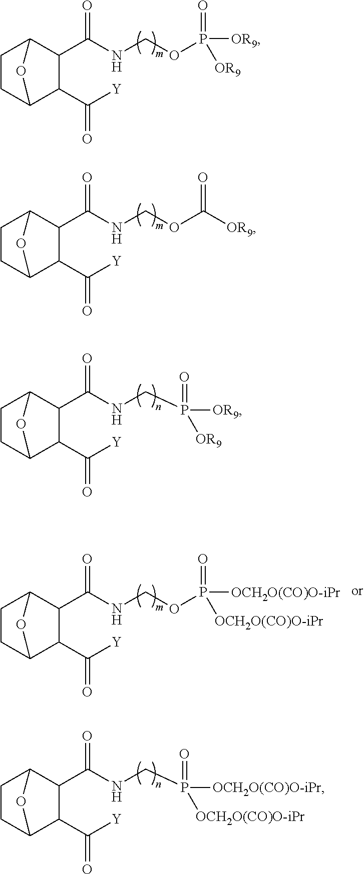

wherein
Each n=0-19, m=1-20,
Each R₉ is independently H, alkyl, alkenyl, or alkynyl; and
Y is

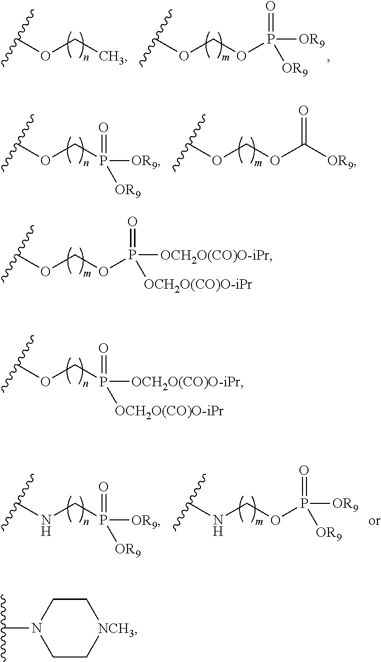

wherein
Each n=0-19, m=1-20,
Each R₉ is independently H, alkyl, alkenyl, or alkynyl.

In some embodiments, the method wherein the compound has the structure:

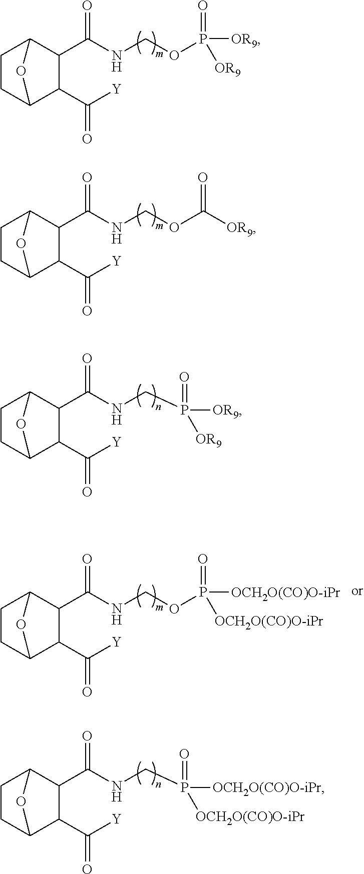

wherein
Each n=0-4 and m=2-4,
Each R₉ is independently H, CH₃, CH₂CH₃, or CH(CH₃)₂; and
Y is

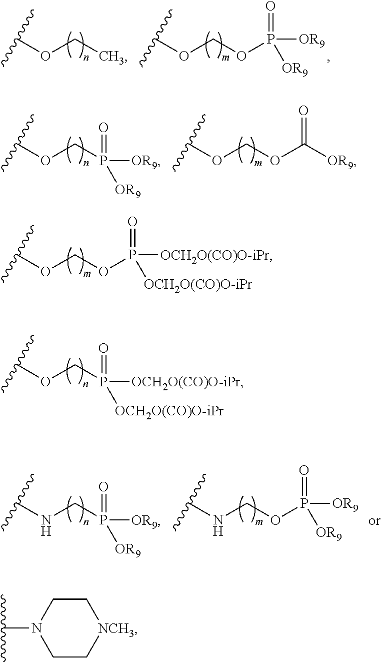

wherein
Each n=0-4 and m=2-4,
Each R₉ is independently H, CH₃, CH₂CH₃, or CH(CH₃)₂.

In some embodiments, the method wherein the compound has the structure:

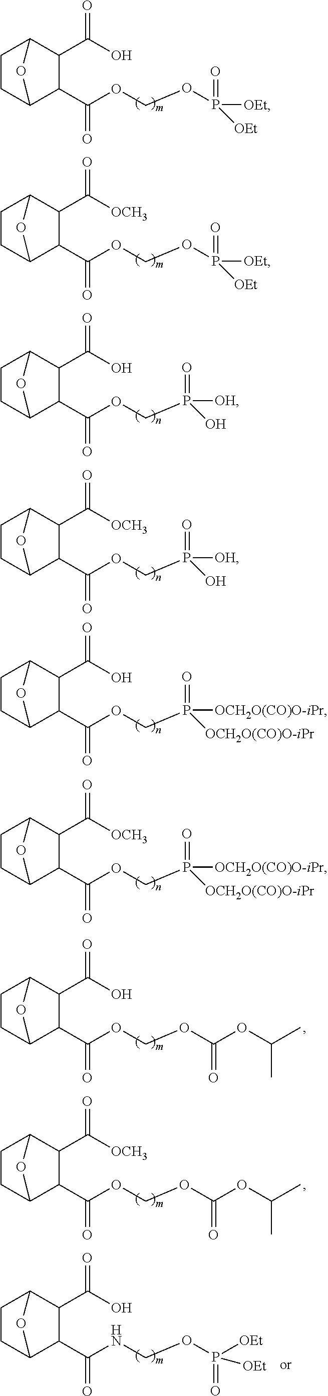

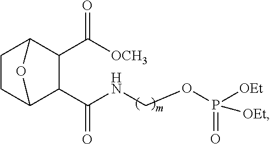

wherein each n=2-4 and each m=2-4.

In some embodiments, the method wherein the compound has the structure:

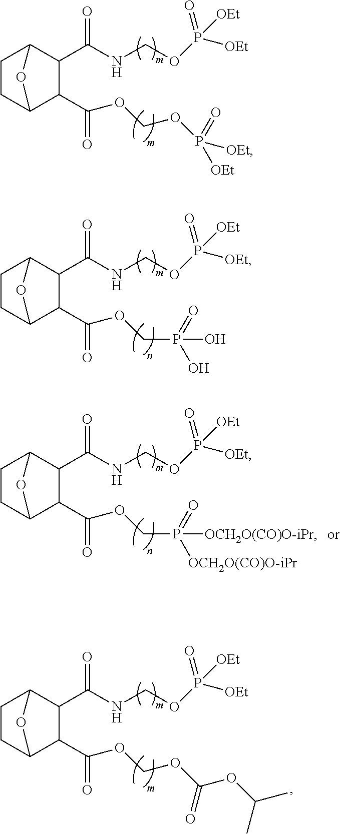

wherein each n=2-4 and each m=2-4.

In some embodiments, the method wherein the compound has the structure:

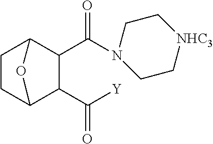

wherein
Y is OR₆,

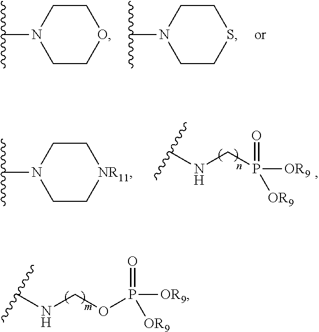

wherein R₆ is H, alkyl, alkenyl, hydroxyalkyl, alkylaryl, alkyl-P(O)(OR₉)₂, alkyl-OP(O)(OR₉)₂, alkyl-O(CO)—OR₁₀, alkyl-P(O)(O-alkyl-O(CO)—OR₁₀)₂, or alkyl-OP(O)(O-alkyl-O(CO)—OR₁₀)₂,
   wherein R₉ and R₁₀ are each independently H, alkyl, alkenyl, or alkynyl,
wherein R₁₁ is H, alkyl, hydroxyalkyl, alkenyl, alkenyl, alkynyl, aryl, alkylaryl, heteroaryl, alkylheteroaryl,

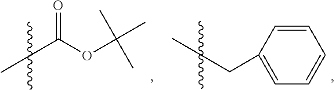

—CH₂CN, —CH₂CO₂R₁₂, —CH₂COR₁₂, —NHR₁₂, or —NH⁺(R₁₂)₂, where each R₁₂ is independently H, alkyl, alkenyl or alkynyl.

In some embodiments, the method wherein
Y is OR₆,

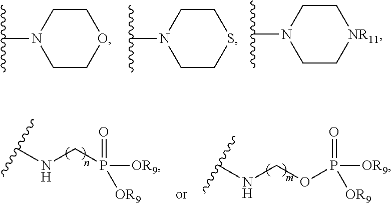

wherein R₆ is H, alkyl, alkenyl, hydroxyalkyl, alkyl-P(O)(OR₉)₂, alkyl-OP(O)(OR₉)₂, alkyl-O(CO)—R₁₀, alkyl-P(O)(C)-alkyl-O(CO)—OR₁₀)₂, or alkyl-OP(O)(O-alkyl-O(CO)—OR₁₀)₂,
   wherein R₉ and R₁₀ are each independently H, alkyl, alkenyl, or alkynyl,
wherein R₁₁ is H, alkyl, hydroxyalkyl, alkenyl, alkenyl, alkynyl, aryl, alkylaryl, heteroaryl, alkylheteroaryl,

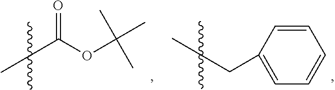

—CH₂CN, —CH₂CO₂R₁₂, —CH₂COR₁₂, —NHR₁₂, or —NH⁺(R₁₂)(R₁₂)₂,
   where each R₁₂ is independently H, alkyl, alkenyl or alkynyl.

In some embodiments, the method wherein
Y is

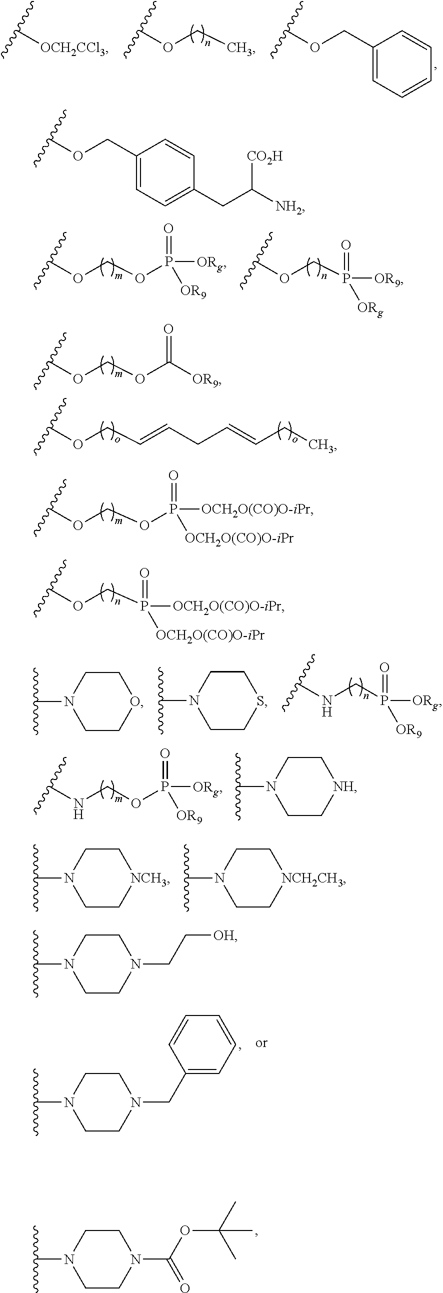

wherein
Each n=0-19, m=1-20, o=0-8 and o′=0-6,
Each R₉ is independently H, alkyl, alkenyl, or alkynyl.

In some embodiments, the method wherein
Y is

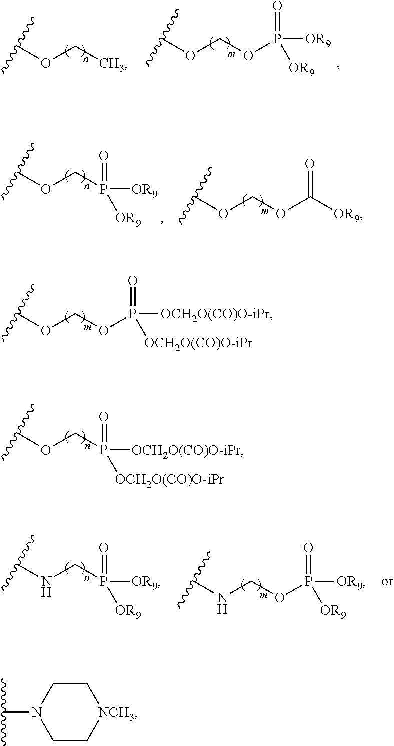

wherein
Each n=0-19, m=1-20,
Each R₉ is independently H, alkyl, alkenyl, or alkynyl.

In some embodiments, the method wherein
Y is

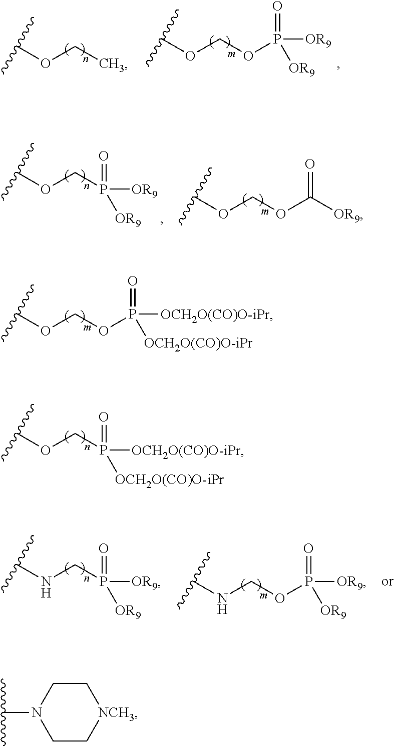

wherein
Each n=0-4 and m=2-4,
Each R₉ is independently H, CH₃, CH₂CH₃, or CH(CH₃)₂.

In some embodiments, the method wherein the compound has the structure:

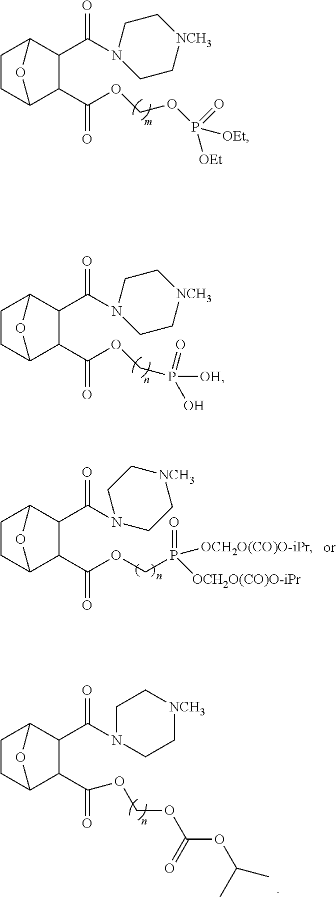

wherein each n=2-4 and each m=2-4.

In some embodiments, the method wherein the compound has the structure:

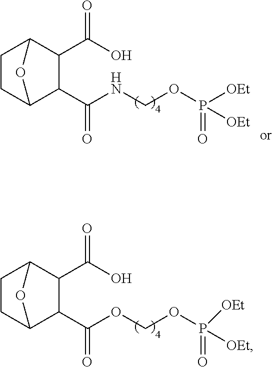

or a pharmaceutically acceptable salt or ester of the compound,

In some embodiments, the method wherein
X is OH, O⁻, OR₁₃, O(CH₂)₁₋₆R₁₃, SH, S⁻, or SR₁₃,
   wherein R₁₃ is H, alkyl, alkenyl, alkynyl or aryl;
Y is

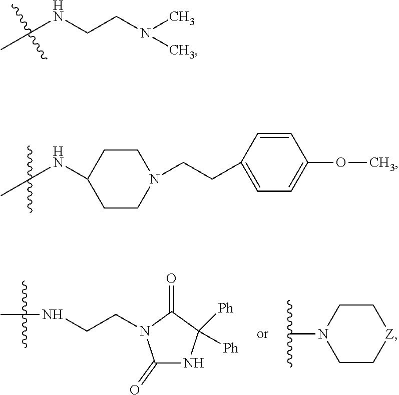

where Z is O, S, NR₁₄, N⁺HR₁₄ or N⁺R₁₄R₁₄,
   where each R₁₀ is independently H, alkyl, alkenyl, alkynyl, aryl,

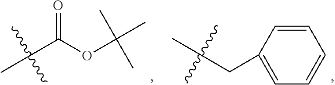

—CH₂CN, —CH₂CO₂R₁₅, or —CH₂COR₁₅,
   wherein each R₁₅ is independently H, alkyl, alkenyl or alkynyl.

In some embodiments, the method wherein
X is OH, O⁻, or OR₁₃,
   wherein R₁₃ is alkyl, alkenyl, alkynyl or aryl;
Y is

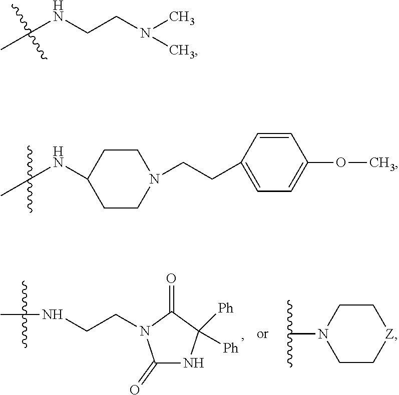

where Z is O, S, NR₁₄, N⁺HR₁₄ or N⁺R₁₄R₁₄,
   where each R₁₄ is independently H, alkyl, alkenyl, alkynyl, aryl,

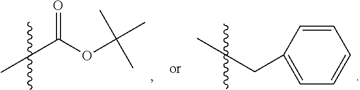

In some embodiments, the method wherein
X is OH, O⁻ or OR₁₃,
   where R₁₃ is H, methyl, ethyl or phenyl.

In some embodiments, the method wherein
Y is

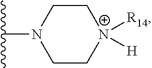

wherein R₁₄ is H, alkyl, alkenyl, alkynyl, aryl, or

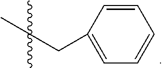 [SMILES: *Cc1ccccc1]

In some embodiments, the method wherein
Y is

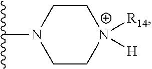

wherein R₁₄ is —H, —CH₃, —CH₂CH₃,
or

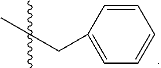 [SMILES: *Cc1ccccc1]

In some embodiments, the method wherein
Y is

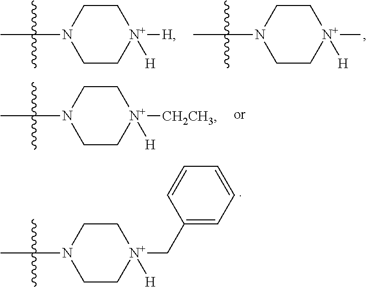

In some embodiments, the method wherein
Y is

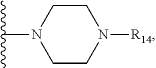

wherein R₁₄ is H, alkyl, alkenyl, alkynyl, aryl,

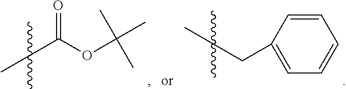

In some embodiments, the method wherein
Y is

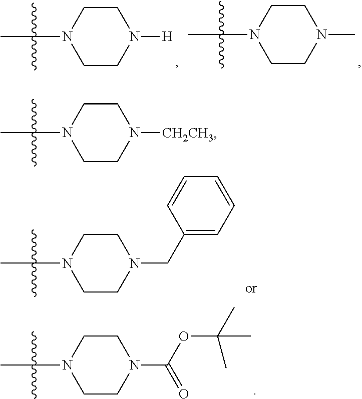

In some embodiments, the method wherein the compound has the structure:

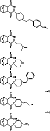

or a pharmaceutically acceptable salt or ester of the compound,

In some embodiments, the method wherein
X is OH; and
Y is

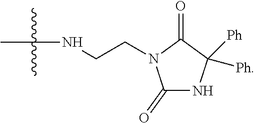 [SMILES: *NCCN1C(=O)NC(c2ccccc2)(c2ccccc2)C1=O]

In some embodiments, the method wherein the compound has the structure:

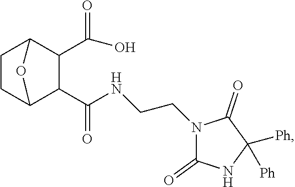 [SMILES: O=C(O)C1C2CCC(O2)C1C(=O)NCCN1C(=O)NC(c2ccccc2)(c2ccccc2)C1=O]

or a pharmaceutically acceptable salt or ester of the compound,

In some embodiments, the method wherein
X is O(CH₂)₁₋₆R₁₆ or OR₁₆
   where each R₁₆ is H, alkyl, C₂-C₁₂ alkyl, substituted alkyl, alkenyl, alkynyl, aryl, (C₆H₅)(CH₂)₁₋₆(CHNHBOC)CO₂H, (C₆H₅)(CH₂)₁₋₆ (CHNH₂) CO₂H, (CH₂)₁₋₆(CHNHBOC)CO₂H, (CH₂)₁₋₆(CHNH₂)CO₂H or (CH₂)₁₋₆CCl₃; and
Y is

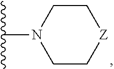

where Z is O, S, NR₁₄, N⁺HR₁₄ or N⁺H₁₄H₁₄,
   where each R₁₄ is independently H, alkyl, hydroxyalkyl, C₂-C₁₂ alkyl, alkenyl, C₄-C₁₂ alkenyl, alkynyl, aryl,

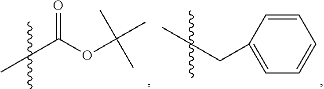

—CH₂CN, —CH₂CO₂R₁₅, or —CH₂COR₁₅,
   wherein each R₁₅ is independently H, alkyl, alkenyl or alkynyl.

In some embodiments, the method wherein
X is O(CH₂)₁₋₆R₁₆ or OR₁₆,
   where R₁₆ is aryl, substituted ethyl or substituted phenyl, wherein the substituent is in the para position of the phenyl.

In some embodiments, the method wherein
Y is

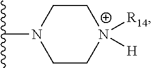

wherein R₁₄ is —H, —CH₃, —CH₂CH₃, —CH₂CH₂OH,
or

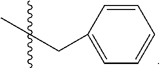 [SMILES: *Cc1ccccc1]

In some embodiments, the method wherein
Y is

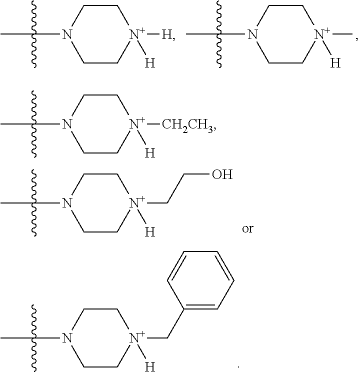

In some embodiments, the method wherein
Y is

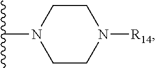

wherein R₁₄ is H, alkyl, hydroxyalkyl, alkenyl, alkynyl, aryl,

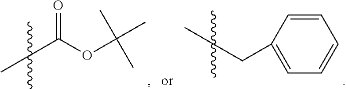

In some embodiments, the method wherein
Y is

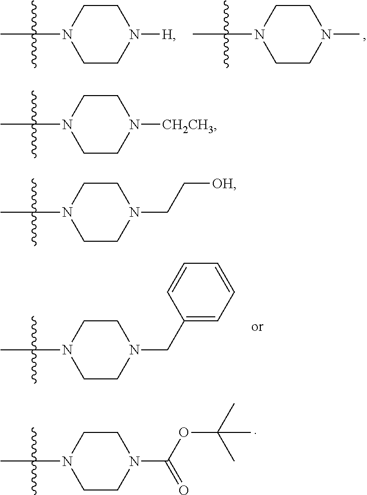

In some embodiments, the method wherein
X is OR₁₆ or O(CH₂)₁₋₂R₁₆,
   where R₁₆ is aryl, substituted ethyl, or substituted phenyl, wherein the substituent is in the para position of the phenyl; and
Y is

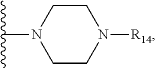

where R₁₄ is alkyl or hydroxyl alkyl.

In some embodiments, the method wherein
X is O(CH₂)R₁₆, or OR₁₆,
   where R₁₆ is phenyl or CH₂CCl₃,

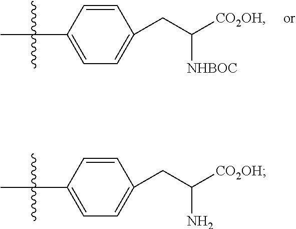

Y is

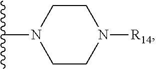

where R₁₀ is CH₃ or CH₃CH₂OH;

In some embodiments, the method wherein R₁₆ is CH₂(CHNHBOC)CO₂H, CH₂ (CHNH₂)CO₂H, CH₂CCl₃, (C₆H₅)(CH₂)(CHNHBOC) CO₂H, or (C₆H₅)(CH₂)(CHNH₂)CO₂H.

In some embodiments, the method wherein the compound has the structure:

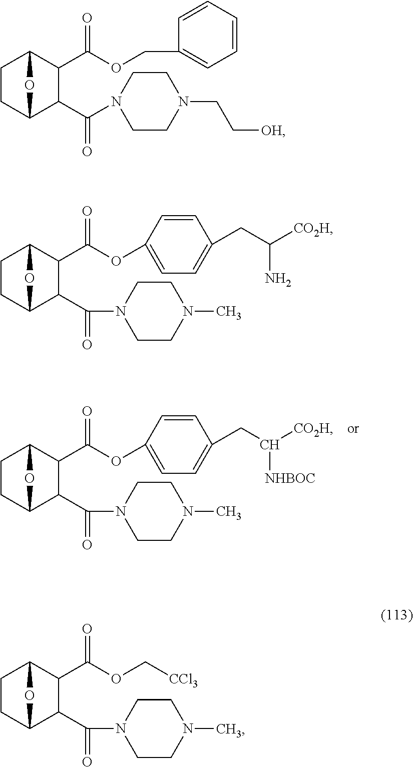

or a pharmaceutically acceptable salt or ester of the compound.

In some embodiments, the method wherein the compound has the structure:

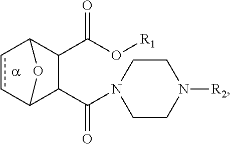

wherein
bond α is absent;
R₁ is C₂-C₂₀ alkyl, C₂-C₂₀ alkenyl, or C₂-C₂₀ alkynyl;
R₂ is H, C₁-C₁₂ alkyl, C₁-C₁₂ alkenyl, C₁-C₁₂ alkynyl, C₁-C₁₂ alkyl-(phenyl), C₁-C₁₂ alkyl-(OH), or C(O)C(CH₃)₃,
   or a pharmaceutically acceptable salt of the compound.

In some embodiments, the method wherein the compound has the structure:

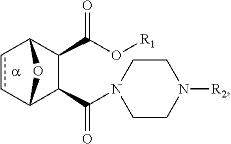

or a pharmaceutically acceptable salt of the compound.

In some embodiments, the method wherein
R₁ is C₂-C₂₀ alkyl or C₂-C₂₀ alkenyl; and
R₂ is C₁-C₁₂ alkyl.

In some embodiments, the method wherein
R₁ is C₂-C₂₀ alkyl or C₂-C₂₀ alkenyl; and
R₂ is C₁-C₁₂ alkyl-(phenyl); or
R₁ is C₂-C₂₀ alkyl or C₂-C₂₀ alkenyl; and
R₂ is C₁-C₁₂ alkyl-(OH); or
R₁ is C₂-C₂₀ alkyl or C₂-C₂₀ alkenyl; and
R₂ is —C(O)C(CH₃)₃.

In some embodiments, the method wherein
R₁ is —CH₂CH₃,
—CH₂CH₂CH₃,
—CH₂CH₂CH₂CH₃,
—CH₂CH₂CH₂CH₂CH₃,
—CH₂CH₂CH₂CH₂CH₂CH₃,
—CH₂CH₂CH₂CH₂CH₂CH₂CH₃
—CH₂CH₂CH₂CH₂CH₂CH₂CH₂CH₃,
—CH₂CH₂CH₂CH₂CH₂CH₂CH₂CH₂CH₃, or
—CH₂CH₂CH₂CH₂CH₂CH₂CH₂CH₂CH₂CH₃

In some embodiments, the method wherein
R₁ is —CH₂CH₂CH₂CH₂CH₂CH₂CH₂CH₂CH₂CH₂CH₂CH₂CH₂CH₂CH₂CH₂CH₃, or
—CH₂CH₂CH₂CH₂CH₂CH₂CH₂CH₂CH═CHCH₂CH═CHCH₂CH₂CH₂CH₂CH₃.

In some embodiments, the method wherein
R₂ is —H, —CH₃, —CH₂CH₃, —CH₂-phenyl, —CH₂CH₂—OH, or
—C(O)C(CH₃)₃.

In some embodiments, the method having the structure:

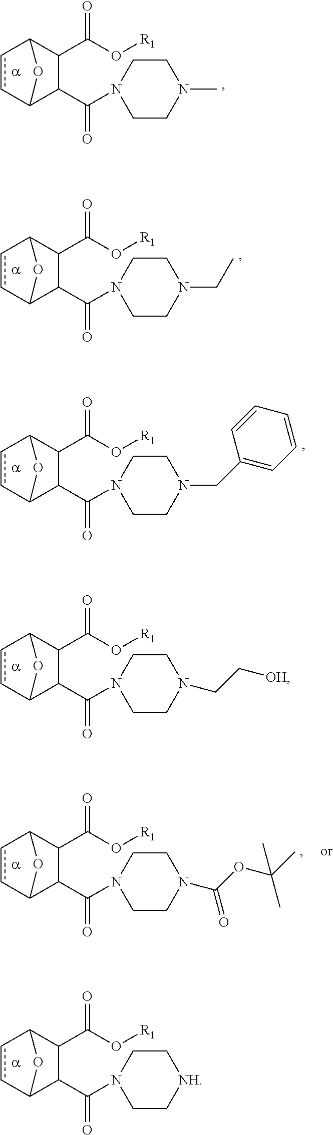

In some embodiments, the method wherein
R₁ is —CH₂CH₃,
—CH₂CH₂CH₃,
—CH₂CH₂CH₂CH₃,
—CH₂CH₂CH₂CH₂CH₃,
—CH₂CH₂CH₂CH₂CH₂CH₃,
—CH₂CH₂CH₂CH₂CH₂CH₂CH₃,
—CH₂CH₂CH₂CH₂CH₂CH₂CH₂CH₃,
—CH₂CH₂CH₂CH₂CH₂CH₂CH₂CH₂CH₃,
—CH₂CH₂CH₂CH₂CH₂CH₂CH₂CH₂CH₂CH₃,
—CH₂CH₂CH₂CH₂CH₂CH₂CH₂CH₂CH₂CH₂CH₂CH₂CH₂CH₂CH₂CH₂CH₃, or
—CH₂CH₂CH₂CH₂CH₂CH₂CH₂CH₂CH═CHCH₂CH═CHCH₂CH₂CH₂CH₂CH₃.

In some embodiments, the method wherein the compound has the structure:

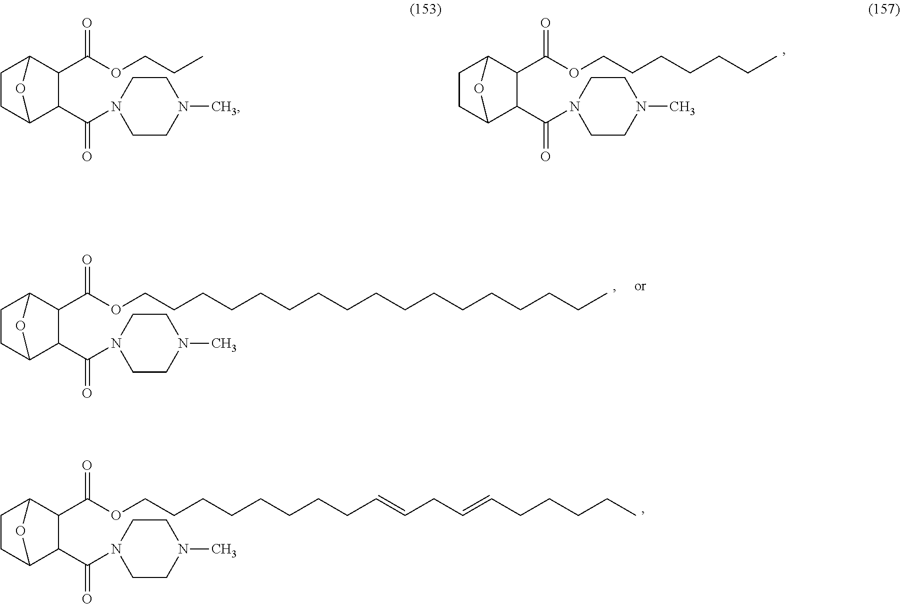 [SMILES: CCCOC(=O)C1C2CCC(O2)C1C(=O)N1CCN(C)CC1 ; CCCCCCCOC(=O)C1C2CCC(O2)C1C(=O)N1CCN(C)CC1 ; CCCCCCCCCCCCCCCCCOC(=O)C1C2CCC(O2)C1C(=O)N1CCN(C)CC1 ; CCCCC/C=C/C/C=C/CCCCCCCCOC(=O)C1C2CCC(O2)C1C(=O)N1CCN(C)CC1]

or a pharmaceutically acceptable salt or ester of the compound.

In some embodiments, the method wherein the delivery of the endothal to the target cell in the subject is effective to treat a disease in the subject afflicted with the disease.

In some embodiments, the method wherein the disease is cancer.

In some embodiments, the method wherein the cancer is a breast cancer, colon cancer, large cell lung cancer, adenocarcinoma of the lung, small cell lung cancer, stomach cancer, liver cancer, ovary adenocarcinoma, pancreas carcinoma, prostate carcinoma, promylocytic leukemia, chronic myelocytic leukemia, acute lymphocytic leukemia, colorectal cancer, ovarian cancer, lymphoma, non-Hodgkin's lymphoma or Hodgkin's lymphoma.

In some embodiments, the method wherein the cancer is a brain cancer.

In some embodiments, the method wherein the brain cancer is a glioma, pilocytic astrocytoma, low-grade diffuse astrocytoma, anaplastic astrocytoma, glioblastoma multiforme, oligodendroglioma, ependymoma, meningioma, pituitary gland tumor, primary CNS lymphoma, medulloblastoma, craniopharyngioma, or diffuse intrinsic pontine glioma.

In some embodiments, the method further comprising administering to the subject an anti-cancer agent.

In some embodiments, the method wherein the anti-cancer agent is selected from x-radiation or ionizing radiation.

In some embodiments, the method wherein the anti-cancer agent is selected from a DNA damaging agent, a DNA intercalating agent, a microtubule stabilizing agent, a microtubule destabilizing agent, a spindle toxin, abarelix, aldesleukin, alemtuzumab, alitertinoin, allopurinol, altretamine, amifostin, anakinra, anastrozole, arsenic trioxide, asparaginase, azacitidine, bevacizumab, bexarotene, bleomycin, bortezomib, busulfan, calusterone, capecitabine, carboplatin, carmustine, celecoxib, cetuximab, chlorambucil, cisplatin, cladribine, clofarabine, cyclophosphamide, cytarabine, dacarbazine, dactinomycin, actinomycin D, dalteparin sodium, darbepoetin alfa, dasatinib, daunorubicin, daunomycin, decitabine, denileukin, dexrazoxane, docetaxel, doxorubicin, dromostanolone propionate, exulizumab, epirubicin, epoetin alfa, erlotinib, estramustine, etoposide phosphate, etoposide, VP-16, exemestane, fentanyl citrate, filgrastim, floxuridine, fludarabine, fluorouracil, fulvestrant, gefitinib, gemcitabine, gosereline acetate, histrelin acetate, hydroxyurea, ibritumomab tiuxetan, idarubicin, ifosfamide, imatinib mesylate, interferon alfa 2a, interferon alfa 2b, irinotecan, lapatinib ditosylate, lenalidomide, letrozole, leucovrin, leuprolide acetate, levamisole, lomustine, meclorethamine, megestrol acetate, melphalan, mercaptopurine, mesna, methotrexate, methoxsalen, mitomycin C, mitotane, mitoxantrone, nandrolone phenpropionate, nelarabine, nofetumomab, oprelvekin, oxaliplatin, paclitaxel, palifermin, pamidronate, panitumumab, pegademase, pegaspargase, pegfilgrastim, peginterferon alfa 2b, pemetrexed disodium, pentostatin, pipobroman, plicamycin, mithramycin, porfimer sodium, procarbazine, quinacrine, rasburicase, rituximab, sargrmostim, sorafenib, streptozocin, sunitinib, sunitinib maleate, talc, tamoxifen, temozolomide, teniposide, VM-26, testolactone, thalidomide, thioguanine, G-TG, thiotepa, topotecan, toremifene, tositumomab, trastuzumab, tretinoin ATRA, uracil mustard, valrunicin, vinblastine, vincristine, vinorelbine, vorinostat, zoledronate, zoledronic acid, abraxane and brentuximab vedotin.

In some embodiments, the method wherein the target cell is a cancer cell.

In some embodiments, the method wherein the cancer cell is a breast cancer, colon cancer, large cell lung cancer, adenocarcinoma of the lung, small cell lung cancer, stomach cancer, liver cancer, ovary adenocarcinoma, pancreas carcinoma, prostate carcinoma, promylocytic leukemia, chronic myelocytic leuemia, acute lymphocytic leukemia, colorectal cancer, ovarian cancer, lymphoma, non-Hodgkin's lymphoma or Hodgkin's lymphoma cell.

In some embodiments, the method wherein the cancer cell is a brain cancer cell.

In some embodiments, the method wherein the brain cancer cell is a glioma, pilocytic astrocytoma, low-grade diffuse astrocytoma, anaplastic astrocytoma, glioblastoma multiforme, oligodendroglioma, ependymoma, meningioma, pituitary gland tumor, primary CNS lymphoma, medulloblastoma, craniopharyngioma, or diffuse intrinsic pontine glioma cell.

In some embodiments, the method wherein the target cell is in the brain of the subject.

In some embodiments, the method wherein the endothal is delivered to a target cell in the brain of the subject.

In some embodiments, the method wherein the hydrolytic cleavage of the β and/or χ bond is facilitated by a carboxylesterase or an amidase in the subject.

The present invention also provides a compound having the structure:

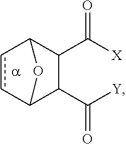

wherein
bond α is absent or present;
X is OR₁, OR₃ or NR₄R₅,
   wherein
   R₁ is C₁-C₂₀ alkyl, C₂-C₂₀ alkenyl, or C₂-C₂₀ alkynyl;
   R₃ is H, alkyl, alkylaryl, alkyl-P(O)(OR₉)₂, alkyl-OP(O)(OR₉)₂, alkyl-O(CO)—R₁₀, alkyl-P(O)(O-alkyl-O(CO)—OR₁₀)₂, or alkyl-OP(O)(O-alkyl-O(CO)—OR₁₀)₂;
   R₄ and R₅ are each independently H, alkyl-P(O)(OR₉)₂, alkyl-OP(O)(OR₉)₂, alkyl-O(CO)—OR₁₀, alkyl-P(O)(O-alkyl-O(CO)—OR₁₀)₂, or alkyl-OP(O)(O-alkyl-O(CO)—OR₁₀)₂,
   or R₄ and R₅ combine to form an unsubstituted or substituted cycloalkyl, cycloalkenyl, cycloalkynyl or heterocycloalkyl,
      wherein R₉ and R₁₀ are each independently H, alkyl, alkenyl, or alkynyl; and
Y is OR₁, OR₆ or NR₇R₈,
   wherein
   R₁ is C₁-C₂₉ alkyl, C₂-C₂₀ alkenyl, or C₂-C₂₉ alkynyl; R₆ is alkyl-P(O)(OR₉)₂, alkyl-OP(O)(OR₉)₂, alkyl-O(CO)—OR₁₀, alkyl-P(O)(O-alkyl-O(CO)—OR₁₀)₂, or alkyl-OP(O)(O-alkyl-O(CO)—OR₁₀)₂; and
   R₇ and R₈ are each independently H, alkyl-P(O)(OR₉)₂, alkyl-OP(O)(OR₉)₂, alkyl-O(CO)—OR₁₀, alkyl-P(O)(O-alkyl-O(CO)—OR₁₀)₂, or alkyl-OP(O)(O-alkyl-O(CO)—OR₁₀)₂,
   or R₇ and R₈ combine to form an unsubstituted or substituted cycloalkyl, cycloalkenyl, cycloalkynyl or heterocycloalkyl,
      wherein R₉ and R₁₀ are each independently H, alkyl, alkenyl, or alkynyl,
wherein one of X is OH, OCH₃ or O-alkylaryl, then Y is other than NR₇R₈ where R₇ and R₈ combine to form an unsubstituted or substituted piperazine, morpholine or thiomorpholine,
or a pharmaceutically acceptable salt or ester of the compound.

In some embodiments, the compound wherein
X is OR₃,

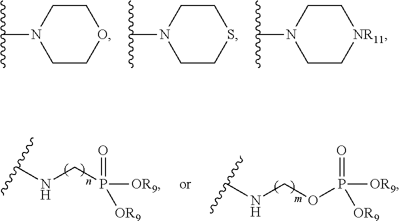

wherein R₃ is H, alkyl, alkylaryl, alkyl-P(O)(OR₉)₂, alkyl-OP(O)(OR₉)₂, alkyl-O(CO)—OR₁₀, alkyl-P(O)(O-alkyl-O(CO)—OR₁₀)₂, or alkyl-OP(O)(O-alkyl-O(CO)—OR₁₀)₂,
   wherein R₉ and R₁₀ are each independently H, alkyl, alkenyl, or alkynyl,
wherein R₁₁ is H, alkyl, hydroxyalkyl, alkenyl, alkenyl, alkynyl, aryl, alkylaryl, heteroaryl, alkylheteroaryl,

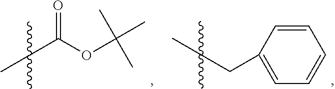

—CH₂CN, —CH₂CO₂R₁₂, —CH₂COR₁₂, —NHR₁₂, or —NH⁺(R₁₂)₂,
where each R₁₂ is independently H, alkyl, alkenyl or alkynyl; and
Y is OR₆,

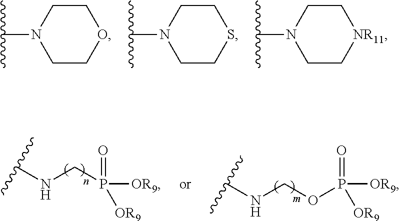

wherein R₆ is alkyl-P(O)(OR₉)₂, alkyl-OP(O)(OR₉)₂, alkyl-O(CO)—OR₁₀, alkyl-P(O)(O-alkyl-O(CO)—OR₁₀)₂, or alkyl-OP(O)(O-alkyl-O(CO)—OR₁₀)₂,
   wherein R₉ and R₁₀ are each independently H, alkyl, alkenyl, or alkynyl,
wherein R₁₁ is H, alkyl, hydroxyalkyl, alkenyl, alkenyl, alkynyl, aryl, alkylaryl, heteroaryl, alkylheteroaryl,

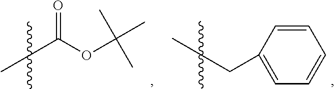

—CH₂CN, —CH₂CO₂R₁₂, —CH₂COR₁₂, —NHR₁₂, or —NH⁺(R₁₂)₂,
wherein R₉ and R₁₀ are each independently H, alkyl, alkenyl, or alkynyl,
   where each R₁₂ is independently H, alkyl, alkenyl or alkynyl.

In some embodiments, the compound having the structure:

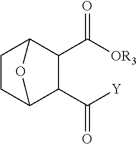

wherein
R₃ is H, alkyl, alkylaryl, alkyl-P(O)(OR₉)₂, alkyl-OP(O)(OR₉)₂, alkyl-O(CO)—OR₁₀, alkyl-P(O)(O-alkyl-O(CO)—OR₁₀)₂, or alkyl-OP(O)(O-alkyl-O(CO)—OR₁₀)₂,
   wherein R₉ and R₁₀ are each independently H, alkyl, alkenyl, or alkynyl; and
Y is OR₆,

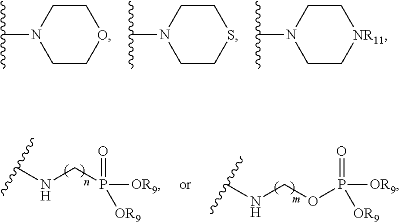

wherein R₆ is alkyl-P(O)(OR₉)₂, alkyl-OP(O)(OR₉)₂, alkyl-O(CO)—OR₁₀, alkyl-P(O)(O-alkyl-O(CO)—OR₁₀)₂, or alkyl-OP(O)(O-alkyl-O(CO)—OR₁₀)₂,
   wherein R₉ and R₁₀ are each independently H, alkyl, alkenyl, or alkynyl,
wherein R₁₁ is H, alkyl, hydroxyalkyl, alkenyl, alkenyl, alkynyl, aryl, alkylaryl, heteroaryl, alkylheteroaryl,

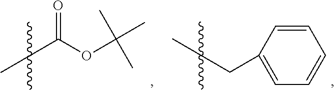

—CH₂CN, —CH₂CO₂R₁₂, —CH₂COR₁₂, —NHR₁₂, or —NH⁺(R₁₂)₂,
   where each R₁₂ is independently H, alkyl, alkenyl or alkynyl.

In some embodiments, the compound having the structure:

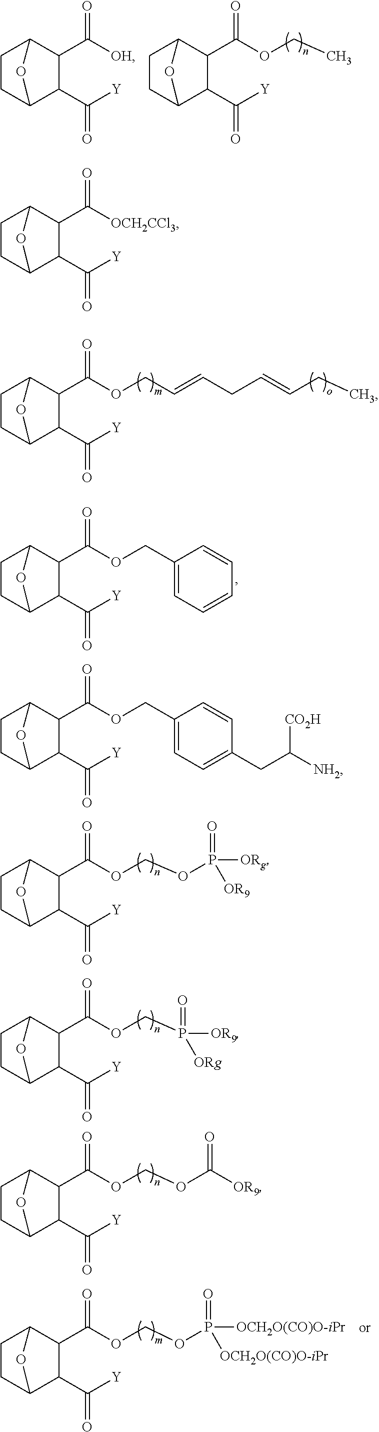

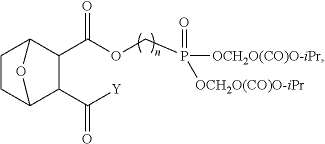

wherein
Each n=0-19, m=1-20, o=0-8 and o′=0-6,
R₉ are each independently H, alkyl, alkenyl, or alkynyl; and
Y is OR₆,

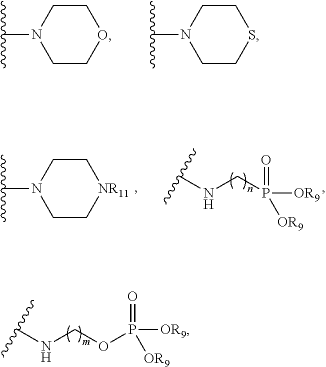

wherein R₆ is H, alkyl-P(O)(OR₉)₂, alkyl-OP(O)(OR₉)₂, alkyl-O(CO)—OR₁₀, alkyl-P(O)(O-alkyl-O(CO)—OR₁₀)₂, or alkyl-OP(O)(O-alkyl-O(CO)—OR₁₀)₂,
   wherein R₉ and R₁₀ are each independently H, alkyl, alkenyl, or alkynyl,
wherein R₁₁ is H, alkyl, hydroxyalkyl, alkenyl, alkenyl, alkynyl, aryl, alkylaryl, heteroaryl, alkylheteroaryl,

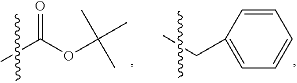

—CH₂CN, —CH₂CO₂R₁₂, —CH₂COR₁₂, —NHR₁₂, or —NH⁺(R₁₂)₂,
   where each R₁₂ is independently H, alkyl, alkenyl or alkynyl.

In some embodiments, the compound having the structure:

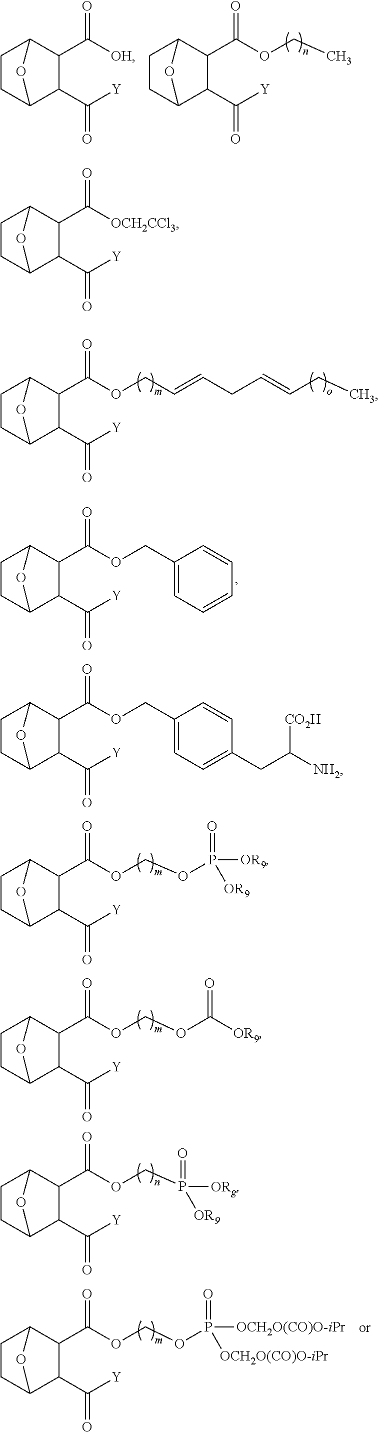

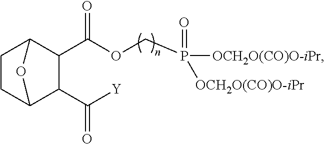

wherein
Each n=0-19, m=1-20, o=0-8 and o′=0-6,
Each R₉ is independently H, alkyl, alkenyl, or alkynyl; and
Y is

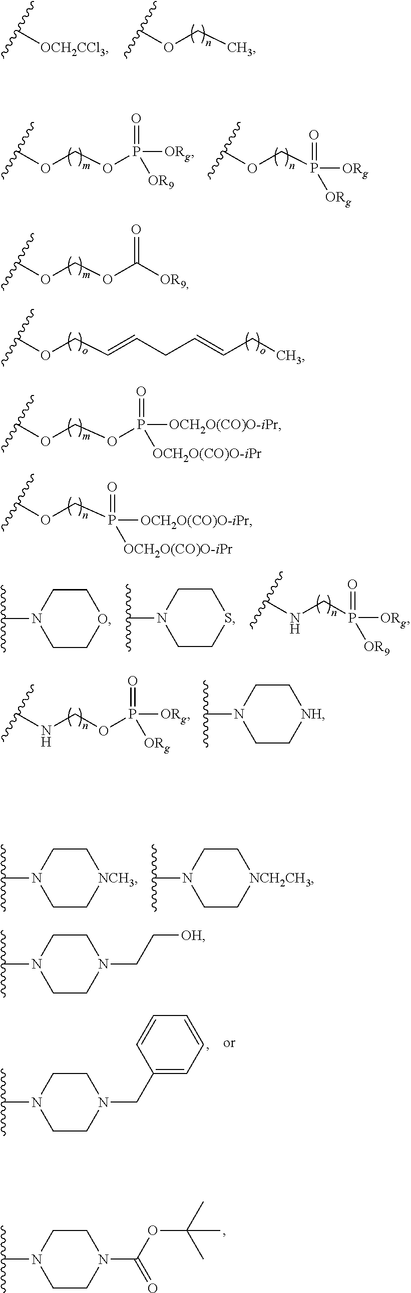

wherein
Each n=0-19, m=1-20, o=0-8 and o′=0-6,
Each R₉ is independently H, alkyl, alkenyl, or alkynyl.

In some embodiments, the compound having the structure:

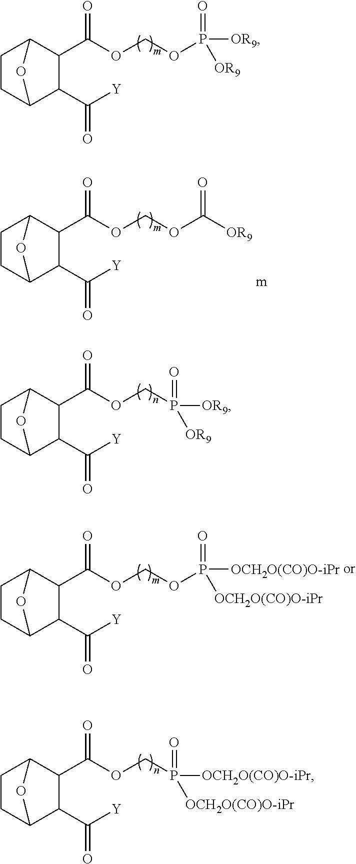

wherein
Each n=0-19, m=1-20, o=0-8 and o′=0-6,
Each R₉ is independently H, alkyl, alkenyl, or alkynyl; and
Y is

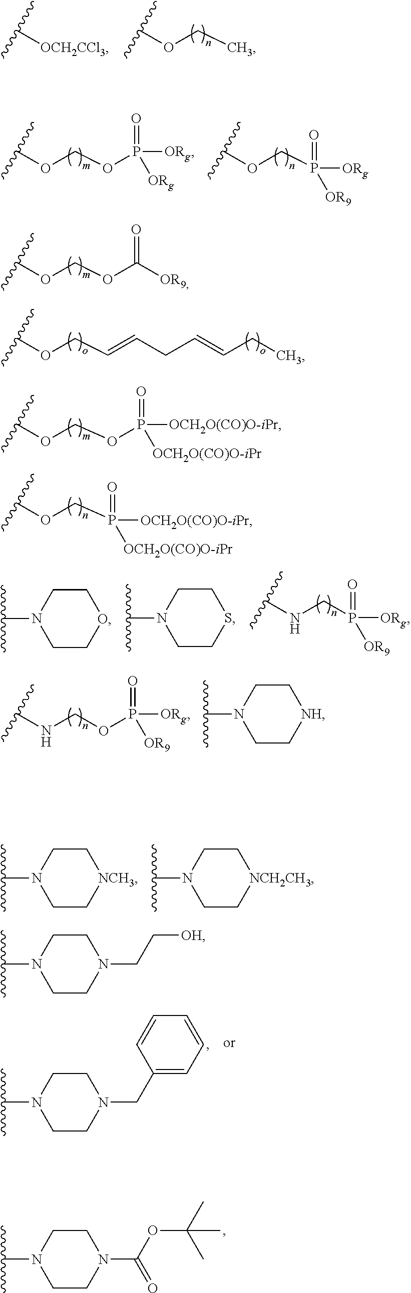

wherein
Each n=0-19, m=1-20, o=0-8 and o′=0-6,
Each R₉ is independently H, alkyl, alkenyl, or alkynyl.

In some embodiments, the compound having the structure:

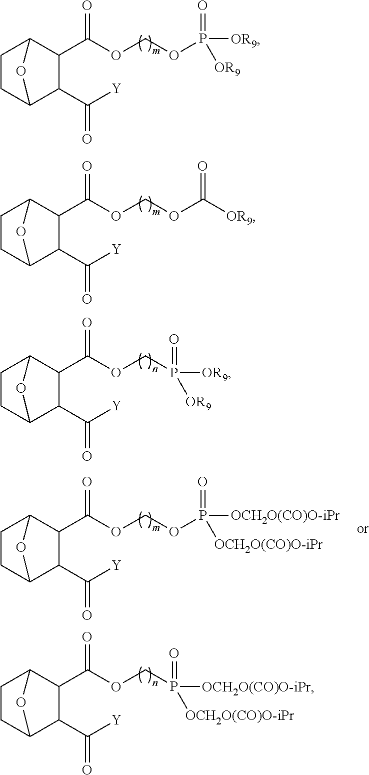

wherein
Each n=0-19, m=1-20,
Each R₉ is independently H, alkyl, alkenyl, or alkynyl; and
Y is

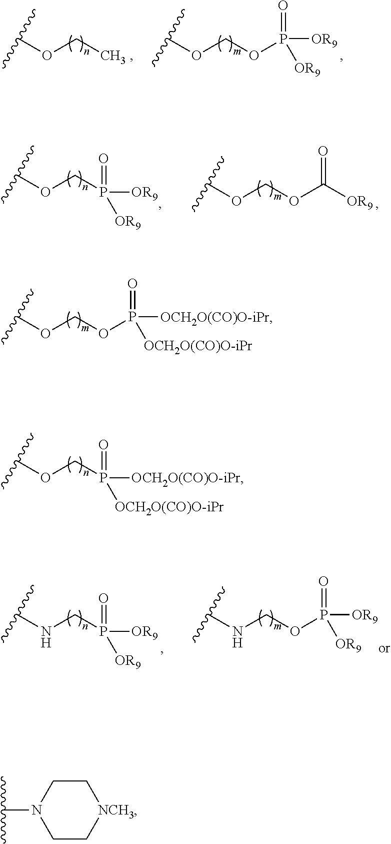

wherein
Each n=0-19, m=1-20,
Each R₉ is independently H, alkyl, alkenyl, or alkynyl.

In some embodiments, the compound having the structure:

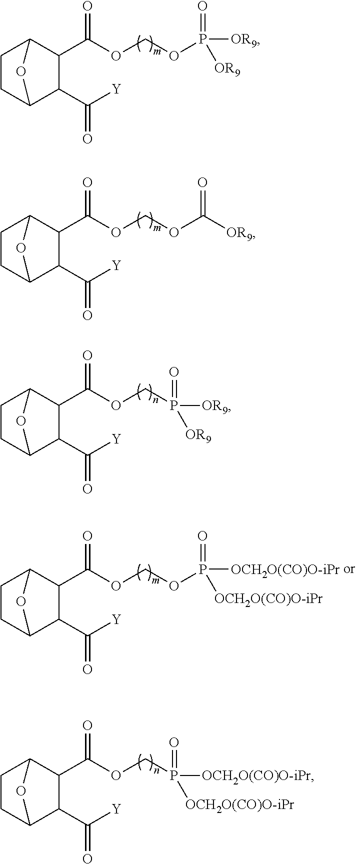

wherein
Each n=0-4 and m=2-4,
Each R₉ is independently H, CH₃, CH₂CH₃, or CH(CH₃)₂; and
Y is

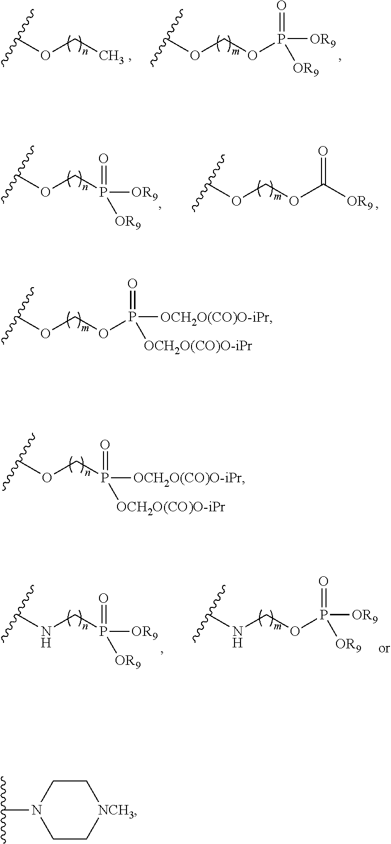

wherein
Each n=0-4 and m=2-4,
Each R₉ is independently H, CH₃, CH₂CH₃, or CH(CH₃)₂.

In some embodiments, the compound having the structure:

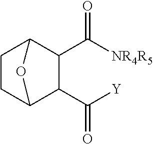

wherein
R₄ and R₅ are each H, alkyl-P(O)(OR₉)₂, alkyl-OP(O)(OR₉)₂, alkyl-O(CO)—OR₁₀, alkyl-P(O)(O-alkyl-O(CO)—OR₁₀)₂, or alkyl-OP(O)(O-alkyl-O(CO)—OR₁₀)₂,
   wherein R₉ and R₁₀ are each independently H, alkyl, alkenyl, or alkynyl; and
Y is OR₆,

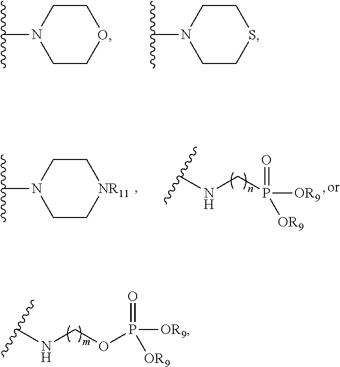

wherein R₆ is H, alkyl, alkylaryl, alkyl-P(O)(OR₉)₂, alkyl-OP(O)(OR₉)₂, alkyl-O(CO)—OR₁₀, alkyl-P(O)(O-alkyl-O(CO)—OR₉₀)₂, or alkyl-OP(O)(O-alkyl-O(CO)—OR₁₀)₂.
   wherein R₉ and R₉₉ are each independently H, alkyl, alkenyl, or alkynyl,
wherein R₁₁ is H, alkyl, hydroxyalkyl, alkenyl, alkenyl, alkynyl, aryl, alkylaryl, heteroaryl, alkylheteroaryl,

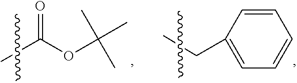

—CH₂CN, —CH₂CO₂R₁₂, —CH₂COR₁₂, —NHR₁₂, or —NH⁺(R₁₂)₂,
   where each R₁₂ is independently H, alkyl, alkenyl or alkynyl.

In some embodiments, the compound having the structure:

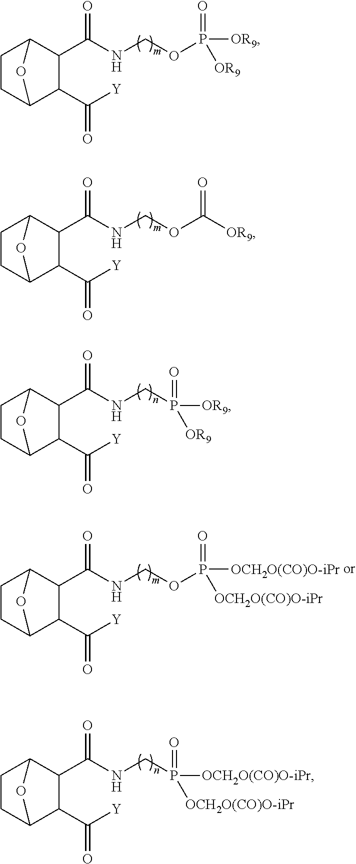

wherein
Each n=0-19, m=1-20, o=0-8 and o′=0-4,
Each R₉ is independently H, alkyl, alkenyl, or alkynyl; and
Y is OR₆,

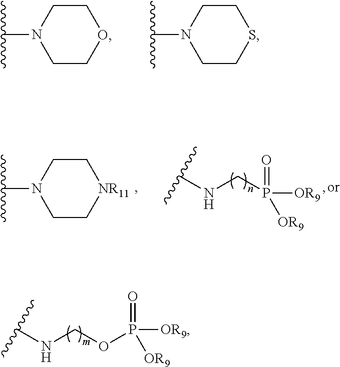

wherein R₆ is alkyl-P(O)(OR₉)₂, alkyl-OP(O)(OR₉)₂, alkyl-O(CO)—OR₁₀, alkyl-P(O)(O-alkyl-O(CO)—OR₁₀)₂, or alkyl-OP(O)(O-alkyl-O(CO)—OR₁₀)₂,
   wherein R₁₁ is H, alkyl, hydroxyalkyl, alkenyl, alkenyl, alkynyl, aryl, alkylaryl, heteroaryl, alkylheteroaryl,

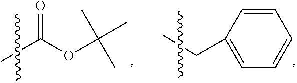

—CH₂CN, —CH₂CO₂R₁₂, —CH₂COR₁₂, —NHR₁₂, or —NH⁺(R₁₂)₂,
wherein R₉ and R₁₀ are each independently H, alkyl, alkenyl, or alkynyl,
   where each R₁₂ is independently H, alkyl, alkenyl or alkynyl.

In some embodiments, the compound having the structure:

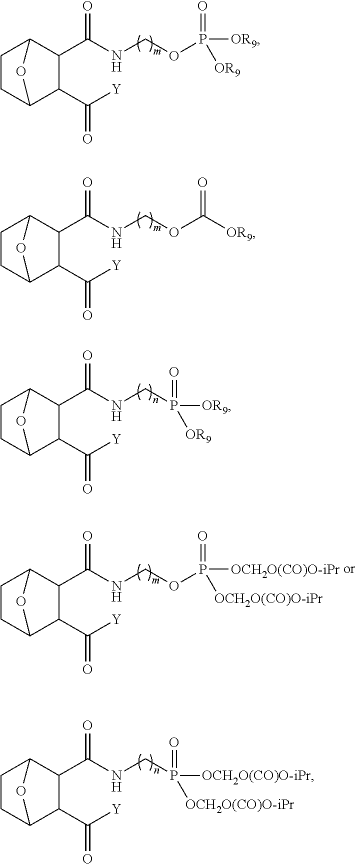

wherein
Each n=0-19, m=1-20, o=0-8 and o′=0-4,
Each R₉ is independently H, alkyl, alkenyl, or alkynyl; and
Y is

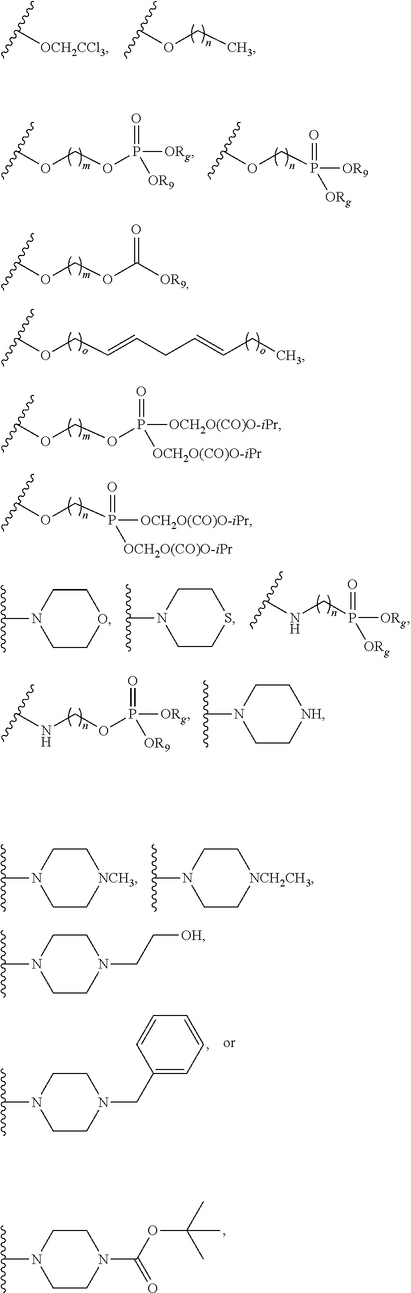

wherein
Each n=0-19, m=1-20, o=0-8 and o′=0-6,
Each R₉ is independently H, alkyl, alkenyl, or alkynyl.

In some embodiments, the compound having the structure:

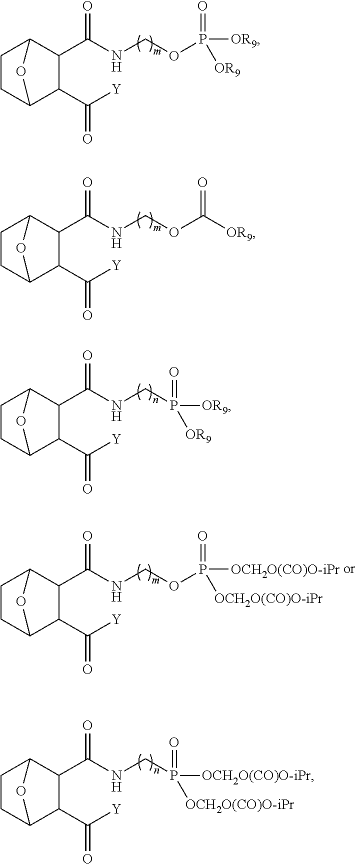

wherein
Each n=0-19, m=1-20, o=0-8 and o′=0-4,
Each R₉ is independently H, alkyl, alkenyl, or alkynyl; and
Y is

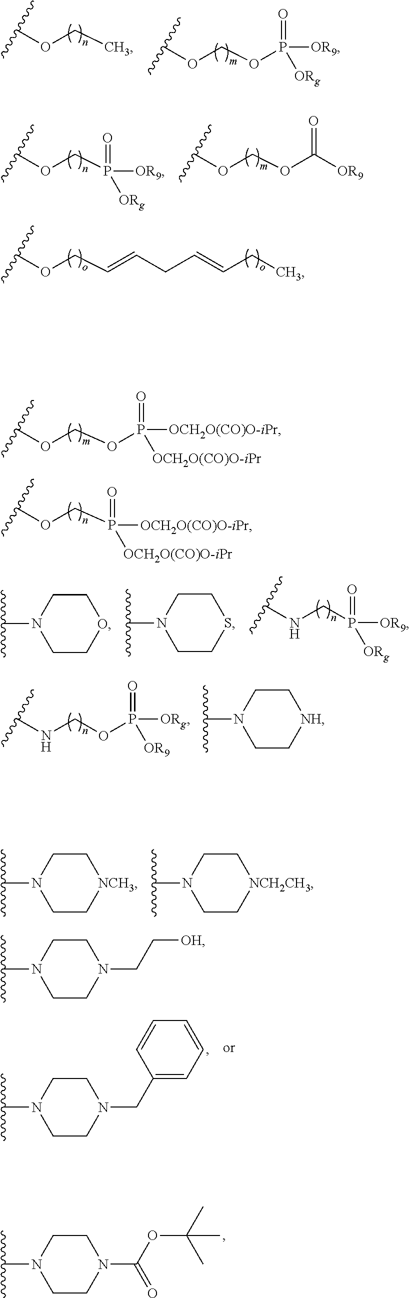

wherein
Each n=0-19, m=1-20, o=0-8 and o′=0-6,
Each R₉ is independently H, alkyl, alkenyl, or alkynyl.

In some embodiments, the compound having the structure:

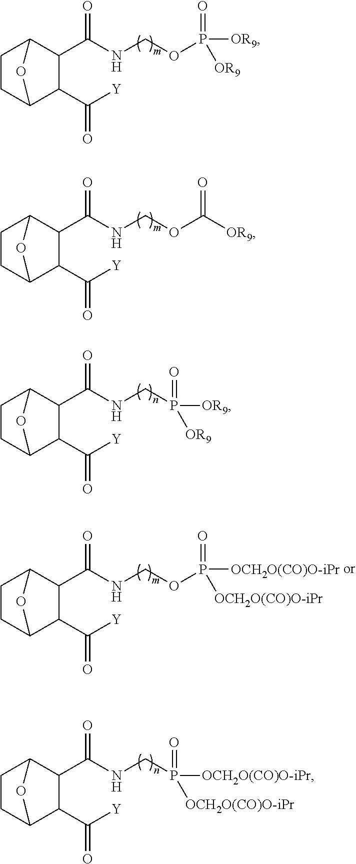

wherein
Each n=0-19, m=1-20,
Each R₉ is independently H, alkyl, alkenyl, or alkynyl; and
Y is

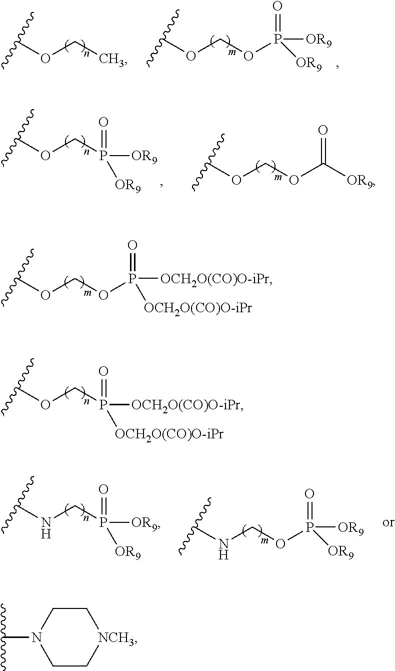

wherein
Each n=0-19, m=1-20,
Each R₉ is independently H, alkyl, alkenyl, or alkynyl.

In some embodiments, the compound having the structure:

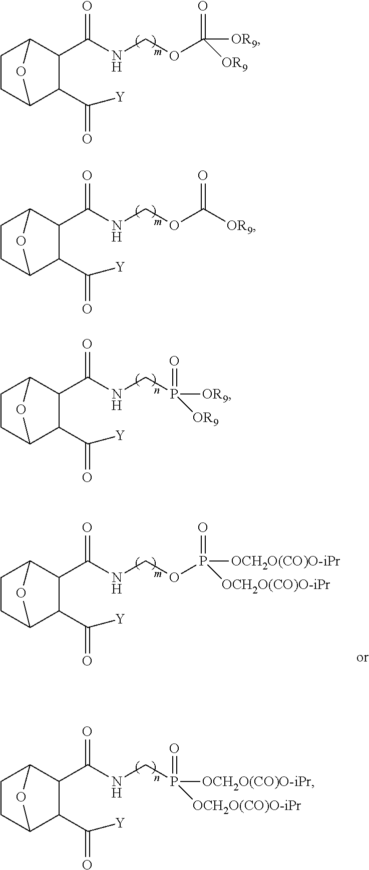

wherein
Each n=0-4 and m=2-4,
Each R₉ is independently H, CH₃, CH₂CH₃, or CH(CH₃)₂; and
Y is

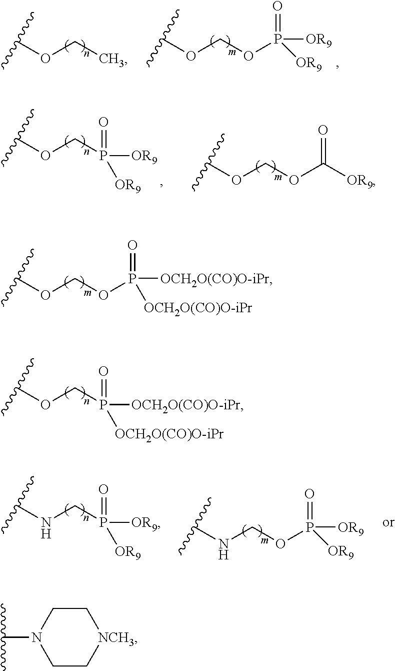

wherein
Each n=0-4 and m=2-4,
Each R₉ is independently H, CH₃, CH₂CH₃, or CH(CH₃)₂.

In some embodiments, the compound having the structure:

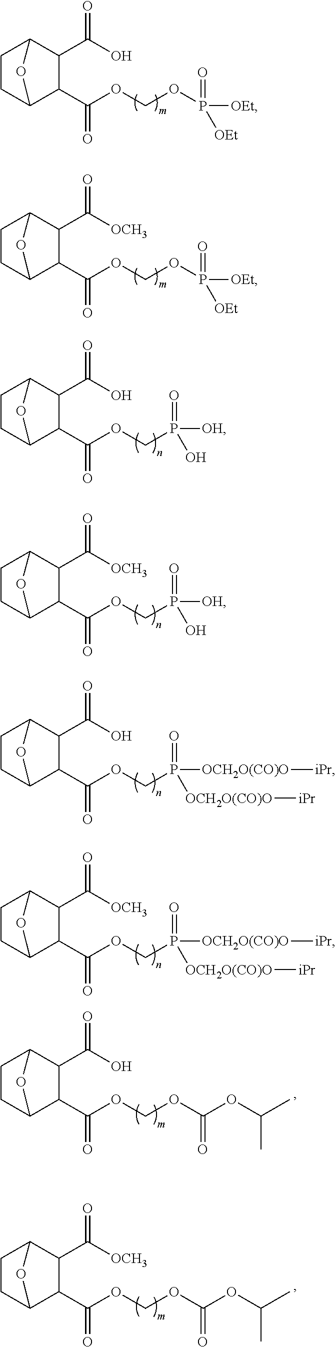

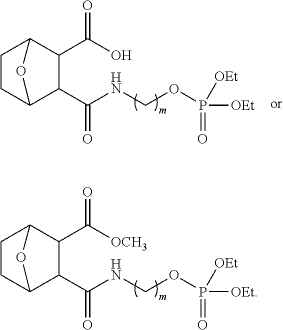

wherein each n=2-4 and each m=2-4.

In some embodiments, the compound having the structure:

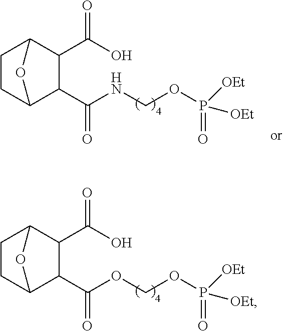

or a pharmaceutically acceptable salt or ester of the compound,

In some embodiments, the compound having the structure:

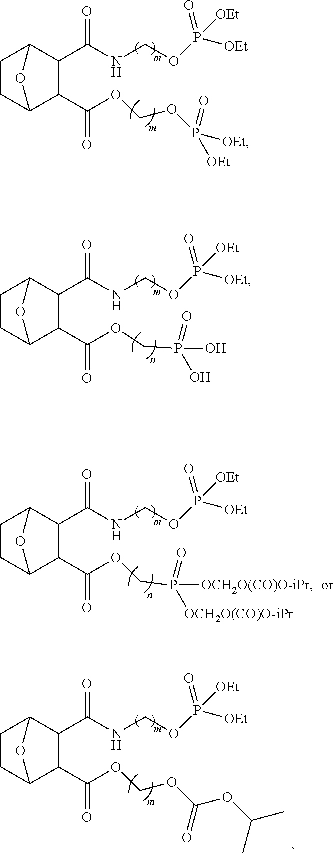

wherein each n=2-4 and each m=2-4.

In some embodiments, the compound having the structure:

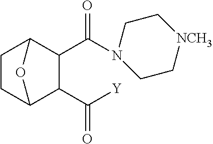

wherein
Y is OR₁, OR₆,

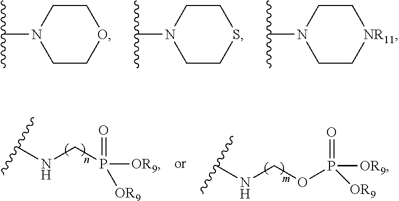

wherein R₁ is C₂-C₂₀ alkyl, C₂-C₂₀ alkenyl, or C₂-C₂₀ alkynyl;
wherein R₆ is alkyl-P(O)(OR₆)₂, alkyl-OP(O)(OR₉)₂, alkyl-O(CO)—R₁₀, alkyl-P(O)(O-alkyl-O(CO)—OR₁₀)₂, or alkyl-OP(O)(O-alkyl-O(CO)—OR₁₀)₂,
   wherein R₉ and R₁₀ are each independently H, alkyl, alkenyl, or alkynyl,
wherein R₁₁ is H, alkyl, hydroxyalkyl, alkenyl, alkenyl, alkynyl, aryl, alkylaryl, heteroaryl, alkylheteroaryl,

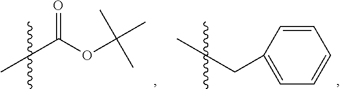

—CH₂CN, —CH₂CO₂R₁₂, —CH₂COR₁₂, —NHR₁₂, or —NH⁺(R₁₂)₂,
   where each R₁₂ is independently H, alkyl, alkenyl or alkynyl.

In some embodiments, the compound wherein
Y is

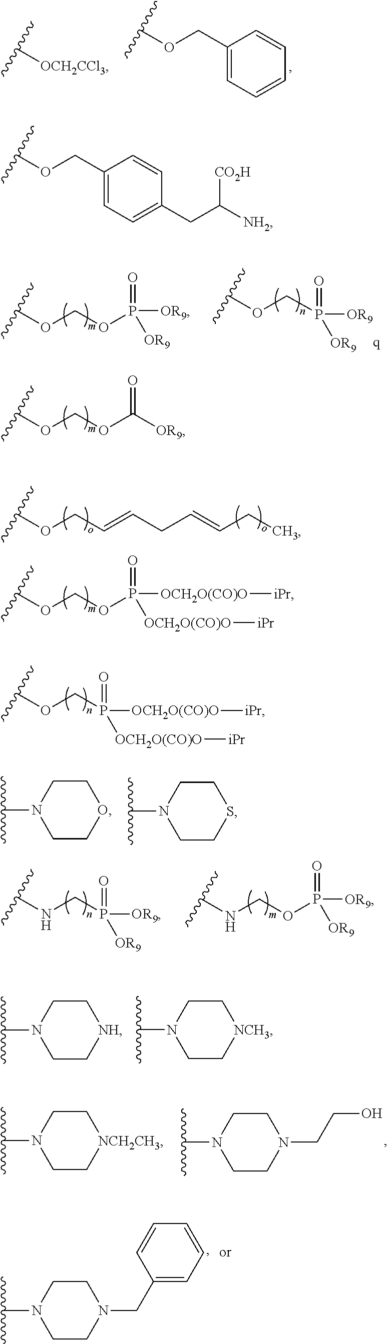

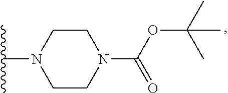

wherein
Each n=0-19, m=1-20, o=0-8 and o′=0-6,
Each R₉ is independently H, alkyl, alkenyl, or alkynyl.

In some embodiments, the compound wherein
Y is

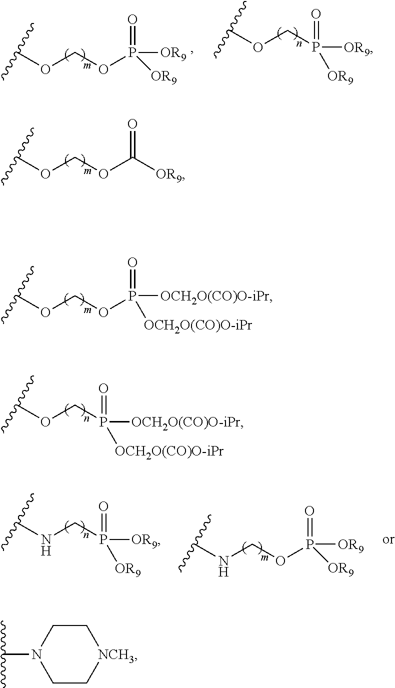

wherein
Each n=0-19, m=1-20,
Each R₉ is independently H, alkyl, alkenyl, or alkynyl.

In some embodiments, the compound wherein
Y is

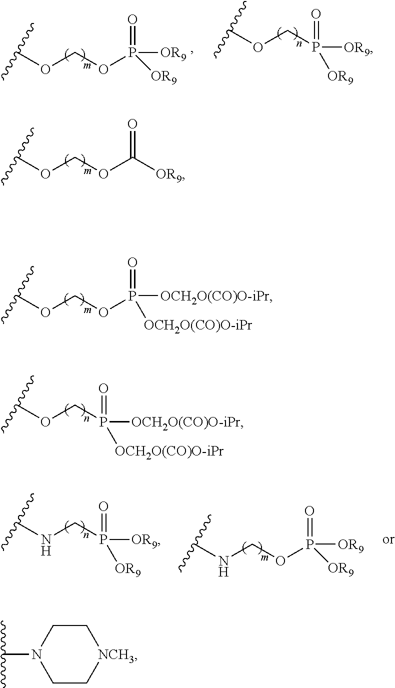

wherein
Each n=0-4 and m=2-4,
Each R₉ is independently H, CH₃, CH₂CH₃, or CH(CH₃)₂.

In some embodiments, the compound having the structure:

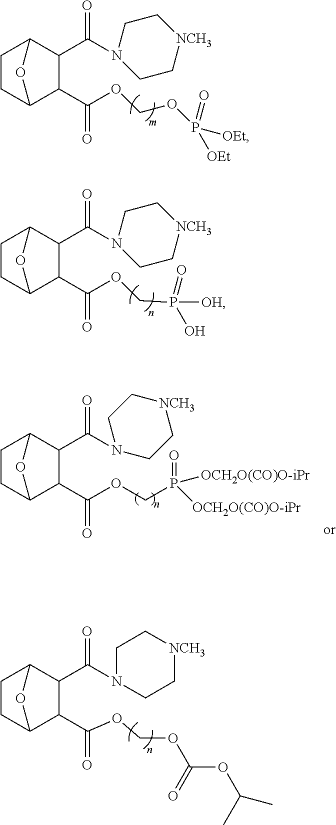

wherein each n=2-4 and each m=2-4.

The present invention further provides a method of treating a subject afflicted with cancer comprising administering a therapeutically effective amount of a compound having the structure:

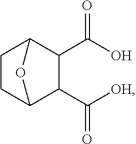 [SMILES: O=C(O)C1C2CCC(O2)C1C(=O)O]

or a pharmaceutically acceptable salt or ester thereof, so as to thereby treat the subject.

The present invention further provides a method of inhibiting proliferation or inducing apoptosis of a cancer cell in a human subject afflicted with cancer comprising administering a therapeutically effective amount of a compound having the structure:

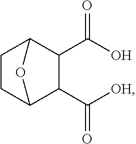 [SMILES: O=C(O)C1C2CCC(O2)C1C(=O)O]

or a pharmaceutically acceptable salt or ester thereof, so as to thereby inhibit proliferation or induce apoptosis of the cancer cell.

The present invention further provides a compound having the structure

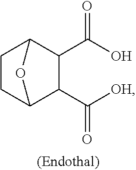 [SMILES: O=C(O)C1C2CCC(O2)C1C(=O)O]

or a pharmaceutically acceptable salt or ester thereof, for use in treating cancer in a subject.

In some embodiments of the above method, the cancer is selected from adrenocortical cancer, bladder cancer, osteosarcoma, cervical cancer, esophageal, gallbladder, head and neck cancer, Hodgkin lymphoma, non-Hodgkin lymphoma, renal cancer, melanoma, pancreatic cancer, rectal cancer, thyroid cancer and throat cancer.

In some embodiments of the above method, the cancer is selected from breast cancer, colon cancer, large cell lung cancer, adenocarcinoma of the lung, small cell lung cancer, stomach cancer, liver cancer, ovary adenocarcinoma, pancreas carcinoma, prostate carcinoma, promylocytic leukemia.

In some embodiments of the above method, the cancer is breast cancer, colon cancer, large cell lung cancer, adenocarcinoma of the lung, small cell lung cancer, stomach cancer, liver cancer, ovary adenocarcinoma, pancreas carcinoma, prostate carcinoma, promylocytic leukemia, chronic myelocytic leukemia, acute lymphocytic leukemia, colorectal cancer, ovarian cancer, lymphoma, non-Hodgkin's lymphoma or Hodgkin's lymphoma.

In some embodiments of the above method, the cancer is brain cancer.

In some embodiments of the above method, the brain cancer is a glioma, pilocytic astrocytoma, low-grade diffuse astrocytoma, anaplastic astrocytoma, glioblastoma multiforme, oligodendroglioma, ependymoma, meningioma, pituitary gland tumor, primary CNS lymphoma, medulloblastoma, craniopharyngioma, or diffuse intrinsic pontine glioma.

In some embodiments of the above method, the compound is co-administered with an anti-cancer agent.

In some embodiments of the above method, the anti-cancer agent is selected from x-radiation, ionizing radiation, a DNA damaging agent, a DNA intercalating agent, a microtubule stabilizing agent, a microtubule destabilizing agent, a spindle toxin, abarelix, aldesleukin, alemtuzumab, alitertinoin, allopurinol, altretamine, amifostin, anakinra, anastrozole, arsenic trioxide, asparaginase, azacitidine, bevacizumab, bexarotene, bleomycin, bortezomib, busulfan, calusterone, capecitabine, carboplatin, carmustine, celecoxib, cetuximab, chlorambucil, cisplatin, cladribine, clofarabine, cyclophosphamide, cytarabine, dacarbazine, dactinomycin, actinomycin D, dalteparin sodium, darbepoetin alfa, dasatinib, daunorubicin, daunomycin, decitabine, denileukin, dexrazoxane, docetaxel, doxorubicin, dromostanolone propionate, exulizumab, epirubicin, epoetin alfa, erlotinib, estramustine, etoposide phosphate, etoposide, VP-16, exemestane, fentanyl citrate, filgrastim, floxuridine, fludarabine, fluorouracil, fulvestrant, gefitinib, gemcitabine, gosereline acetate, histrelin acetate, hydroxyurea, ibritumomab tiuxetan, idarubicin, ifosfamide, imatinib mesylate, interferon alfa 2a, interferon alfa 2b, irinotecan, lapatinib ditosylate, lenalidomide, letrozole, leucovrin, leuprolide acetate, levamisole, lomustine, meclorethamine, megestrol acetate, melphalan, mercaptopurine, mesna, methotrexate, methoxsalen, mitomycin C, mitotane, mitoxantrone, nandrolone phenpropionate, nelarabine, nofetumomab, oprelvekin, oxaliplatin, paclitaxel, palifermin, pamidronate, panitumumab, pegademase, pegaspargase, pegfilgrastim, peginterferon alfa 2b, pemetrexed disodium, pentostatin, pipobroman, plicamycin, mithramycin, porfimer sodium, procarbazine, quinacrine, rasburicase, rituximab, sargrmostim, sorafenib, streptozocin, sunitinib, sunitinib maleate, talc, tamoxifen, temozolomide, teniposide, VM-26, testolactone, thalidomide, thioguanine, G-TG, thiotepa, topotecan, toremifene, tositumomab, trastuzumab, tretinoin ATRA, ruacil mustard, valrunicin, vinblastine, vincristine, vinorelbine, vorinostat, zoledronate, and zoledronic acid.

In some embodiments of the above method, the anti-cancer agent is x-radiation.

In some embodiments of the above method, the anti-cancer agent is ionizing radiation.

In some embodiments of the above method, the anti-cancer agent is a DNA damaging agent, a DNA intercalating agent, a microtubule stabilizing agent, a microtubule destabilizing agent or a spindle toxin.

The present invention provides a compound having the structure:

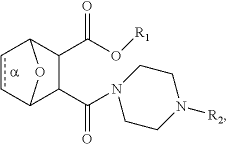

wherein
bond α is absent or present;
R₁ is C₂-C₂₀ alkyl, C₂-C₂₀ alkenyl, or C₂-C₂₀ alkynyl;
R₂ is H, C₁-C₁₂ alkyl, C₁-C₁₂ alkenyl, C₁-C₁₂ alkynyl, C₁-C₁₂ alkyl-(phenyl), C₁-C₁₂ alkyl-(OH), or C(O)C(CH₃)₃,
   or a pharmaceutically acceptable salt of the compound.

In some embodiments, a compound having the structure:

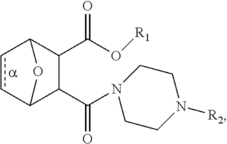

wherein
bond α is absent or present;
R₁ is C₃-C₂₀ alkyl, C₂-C₂₀ alkenyl, or C₂-C₂₀ alkynyl;
R₂ is H, C₁-C₁₂ alkyl, C₁-C₁₂ alkenyl, C₁-C₁₂ alkynyl, C₁-C₁₂ alkyl-(phenyl), C₁-C₁₂ alkyl-(OH), or C(O)C(CH₃)₃,
   or a pharmaceutically acceptable salt of the compound.

In some embodiments, a compound having the structure:

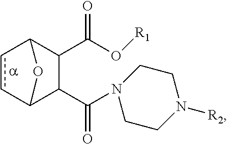

wherein
bond α is absent or present;
R₁ is C₄-C₂₀ alkyl, C₂-C₂₀ alkenyl, or C₂-C₂₀ alkynyl;
R₂ is H, C₁-C₁₂ alkyl, C₁-C₁₂ alkenyl, C₁-C₁₂ alkynyl, C₁-C₁₂ alkyl-(phenyl), C₁-C₁₂ alkyl-(OH), or C(O)C(CH₃)₃,
   or a pharmaceutically acceptable salt of the compound.

In some embodiments, the compound having the structure:

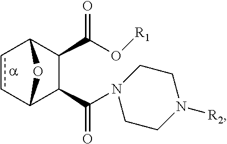

or a pharmaceutically acceptable salt of the compound.

In some embodiments, wherein R₁ is C₂-C₂₀ alkyl or C₂-C₂₀ alkenyl; and R₂ is C₁-C₁₂ alkyl. In some embodiments, wherein R₁ is C₂-C₂₀ alkyl or C₂-C₂₀ alkenyl; and R₂ is C₁-C₁₂ alkyl-(phenyl). In some embodiments, wherein R₁ is C₂-C₂₀ alkyl or C₂-C₂₀ alkenyl; and R₂ is C₁-C₁₂ alkyl-(OH). The compound of claim 1 or 2, wherein R₁ is C₂-C₂₀ alkyl or C₂-C₂₀ alkenyl; and R₂ is —C(O)C(CH₃)₃.

In some embodiments, wherein R₁ is C₃-C₂₀ alkyl or C₂-C₂₀ alkenyl; and R₂ is C₁-C₁₂ alkyl. In some embodiments, wherein R₁ is C₃-C₂₀ alkyl or C₂-C₂₀ alkenyl; and R₂ is C₁-C₁₂ alkyl-(phenyl). In some embodiments, wherein R₁ is C₃-C₂₀ alkyl or C₂-C₂₀ alkenyl; and R₂ is C₁-C₁₂ alkyl-(OH). The compound of claim 1 or 2, wherein R₃ is C₂-C₂₀ alkyl or C₂-C₂₀ alkenyl; and R₂ is —C(O)C(CH₃)₃.

In some embodiments, wherein R₁ is C₄-C₂₀ alkyl or C₂-C₂₀ alkenyl; and R₂ is C₁-C₁₂ alkyl. In some embodiments, wherein R₁ is C₄-C₂₀ alkyl or C₂-C₂₀ alkenyl; and R₂ is C₁-C₁₂ alkyl-(phenyl). In some embodiments, wherein R₁ is C₄-C₂₀ alkyl or C₂-C₂₀ alkenyl; and R₂ is C₁-C₁₂ alkyl-(OH). The compound of claim 1 or 2, wherein R₁ is C₄-C₂₀ alkyl or C₂-C₂₀ alkenyl; and R₂ is —C(O)C(CH₃)₃.

In some embodiments, the compound wherein
R₁ is —CH₂CH₃,
—CH₂CH₂CH₃,
—CH₂CH₂CH₂CH₃,
—CH₂CH₂CH₂CH₂CH₃,
—CH₂CH₂CH₂CH₂CH₂CH₃,
—CH₂CH₂CH₂CH₂CH₂CH₂CH₃,
—CH₂CH₂CH₂CH₂CH₂CH₂CH₂CH₃,
—CH₂CH₂CH₂CH₂CH₂CH₂CH₂CH₂CH₃, or
—CH₂CH₂CH₂CH₂CH₂CH₂CH₂CH₂CH₂CH₃.

In some embodiments, the compound wherein
R₁ is —CH₂CH₂CH₂CH₂CH₂CH₂CH₂CH₂CH₂CH₂CH₂CH₂CH₂CH₂CH₂CH₂CH₃, or
—CH₂CH₂CH₂CH₂CH₂CH₂CH₂CH₂CH═CHCH₂CH═CHCH₂CH₂CH₂CH₂CH₃.

In some embodiments, the compound wherein

R₂ is —H, —CH₃, —CH₂CH₃, —CH₂-phenyl, —CH₂CH₂—OH, or —C(O)C(CH₃)₃.

In some embodiments, the compound having the structure:

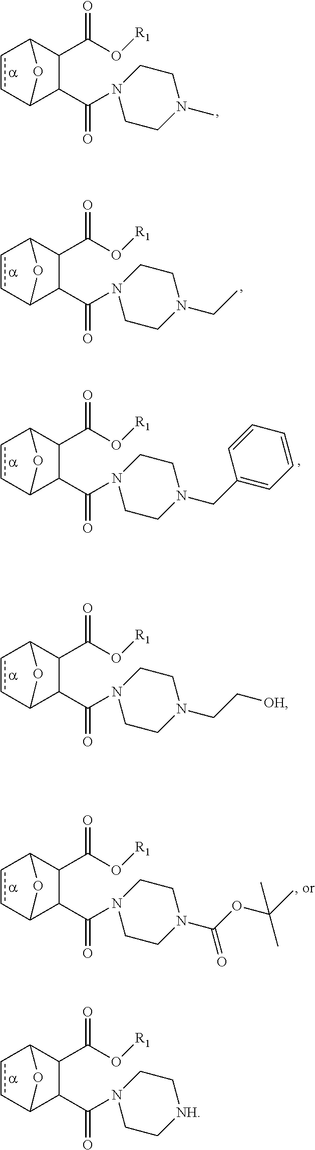

In some embodiments of any of the above compounds, the compound wherein
R₁ is —CH₂CH₃,
—CH₂CH₂CH₃,
—CH₂CH₂CH₂CH₃,
—CH₂CH₂CH₂CH₂CH₃,
—CH₂CH₂CH₂CH₂CH₂CH₃,
—CH₂CH₂CH₂CH₂CH₂CH₂CH₃,
—CH₂CH₂CH₂CH₂CH₂CH₂CH₂CH₃,
—CH₂CH₂CH₂CH₂CH₂CH₂CH₂CH₂CH₃,
—CH₂CH₂CH₂CH₂CH₂CH₂CH₂CH₂CH₂CH₃,
—CH₂CH₂CH₂CH₂CH₂CH₂CH₂CH₂CH₂CH₂CH₂CH₂CH₂CH₂CH₂CH₂CH₃, or
—CH₂CH₂CH₂CH₂CH₂CH₂CH₂CH₂CH═CHCH₂CH═CHCH₂CH₂CH₂CH₂CH₃.

In some embodiments, the compound wherein α is absent.

In some embodiments, the compound wherein α is present.

In some embodiments, the compound having the structure:

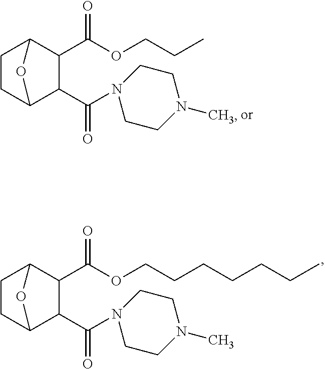 [SMILES: CCCOC(=O)C1C2CCC(O2)C1C(=O)N1CCN(C)CC1 ; "or" ; CCCCCCCOC(=O)C1C2CCC(O2)C1C(=O)N1CCN(C)CC1]

or a pharmaceutically acceptable salt of the compound.

In some embodiments, the compound having the structure:

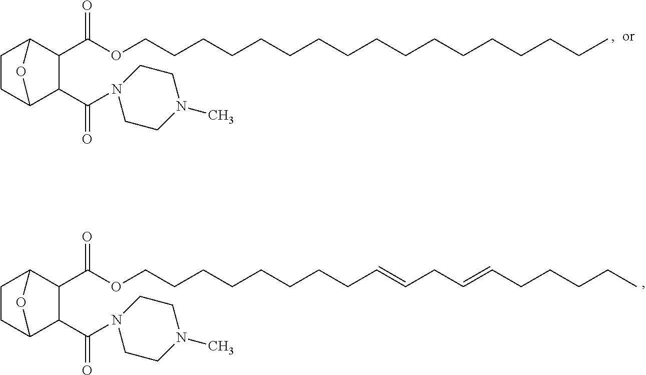 [SMILES: CCCCCCCCCCCCCCCCCOC(=O)C1C2CCC(O2)C1C(=O)N1CCN(C)CC1 ; CCCCC/C=C/C/C=C/CCCCCCCCOC(=O)C1C2CCC(O2)C1C(=O)N1CCN(C)CC1]

or a pharmaceutically acceptable salt of the compound.

In some embodiments, the compound having the structure:

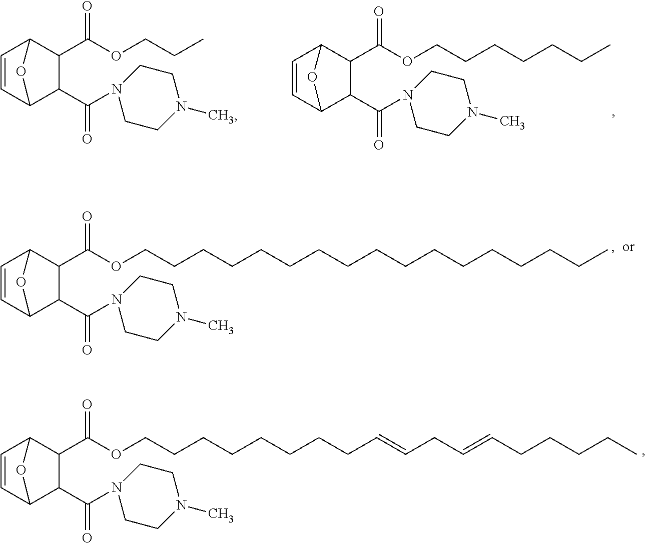 [SMILES: CCCOC(=O)C1C2C=CC(O2)C1C(=O)N1CCN(C)CC1 ; CCCCCCCOC(=O)C1C2C=CC(O2)C1C(=O)N1CCN(C)CC1 ; "," ; CCCCCCCCCCCCCCCCCOC(=O)C1C2C=CC(O2)C1C(=O)N1CCN(C)CC1 ; CCCCC/C=C/C/C=C/CCCCCCCCOC(=O)C1C2C=CC(O2)C1C(=O)N1CCN(C)CC1]

or a pharmaceutically acceptable salt of the compound.

The present invention provides a pharmaceutical composition comprising a compound of the present application and a pharmaceutically acceptable carrier.

The present invention provides a pharmaceutical composition comprising a compound of the present application or a pharmaceutically acceptable salt thereof and an anticancer agent, and at least one pharmaceutically acceptable carrier.

In some embodiments, the pharmaceutical composition wherein the pharmaceutically acceptable carrier comprises a liposome.

In some embodiments, the pharmaceutical composition wherein the compound is contained in a liposome or microsphere, or the compound and the anti-cancer agent are contained in a liposome or microsphere.

In some embodiments, the pharmaceutical composition wherein the compound has the structure:

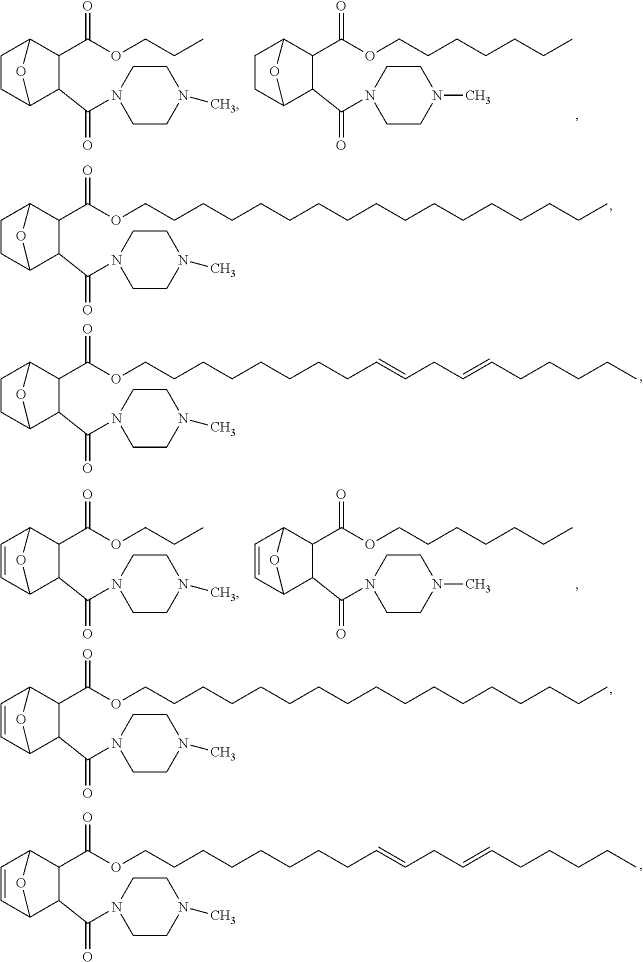

or
a pharmaceutically acceptable salt of the compound.

In some embodiments, the compound having the structure:

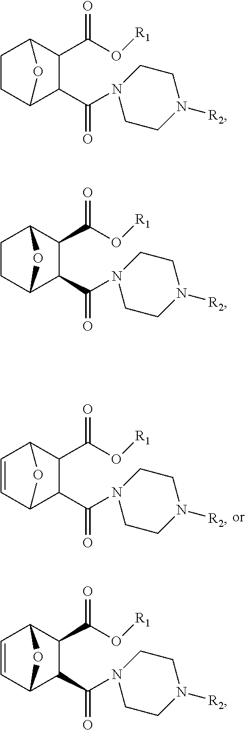

or a pharmaceutically acceptable salt of the compound.

In some embodiments, a compound having the structure:

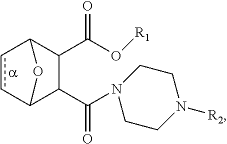

wherein
bond α is absent or present;
R₁ is C₃-C₂₀ alkyl, C₃-C₂₀ alkenyl, or C₃-C₂₀ alkynyl;
R₂ is H, C₁-C₁₂ alkyl, C₁-C₁₂ alkenyl, C₁-C₁₂ alkynyl, C₁-C₁₂
alkyl-(phenyl), C₁-C₁₂ alkyl-(OH), or C(O)C(CH₃)₃,
   or a pharmaceutically acceptable salt of the compound.

In some embodiments, the compound having the structure:

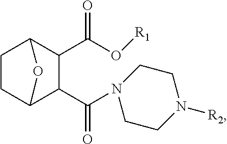

wherein
R₁ is C₂-C₂₀ alkyl or C₂-C₂₀ alkenyl; and
R₂ is C₁-C₁₂ alkyl,
   or a pharmaceutically acceptable salt of the compound.

In some embodiments, the compound having the structure:

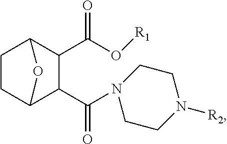

wherein
R₁ is C₃-C₂₀ alkyl or C₃-C₂₀ alkenyl; and
R₂ is C₁-C₁₂ alkyl,
   or a pharmaceutically acceptable salt of the compound.

In some embodiments, the pharmaceutical composition wherein the anti-cancer agent is selected from a DNA damaging agent, a DNA intercalating agent, a microtubule stabilizing agent, a microtubule destabilizing agent, a spindle toxin, abarelix, aldesleukin, alemtuzumab, alitertinoin, allopurinol, altretamine, amifostin, anakinra, anastrozole, arsenic trioxide, asparaginase, azacitidine, bevacizumab, bexarotene, bleomycin, bortezomib, busulfan, calusterone, capecitabine, carboplatin, carmustine, celecoxib, cetuximab, chlorambucil, cisplatin, cladribine, clofarabine, cyclophosphamide, cytarabine, dacarbazine, dactinomycin, actinomycin D, dalteparin sodium, darbepoetin alfa, dasatinib, daunorubicin, daunomycin, decitabine, denileukin, dexrazoxane, docetaxel, doxorubicin, dromostanolone propionate, exulizumab, epirubicin, epoetin alfa, erlotinib, estramustine, etoposide phosphate, etoposide, VP-16, exemestane, fentanyl citrate, filgrastim, floxuridine, fludarabine, fluorouracil, fulvestrant, gefitinib, gemcitabine, gosereline acetate, histrelin acetate, hydroxyurea, ibritumomab tiuxetan, idarubicin, ifosfamide, imatinib mesylate, interferon alfa 2a, interferon alfa 2b, irinotecan, lapatinib ditosylate, lenalidomide, letrozole, leucovrin, leuprolide acetate, levamisole, lomustine, meclorethamine, megestrol acetate, melphalan, mercaptopurine, mesna, methotrexate, methoxsalen, mitomycin C, mitotane, mitoxantrone, nandrolone phenpropionate, nelarabine, nofetumomab, oprelvekin, oxaliplatin, paclitaxel, palifermin, pamidronate, panitumumab, pegademase, pegaspargase, pegfilgrastim, peginterferon alfa 2b, pemetrexed disodium, pentostatin, pipobroman, plicamycin, mithramycin, porfimer sodium, procarbazine, quinacrine, rasburicase, rituximab, sargrmostim, sorafenib, streptozocin, sunitinib, sunitinib maleate, talc, tamoxifen, temozolomide, teniposide, VM-26, testolactone, thalidomide, thioguanine, G-TG, thiotepa, topotecan, toremifene, tositumomab, trastuzumab, tretinoin ATRA, uracil mustard, valrunicin, vinblastine, vincristine, vinorelbine, vorinostat, zoledronate, zoledronic acid, abraxane and brentuximab vedotin.

The present invention provides a method of treating a subject afflicted with cancer comprising administering to the subject a therapeutically effective amount of the compound of the present invention.

The present invention provides a method of enhancing the anti-cancer activity of an anti-cancer agent in a subject afflicted with a cancer, comprising administering to the subject the compound of the present invention in an amount effective to enhance the anti-cancer activity of the anti-cancer agent.

The present invention provides a method of treating a subject afflicted with cancer comprising periodically administering to the subject

a) an amount of the compound of the present invention or a pharmaceutically acceptable salt thereof, and
b) an anti-cancer agent,
wherein the amounts when taken together are more effective to treat the subject than when each agent at the same amount is administered alone.

The present invention provides for the use of the compound of the present invention or a pharmaceutically acceptable salt thereof and an anti-cancer agent in the preparation of a combination for treating a subject afflicted with cancer wherein the amount of the compound and the amount of the anti-cancer agent are administered simultaneously or contemporaneously.

The present invention provides a pharmaceutical composition comprising an amount of the compound of the present invention or a pharmaceutically acceptable salt thereof for use in treating a subject afflicted with cancer as an add-on therapy or in combination with, or simultaneously, contemporaneously or concomitantly with an anti-cancer agent.

In some embodiments, the compound of the present invention or a pharmaceutically acceptable salt thereof for use as an add-on therapy or in combination with an anti-cancer agent in treating a subject afflicted with cancer.

In some embodiments, the compound of the present invention or a pharmaceutically acceptable salt thereof and an anti-cancer agent for the treatment of a subject afflicted with cancer wherein the compound and the anti-cancer agent are administered simultaneously, separately or sequentially.

In some embodiments, a product containing an amount of the compound of the present invention or a pharmaceutically acceptable salt thereof and an amount of an anti-cancer agent for simultaneous, separate or sequential use in treating a subject afflicted cancer.

In some embodiments, the compound of the present invention or a pharmaceutically acceptable salt thereof for use in treating cancer.

In some embodiments, the compound of the present invention or a pharmaceutically acceptable salt thereof in combination with an anti-cancer agent for use in treating cancer.

In some embodiments of any of the above methods, uses, pharmaceutical compositions, compounds or products, the cancer is breast cancer, colon cancer, large cell lung cancer, adenocarcinoma of the lung, small cell lung cancer, stomach cancer, liver cancer, ovary adenocarcinoma, pancreas carcinoma, prostate carcinoma, promylocytic leukemia, chronic myelocytic leukemia, acute lymphocytic leukemia, colorectal cancer, ovarian cancer, lymphoma, non-Hodgkin's lymphoma or Hodgkin's lymphoma.

In some embodiments of any of the above methods, uses, pharmaceutical compositions, compounds or products, the cancer is brain cancer.

In some embodiments of any of the above methods, uses, pharmaceutical compositions, compounds or products, the brain cancer is a glioma, pilocytic astrocytoma, low-grade diffuse astrocytoma, anaplastic astrocytoma, glioblastoma multiforme, oligodendroglioma, ependymoma, meningioma, pituitary gland tumor, primary CNS lymphoma, medulloblastoma, craniopharyngioma, or diffuse intrinsic pontine glioma.

In some embodiments of any of the above methods, uses, pharmaceutical compositions, compounds or products, the compound crosses the blood brain barrier of the subject.

In some embodiments of any of the above methods, uses, pharmaceutical compositions, compounds or products, the compound and/or a metabolite of the compound crosses the blood brain barrier of the subject.

The present invention provides a method of inhibiting proliferation or inducing apoptosis of a cancer cell in a human subject, comprising administering to the subject:

a) the compound of the present invention, or a salt of the compound, in an amount effective to inhibit the proliferation or to induce apoptosis of the cancer cell, and
b) an anti-cancer agent in an amount effective to inhibit the proliferation or to induce apoptosis of the cancer cell.

The present invention provides a method of inhibiting proliferation or inducing apoptosis of a cancer cell in a human subject which overexpresses translationally controlled tumour protein (TCTP) comprising administering to the subject

a) the compound of the present invention, or a salt of the compound, in an amount effective to inhibit the proliferation or to induce apoptosis of the cancer cell, and
b) an anti-cancer agent in an amount effective to inhibit the proliferation or to induce apoptosis of the cancer cell.

In some embodiments of the above methods, the cancer cell does not overexpress N—CoR.

In some embodiments of any of the above methods, uses, pharmaceutical compositions, compounds or products, the anti-cancer agent is selected from x-radiation or ionizing radiation.

In some embodiments of any of the above methods, uses, pharmaceutical compositions, compounds or products, the anti-cancer agent is selected from a DNA damaging agent, a DNA intercalating agent, a microtubule stabilizing agent, a microtubule destabilizing agent, a spindle toxin, abarelix, aldesleukin, alemtuzumab, alitertinoin, allopurinol, altretamine, amifostin, anakinra, anastrozole, arsenic trioxide, asparaginase, azacitidine, bevacizumab, bexarotene, bleomycin, bortezomib, busulfan, calusterone, capecitabine, carboplatin, carmustine, celecoxib, cetuximab, chlorambucil, cisplatin, cladribine, clofarabine, cyclophosphamide, cytarabine, dacarbazine, dactinomycin, actinomycin D, dalteparin sodium, darbepoetin alfa, dasatinib, daunorubicin, daunomycin, decitabine, denileukin, dexrazoxane, docetaxel, doxorubicin, dromostanolone propionate, exulizumab, epirubicin, epoetin alfa, erlotinib, estramustine, etoposide phosphate, etoposide, VP-16, exemestane, fentanyl citrate, filgrastim, floxuridine, fludarabine, fluorouracil, fulvestrant, gefitinib, gemcitabine, gosereline acetate, histrelin acetate, hydroxyurea, ibritumomab tiuxetan, idarubicin, ifosfamide, imatinib mesylate, interferon alfa 2a, interferon alfa 2b, irinotecan, lapatinib ditosylate, lenalidomide, letrozole, leucovrin, leuprolide acetate, levamisole, lomustine, meclorethamine, megestrol acetate, melphalan, mercaptopurine, mesna, methotrexate, methoxsalen, mitomycin C, mitotane, mitoxantrone, nandrolone phenpropionate, nelarabine, nofetumomab, oprelvekin, oxaliplatin, paclitaxel, palifermin, pamidronate, panitumumab, pegademase, pegaspargase, pegfilgrastim, peginterferon alfa 2b, pemetrexed disodium, pentostatin, pipobroman, plicamycin, mithramycin, porfimer sodium, procarbazine, quinacrine, rasburicase, rituximab, sargrmostim, sorafenib, streptozocin, sunitinib, sunitinib maleate, talc, tamoxifen, temozolomide, teniposide, VM-26, testolactone, thalidomide, thioguanine, G-TG, thiotepa, topotecan, toremifene, tositumomab, trastuzumab, tretinoin ATRA, uracil mustard, valrunicin, vinblastine, vincristine, vinorelbine, vorinostat, zoledronate, zoledronic acid, abraxane and brentuximab vedotin.

In some embodiments of any of the above methods, uses, pharmaceutical compositions, compounds or products, the subject is a human.

In some embodiments of any of the above methods, uses, pharmaceutical compositions, compounds or products, the compound has the structure:

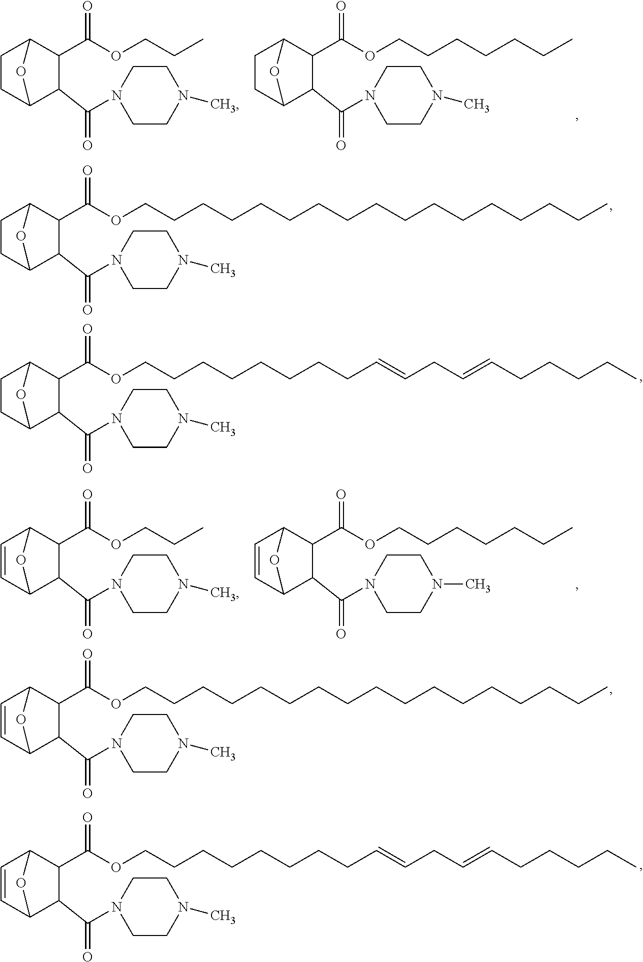

or
a pharmaceutically acceptable salt of the compound.

In some embodiments of any of the above methods, the cancer is adrenocortical cancer, bladder cancer, osteosarcoma, cervical cancer, esophageal, gallbladder, head and neck cancer, lymphoma, Hodgkin's lymphoma, non-Hodgkin's lymphoma, renal cancer, melanoma, pancreatic cancer, rectal cancer, thyroid cancer or throat cancer. In some embodiments of any of the above methods, the cancer is selected from brain cancer, breast cancer, lung cancer, prostate cancer, and head or neck cancer.

In some embodiments of any of the above methods or uses, the subject is a human.

In one embodiment, a pharmaceutical composition comprising the compound of the present invention. In one embodiment, a pharmaceutical composition comprising the compound of the present invention and a pharmaceutically acceptable carrier.

In one embodiment of the method, the compound of the present invention inhibits PP2A activity in the subject. In one embodiment of the method, the compound of the present invention inhibits PP2A activity in the brain of the subject. In one embodiment of the method, the compound of the present invention crosses the blood brain barrier of the subject.

In some embodiments, the compounds of the present invention are ester derivatives of compound 100 and serve as pro-drugs of compound 100.

In some embodiments, the compounds of the present invention are ester derivatives of 100 and serve as pro-drugs that can be converted into 100 by serum esterases and/or brain esterases.

In some embodiments, the compounds of the present invention are derivatives of compound 100 and serve as pro-drugs of endothal.

In some embodiments, the compounds of the present invention are derivatives of compound 100 and serve as pro-drugs that can be converted into endothal by serum esterases and/or brain esterases.

In some embodiments, the compounds of the present invention are derivatives of compound 100 and serve as pro-drugs that cross the blood brain barrier and deliver endothal to the brain.

Administration of a pro-drug of endothal is more effective at delivering endothal to targets cells in a subject than administration of endothal itself.

The metabolic profile of endothal is such that administration of a pro-drug of endothal is more effective at delivering endothal to targets cells in a subject than administration of endothal itself.

In some embodiments, the method wherein the compound is first converted to compound 100 in vivo, which in turn is converted to endothal in vivo.

The compounds disclosed herein act as prodrugs of endothal, altering metabolism by masking one or two acid groups with an amide or an ester moiety. The design of the prodrug will result in reduced toxicity and increased systemic exposure of endothal in the subject.

In some embodiments of the delivery method, a pharmaceutical composition comprising the compound and a pharmaceutically acceptable carrier.

As used herein, a “symptom” associated with a disease includes any clinical or laboratory manifestation associated with the disease and is not limited to what the subject can feel or observe.

As used herein, “treatment of the diseases”, “treatment of the injury” or “treating”, e.g. of a disease encompasses inducing inhibition, regression, or stasis of the disease or injury, or a symptom or condition associated with the disease or injury.

As used herein, “inhibition” of disease encompasses preventing or reducing the disease progression and/or disease complication in the subject.

As used herein, “overexpressing N—CoR” means that the level of the Nuclear receptor co-repressor (N—CoR) expressed in cells of the tissue tested are elevated in comparison to the levels of N—CoR as measured in normal healthy cells of the same type of tissue under analogous conditions. The nuclear receptor co-repressor (N—CoR) of the subject invention may be any molecule that binds to the ligand binding domain of the DNA-bound thyroid hormone receptor (T3R) and retinoic acid receptor (RAR) (U.S. Pat. No. 6,949,624, Liu et al.). Examples of tumors that overexpress N—CoR may include glioblastoma multiforme, breast cancer (Myers et al. 2005), colorectal cancer (Giannini and Cavallini 2005), small cell lung carcinoma (Waters et al 2004) or ovarian cancer (Havrilesky et al. 2001).

As used herein, “alkyl” is intended to include both branched and straight-chain saturated aliphatic hydrocarbon groups having the specified number of carbon atoms. Thus, C₁-Cₙ as in “C₁-Cₙ alkyl” is defined to include groups having 1, 2, . . . , n−1 or n carbons in a linear or branched arrangement, and specifically includes methyl, ethyl, propyl, butyl, pentyl, hexyl, heptyl, isopropyl, isobutyl, sec-butyl and so on. An embodiment can be C₁-C₂₀ alkyl, C₂-C₂₀ alkyl, C₃-C₂₀ alkyl, C₄-C₂₀ alkyl and so on. An embodiment can be C₁-C₃₀ alkyl, C₂-C₃₀ alkyl, C₃-C₃₀ alkyl, C₄-C₃₀ alkyl and so on. “Alkoxy” represents an alkyl group as described above attached through an oxygen bridge.

The term “alkenyl” refers to a non-aromatic hydrocarbon radical, straight or branched, containing at least 1 carbon to carbon double bond, and up to the maximum possible number of non-aromatic carbon-carbon double bonds may be present. Thus, C₂-Cₙ alkenyl is defined to include groups having 1, 2 . . . , n−1 or n carbons. For example, “C₂-C₆ alkenyl” means an alkenyl radical having 2, 3, 4, 5, or 6 carbon atoms, and at least 1 carbon-carbon double bond, and up to, for example, 3 carbon-carbon double bonds in the case of a C₆ alkenyl, respectively. Alkenyl groups include ethenyl, propenyl, butenyl and cyclohexenyl. As described above with respect to alkyl, the straight, branched or cyclic portion of the alkenyl group may contain double bonds and may be substituted if a substituted alkenyl group is indicated. An embodiment can be C₂-C₁₂ alkenyl, C₂-C₁₂ alkenyl, C₂-C₂₀ alkenyl, C₃-C₂₀ alkenyl, C₂-C₃₀ alkenyl, or C₃-C₃₀ alkenyl.

The term “alkynyl” refers to a hydrocarbon radical straight or branched, containing at least 1 carbon to carbon triple bond, and up to the maximum possible number of non-aromatic carbon-carbon triple bonds may be present. Thus, C₂-Cₙ alkynyl is defined to include groups having 1, 2 . . . , n−1 or n carbons. For example, “C₂-C₆ alkynyl” means an alkynyl radical having 2 or 3 carbon atoms, and 1 carbon-carbon triple bond, or having 4 or 5 carbon atoms, and up to 2 carbon-carbon triple bonds, or having 6 carbon atoms, and up to 3 carbon-carbon triple bonds. Alkynyl groups include ethynyl, propynyl and butynyl. As described above with respect to alkyl, the straight or branched portion of the alkynyl group may contain triple bonds and may be substituted if a substituted alkynyl group is indicated. An embodiment can be a C₂-Cₙ alkynyl. An embodiment can be C₂-C₁₂ alkynyl or C₃-C₁₂ alkynyl, C₂-C₂₀ alkynyl, C₃-C₂₀ alkynyl, C₂-C₃₀ alkynyl, or C₃-C₃₀ alkynyl.

As used herein, “aryl” is intended to mean any stable monocyclic or bicyclic carbon ring of up to 10 atoms in each ring, wherein at least one ring is aromatic. Examples of such aryl elements include phenyl, naphthyl, tetrahydro-naphthyl, indanyl, biphenyl, phenanthryl, anthryl or acenaphthyl. In cases where the aryl substituent is bicyclic and one ring is non-aromatic, it is understood that attachment is via the aromatic ring. The substituted aryls included in this invention include substitution at any suitable position with amines, substituted amines, alkylamines, hydroxys and alkylhydroxys, wherein the “alkyl” portion of the alkylamines and alkylhydroxys is a C₂-Cₙ alkyl as defined hereinabove. The substituted amines may be substituted with alkyl, alkenyl, alkynl, or aryl groups as hereinabove defined.

The alkyl, alkenyl, alkynyl, and aryl substituents may be unsubstituted or unsubstituted, unless specifically defined otherwise. For example, a (C₁-C₆) alkyl may be substituted with one or more substituents selected from OH, oxo, halogen, alkoxy, dialkylamino, or heterocyclyl, such as morpholinyl, piperidinyl, and so on.

In the compounds of the present invention, alkyl, alkenyl, and alkynyl groups can be further substituted by replacing one or more hydrogen atoms by non-hydrogen groups described herein to the extent possible. These include, but are not limited to, halo, hydroxy, mercapto, amino, carboxy, cyano and carbamoyl.

The term “substituted” as used herein means that a given structure has a substituent which can be an alkyl, alkenyl, or aryl group as defined above. The term shall be deemed to include multiple degrees of substitution by a named substitutent. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

Examples of substituent groups include the functional groups described above, and halogens (i.e., F, Cl, Br, and I); alkyl groups, such as methyl, ethyl, n-propyl, isopropryl, n-butyl, tert-butyl, and trifluoromethyl; hydroxyl; alkoxy groups, such as methoxy, ethoxy, n-propoxy, and isopropoxy; aryloxy groups, such as phenoxy; arylalkyloxy, such as benzyloxy (phenylmethoxy) and p-trifluoromethylbenzyloxy (4-trifluoromethylphenylmethoxy); heteroaryloxy groups; sulfonyl groups, such as trifluoromethanesulfonyl, methanesulfonyl, and p-toluenesulfonyl; nitro, nitrosyl; mercapto; sulfanyl groups, such as methylsulfanyl, ethylsulfanyl and propylsulfanyl; cyano; amino groups, such as amino, methylamino, dimethylamino, ethylamino, and diethylamino; and carboxyl. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

In the compounds of the present invention, the substituents may be substituted or unsubstituted, unless specifically defined otherwise.

In the compounds of the present invention, alkyl, heteroalkyl, monocycle, bicycle, aryl, heteroaryl and heterocycle groups can be further substituted by replacing one or more hydrogen atoms with alternative non-hydrogen groups. These include, but are not limited to, halo, hydroxy, mercapto, amino, carboxy, cyano and carbamoyl.

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, a “compound” is a small molecule that does not include proteins, peptides or amino acids.

As used herein, an “isolated” compound is a compound isolated from a crude reaction mixture or from a natural source following an affirmative act of isolation. The act of isolation necessarily involves separating the compound from the other components of the mixture or natural source, with some impurities, unknown side products and residual amounts of the other components permitted to remain. Purification is an example of an affirmative act of isolation.

“Administering to the subject” or “administering to the (human) patient” means the giving of, dispensing of, or application of medicines, drugs, or remedies to a subject/patient to relieve, cure, or reduce the symptoms associated with a condition, e.g., a pathological condition. The administration can be periodic administration. As used herein, “periodic administration” means repeated/recurrent administration separated by a period of time. The period of time between administrations is preferably consistent from time to time. Periodic administration can include administration, e.g., once daily, twice daily, three times daily, four times daily, weekly, twice weekly, three times weekly, four times weekly and so on, etc.

As used herein, “administering” an agent may be performed using any of the various methods or delivery systems well known to those skilled in the art. The administering can be performed, for example, orally, parenterally, intraperitoneally, intravenously, intraarterially, transdermally, sublingually, intramuscularly, rectally, transbuccally, intranasally, liposomally, via inhalation, vaginally, intraoccularly, via local delivery, subcutaneously, intraadiposally, intraarticularly, intrathecally, into a cerebral ventricle, intraventicularly, intratumorally, into cerebral parenchyma or intraparenchchymally.

As used herein, “combination” means an assemblage of reagents for use in therapy either by simultaneous or contemporaneous administration. Simultaneous administration refers to administration of an admixture (whether a true mixture, a suspension, an emulsion or other physical combination) of the compound and the anti-cancer agent. The combination may be the admixture or separate containers that are combined just prior to administration. Contemporaneous administration refers to the separate administration, or at times sufficiently close together that a synergistic activity relative to the activity of either the alone is observed.

As used herein, “concomitant administration” or administering “concomitantly” means the administration of two agents given in close enough temporal proximately to allow the individual therapeutic effects of each agent to overlap.

As used herein, “add-on” or “add-on therapy” means an assemblage of reagents for use in therapy, wherein the subject receiving the therapy begins a first treatment regimen of one or more reagents prior to beginning a second treatment regimen of one or more different reagents in addition to the first treatment regimen, so that not all of the reagents used in the therapy are started at the same time.

The following delivery systems, which employ a number of routinely used pharmaceutical carriers, may be used but are only representative of the many possible systems envisioned for administering compositions in accordance with the invention.

Injectable drug delivery systems include solutions, suspensions, gels, microspheres and polymeric injectables, and can comprise excipients such as solubility-altering agents (e.g., ethanol, propylene glycol and sucrose) and polymers (e.g., polycaprylactones and PLGA's).

Other injectable drug delivery systems include solutions, suspensions, gels. Oral delivery systems include tablets and capsules. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc).

Implantable systems include rods and discs, and can contain excipients such as PLGA and polycaprylactone.

Oral delivery systems include tablets and capsules. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc).

Transmucosal delivery systems include patches, tablets, suppositories, pessaries, gels and creams, and can contain excipients such as solubilizers and enhancers (e.g., propylene glycol, bile salts and amino acids), and other vehicles (e.g., polyethylene glycol, fatty acid esters and derivatives, and hydrophilic polymers such as hydroxypropylmethylcellulose and hyaluronic acid).

Dermal delivery systems include, for example, aqueous and nonaqueous gels, creams, multiple emulsions, microemulsions, liposomes, ointments, aqueous and nonaqueous solutions, lotions, aerosols, hydrocarbon bases and powders, and can contain excipients such as solubilizers, permeation enhancers (e.g., fatty acids, fatty acid esters, fatty alcohols and amino acids), and hydrophilic polymers (e.g., polycarbophil and polyvinylpyrolidone). In one embodiment, the pharmaceutically acceptable carrier is a liposome or a transdermal enhancer.

Solutions, suspensions and powders for reconstitutable delivery systems include vehicles such as suspending agents (e.g., gums, zanthans, cellulosics and sugars), humectants (e.g., sorbitol), solubilizers (e.g., ethanol, water, PEG and propylene glycol), surfactants (e.g., sodium lauryl sulfate, Spans, Tweens, and cetyl pyridine), preservatives and antioxidants (e.g., parabens, vitamins E and C, and ascorbic acid), anti-caking agents, coating agents, and chelating agents (e.g., EDTA).

As used herein, “pharmaceutically acceptable carrier” refers to a carrier or excipient that is suitable for use with humans and/or animals without undue adverse side effects (such as toxicity, irritation, and allergic response) commensurate with a reasonable benefit/risk ratio. It can be a pharmaceutically acceptable solvent, suspending agent or vehicle, for delivering the instant compounds to the subject.

The compounds used in the method of the present invention may be in a salt form. As used herein, a “salt” is a salt of the instant compounds which has been modified by making acid or base salts of the compounds. In the case of compounds used to treat an infection or disease, the salt is pharmaceutically acceptable. Examples of pharmaceutically acceptable salts include, but are not limited to, mineral or organic acid salts of basic residues such as amines; alkali or organic salts of acidic residues such as phenols. The salts can be made using an organic or inorganic acid. Such acid salts are chlorides, bromides, sulfates, nitrates, phosphates, sulfonates, formates, tartrates, maleates, malates, citrates, benzoates, salicylates, ascorbates, and the like. Phenolate salts are the alkaline earth metal salts, sodium, potassium or lithium. The term “pharmaceutically acceptable salt” in this respect, refers to the relatively non-toxic, inorganic and organic acid or base addition salts of compounds of the present invention. These salts can be prepared in situ during the final isolation and purification of the compounds of the invention, or by separately reacting a purified compound of the invention in its free base or free acid form with a suitable organic or inorganic acid or base, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J. Pharm. Sci. 66:1-19).

As used herein, an “amount” or “dose” of an agent measured in milligrams refers to the milligrams of agent present in a drug product, regardless of the form of the drug product.

As used herein, the term “therapeutically effective amount” or “effective amount” refers to the quantity of a component that is sufficient to yield a desired therapeutic response without undue adverse side effects (such as toxicity, irritation, or allergic response) commensurate with a reasonable benefit/risk ratio when used in the manner of this invention. The specific effective amount will vary with such factors as the particular condition being treated, the physical condition of the patient, the type of mammal being treated, the duration of the treatment, the nature of concurrent therapy (if any), and the specific formulations employed and the structure of the compounds or its derivatives.

Where a range is given in the specification it is understood that the range includes all integers and 0.1 units within that range, and any sub-range thereof. For example, a range of 77 to 90% is a disclosure of 77, 78, 79, 80, and 81% etc.

As used herein, “about” with regard to a stated number encompasses a range of +one percent to −one percent of the stated value. By way of example, about 100 mg/kg therefore includes 99, 99.1, 99.2, 99.3, 99.4, 99.5, 99.6, 99.7, 99.8, 99.9, 100, 100.1, 100.2, 100.3, 100.4, 100.5, 100.6, 100.7, 100.8, 100.9 and 101 mg/kg. Accordingly, about 100 mg/kg includes, in an embodiment, 100 mg/kg.

It is understood that where a parameter range is provided, all integers within that range, and tenths thereof, are also provided by the invention. For example, “0.2-5 mg/kg/day” is a disclosure of 0.2 mg/kg/day, 0.3 mg/kg/day, 0.4 mg/kg/day, 0.5 mg/kg/day, 0.6 mg/kg/day etc. up to 5.0 mg/kg/day.

Each embodiment disclosed herein is contemplated as being applicable to each of the other disclosed embodiments. Thus, all combinations of the various elements described herein are within the scope of the invention.

This invention will be better understood by reference to the Experimental Details which follow, but those skilled in the art will readily appreciate that the specific experiments detailed are only illustrative of the invention as described more fully in the claims which follow thereafter.

### Experimental Details

### Abbreviations

ACN—Acetonitrile; AUCₗₐₛₜ—Area under concentration-time curve from time 0 to the last quantifiable concentration; AUC_{INF}—Area under concentration—time curve from time 0 to infinity; SQL—Below quantifiable limit; CL—Clearance; Cₘₐₓ—Maximum plasma concentration; hr or Hr—Hour; IV Intravenous; kg—Kilogram; L—Liter; LC Liquid chromatography; LLOQ—Lower limit of quantification; MeOH Methanol; mg Milligram; MS—mass spectrometry; NH₄OAc—Ammonium acetate; PK—Pharmacokinetics PO—Oral; SD Standard deviation; t_{1/2}—Terminal half—life; Tₘₐₓ—Time to reach maximum plasma concentration; Vₛₛ—Volume of distribution at steady-state

### Materials and Methods

### General Method of Preparation of Alkyl Esters

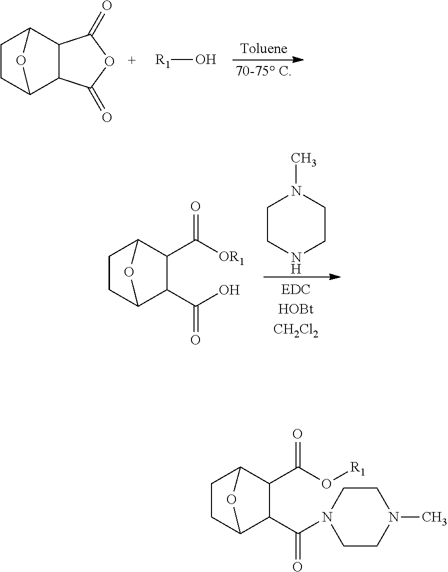

A mixture of exo-7-oxabicyclo[2.2.1]heptane-2,3-dicarboxylic anhydride (50.0 mmol) and the appropriate alkyl alcohol (110.0 mmol) in toluene is heated at 70-75° C. overnight. The reaction mixture is concentrated on rotary evaporator and the crude solid is triturated with 20 mL of isopropyl ether while heating, and filtered to give a solid. To the mixture of alkyl ester in methylene chloride is added N-hydroxybenzotriazole (5 mmol) followed by N-methylpiperazine (200 mmol) and EDC (75 mmol). The reaction mixture is stirred overnight at room temperature and evaporated to dryness. The product is purified by column chromatography and recrystallization.

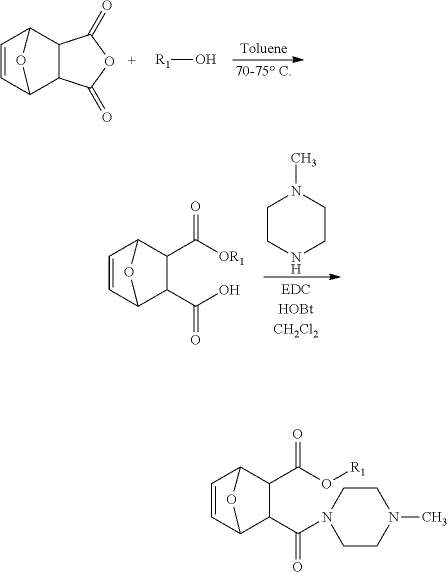

A mixture of exo-3,6-Epoxy-1,2,3,6-tetrahydrophthalic anhydride (50.0 mmol) and the appropriate alkyl alcohol (110.0 mmol) in toluene is heated at 70-75° C. overnight. The reaction mixture is concentrated on rotary evaporator and the crude solid is triturated with 20 mL of isopropyl ether while heating, and filtered to give a solid. To the mixture of alkyl ester in methylene chloride is added N-hydroxybenzotriazole (5 mmol) followed by N-methylpiperazine (200 mmol) and EDC (75 mmol). The reaction mixture is stirred overnight at room temperature and evaporated to dryness. The product is purified by column chromatography and recrystallization.

### Preparation of Propyl Ester

### 7-Oxa-bicyclo[2,2,1]heptane-2,3dicarboxylic acid monopropyl ester

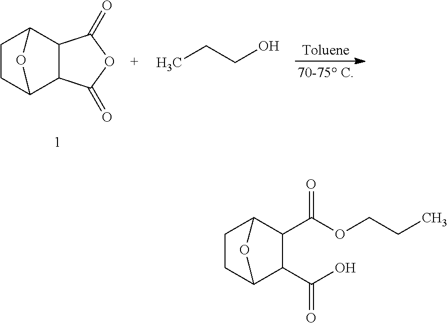

A mixture of exo-7-oxabicyclo[2.2.1]heptane-2,3-dicarboxylic anhydride (1, 8.4 g, 50.0 mmol) and n-propanol (6.6 g, 110.0 mmol) in 20 mL of toluene were heated at 70-75° C. overnight. The reaction mixture was concentrated on rotary evaporator and the crude solid was triturated with 20 mL of isopropyl ether while heating. This was cooled in ice-bath and filtered to give the propyl ester as a white solid (8.5 g, 75%). ¹H NMR (δ, ppm, CDCl₃, 300 MHz) 4.97-4.87 (m, 2H), 4.01 (m, 2H), 3.01 (m, 2H), 1.82 (m, 2H), 1.66-1.52 (m, 4H), 0.91 (t, J=7.5 Hz, 3H).

### 3-(4-Methylpiperazine-1-carbonyl)-7-oxa-bicyclo[2,2,1]heptane-2-carboxylic acid propyl ester (3, Compound 153)

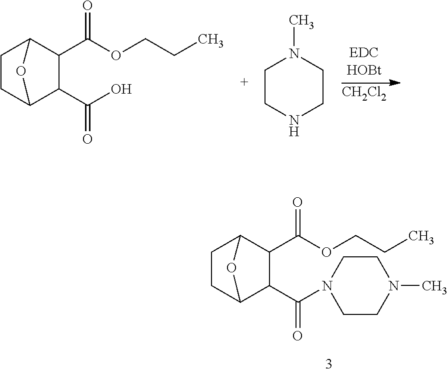

To a mixture of propyl ester 3 (5.00 g, 22.3 mmol) in methylene chloride was added N-hydroxybenzotriazole (0.30 g, 2.23 mmol) followed by N-methylpiperazine (4, 5.25 g, 100.16 mmol) and EDC (5.19 g, 33.45 mmol). The reaction mixture was stirred overnight at room temperature and was evaporated to dryness. The product was purified by column chromatography using 5% methanol in methylene chloride to give 5.9 g of oil. Recrystallization from dichloromethane and hexanes at 0-5° C. gave a crystalline solid. This was filtered to give pure ester 3 (5.1 g, 71%). ¹H NMR (δ, ppm, CDCl₃, 300 MHz) 4.91 (bs, 2H), 3.99 (m, 2H), 3.75 (m, 1H), 3.51-3.71 (m, 3H), 3.06 (d, J=9.3 Hz, 1H), 2.92 (d, J=9.3 Hz, 1H), 2.44 (m, 2H), 2.29 (m, 5H), 1.82-1.75 (m, 2H), 1.62 (q, J=7.2, 7.2, 2H), 1.51 (m, 2H), 0.91 (t, J=7.5, 3H). mp=94-95° C. ESI-MS (m/z): 311.2 [M+H]⁺.

### Preparation of Heptyl Ester

### 7-Oxa-bicyclo [2,2,1]heptane-2,3dicarboxylic acid monoheptyl ester

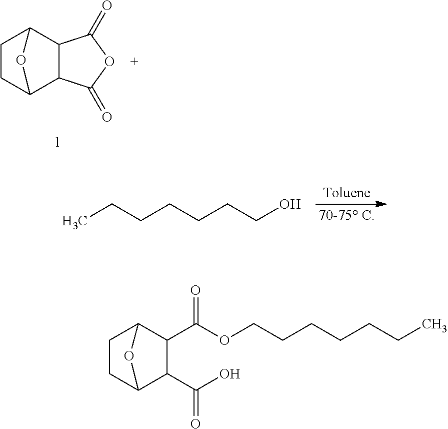

A mixture of exo-7-oxabicyclo[2.2.1]heptane-2,3-dicarboxylic anhydride (1, 8.4 g, 50.0 mmol) and n-heptyl alcohol (6.6 g, 57.6 mmol) in 20 mL of toluene was heated at ˜75° C. overnight. The reaction mixture was concentrated and the crude solid was triturated with 6 mL of toluene while heating. This was cooled in an ice-bath and filtered to give the heptyl ester as a white solid (8.5 g, 60%). ¹H NMR (δ, ppm, CDCl₃, 300 MHz) 4.94 (m, 2H), 4.03 (m, 2H), 3.00 (m, 2H), 1.84 (m, 2H), 1.58 (m, 4H), 1.52 (m, 8H), 0.87 (m, 3H)

### 3-(4-Methylpiperazine-1-carbonyl)-7-oxa-bicyclo[2,2,1]heptane-2-carboxylic acid heptyl ester (7, Compound 157)

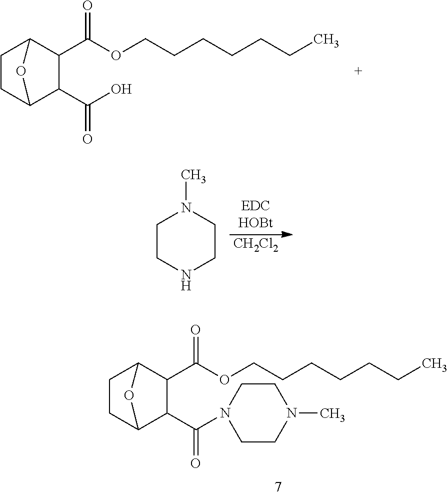

To a mixture of heptyl ester (5.68 g, 20.0 mmol) in methylene chloride was added N-hydroxybenzotriazole (0.27 g, 2.00 mmol) followed by N-methylpiperazine (4.7 g, 47.0 mmol) and EDC (5.75 g, 30.0 mmol). The reaction mixture was stirred overnight at room temperature and evaporated to dryness. The product was purified by column chromatography using 5% methanol in methylene chloride to give 6.5 g of oil which was recrystallized from a mixture of diisopropyl ether and hexanes at 0-5° C. to give colorless crystals of 8. It was filtered to give pure ester 7 (4.96 g, 71%). ¹H NMR (δ, ppm, CDCl₃, 300 MHz) 4.90 (m, 2H), 4.03 (m, 2H), 3.76 (m, 1H), 3.49 (m, 1H), 3.37 (m, 2H), 3.06 (d, J=9.3, 1H), 2.91 (d, J=9.3, 1H), 2.44 (m, 2H), 2.32 (m, 5H), 1.80 (m, 2H), 1.61-1.46 (m, 4H), 1.26 (m, 8H), 0.87 (t, J=6.3, 3H). mp 68-69° C. ESI-MS (m/z): 367.3 [M+H]⁺.

### Preparation of Heptadecyl Ester

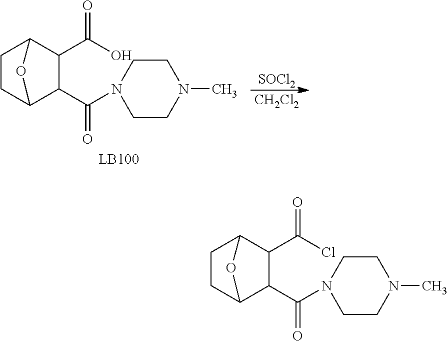

To an ice-cold slurry of 3-(4-methylpiperazine-1-carbonyl)-7-oxa-bicyclo[2,2,1]-heptane-2-carboxylic acid (Compound 100, 2.5 g, 9.3 mole) in methylene chloride (40 mL) was added thionyl chloride (2.5 mL) followed by a few drops of DMF. After stirring at ice-cold temperature for 30 min, the ice-bath was removed and stirring continued at room temperature overnight. The excess thionyl chloride was removed using oil-free vacuum pump at ˜50° C. and to the residue was added methylene chloride (10 mL). The resulted thin slurry of acid chloride was used as such in the next reaction.

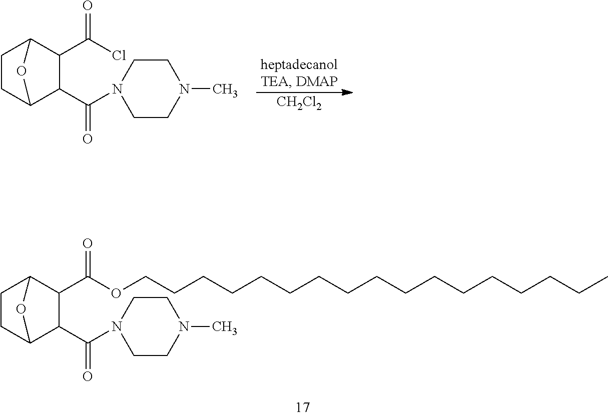

### 3-(4-Methylpiperazine-1-carbonyl)-7-oxa-bicyclo [2,2,1]heptane-2-carboxylic acid linoleyl ester (17, Compound 159)

To an ice-cold solution of heptadecanol (2.0 g, 7.8 mmole) in methylene chloride (20 mL) and TEA (3 mL, 20 mmole) was added the above suspension of acid chloride (9.3 mmole) in methylene chloride (20 mL). After stirring for 10 minutes at ice bath temperature, the ice-bath was removed and stirring continued at room temperature for 4 h. The reaction mixture was then washed with water (2×8 mL) followed by brine (10 mL), dried over anhydrous sodium sulfate, filtered and concentrated. The crude residue was purified by column chromatography using 5% methanol in methylene chloride to give the pure required compound 17 (1.1 g, 27%) as a off white solid, m.p. 110-112° C. ¹H NMR (CDCl₃) δ 0.87 (t, d, J=7.2 Hz, 3H), 1.24 (m, 28H), 1.40-1.51 (m, 4H), 1.60-1.79 (m, 2H), 2.55 (s, 3H), 2.75 (m, 3H), 2.85-3.06 (m, 4H), 3.60-3.85 (m, 3H), 4.03 (t, d, J=7.2 Hz, 2H), 4.88 (m, 1H), 4.94 (m, 1H); ESMS: 507 (M+H).

### Preparation of Linoleyl Ester

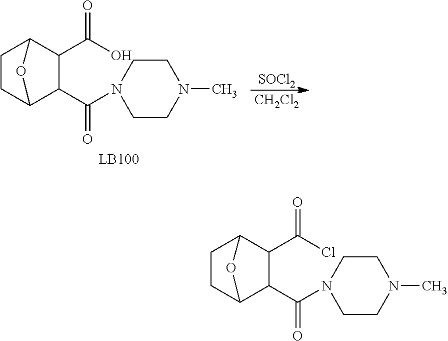

To an ice-cold slurry of 3-(4-methylpiperazine-1-carbonyl)-7-oxa-bicyclo[2,2,1]-heptane-2-carboxylic acid (Compound 100, 2.5 g, 9.3 mole) in methylene chloride (40 mL) was added thionyl chloride (2.5 mL) followed by a few drops of DMF. After stirring at ice-cold temperature for 30 min, the ice-bath was removed and stirring continued at room temperature overnight. The excess thionyl chloride was removed using oil-free vacuum pump at ˜50° C. and to the residue was added methylene chloride (10 mL). The resulted thin slurry of acid chloride was used as such in the next reaction.

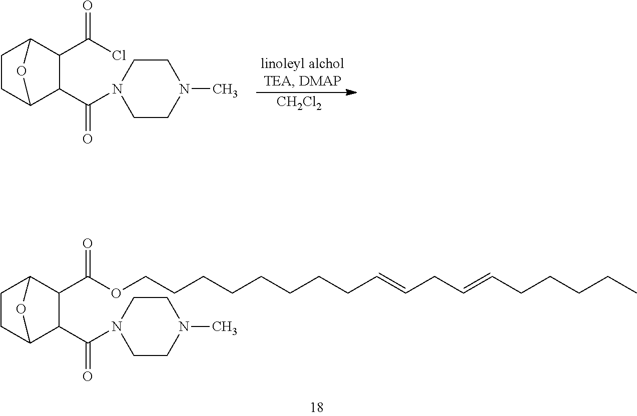

### 3-(4-Methylpiperazine-1-carbonyl)-7-oxa-bicyclo [2,2,1] heptane-2-carboxylic acid linoleyl ester (18, Compound 158)

To an ice-cold solution of linoleyl alcohol (3, 2.0 g, 7.5 mmole) in methylene chloride (20 mL) and TEA (3 mL, 20 mmole) was added the above suspension of acid chloride (9.3 mmole) in methylene chloride (20 mL). After stirring for 10 minutes at ice bath temperature, the ice-bath was removed and stirring continued at room temperature for 1 h. At this time the TLC (95:7:CH₂C₁₂:MeOH) showed the disappearance of linoleyl alcohol. The reaction mixture was then washed with water (2×10 mL) followed by brine (10 mL), dried over anhydrous sodium sulfate, filtered and concentrated. The crude residue was purified by column chromatography using 5% methanol in methylene chloride to give the pure required compound 18 (0.2 g, 5.2%) as an oil. ¹H NMR (CDCl₃) δ 0.86 (t, d, J=6.9 Hz, 3H), 1.29 (m, 17H), 1.55 (m, 2H), 1.81 (m, 2H), 2.03 (m, 4H), 2.30 (s, 3H), 2.45 (m, 2H), 2.77 (t, J=6 Hz, 2H), 2.89-3.07 (m, 3H), 3.40 (m, 2H), 3.49 (m, 2H), 3.99 (m, 2H), 4.05 (m, 2H), 4.91 (m, 2H), 5.30-5.37 (m, 4H); ESMS: 517 (M+H).

The following are additional synthetic routes used to prepare the compounds of the present application. The following synthetic routes may be modified by one of ordinary skill in the art to prepare additional compounds disclosed herein.

A mixture of exo-7-oxabicyclo[2.2.1]heptane-2,3-dicarboxylic anhydride and the appropriate diethyl phosphate derived amine or alcohol is dissolved in toluene was heated at ˜75° C. overnight. The reaction mixture is concentrated and purified by chromatography to afford the desired acids derivative.

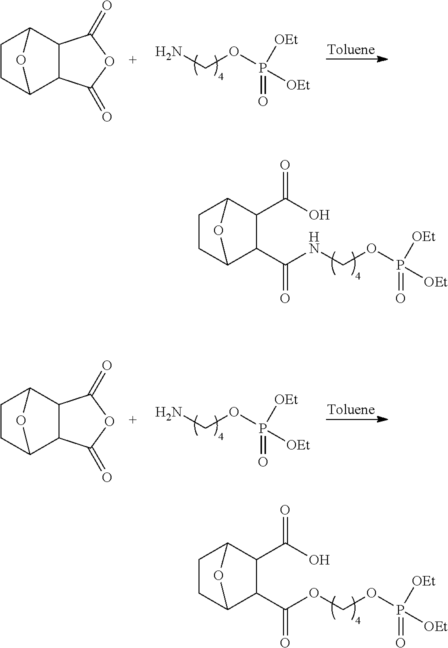

The above acids are further derivatized by conversion to the corresponding acid chloride with thionyl chloride followed by addition of methanol in the presence of base.

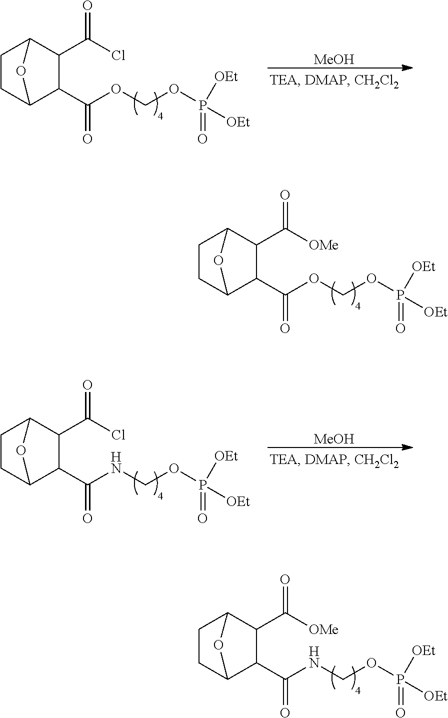

To an ice-cold solution of the appropriate diethyl phosphate derived amine or alcohol and TEA was added the acid chloride derivative of compound 100 in methylene chloride, After stirring for 10 minutes at ice bath temperature, the ice-bath was removed and stirring continued at room temperature for 4 h. The reaction mixture was then washed with water (2×8 mL) followed by brine (10 ml), dried over anhydrous sodium sulfate, filtered and concentrated. The crude residue was purified by chromatography to give the desired compound.

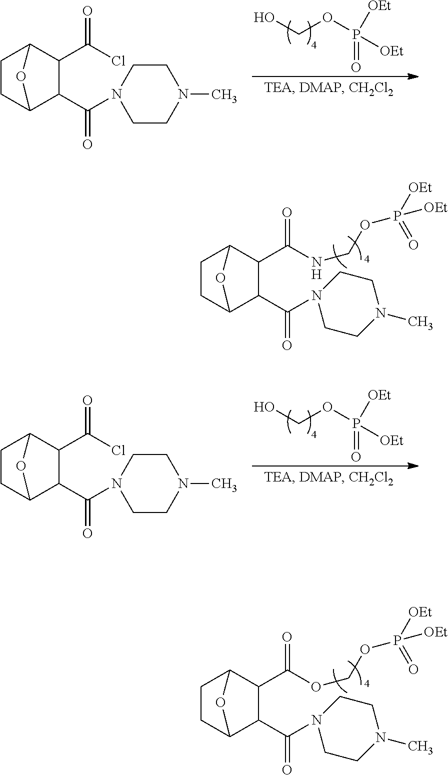

### Reagents

Coomassie (Bradford) Protein Assay Kit (Pierce); PP2A Immunoprecipitation Phosphatase Assay Kit (Millipore); Lysate preparation for low endogenous phosphate: 20 mM imidazole-HCl, 2 mM EDTA, 2 mM EGTA, 1 mM PMSF, 1 mM benzamidine, 10 ug/ml each of aprotinin, leupeptin, antipain, soybean trypsin inhibitor; Normal Mouse IgG (Millipore); Okadaic acid (OA) (Tocris); DMSO (Sigma).

### Animals

### Animal Specifications

Species: Mus Musculus
Strain: Balb/c mice
Age: 6-8 weeks
Sex: female
Body weight: 18-22 g
Vendor: Shanghai Laboratory Animal Center, Shanghai, China;
Number of animals: 66 Balb/c mice plus spare

### Animal Husbandry

The mice were kept in laminar flow rooms at constant temperature and humidity with 4 animals in each cage.
Temperature: 20-25
Humidity: 40-70%.
Light cycle: 12 hours light and 12 hours dark.
Cages: Made of polycarbonate. The size is 29 cm×17.5 cm×12 cm (L×w×H). The bedding material is wood debris, which is changed once per week.
Diet: Animals had free access to irradiation sterilized dry granule food during the entire study period.
Water: Animals had free access to sterile drinking water.
Cage identification: the identification labels for each cage contained the following information: number of animals, sex, strain, date arrival, treatment, study number, group number, and the starting date of the treatment.
Animal identification: Animals were marked by ear punch.

### Animal Procedure

All the procedures related to animal handling, care, and the treatment in this study were performed according to guidelines approved by the Institutional Animal Care and Use Committee (IACUC) of WuXi AppTec (Shanghai), following the guidance of the Association for Assessment and Accreditation of Laboratory Animal Care (AAALAC). At the time of routine monitoring, the animals were checked and recorded for any effects of tumor growth on normal behavior such as mobility, food and water consumption (by looking only), body weight gain/loss, eye/hair matting and any other abnormal effect.

### Formulation and Administration

1. Proper amount of the compounds were weighed.
2. The compounds were dissolved in 4% sterile sodium bicarbonate.
3. All of the compounds should be well dissolved and clear.
4. The compounds should be kept cold once in solution and injected within an hour.
5. The mice were administered intraperitoneally according to their body weights, 20 g mouse was treated with 0.2 ml compound solution.
6. Mice were treated with vehicle and compounds according to Table 1.
7. 3 hours after the dose, 3 mice from each group were euthanized by

CO₂ exposure, and the brains and left lobe of livers were taken and snap-frozen in liquid nitrogen immediately. 6 hours after the dose, the other 3 mice from each group were euthanized by CO₂ exposure, and the brains and left lobe of livers were taken and snap-frozen in liquid nitrogen immediately.

TABLE 1
Experimental Design

| | Dosage | Dosing | Dosing | Dosing | Animal |
|---|---|---|---|---|---|
| Treatment | (mg/kg) | Route | volume | Schedule | number |
| Vehicle | — | IP | 10 mL/kg | Once | 6 |
| (4% NaHCO₃) | | | | | |
| 100 | 0.75 | IP | 10 mL/kg | Once | 6 |
| 100 | 1.5 | IP | 10 mL/kg | Once | 6 |
| 113 | 1.1 | IP | 10 mL/kg | Once | 6 |
| 113 | 2.2 | IP | 10 mL/kg | Once | 6 |
| 151 | 0.8 | IP | 10 mL/kg | Once | 6 |
| 151 | 1.6 | IP | 10 mL/kg | Once | 6 |
| 153 | 0.85 | IP | 10 mL/kg | Once | 6 |
| 153 | 1.7 | IP | 10 mL/kg | Once | 6 |
| 157 | 1.0 | IP | 10 mL/kg | Once | 6 |
| 157 | 2.0 | IP | 10 mL/kg | Once | 6 |

Compounds 100 and 151 are disclosed in U.S. Pat. No. 7,998,957, the contents of which are hereby incorporated by reference. Compound 151 is identical to compound 107 disclosed in U.S. Pat. No. 7,998,957. Compound 113 is disclosed in U.S. Pat. No. 8,227,473, the contents of which are hereby incorporated by reference. Compound 105 is also disclosed in U.S. Pat. No. 7,998,957.

### PP2A Activity Detecting

Preparation of malachite green phosphate detection solution Added 10 mL of Solution B to each 1 mL of Solution A, kept at room temperature during use. 100 mL of mixed solution AB is used per assay well.

### Phosphate Standard Curve

Diluted 125 mL Phosphate Standard (Solution C) with 1125 mL of distilled water to make 0.1 mM working solution. The solution was used to prepare a phosphate standard curve as described in the table below.

TABLE 2
Phosphate standard curve

| | | | | | | | | | | | |
|---|---|---|---|---|---|---|---|---|---|---|---|
| Volume of | 200 uL | 180 uL | 160 uL | 140 uL | 120 uL | 100 uL | 80 uL | 60 uL | 40 uL | 20 uL | 0 uL |
| diluted stock | | | | | | | | | | | |
| Volume of | 50 uL | 70 uL | 90 uL | 110 uL | 130 uL | 150 uL | 170 uL | 190 uL | 210 uL | 230 uL | 250 uL |
| distilled water | | | | | | | | | | | |
| Picomoles of | 2000 | 1800 | 1600 | 1400 | 1200 | 1000 | 800 | 600 | 400 | 200 | 0 |
| Phosphate | | | | | | | | | | | |
| per 25 mL | | | | | | | | | | | |

1. 25 μL of each phosphate standards was transferred to wells of microliter plate.
2. Added 100 μL of Malachite Green Solution AB. Mixed carefully without creating bubbles.
3. Cultured for 15 minutes at RT.
4. Measured absorbance at a wavelength between 650 nm in a microliter plate reader.

### Phosphopeptide Preparation

1. Dissolved 1 mg Threonine Phosphopeptide (Catalog #12-219) in 1.10 mL of distilled water to prepare a 1 mM solution.
2. Aliquot peptide solution and stored at ˜20° C. as necessary.

### Enzyme Assay

1. Mouse brain or liver was homogenized using lysis buffer (25 g/L), centrifuged at 12000 g for 10 minutes at 4M, and the supernatants were collected.
2. The protein was quantitated, and 240 μg of mouse brain or liver lysate was taken to assay phosphatase activity.
3. Added 4 μg of Anti-PP2A or 4 μg Normal mouse IgG as an IP control.
4. Added 30 μl Protein A agarose slurry.
5. Brought volume to 500 μl with pNPP Ser/Thr Assay Buffer.
6. Incubated for 2 h at 4° C. with constant rocking.
7. Washed beads 3 times with 700 μl TBS, followed by one wash with 500 μl Ser/Thr Assay Buffer.
8. Added 20 μl of Ser/Thr Assay Buffer. (In order to determine the linear range of PP2A amount for enzymatic reaction, different amounts of precipitated PP2A were used for subsequent phosphatase assay.)
9. Added 60 μl of diluted phosphopeptide (final concentration would be 750 μM)
10. Added OA (5 μM & 5 nM) or DMSO to the reaction system.
11. Incubated for 10 minutes at 30° C. in a shaking incubator.
12. Centrifuged briefly and transferred 25 μl into each well of the microliter plate to be used.

### 13. Added 100 μl of Malachite Green Phosphate Detection Solution AB.

14. Let color develop for 10-15 minutes at RT.
15. Measured absorbance at a wavelength between 650 not in a microliter plate reader.

### Analysis of PP2A Activity in Mouse Livers

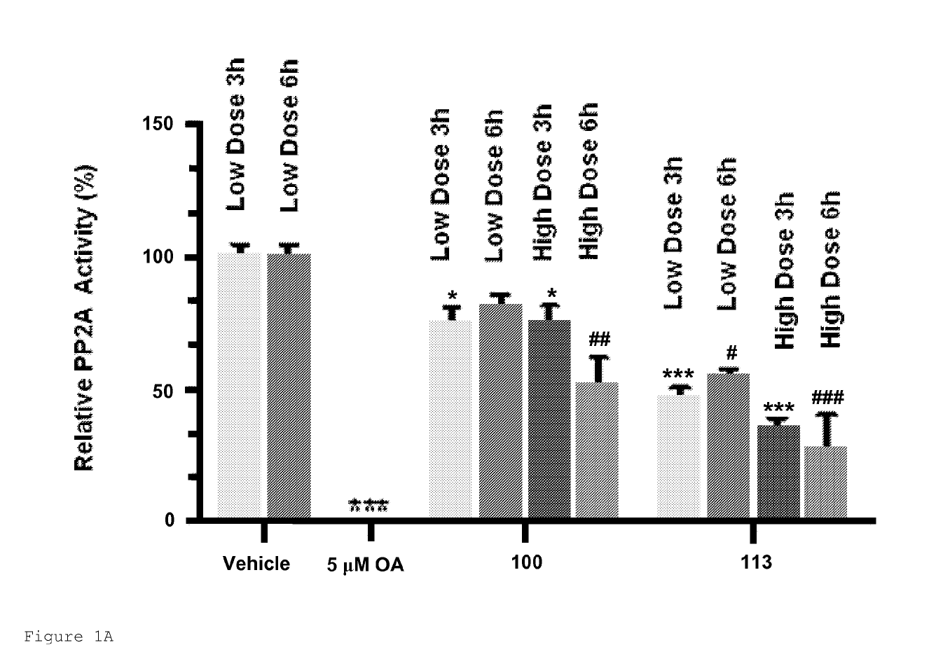

The activity of PP2A was assessed by the concentration of phosphate. As shown in Table 3 and FIG. 1, the results revealed that all the compounds at high doses significantly inhibited the activity of PP2A in livers at 6 h post treatment as compared with vehicle, compound 113 at both low and high doses significantly inhibited the activity of PP2A in livers at both 3 h and 6 h, positive control OA significantly inhibited the activity of PP2A in livers.

TABLE 3
PP2A activity in mouse livers

| | | | | Mean | | Average | Relative | Relative | Average |
|---|---|---|---|---|---|---|---|---|---|
| Group | | Sample ID | Conc. | Conc. | | Conc. | Conc. | Activity | Acti |
| IgG | | M1-1 | 51.02 | 50.37 | 0 | 0 | 0 | 0 | 0 |
| | | | 49.73 | | | | | | |
| 5 μM OA | | M1-1 | 52.75 | 52.21 | 1.84 | 1.84 | 0 | 0.47 | 0.47 |
| | | | 51.67 | | | | | | |
| Vehicle | 3 h | M1-1 | 460.01 | 457.64 | 407.26 | 392.4 | 1.04 | 100 | 100 |
| | | | 455.26 | | | | | | |
| | | M1-2 | 407.97 | 406.35 | 355.98 | | 0.91 | | |
| | | | 404.73 | | | | | | |
| | | M1-3 | 445.98 | 464.33 | 413.96 | | 1.05 | | |
| | | | 482.69 | | | | | | |
| | 6 h | M2-1 | 479.45 | 466.17 | 415.79 | 391.61 | 1.06 | 100 | 100 |
| | | | 452.89 | | | | | | |
| | | M2-2 | 402.57 | 406.46 | 356.09 | | 0.91 | | |
| | | | 410.35 | | | | | | |
| | | M2-3 | 449.86 | 453.32 | 402.95 | | 1.03 | | |
| | | | 456.77 | | | | | | |
| 100 | 3 h | M3-1 | 292.01 | 300.43 | 250.06 | 289.29 | 0.64 | 63.73 | 73.7 |
| (0.75 mpk) | | | 308.85 | | | | | | |
| | | M3-2 | 318.57 | 324.08 | 273.7 | | 0.7 | 69.75 | |
| | | | 329.58 | | | | | | |
| | | M3-3 | 359.6 | 394.47 | 344.1 | | 0.88 | 87.69 | |
| | | | 429.35 | | | | | | |
| | 6 h | M4-1 | 391.13 | 403 | 352.63 | 316.5 | 0.9 | 90.05 | 80.8 |
| | | | 414.88 | | | | | | |
| | | M4-2 | 358.3 | 358.74 | 308.36 | | 0.79 | 78.74 | |
| | | | 359.17 | | | | | | |
| | | M4-3 | 328.94 | 338.87 | 288.5 | | 0.74 | 73.67 | |
| | | | 348.8 | | | | | | |
| 100 | 3 h | M5-1 | 340.38 | 347.18 | 296.81 | 290.15 | 0.76 | 75.64 | 73.9 |
| (1.5 mpk) | | | 353.99 | | | | | | |
| | | M5-2 | 282.08 | 387.56 | 337.19 | | 0.86 | 85.93 | |
| | | | 493.05 | | | | | | |
| | | M5-3 | 279.92 | 286.83 | 236.46 | | 0.6 | 60.26 | |
| | | | 293.74 | | | | | | |
| | 6 h | M6-1 | 154.46 | 160.83 | 110.45 | 197.91 | 0.28 | 28.21 | 50.5 |
| | | | 167.2 | | | | | | |
| | | M6-2 | 290.5 | 307.23 | 256.86 | | 0.66 | 65.59 | |
| | | | 323.97 | | | | | | |
| | | M6-3 | 262.21 | 276.79 | 226.41 | | 0.58 | 57.82 | |
| | | | 291.36 | | | | | | |
| 113 | 3 h | M7-1 | 258.54 | 262.43 | 212.05 | 179.91 | 0.54 | 54.04 | 45.9 |
| (1.1 mpk) | | | 266.31 | | | | | | |
| | | M7-2 | 211.9 | 201.1 | 150.73 | | 0.38 | 38.41 | |
| | | | 190.3 | | | | | | |
| | | M7-3 | 226.36 | 227.34 | 176.96 | | 0.45 | 45.1 | |
| | | | 228.31 | | | | | | |
| | 6 h | M8-1 | 229.82 | 242.13 | 191.76 | 212.74 | 0.49 | 48.97 | 54.3 |
| | | | 254.44 | | | | | | |
| | | M8-2 | 279.7 | 285.64 | 235.27 | | 0.6 | 60.08 | |
| | | | 291.58 | | | | | | |
| | | M8-3 | 266.31 | 261.56 | 211.19 | | 0.54 | 53.93 | |
| | | | 256.81 | | | | | | |
| 113 | 3 h | M9-1 | 200.67 | 202.61 | 152.24 | 138.31 | 0.39 | 38.8 | 35.3 |
| (2.2 mpk) | | | 204.55 | | | | | | |
| | | M9-2 | 192.25 | 204.23 | 153.86 | | 0.39 | 39.21 | |
| | | | 216.22 | | | | | | |
| | | M9-3 | 155.1 | 159.21 | 108.83 | | 0.28 | 27.74 | |
| | | | 163.31 | | | | | | |
| | 6 h | M10-1 | 246.88 | 258.43 | 208.06 | 104.16 | 0.53 | 53.13 | 26.6 |
| | | | 269.98 | | | | | | |
| | | M10-2 | 80.17 | 79.31 | 28.94 | | 0.07 | 7.39 | |
| | | | 78.45 | | | | | | |
| | | M10-3 | 124.87 | 125.84 | 75.47 | | 0.19 | 19.27 | |
| | | | 126.82 | | | | | | |
| | | | | | | | | Relative | Average |
| | | | | Mean | | Average | Relative | PP2A | Relative |
| Group | | Sample ID | Conc. | Conc. | | Conc. | Conc. | Activity | Activity |
| IgG | | M1-1 | 45.7 | 45.48 | 0 | 0 | 0.00 | 0 | 0 |
| | | | 45.26 | | | | | | |
| 5 μM OA | | M1-1 | 66.6 | 62.68 | 12.31 | 0.03 | 0.03 | 2.93 | 2.93 |
| | | | 58.76 | | | | | | |
| Vehicle | 3 h | M1-1 | 318.73 | 319.6 | 269.23 | 419.97 | 0.64 | 100 | 100 |
| | | | 320.47 | | | | | | |
| | | M1-2 | 539.07 | 551.04 | 500.67 | | 1.19 | | |
| | | | 563.02 | | | | | | |
| | | M1-3 | 537.33 | 540.37 | 490 | | 1.17 | | |
| | | | 543.42 | | | | | | |
| | 6 h | M2-1 | 485.51 | 507.5 | 457.12 | 444.5 | 1.03 | 100 | 100 |
| | | | 529.49 | | | | | | |
| | | M2-2 | 543.42 | 554.96 | 504.59 | | 1.14 | | |
| | | | 566.5 | | | | | | |
| | | M2-3 | 415.4 | 422.15 | 371.77 | | 0.84 | | |
| | | | 428.9 | | | | | | |
| 151 | 3 h | M11-1 | 430.64 | 440 | 394.52 | 423.92 | 0.94 | 93.94 | 100.94 |
| (0.8 mpk) | | | 449.36 | | | | | | |
| | | M11-2 | 437.61 | 442.18 | 396.7 | | 0.94 | 94.46 | |
| | | | 446.75 | | | | | | |
| | | M11-3 | 521.21 | 526 | 480.53 | | 1.14 | 114.42 | |
| | | | 530.8 | | | | | | |
| | 6 h | M12-1 | 428.03 | 443.27 | 397.79 | 307.58 | 0.89 | 89.49 | 69.2 |
| | | | 458.51 | | | | | | |
| | | M12-2 | 260.81 | 270.39 | 224.91 | | 0.51 | 50.6 | |
| | | | 279.97 | | | | | | |
| | | M12-3 | 333.1 | 345.51 | 300.03 | | 0.67 | 67.5 | |
| | | | 357.92 | | | | | | |
| 151 | 3 h | M13-1 | 387.96 | 402.99 | 357.51 | 472.47 | 0.85 | 85.13 | 112.5 |
| (1.6 mpk) | | | 418.01 | | | | | | |
| | | M13-2 | 367.5 | 386.01 | 340.53 | | 0.81 | 81.08 | |
| | | | 404.51 | | | | | | |
| | | M13-3 | 757.23 | 764.85 | 719.38 | | 1.71 | 171.29 | |
| | | | 772.47 | | | | | | |
| | 6 h | M14-1 | 320.03 | 322.65 | 277.17 | 241.1 | 0.62 | 62.36 | 54.24 |
| | | | 325.26 | | | | | | |
| | | M14-2 | 287.37 | 299.35 | 253.87 | | 0.57 | 57.11 | |
| | | | 311.32 | | | | | | |
| | | M14-3 | 231.2 | 237.73 | 192.25 | | 0.43 | 43.25 | |
| | | | 244.26 | | | | | | |
| 153 | 3 h | M15-1 | 445.88 | 453.94 | 408.46 | 387.63 | 0.97 | 97.26 | 92.3 |
| (0.85 mpk) | | | 461.99 | | | | | | |
| | | M15-2 | 441.96 | 440.22 | 394.74 | | 0.94 | 93.99 | |
| | | | 438.48 | | | | | | |
| | | M15-3 | 396.24 | 405.17 | 359.69 | | 0.86 | 85.65 | |
| | | | 414.09 | | | | | | |
| | | M16-1 | 479.41 | 461.77 | 416.3 | 364.7 | 0.94 | 93.66 | 82.05 |
| | | | 444.14 | | | | | | |
| | | M16-2 | 471.14 | 452.63 | 407.15 | | 0.92 | 91.6 | |
| | 6 h | | 434.12 | | | | | | |
| | | M16-3 | 338.76 | 316.11 | 270.64 | | 0.61 | 60.89 | |
| | | | 293.47 | | | | | | |
| 157 | | M19-1 | 336.15 | 338.1 | 292.63 | 328.84 | 0.7 | 69.68 | 78.3 |
| (1.0 mpk) | 3 h | | 340.06 | | | | | | |
| | | M19-2 | 323.95 | 324.17 | 278.69 | | 0.66 | 66.36 | |
| | | | 324.39 | | | | | | |
| | | M19-3 | 475.49 | 460.69 | 415.21 | | 0.99 | 98.87 | |
| | | | 445.88 | | | | | | |
| | | M20-1 | 360.53 | 370.98 | 325.51 | 359.62 | 0.73 | 73.23 | 80.9 |
| | 6 h | | 381.43 | | | | | | |
| | | M20-2 | 422.37 | 401.68 | 356.2 | | 0.8 | 80.14 | |
| | | | 381 | | | | | | |
| | | M20-3 | 439.78 | 442.61 | 397.14 | | 0.89 | 89.35 | |
| | | | 445.45 | | | | | | |
| 157 | 3 h | M21-1 | 344.85 | 352.69 | 307.22 | 234.28 | 0.73 | 73.15 | 55.78 |
| (2.0 mpk) | | | 360.53 | | | | | | |
| | | M21-2 | 257.33 | 272.57 | 227.09 | | 0.54 | 54.07 | |
| | | | 287.81 | | | | | | |
| | | M21-3 | 202.46 | 214 | 168.52 | | 0.4 | 40.13 | |
| | | | 225.54 | | | | | | |
| | 6 h | M22-1 | 266.91 | 240.34 | 194.87 | 232.03 | 0.44 | 43.84 | 52.2 |
| | | | 213.78 | | | | | | |
| | | M22-2 | 382.3 | 397.11 | 351.63 | | 0.79 | 79.11 | |
| | | | 411.92 | | | | | | |
| | | M22-3 | 190.7 | 195.06 | 149.58 | | 0.34 | 33.65 | |
| | | | 199.41 | | | | | | |

### Analysis of PP2A Activity in Mouse Brains

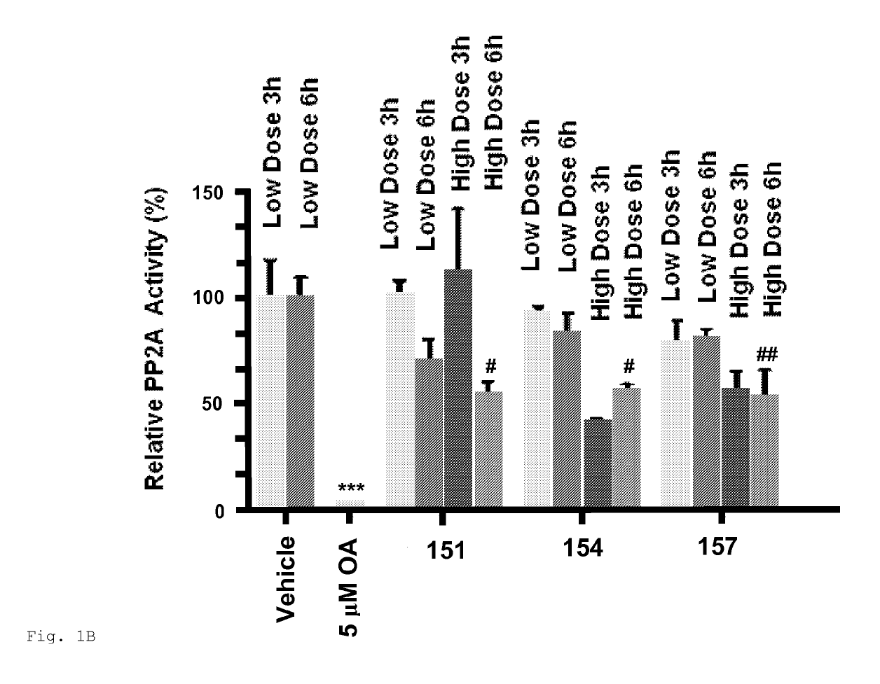

The activity of PP2A was assessed by the concentration of phosphate. As shown in Table 4 and FIG. 2, the results indicated that all the compounds inhibited the activity of PP2A in brains to some extent while the most potent ones were compound 113 high dose at 3 h and compound 157 low dose at 6 h and high dose at both 3 h and 6 h, positive control OA significantly inhibited the activity of PP2A in brains.

TABLE 4
PP2A activity in mouse brains

| | | | | | | | | Relative | Average |
|---|---|---|---|---|---|---|---|---|---|
| | | | | Mean | | Average | Relative | PP2A | Relative |
| Group | | Sample ID | Conc. | Conc. | | Conc. | Conc. | Activity | Activity |
| IgG | | M1-1 | 2.27 | 6.37 | 0.00 | 0.00 | 0.00 | 0.00 | 0.00 |
| | | | 10.46 | | | | | | |
| 5 μM OA | | M1-1 | 2.99 | 0.82 | −5.54 | −5.54 | 0.00 | −0.38 | −0.38 |
| | | | −1.35 | | | | | | |
| Vehicle | 3 h | M1-1 | 1468.05 | 1475.40 | 1469.04 | 1453.17 | 1.01 | 101.09 | 100.00 |
| | | | 1482.75 | | | | | | |
| | | M1-2 | 1455.28 | 1458.17 | 1451.80 | | 1.00 | 99.91 | |
| | | | 1461.06 | | | | | | |
| | | M1-3 | 1438.65 | 1445.03 | 1438.67 | | 0.99 | 99.00 | |
| | | | 1451.42 | | | | | | |
| | 6 h | M2-1 | 1268.98 | 1276.81 | 1270.45 | 1379.74 | 0.92 | 92.08 | 100.00 |
| | | | 1284.65 | | | | | | |
| | | M2-2 | 1356.47 | 1383.58 | 1377.21 | | 1.00 | 99.82 | |
| | | | 1410.69 | | | | | | |
| | | M2-3 | 1445.88 | 1497.94 | 1491.57 | | 1.08 | 108.10 | |
| | | | 1549.99 | | | | | | |
| 100 (0.75 mpk) | 3 h | M3-1 | 1131.61 | 1140.17 | 1133.80 | 1143.44 | 0.78 | 77.18 | 77.84 |
| | | | 1148.72 | | | | | | |
| | | M3-2 | 982.91 | 1014.72 | 1008.36 | | 0.69 | 68.64 | |
| | | | 1046.54 | | | | | | |
| | | M3-3 | 1270.67 | 1294.53 | 1288.16 | | 0.89 | 87.69 | |
| | | | 1318.39 | | | | | | |
| | 6 h | M4-1 | 996.89 | 1010.51 | 1004.14 | 1009.84 | 0.73 | 71.99 | 72.40 |
| | | | 1024.12 | | | | | | |
| | | M4-2 | 978.81 | 1026.17 | 1019.81 | | 0.74 | 73.11 | |
| | | | 1073.53 | | | | | | |
| | | M4-3 | 990.38 | 1011.95 | 1005.59 | | 0.73 | 72.09 | |
| | | | 1033.52 | | | | | | |
| 100 (1.5 mpk) | 3 h | M5-1 | 1090.16 | 1118.11 | 1111.75 | 1176.58 | 0.77 | 75.68 | 80.09 |
| | | | 1146.07 | | | | | | |
| | | M5-2 | 1273.56 | 1311.40 | 1305.03 | | 0.90 | 88.84 | |
| | | | 1349.24 | | | | | | |
| | | M5-3 | 1116.43 | 1119.32 | 1112.95 | | 0.77 | 75.76 | |
| | | | 1122.21 | | | | | | |
| | 6 h | M6-1 | 1524.93 | 1550.84 | 1544.47 | 1430.23 | 1.12 | 110.73 | 102.54 |
| | | | 1576.74 | | | | | | |
| | | M6-2 | 1180.05 | 1201.98 | 1195.62 | | 0.87 | 85.72 | |
| | | | 1223.91 | | | | | | |
| | | M6-3 | 1496.73 | 1556.98 | 1550.62 | | 1.12 | 111.17 | |
| | | | 1617.23 | | | | | | |
| 113 (1.1 mpk) | 3 h | M7-1 | 1602.05 | 1611.21 | 1604.84 | 1540.65 | 1.10 | 109.24 | 104.88 |
| | | | 1620.37 | | | | | | |
| | | M7-2 | 1450.70 | 1514.81 | 1508.44 | | 1.04 | 102.68 | |
| | | | 1578.91 | | | | | | |
| | | M7-3 | 1521.79 | 1515.05 | 1508.68 | | 1.04 | 102.70 | |
| | | | 1508.30 | | | | | | |
| | 6 h | M8-1 | 1379.36 | 1417.80 | 1411.44 | 1212.05 | 1.02 | 101.19 | 86.90 |
| | | | 1456.24 | | | | | | |
| | | M8-2 | 1108.96 | 1126.31 | 1119.94 | | 0.81 | 80.29 | |
| | | | 1143.66 | | | | | | |
| | | M8-3 | 1072.56 | 1111.12 | 1104.76 | | 0.80 | 79.21 | |
| | | | 1149.69 | | | | | | |
| 113 (2.2 mpk) | 3 h | M9-1 | 1058.59 | 1044.25 | 1037.88 | 892.56 | 0.71 | 70.65 | 60.76 |
| | | | 1029.91 | | | | | | |
| | | M9-2 | 726.72 | 720.82 | 714.45 | | 0.49 | 48.63 | |
| | | | 714.92 | | | | | | |
| | | M9-3 | 925.07 | 931.70 | 925.33 | | 0.64 | 62.99 | |
| | | | 938.33 | | | | | | |
| | 6 h | M10-1 | 366.91 | 342.32 | 335.96 | 897.74 | 0.24 | 24.09 | 64.36 |
| | | | 317.74 | | | | | | |
| | | M10-2 | 1221.50 | 1136.55 | 1130.19 | | 0.82 | 81.03 | |
| | | | 1051.60 | | | | | | |
| | | M10-3 | 1382.49 | 1233.43 | 1227.07 | | 0.89 | 87.97 | |
| | | | 1084.37 | | | | | | |
| IgG | | M1-1 | 39.34 | 38.03 | 0.00 | 0.00 | 0.00 | 0.00 | 0.00 |
| | | | 36.72 | | | | | | |
| 5 nM OA | | M1-1 | 282.72 | 276.96 | 270.60 | 270.60 | 0.12 | 12.46 | 12.46 |
| | | | 271.21 | | | | | | |
| Vehicle | 3 h | M1-1 | 2557.60 | 2471.02 | 2464.65 | 2171.76 | 1.13 | 113.49 | 100.00 |
| | | | 2384.43 | | | | | | |
| | | M1-2 | 2312.07 | 2353.33 | 2346.97 | | 1.08 | 108.07 | |
| | | | 2394.59 | | | | | | |
| | | M1-3 | 1806.17 | 1710.03 | 1703.66 | | 0.78 | 78.45 | |
| | | | 1613.89 | | | | | | |
| | 6 h | M2-1 | 2857.41 | 2736.77 | 2730.41 | 2447.31 | 1.12 | 111.57 | 100.00 |
| | | | 2616.14 | | | | | | |
| | | M2-2 | 3164.93 | 2911.88 | 2905.51 | | 1.19 | 118.72 | |
| | | | 2658.82 | | | | | | |
| | | M2-3 | 1791.74 | 1712.37 | 1706.00 | | 0.70 | 69.71 | |
| | | | 1633.00 | | | | | | |
| 151 (0.8 mpk) | 3 h | M11-1 | 1332.79 | 1414.09 | 1376.06 | 1599.57 | 0.63 | 55.83 | 64.90 |
| | | | 1495.39 | | | | | | |
| | | M11-2 | 2603.34 | 2264.51 | 2226.48 | | 1.03 | 90.34 | |
| | | | 1925.68 | | | | | | |
| | | M11-3 | 1273.84 | 1234.21 | 1196.18 | | 0.55 | 48.53 | |
| | | | 1194.57 | | | | | | |
| | 6 h | M12-1 | 1514.30 | 1397.42 | 1359.40 | 1662.31 | 0.56 | 48.95 | 59.85 |
| | | | 1280.55 | | | | | | |
| | | M12-2 | 1980.77 | 1930.66 | 1892.63 | | 0.77 | 68.15 | |
| | | | 1880.56 | | | | | | |
| | | M12-3 | 1846.62 | 1772.94 | 1734.91 | | 0.71 | 62.47 | |
| | | | 1699.26 | | | | | | |
| 151 (1.6 mpk) | 3 h | M13-1 | 1991.94 | 2580.16 | 2542.14 | 2166.08 | 1.17 | 103.14 | 87.89 |
| | | | 3168.39 | | | | | | |
| | | M13-2 | 2285.85 | 2310.34 | 2272.32 | | 1.05 | 92.20 | |
| | | | 2334.84 | | | | | | |
| | | M13-3 | 1781.17 | 1721.82 | 1683.79 | | 0.78 | 68.32 | |
| | | | 1662.47 | | | | | | |
| | 6 h | M14-1 | 1999.47 | 1972.43 | 1934.40 | 2082.71 | 0.79 | 69.65 | 74.99 |
| | | | 1945.40 | | | | | | |
| | | M14-2 | 1958.41 | 2050.48 | 2012.45 | | 0.82 | 72.46 | |
| | | | 2142.56 | | | | | | |
| | | M14-3 | 2296.42 | 2339.31 | 2301.28 | | 0.94 | 82.86 | |
| | | | 2382.19 | | | | | | |
| 153 (0.85 mpk) | 3 h | M15-1 | 2670.21 | 2620.61 | 2582.58 | 2159.78 | 1.19 | 104.78 | 87.63 |
| | | | 2571.02 | | | | | | |
| | | M15-2 | 1916.94 | 1867.04 | 1829.02 | | 0.84 | 74.21 | |
| | | | 1817.15 | | | | | | |
| | | M15-3 | 2140.93 | 2105.77 | 2067.74 | | 0.95 | 83.90 | |
| | | | 2070.60 | | | | | | |
| | 6 h | M16-1 | 1969.99 | 2020.30 | 1982.27 | 2104.97 | 0.81 | 71.37 | 75.79 |
| | | | 2070.60 | | | | | | |
| | | M16-2 | 2070.20 | 2074.57 | 2036.54 | | 0.83 | 73.33 | |
| | | | 2078.94 | | | | | | |
| | | M16-3 | 2387.89 | 2334.12 | 2296.10 | | 0.94 | 82.67 | |
| | | | 2280.36 | | | | | | |
| 153 (1.7 mpk) | 3 h | M17-1 | 2278.33 | 2226.40 | 2188.37 | 1620.00 | 1.01 | 88.79 | 65.73 |
| | | | 2174.47 | | | | | | |
| | | M17-2 | 1309.82 | 1281.36 | 1243.34 | | 0.57 | 50.45 | |
| | | | 1252.91 | | | | | | |
| | | M17-3 | 1475.88 | 1466.33 | 1428.30 | | 0.66 | 57.95 | |
| | | | 1456.77 | | | | | | |
| | 6 h | M18-1 | 1822.84 | 1800.78 | 1762.76 | 1696.22 | 0.72 | 63.47 | 61.07 |
| | | | 1778.73 | | | | | | |
| | | M18-2 | 1685.64 | 1744.07 | 1706.05 | | 0.70 | 61.43 | |
| | | | 1802.51 | | | | | | |
| | | M18-3 | 1690.72 | 1657.89 | 1619.87 | | 0.66 | 58.32 | |
| | | | 1625.07 | | | | | | |
| 157 (1.0 mpk) | 3 h | M19-1 | 1696.01 | 1714.09 | 1676.07 | 1523.35 | 0.77 | 68.00 | 61.81 |
| | | | 1732.18 | | | | | | |
| | | M19-2 | 1420.80 | 1383.40 | 1345.37 | | 0.62 | 54.59 | |
| | | | 1346.00 | | | | | | |
| | | M19-3 | 1604.74 | 1586.65 | 1548.63 | | 0.71 | 62.83 | |
| | | | 1568.56 | | | | | | |
| | 6 h | M20-1 | 1975.68 | 1879.04 | 1841.01 | 1413.26 | 0.75 | 66.29 | 50.88 |
| | | | 1782.39 | | | | | | |
| | | M20-2 | 1408.40 | 1394.48 | 1356.45 | | 0.55 | 48.84 | |
| | | | 1380.55 | | | | | | |
| | | M20-3 | 1075.87 | 1080.34 | 1042.32 | | 0.43 | 37.53 | |
| | | | 1084.82 | | | | | | |
| 157 (2.0 mpk) | 3 h | M21-1 | 1589.30 | 1657.89 | 1619.87 | 1343.13 | 0.75 | 65.72 | 54.50 |
| | | | 1726.49 | | | | | | |
| | | M21-2 | 1634.83 | 1605.56 | 1567.53 | | 0.72 | 63.60 | |
| | | | 1576.29 | | | | | | |
| | | M21-3 | 896.81 | 880.04 | 842.01 | | 0.39 | 34.16 | |
| | | | 863.27 | | | | | | |
| | 6 h | M22-1 | 1929.55 | 2015.93 | 1977.90 | 1748.29 | 0.81 | 71.22 | 62.95 |
| | | | 2102.31 | | | | | | |
| | | M22-2 | 1375.88 | 1342.14 | 1304.11 | | 0.53 | 46.96 | |
| | | | 1308.40 | | | | | | |
| | | M22-3 | 1958.00 | 2000.89 | 1962.86 | | 0.80 | 70.67 | |
| | | | 2043.77 | | | | | | |

### Example 1

### Protein Phosphatase 2A Activity in Mice Liver and Brain

Compounds 100, 113, 151, 153 and 157 were intraperitoneally administered to mice and PP2A activity was measured in the liver and brain. 153 and 157 inhibited PP2A activity in the liver and brain of mice (FIGS. 1 and 2). Both compounds at high doses significantly inhibited the activity of PP2A in livers at 6 h post treatment as compared with vehicle. 153 at high doses significantly inhibited the activity of PP2A in brains at 6 h post treatment (61% PP2A activity as compared with vehicle). Compound 157 at high doses significantly inhibited the activity of PP2A in brains at 3 h and 6 h post treatment (51% an 63% PP2A activity, respectively, as compared with vehicle). Compounds 153 and 157 inhibited PP2A activity in the brain more effectively than compound 100 at high doses at 3 h and 6 h post treatment.

### Example 2

### Activity Against Cancer Cell Lines

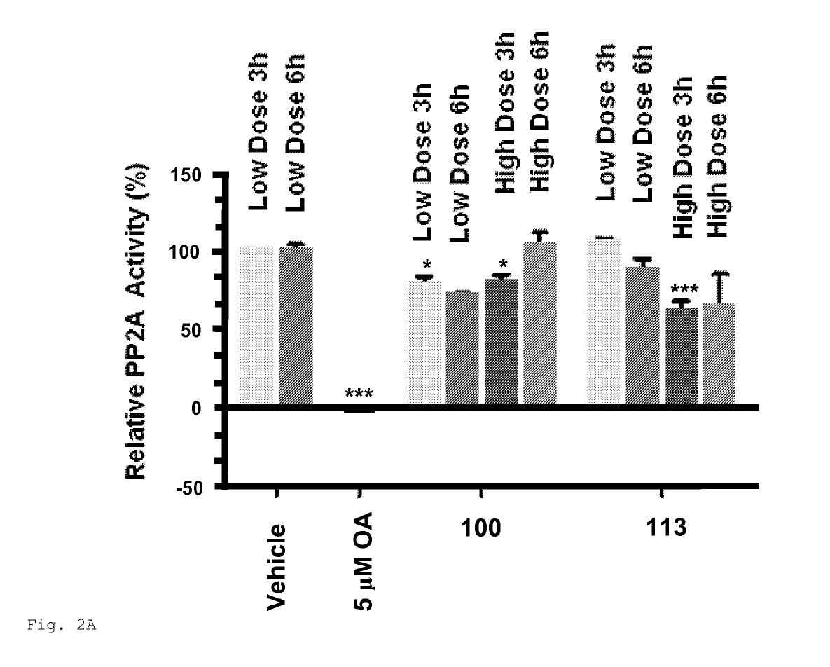

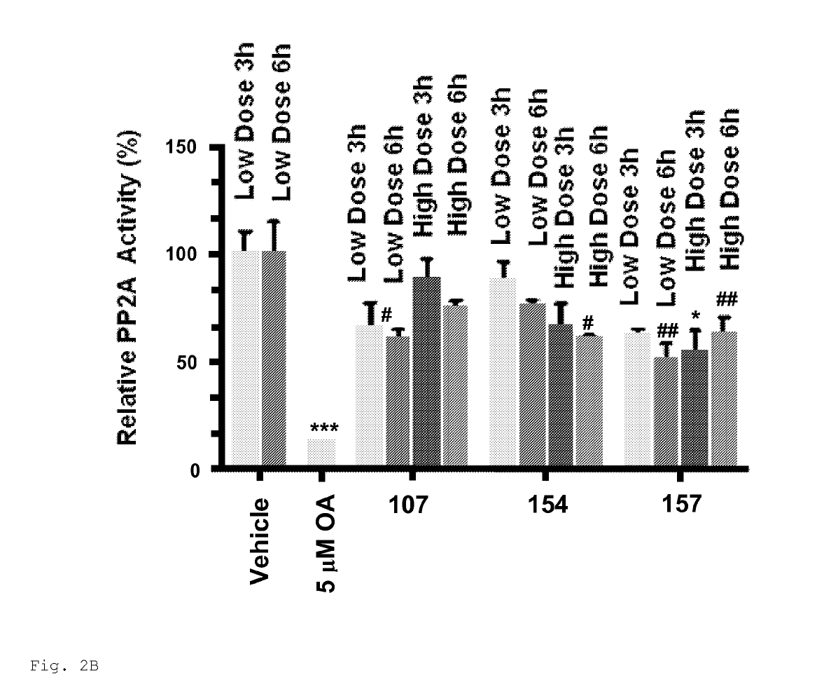

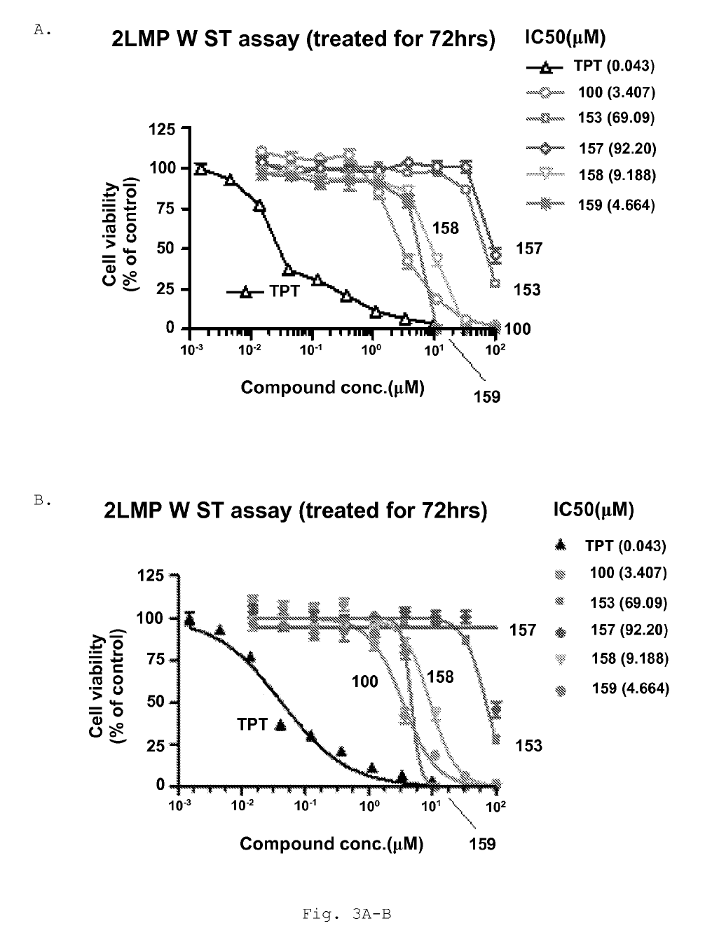

Compounds 100, 153, 157, 158 and 159 were tested in WST cell viability assays. IC₅₀ values were obtained for cytotoxicity against breast cancer (2LMP), glioblastoma (U-87) and lung cancer (A549) cells (See Table 5 and FIGS. 3-5). 153 and 154 were cytotoxic against breast cancer cells. 158 and 159 were cytotoxic against breast cancer, glioblastoma and lung cancer cells. 158 and 159 had increased cytotoxicity relative to 100.

TABLE 5
Cell Viability Assays

| | 2LMP(WST- | U-87 MG(WST- | A549(WST- |
|---|---|---|---|
| IC₅₀(μM) | 20131015//20131025) | 20131017//20131024) | 20131028) |
| TPT | 0.043//0.073 | 0.449//0.422 | 0.309 |
| 100 | 3.407//6.981 | 19.85//25.25 | 10.33 |
| 153 | 69.09//61.98 | >100//>100 | >100 |
| 157 | 92.20//96.37 | >100//>100 | >100 |
| 158 | 9.188//12.83 | 15.19//8.282 | 8.743 |
| 159 | 4.664//4.616 | 5.413//5.071 | 4.710 |

### Example 3

### Administration of Compound 153 or 157

An amount of compound 153 or 157 is administered to a subject afflicted with brain cancer. The amount of the compound is effective to treat the subject.

An amount of compound 153 or 157 is administered to a subject afflicted with diffuse intrinsic pontine glioma. The amount of the compound is effective to treat the subject.

An amount of compound 153 or 157 is administered to a subject afflicted with glioblastoma multiforme. The amount of the compound is effective to treat the subject.

An amount of compound 153 or 157 is administered to a subject afflicted with brain cancer. The amount of the compound is effective to cross the blood brain barrier of the subject and treat the subject.

An amount of compound 153 or 157 is administered to a subject afflicted with diffuse intrinsic pontine glioma. The amount of the compound is effective to cross the blood brain barrier of the subject and treat the subject.

An amount of compound 153 or 157 is administered to a subject afflicted with glioblastoma multiforme. The amount of the compound is effective to cross the blood brain barrier of the subject and treat the subject.

### Example 4

### Administration of Compound 153 or 157 in Combination with an Anti-Cancer Agent

An amount of compound 153 or 157 in combination with an anti-cancer agent is administered to a subject afflicted with brain cancer. The amount of the compound is effective to enhance the anti-cancer activity of the anti-cancer agent.

An amount of compound 153 or 157 in combination with ionizing radiation, x-radiation, docetaxel or temozolomide is administered to a subject afflicted with brain cancer. The amount of the compound is effective to enhance the anti-cancer activity of the ionizing radiation, x-radiation, docetaxel or temozolomide.

An amount of compound 153 or 157 in combination with an anti-cancer agent is administered to a subject afflicted with diffuse intrinsic pontine glioma or glioblastoma multiforme. The amount of the compound is effective to enhance the anti-cancer activity of the anti-cancer agent.

An amount of compound 153 or 157 in combination with ionizing radiation, x-radiation, docetaxel or temozolomide is administered to a subject afflicted with diffuse intrinsic pontine glioma or glioblastoma multiforme. The amount of the compound is effective to enhance the anti-cancer activity of the ionizing radiation, x-radiation, docetaxel or temozolomide.

### Example 5

### Administration of Compound 158 or 159

An amount of compound 158 or 159 is administered to a subject afflicted with brain cancer. The amount of the compound is effective to treat the subject.

An amount of compound 158 or 159 is administered to a subject afflicted with diffuse intrinsic pontine glioma. The amount of the compound is effective to treat the subject.

An amount of compound 158 or 159 is administered to a subject afflicted with glioblastoma multiforme. The amount of the compound is effective to treat the subject.

An amount of compound 158 or 159 is administered to a subject afflicted with brain cancer. The amount of the compound is effective to cross the blood brain barrier of the subject and treat the subject.

An amount of compound 158 or 159 is administered to a subject afflicted with diffuse intrinsic pontine glioma. The amount of the compound is effective to cross the blood brain barrier of the subject and treat the subject.

An amount of compound 158 or 159 is administered to a subject afflicted with glioblastoma multiforme. The amount of the compound is effective to cross the blood brain barrier of the subject and treat the subject.

### Example 6

### Administration of Compound 158 or 159 in Combination with an Anti-Cancer Agent

An amount of compound 158 or 159 in combination with an anti-cancer agent is administered to a subject afflicted with brain cancer. The amount of the compound is effective to enhance the anti-cancer activity of the anti-cancer agent.

An amount of compound 158 or 159 in combination with ionizing radiation, x-radiation, docetaxel or temozolomide is administered to a subject afflicted with brain cancer. The amount of the compound is effective to enhance the anti-cancer activity of the ionizing radiation, x-radiation, docetaxel or temozolomide.

An amount of compound 158 or 159 in combination with an anti-cancer agent is administered to a subject afflicted with diffuse intrinsic pontine glioma or glioblastoma multiforme. The amount of the compound is effective to enhance the anti-cancer activity of the anti-cancer agent.

An amount of compound 158 or 159 in combination with ionizing radiation, x-radiation, docetaxel or temozolomide is administered to a subject afflicted with diffuse intrinsic pontine glioma or glioblastoma multiforme. The amount of the compound is effective to enhance the anti-cancer activity of the ionizing radiation, x-radiation, docetaxel or temozolomide.

### Example 7

### Additional Protein Phosphatase 2A Inhibitors

The compounds used in the method of the present invention are PP2A inhibitors. An additional aspect of the invention provides analogues of 153, 157, 158 and 159, which are inhibitors of PP2A in vitro in human cancer cells and in xenografts of human tumor cells in mice when given parenterally in mice. These compounds inhibit the growth of cancer cells in mouse model systems. The analogues of 153, 157, 158 and 159 are intraperitoneally administered to mice and PP2A activity is measured in the liver and brain. The analogues of B153, 157, 158 and 159 reduce PP2A activity in the liver and brain.

An amount of an analogue of 153, 157, 158 or 159 is administered to a subject afflicted with brain cancer. The amount of the compound is effective to treat the subject.

An amount of an analogue of 153, 157, 158 or 159 is administered to a subject afflicted with diffuse intrinsic pontine glioma or glioblastoma multiforme. The amount of the compound is effective to treat the subject.

An amount of an analogue of 153, 157, 158 or 159 is administered to a subject afflicted with brain cancer. The amount of the compound is effective to cross the blood brain barrier of the subject and treat the subject.

An amount of an analogue of 153, 157, 158 or 159 is administered to a subject afflicted with diffuse intrinsic pontine glioma or glioblastoma multiforme. The amount of the compound is effective to cross the blood brain barrier of the subject and treat the subject.

An amount of an analogue of 153, 157, 158 or 159, in combination with an anti-cancer agent is administered to a subject afflicted with brain cancer. The amount of the compound is effective to enhance the anti-cancer activity of the anti-cancer agent.

An amount of an analogue of 153, 157, 158 or 159 in combination with ionizing radiation, x-radiation, docetaxel or temozolomide is administered to a subject afflicted with brain cancer. The amount of the compound is effective to enhance the anti-cancer activity of the ionizing radiation, x-radiation, docetaxel or temozolomide.

An amount of an analogue of 153, 157, 158 or 159 in combination with an anti-cancer agent is administered to a subject afflicted with diffuse intrinsic pontine glioma or glioblastoma multiforme. The amount of the compound is effective to enhance the anti-cancer activity of the anti-cancer agent.

An amount of an analogue of 153, 157, 158 or 159 in combination with ionizing radiation, x-radiation, docetaxel or temozolomide is administered to a subject afflicted with diffuse intrinsic pontine glioma or glioblastoma multiforme. The amount of the compound is effective to enhance the anti-cancer activity of the ionizing radiation, x-radiation, docetaxel or temozolomide.

### Example 8

### Pharmacokinetic Study of Compounds 153 and 157

The pharmacokinetic studies on 153, 157 and its metabolite endothal were conducted in SD rats. 153 at 1.25 mg/kg and 157 at 1.5 mg/kg were administrated via iv and po route into SD rats. The blood, liver and brain tissue samples were collected at predetermined times from rats. The LC/MS/MS methods were developed to determine 153, 157 and endothal in plasma, liver and brain samples. In the report, the concentrations of 153, 157 and endothal in plasma, liver and brain samples after iv dose were presented. The bioavailability of 153 and 157 was also calculated. Compound were diluted shortly before use in 4% sodium bicarbonate for sterile injection (this is the standard pediatric solution of NaHCO₃ with a pH of about 8.5).

A total of 30 female SD rats were assigned to this study as shown in the table below:

| | | Animal | | Dose | Volume | | |
|---|---|---|---|---|---|---|---|
| Group | Cpds | number | Route | (mg/kg) | (ml/kg) | 2 rats/Timepoint | Sampling |
| 1 | Control | 2 | | | | | |
| 2 | 153 | 12 | IV | 1.25 mg/kg | 5 ml/kg | 15 min, 1 hr, 2 hr, 6 hr | Plasma, liver and brain |
| | | | | | | 10 hr, 24 hr | tissue |
| 3 | 157 | 12 | IV | 1.5 mg/kg | 5 ml/kg | 15 min, 1 hr, 2 hr, 6 hr | Plasma, liver and brain |
| | | | | | | 10 hr, 24 hr | tissue |
| 4 | 153 | 2 | PO | 1.25 mg/kg | 5 ml/kg | 30 min, 1 hr, 2 hr, 6 hr | Plasma |
| | | | | | | 10 hr, 24 hr | |
| 5 | 157 | 2 | PO | 1.5 mg/kg | 5 ml/kg | 30 min, 1 hr, 2 hr, 6 hr | Plasma |
| | | | | | | 10 hr, 24 hr | |

Compound 153 was freshly prepared by diluting the drugs shortly before use in 4% sodium bicarbonate for sterile injection (this is the standard pediatric solution of NaHCO₃ with a pH of about 8.5). The final concentrations of 153 solutions were 0.25 mg/mL. The 153 solutions were administered via iv or po route at dose volume of 5 ml/kg according to the latest body weight. Compound 157 was freshly prepared by diluting the drugs shortly before use in 4% sodium bicarbonate for sterile injection (this is the standard pediatric solution of NaHCO₃ with a pH of about 8.5). The final concentrations of 153 solutions were 0.3 mg/mL. The 157 solutions were administered via iv or po route at dose volume of 5 ml/kg according to the latest body weight.

Twelve (12) female SD rats per group were dosed by iv with 153 or 157. The rats were fasted overnight prior to dosing, with free access to water. Foods were withheld for 2 hours post-dose. Blood, liver and brain tissue samples in two animals each group were collected at each time point, within 10% of the scheduled time for each time point. Two extra animals were used for analytic method development.

Blood (>0.3 mL) were collected via aorta abdominalis in anaesthetic animals into tubes containing heparin at 15 min, 1, 2, 6, 10 and 24 hours after iv administration. Liver and brain tissues were collected immediately after animal death. The liver and brain tissues were excised and rinsed with cold saline to avoid blood residual. Upon collection, each sample was placed on ice and the blood samples were subsequently centrifuged (4° C., 11000 rpm, 5 min) to separate plasma. The obtained plasma, liver and brain tissue samples were stored at −70° C. until LC-MS/MS analysis.

Two (2) female SD rats per group were dosed by po with 153 or 157. The rats were fasted overnight prior to dosing, with free access to water. Foods were withheld for 2 hours post-dose. Blood samples (>0.3 mL) were collected via aorta abdominalis in anaesthetic animals into tubes containing heparin at 30 min, 1, 2, 6, 10 and 24 hours after po administration.

### Preparation of Plasma, Liver and Brain Samples for Compound 153

Frozen unknown plasma samples were thawed at room temperature and vortexed thoroughly. With a pipette, 50 μL of plasma was transferred into a 1.5 mL Eppendorf tube. To each sample, 20 μL IS-D (for blank samples, 20 μL acetonitrile:water (1:1) was added) and 300 μl acetonitrile was added. The sample mixture was vortexed for approximately 3 min. After centrifugation at 10000 rpm for 5 min at 4° C., 100 μL of the upper layer was transferred to a new tube and added 200 μL 0.4% formic acid in water (pH 6.0). The mixture was vortexed for approximately 3 min before injected onto the LC/MS/MS system for analysis.

On the day of the assay, the frozen liver and brain samples were thawed unassisted at room temperature. An about 200 mg weighed sample of each thawed tissue was placed into a plastic tube with water (0.6 mL) to facilitate homogenization. Tissue processing was conducted using a homogenizer for approximately 1 min, 200 μl homogenate was transferred into a fresh Eppendorf tube. To each tube, 50 μL IS-D was added and mixed. Then 600 μl acetonitrile was added and the sample mixture was vortexed for approximately 3 min. After centrifugation at 10000 rpm for 5 min at 4° C., 400 μL of the upper layer was transferred to a new tube and evaporate the supernatant to dryness at 35° C. Reconstitute the residue with 200 μL of 0.4% formic acid in water (pH6.0), and vortex for 3 min, submit for LC-MS/MS analysis.

### Preparation of Plasma, Liver and Brain Samples for Compound 157

Frozen unknown plasma samples were thawed at room temperature and vortexed thoroughly. With a pipette, 50 μL of plasma was transferred into a 1.5 mL Eppendorf tube. To each sample, 30 μL IS-D (for blank samples, 20 μL acetonitrile:water (1:1) was added) and 300 μl acetonitrile was added. The sample mixture was vortexed for approximately 3 min. After centrifugation at 10000 rpm for 5 min at 4° C., 100 μL of the upper layer was transferred to a new tube and added 200 μL 0.4% formic acid in water (pH6.0). The mixture was vortexed for approximately 3 min before injected onto the LC/MS/MS system for analysis.

On the day of the assay, the frozen liver and brain samples were thawed unassisted at room temperature. An about 200 mg weighed sample of each thawed tissue was placed into a plastic tube with water (0.6 mL) to facilitate homogenization. Tissue processing was conducted using a homogenizer for approximately 1 min, 100 μl homogenate was transferred into a fresh Eppendorf tube. To each tube, 50 μL IS-D was added and mixed. Then 500 μl acetonitrile was added and the sample mixture was vortexed for approximately 3 min. After centrifugation at 10000 rpm for 5 min at 4° C., 100 μL of the upper layer was transferred to a new tube and evaporate the supernatant to dryness at 35° C. Reconstitute the residue with 200 μL of 0.4% formic acid in water (pH 6.0), and vortex for 3 min, submit for LC-MS/MS analysis.

### Preparation of Plasma, Liver and Brain Samples for Endothal

Frozen unknown plasma samples were completely thawed at room temperature and vortexed thoroughly. With a pipette, 50 μL of plasma was transferred into a 2.0 mL Eppendorf tube. 50 μL of 0.1N HCl and 800 μL ethyl acetate were added into each sample. The sample mixture was vortexed for approximately 3 min. After centrifugation at 10000 rpm for 5 min at 4° C., the 600 μl supernatant was transferred into a 1.5 mL Eppendorf tube. The precipitate were extracted with 800 μL ethyl acetate again and 600 μl supernatant was transferred into the same tube, and evaporated into dryness. The residue was reconstituted with 150 μL IS-D (for blank samples, 0.05% formic acid in acetonitrile), and vortexed for 3 min. submit for LC/MS/MS analysis. On the day of the assay, the frozen liver and brain tissues samples were thawed unassisted at room temperature. An about 200 mg weighed sample of each thawed tissue was placed into a plastic tube with water (0.6 mL) to facilitate homogenization. 150 μL of each homogenate was transferred into a fresh Eppendorf tube, 150 μL of 0.1N HCl and 800 μL of acetic ether were added into each homogenate sample. The sample mixture was vortexed and centrifuged at 10000 rpm for 5 min at 4° C. 600 μl supernatant was transferred into a 1.5 mL Eppendorf tube, the precipitate were extracted with 800 μL ethyl acetate again and 600 μl supernatant was transferred into the same tube, and evaporated into dryness. The residue was reconstituted with 200 μL IS-D (for blank samples, 0.05% formic acid in acetonitrile), and vortexed for 3 min. submit for LC/MS/MS analysis.

### Preparation of Calibration Samples for Compound 153

### 1) Preparation of Calibration Samples for Plasma Samples Analysis

Calibration standards were prepared by spiking 25 μL of the 153 standard solutions into 25 μL of heparinized blank rat plasma. The nominal standard concentrations in mouse plasma were 2.00, 4.00, 10.0, 50.0, 100, 500, 900 and 1000 ng/mL.

### 2) Preparation of Calibration Samples for Liver and Brain Tissue Samples Analysis

In order to quantify 153 in liver and brain tissue samples, a calibration curve consisting of 8 standard samples was prepared, using the same blank tissue homogenate as sample matrix analyzed (final concentrations: 1.00, 2.00, 5.00, 25.0, 50.0, 250, 450 and 500 ng/g).

### Preparation of Calibration Samples for Compound 157

### 1) Preparation of Calibration Samples for Plasma Samples Analysis

Calibration standards were prepared by spiking 25 μL of the 157 standard solutions into 25 μL of heparinized blank rat plasma. The nominal standard concentrations in mouse plasma were 0.500, 1.00, 2.50, 12.5, 25.0, 125, 225 and 250 ng/mL.

### 2) Preparation of Calibration Samples for Liver and Brain Tissue Samples Analysis

In order to quantify 157 in liver and brain tissue samples, a calibration curve consisting of 8 standard samples was prepared, using the same blank tissue homogenate as sample matrix analyzed (final concentrations: 0.500, 1.00, 2.50, 12.5, 25.0, 125, 225 and 250 ng/mL).

### Preparation of Calibration Samples for Endothal

### 1) Preparation of Calibration Samples for Plasma Samples Analysis

Calibration standards were prepared by spiking 25 μL of the endothal standard solutions into 25 μL of heparinized blank rat plasma. The nominal standard concentrations in rat plasma were 20.0, 40.0, 100, 200, 400, 2000, 3600 and 4000 ng/mL.

### 2) Preparation of Calibration Samples for Liver Tissue Samples Analysis

In order to quantify endothal in liver tissue samples, a calibration curve consisting of 8 standard samples was prepared, using the same blank tissue homogenate as sample matrix analyzed (final concentrations: 20.0, 40.0, 100, 200, 400, 2000, 3600 and 4000 ng/g).

### LC/MS/MS System

The analysis was performed using a LC-MS/MS system consisting of the following components: HPLC system: Shimadzu UFLC 20-AD XR; MS/MS system: API-5000 triple quadrupole mass spectrometer (Applied Biosystems); Data system: Watson LIMS version 7.2.

### 1) Chromatographic Conditions for Compound 153

| | |
|---|---|
| Analytical column: | Luna C18 5 μm, 50 × 2.0 mm |
| Mobile phase: | A: 0.4% formic acid in water (pH 6.0) |
| | B: Acetonitrile |
| Injection volume: | 20~30 μl |
| Run Time: | ~4.5 min |
| Flow Rate: | 0.5 mL/min |

| | Time | | | | | | | |
|---|---|---|---|---|---|---|---|---|
| | 0 | 0.5 | 0.6 | 2.0 | 2.1 | 3.0 | 3.1 | 4.5 |
| % B | 15 | 15 | 45 | 45 | 95 | 95 | 15 | Stop |
| Divert | Waste | MS | MS | MS | MS | Waste | | Waste |
| Valve | | | | | | | | |
| Position | | | | | | | | |

### 2) Mass Spectrometric Conditions for Compound 153

| | |
|---|---|
| Parameters | 153 |
| Ion Spray (IS) | 5000 V |
| Curtain Gas (CUR) | 15 |
| Temperature (TEM) | 500° C. |
| Entrance Potential (EP) | 10 |
| Collision Gas (CAD) | 6 |
| Collision Cell Exit Potential (CXP) | 15 |
| Dwell Time (ms) | 100 |
| Gas 1 | 40 |
| Gas 2 | 40 |
| Declustering potential (DP) | 120 |
| Ionization Mode: | (+) ESI |

### (CE):

| | Precursor | Product | |
|---|---|---|---|
| | ion | ion | CE |
| Compound | (m/z) | (m/z) | (eV) |
| 153 | 311.1 | 169.2 | 30 |
| Irbesartan (IS) | 429.4 | 207.2 | 30 |

### 1) Chromatographic Conditions for Compound 157

| Analytical column: | Luna C18 5 μm, 50 × 2.0 mm | |
|---|---|---|
| Mobile phase: | A: 0.4% formic acid in water (pH 6.0) | |
| | B: Acetonitrile | |
| Injection volume: | 10 | μL |
| Run Time: | ~4.5 | min |
| Flow Rate: | 0.5 | mL/min |

| | Time | | | | | | |
|---|---|---|---|---|---|---|---|
| | 0 | 0.5 | 2.0 | 2.1 | 3.0 | 3.1 | 4.0 |
| % B | 45 | 45 | 45 | 95 | 95 | 45 | Stop |
| Divert | Waste | MS | MS | MS | Waste | | Waste |
| Valve | | | | | | | |
| Position | | | | | | | |

### 2) Mass Spectrometric Conditions for Compound 157

| | |
|---|---|
| Parameters | 157 |
| Ion Spray (IS) | 5000 V |
| Curtain Gas (CUR) | 15 |
| Temperature (TEM) | 450° C. |
| Entrance Potential (EP) | 10 |
| Collision Gas (CAD) | 6 |
| Collision Cell Exit Potential (CXP) | 15 |
| Dwell Time (ms) | 100 |
| Gas 1 | 40 |
| Gas 2 | 40 |
| Declustering potential (DP) | 120 |
| Ionization Mode: | (+) ESI |

### (CE):

| | Precursor | Product | |
|---|---|---|---|
| | ion | ion | CE |
| Compound | (m/z) | (m/z) | (eV) |
| 157 | 367.3 | 251.0 | 25 |
| Verapamil (IS) | 455.1 | 303.3 | 25 |

### 1) Chromatographic Conditions for Endothal

Chromatographic separation was carried out at room temperature.

| Analytical column: | Luna HILIC 5 μm, 100 × 2.0 mm | |
|---|---|---|
| Mobile phase: | A: 0.1% formic acid in water | |
| | B: Acetonitrile | |
| Injection volume: | 5 | μL |
| Run Time: | ~2.5 | min |
| Flow Rate: | 0.6 | mL/min |

| | | | | |
|---|---|---|---|---|
| Time | 0 | 0.4 | 2.0 | 2.5 |
| % B | 88 | 88 | 88 | Stop |
| Divert Valve | Waste | MS | Waste | Waste |
| Position | | | | |

### 2) Mass Spectrometric Conditions for Endothal

| | |
|---|---|
| Parameters | endothal |
| Ion Spray (IS) | −4500 V |
| Curtain Gas (CUR) | 20 |
| Temperature (TEM) | 450° C. |
| Entrance Potential (EP) | −10 |
| Collision Gas (CAD) | 6 |
| Collision Cell Exit Potential (CXP) | −10 |
| Dwell Time (ms) | 150 |
| Gas 1 | 45 |
| Gas 2 | 45 |
| Declustering potential (DP) | −80 |
| Ionization Mode: | (−) ESI |

### (CE):

| | Precursor | Product | |
|---|---|---|---|
| | ion | ion | CE |
| Compound | (m/z) | (m/z) | (eV) |
| Endothal | 185 | 141 | −30 |
| PAH(IS) | 192.9 | 149 | −20 |

### Quantification

Quantification was achieved by the external standard method for 153, 157 and endothal. Concentrations of the test article were calculated using a weighted least-squares linear regression (W=1/x²).

### Pharmacokinetic Interpretation

The pharmacokinetic parameters were evaluated using Watson LIMS (version 7.2), assuming a non-compartmental model for drug absorption and distribution.
AUC₀₋ₜ (AUCₗₐₛₜ) is the area under the plasma concentration-time curve from time zero to last sampling time, calculated by the linear trapezoidal rule.
AUC_{0-∞} (AUC^{INF}) is the area under the plasma concentration-time curve with last concentration extrapolated based on the elimination rate constant.

### Results

The calibration curve of 153 in rat plasma was linear throughout the study in the range of 2.00-1000 ng/mL. The linear equation and the correlation coefficient of calibration curve is y=0.0252x+0.0127 and R²=0.9957.

The calibration curve of 100 in the tested tissues was linear throughout the study in the range of 1.00-500 ng/g. The linear equation and the correlation coefficient of calibration curve is y=0.0233x+0.0213 and R²=0.9939.

The calibration curve of 157 in rat plasma was linear throughout the study in the range of 0.50-250 ng/mL. The linear equation and the correlation coefficient of calibration curve is y=0.333x−0.0136 and R²=0.9986.

The calibration curve of 157 in the tested tissues was linear throughout the study in the range of 0.50-250 ng/g. The linear equation and the correlation coefficient of calibration curve is y=0.0467x+0.0034 and R²=0.9989.

The calibration curves of endothal in rat plasma were linear throughout the study in the range of 20.0-4000 ng/mL. The linear equation and the correlation coefficient of calibration curve is y=0.00155x−0.00162 and R²=0.9986.

The calibration curves of endothal in rat liver tissues were linear throughout the study in the range of 20.0-4000 ng/g. The linear equation and the correlation coefficient of calibration curve are y=0.00349x+0.0177 and R²=0.997.

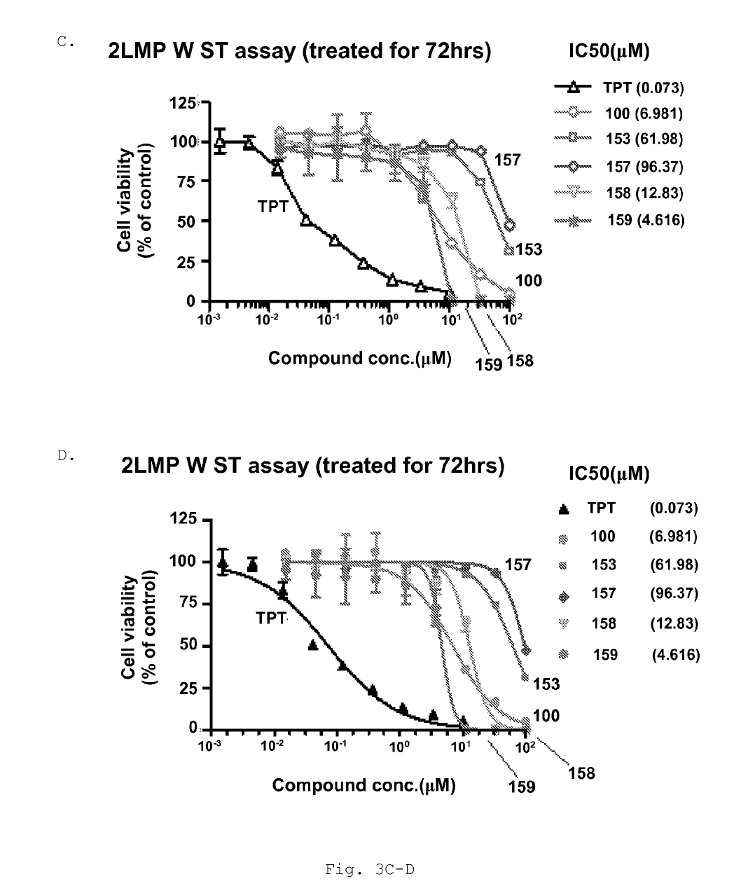

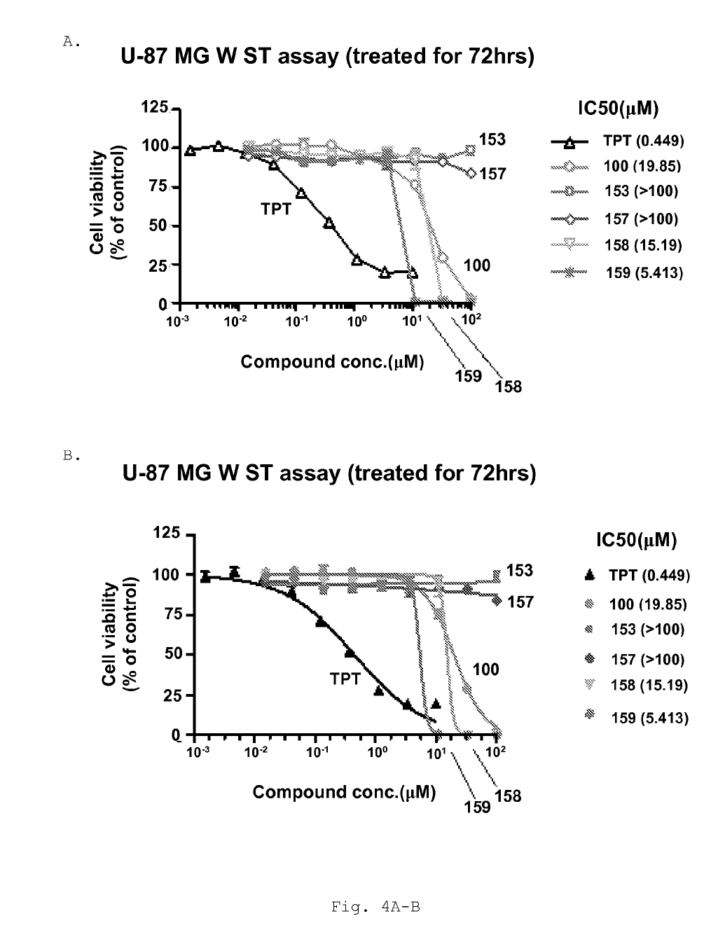

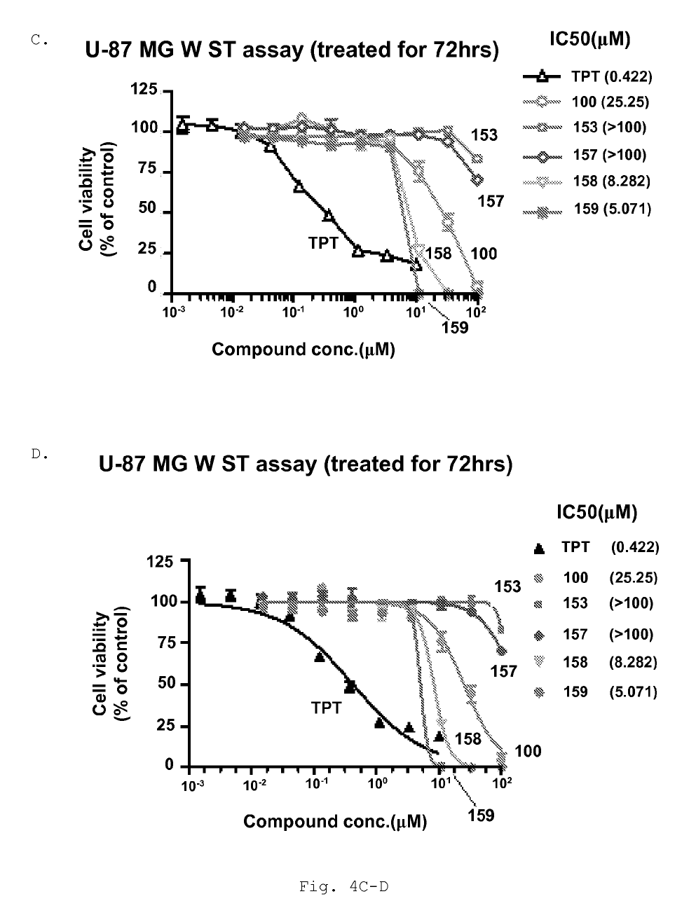

Following single iv & po administration of 153 to SD rats, plasma, liver and brain tissue concentrations of both 153 and endothal were determined by the LC/MS/MS method described above. The plasma, liver and brain tissue concentrations at each sampling time are listed in Tables 6.1-6.8 and FIGS. 6A-6B. The calculated pharmacokinetic parameters are listed in Table 6.9-6.12.

153 was orally available at 1.25 mg/kg to SD rats, the Cₘₐₓ was 239 ng/mL, AUC was 164 ng·h/ml, and the BA is 55.41%.

The mean Cₘₐₓ in plasma was 557 ng/ml following iv administration of 153. The mean Cₘₐₓ in liver and brain were 762.0 ng/kg and 42.7 ng/kg, respectively. AUCₗₐₛₜ in plasma was 295 ng·h/ml, with 500 ng·h/g in liver and 39.4 ng·h/g in brain, respectively. T_{1/2} in plasma, liver and brain were 0.921 h, 0.626 h and 0.596 h, respectively.

As shown in Table 6.5-6.8 and figure 6.2, endothal was detectable in plasma and liver samples following single iv administration of 153 at 1.25 mg/kg, whereas not detectable in brain samples. The mean Cₘₐₓ in plasma and liver were 70.5 ng/ml and 2068 ng/ml, respectively. AUCₗₐₛₜ in plasma and liver were 378 ng·h/ml and 10820 ng·h/g, respectively. T_{1/2} in plasma and liver were 5.20 h and 2.79 h, respectively.

Following single iv & po administration of 157 to SD rats, plasma, liver and brain tissue concentrations of both 157 and endothal were determined by the LC/MS/MS method described above. The plasma, liver and brain tissue concentrations at each sampling time are listed in Tables 6.13-6.20 and FIG. 6C-6D. The calculated pharmacokinetic parameters are listed in Table 6.21-6.24. 157 was poorly orally available at 1.5 mg/kg to SD rats, the Cₘₐₓ was 6.14 ng/mL, AUC was 3.2 ng·h/ml, and the BA was 6.98%.

The mean Cₘₐₓ in plasma was 115 ng/ml following iv administration of 157 at 1.5 mg/kg to SD rats. The mean Cₘₐₓ in liver and brain were 297 ng/kg and 60.0 ng/kg, respectively. AUCₗₐₛₜ in plasma was 47.2 ng·h/ml, with 152 ng·h/g in liver and 24.6 ng·h/g in brain, respectively. T_{1/2} in plasma, liver and brain were 0.391 h, 0.813 h and 0.162 h, respectively.

As shown in table 6.17-6.20 and figure. 6.4, endothal was detectable in plasma and liver samples following single iv administration of 157 at 1.5 mg/kg, whereas endothal was not detectable in brain samples. The mean Cₘₐₓ in plasma and liver were 98.1 ng/ml and 3720 ng/ml, respectively. AUCₗₐₛₜ in plasma and liver were 374 ng·h/ml and 15025 ng·h/g, respectively. T_{1/2} in plasma and liver were 5.94 h and 2.61 h, respectively.

153 was orally available at 1.25 mg/kg to SD rats, the Cₘₐₓ was 239 ng/mL, AUC was 164 ng·h/ml, and the BA was 55.41%. The mean Cₘₐₓ in plasma was 557 ng/ml following iv administration of 153. The mean Cₘₐₓ in liver and brain were 762.0 ng/kg and 42.7 ng/kg, respectively. AUC_{Iast} in plasma was 295 ng·h/ml, with 500 ng·h/g in liver and 39.4 ng·h/g in brain, respectively. T_{1/2} in plasma, liver and brain were 0.921 h, 0.626 h and 0.596 h, respectively.

Endothal was detectable in plasma and liver samples following single iv administration of 153 at 1.25 mg/kg. The mean Cₘₐₓ in plasma and liver were 70.5 ng/ml and 2068 ng/ml, respectively. AUCₗₐₛₜ in plasma and liver were 378 ng·h/ml and 10820 ng·h/g, respectively. T_{1/2} in plasma and liver were 5.20 h and 2.79 h, respectively. However, endothal was undetectable in brain tissue.

157 was poorly orally available at 1.5 mg/kg to SD rats, the Cₘₐₓ was 6.14 ng/mL, AUC was 3.2 ng·h/ml, and the BA was 6.98%.

The mean Cₘₐₓ in plasma was 115 ng/ml following iv administration of 157 at 1.5 mg/kg to SD rats. The mean Cₘₐₓ in liver and brain were 297 ng/kg and 60.0 ng/kg, respectively. AUCₗₐₛₜ in plasma was 47.2 ng·h/ml, with 152 ng·h/g in liver and 24.6 ng·h/g in brain, respectively. T_{1/2} in plasma, liver and brain were 0.391 h, 0.813 h and 0.162 h, respectively.

Endothal was detectable in plasma and liver samples following single iv administration of 157 at 1.5 mg/kg. The mean Cₘₐₓ in plasma and liver were 98.1 ng/ml and 3720 ng/ml, respectively. AUCₗₐₛₜ in plasma and liver were 374 ng·h/ml and 15025 ng·h/g, respectively. T_{1/2} in plasma and liver were 5.94 h and 2.61 h, respectively. However, endothal was undetectable in brain tissue.

TABLE 6.1
Analytical data of 153 plasma concentration (ng/mL) in SD
rats following PO administration.
1.25 mg/kg Liver concentration (ng/g)

| Time | | | | |
|---|---|---|---|---|
| (hr) | Rat 1 | Rat 2 | Mean | SD |
| 0.25 | 872 | 652 | 762 | 155.6 |
| 1 | 131 | 121 | 126 | 7.1 |
| 2 | 42 | 41.2 | 41.6 | 0.6 |
| 6 | BLQ | BLQ | NA | NA |
| 10 | BLQ | ND | NA | NA |
| 24 | ND | ND | NA | NA |

TABLE 6.2
Analytical data of 153 plasma concentration (ng/mL) in SD
rats following iv administration.
1.25 mg/kg Plasma concentration (ng/ml)

| Time | | | | |
|---|---|---|---|---|
| (hr) | Rat 1 | Rat 2 | Mean | SD |
| 0.25 | 563 | 550 | 557 | 9.2 |
| 1 | 58 | 51.4 | 54.7 | 4.7 |
| 2 | 14.8 | 13 | 13.9 | 1.3 |
| 6 | 1.04 | 1.02 | 1.03 | 0 |
| 10 | ND | 9.42* | NA | NA |
| 24 | ND | ND | NA | NA |

*Conc. was 9.42 ng/mL which was abnormal and did not include in the calculation.

TABLE 6.3
Analytical data of 153 liver concentration (ng/g) in SD
rats following iv administration.
1.25 mg/kg Liver concentration (ng/g)

| Time | | | | |
|---|---|---|---|---|
| (hr) | Rat 1 | Rat 2 | Mean | SD |
| 0.25 | 872 | 652 | 762 | 155.6 |
| 1 | 131 | 121 | 126 | 7.1 |
| 2 | 42 | 41.2 | 41.6 | 0.6 |
| 6 | BLQ | BLQ | NA | NA |
| 10 | BLQ | ND | NA | NA |
| 24 | ND | ND | NA | NA |

TABLE 6.4
Analytical data of 153 brain concentration (ng/g) in SD
rats following iv administration.
1.25 mg/kg Brain concentration (ng/g)

| Time | | | | |
|---|---|---|---|---|
| (hr) | Rat 1 | Rat 2 | Mean | SD |
| 0.25 | 45 | 40.3 | 42.7 | 3.3 |
| 1 | 13.9 | 14.3 | 14.1 | 0.3 |
| 2 | 4.05 | 4.75 | 4.4 | 0.5 |
| 6 | ND | ND | NA | NA |
| 10 | ND | ND | NA | NA |
| 24 | ND | ND | NA | NA |

TABLE 6.5
Analytical data of endothal plasma concentration
(ng/ml) in SD rats following po administration of 153.
Endothal plasma concentration (ng/ml)

| Time | | | | |
|---|---|---|---|---|
| (hr) | Rat 1 | Rat 2 | Mean | SD |
| 0.25 | 41.4 | 40.2 | 40.8 | 0.8 |
| 1 | 53.6 | 38.9 | 46.3 | 10.4 |
| 2 | 34.5 | 35.3 | 34.9 | 0.6 |
| 6 | 25.8 | 20.8 | 23.3 | 3.5 |
| 10 | BLQ | ND | NA | NA |
| 24 | ND | ND | NA | NA |

TABLE 6.6
Analytical data of endothal plasma concentration (ng/ml)
in SD rats following iv administration of 153:
Endothal plasma concentration (ng/ml)

| Time | | | | |
|---|---|---|---|---|
| (hr) | Rat 1 | Rat 2 | Mean | SD |
| 0.25 | 70.9 | 63.8 | 67.4 | 5 |
| 1 | 57.1 | 44.3 | 50.7 | 9.1 |
| 2 | 77.1 | 56.1 | 66.6 | 14.8 |
| 6 | 42.2 | 35.4 | 38.8 | 4.8 |
| 10 | 21.7 | BLQ | NA | NA |
| 24 | BLQ | BLQ | NA | NA |

TABLE 6.7
Analytical data of endothal liver concentration (ng/g) in
SD rats following iv administration of 153.
Endothal liver concentration (ng/g)

| Time | | | | |
|---|---|---|---|---|
| (hr) | Rat 1 | Rat 2 | Mean | SD |
| 0.25 | 1524 | 956 | 1240 | 401.6 |
| 1 | 1836 | 2012 | 1924 | 124.5 |
| 2 | 1912 | 2224 | 2068 | 220.6 |
| 6 | 492 | 980 | 736 | 345.1 |
| 10 | 301 | 256 | 279 | 31.8 |
| 24 | ND | ND | NA | NA |

TABLE 6.8
Analytical data of endothal brain concentration (ng/g) in
SD rats following iv administration of 153.
Endothal brain concentration (ng/g)

| Time | | | | |
|---|---|---|---|---|
| (hr) | Rat 1 | Rat 2 | Mean | SD |
| 0.25 | ND | ND | NA | NA |
| 1 | ND | ND | NA | NA |
| 2 | ND | ND | NA | NA |
| 6 | ND | ND | NA | NA |
| 10 | ND | ND | NA | NA |
| 24 | ND | ND | NA | NA |

TABLE 6.9
Main pharmacokinetic parameters of 153 in SD rats
following iv or po administration.

| | Plasma | | | AUC | AUC_{0-∞} | MRT | | |
|---|---|---|---|---|---|---|---|---|
| | PK | Cₘₐₓ | Tₘₐₓ | ng * Hours/ | ng * Hours/ | (0-t) | T_{1/2} | F |
| Dosage | Parameters | ng/mL | Hours | mL | mL | Hours | Hours | % |
| 1.25 mg/kg | 1 | 249 | 0.5 | 163 | 163 | 0.987 | 0.33 | |
| (PO | 2 | 229 | 0.5 | 164 | 164 | 1.04 | 0.355 | |
| Group) | Mean | 239 | 0.5 | 164 | 164 | 1.01 | 0.343 | 55.41 |
| 1.25 mg/kg | 1 | 563 | 0.25 | 303 | 303 | 0.666 | 0.907 | |
| (IV | 2 | 550 | 0.25 | 288 | 288 | 0.647 | 0.934 | |
| Group) | Mean | 557 | 0.25 | 295 | 296 | 0.657 | 0.921 | |

TABLE 6.10
Main pharmacokinetic parameters of 153 in liver & brain
of SD rats following iv or po administration.

| | | | Plasma | | | AUC | AUC_{0-∞} | MRT | |
|---|---|---|---|---|---|---|---|---|---|
| | | | PK | Cₘₐₓ | Tₘₐₓ | ng * Hrs/ | ng * Hrs/ | (0-t) | T_{1/2} |
| TA | Dosage | Group | Parameters | ng/mL | Hrs | mL | mL | Hrs | Hrs |
| End. | 153 | PO | 1 | 53.6 | 1 | 189 | 395 | 2.8 | 5.53 |
| | 1.25 mg/kg | | 2 | 40.2 | 0.5 | 169 | 333 | 2.72 | 5.45 |
| | | | Mean | 46.9 | 0.75 | 179 | 364 | 2.76 | 5.49 |
| | 153 | IV | 1 | 77.1 | 2 | 482 | 618 | 3.93 | 4.37 |
| | 1.25 mg/kg | | 2 | 63.8 | 0.25 | 274 | 581 | 2.74 | 6.02 |
| | | | Mean | 70.5 | 1.13 | 378 | 600 | 3.34 | 5.2 |

| | | | | AUC | AUC_{0-∞} | MRT | |
|---|---|---|---|---|---|---|---|
| | PK | Cₘₐₓ | Tₘₐₓ | ng * Hours/ | ng * Hours/ | (0-t) | T_{1/2} |
| Group | Parameters | ng/mL | Hours | mL | mL | Hours | Hours |
| Liver | 1 | 872 | 0.25 | 547 | 547 | 0.745 | 0.609 |
| 1.25 mg/kg | 2 | 652 | 0.25 | 453 | 453 | 0.825 | 0.643 |
| IV | Mean | 762 | 0.25 | 500 | 500 | 0.785 | 0.626 |
| Brain | 1 | 45 | 0.25 | 39.2 | 39.2 | 0.934 | 0.562 |
| 1.25 mg/kg | 2 | 40.3 | 0.25 | 39.5 | 39.5 | 1.01 | 0.629 |
| IV | Mean | 42.7 | 0.25 | 39.4 | 39.35 | 0.972 | 0.596 |

TABLE 6.12
Main pharmacokinetic parameters of Endothal in SD rats
liver & brain following single iv administration of 153.

| | | | | | AUC | | MRT | |
|---|---|---|---|---|---|---|---|---|
| | | PK | Cₘₐₓ | Tₘₐₓ | ng * Hrs/ | AUC_{0-∞} | (0-t) | T_{1/2} |
| TA | Dosage | Parameters | ng/mL | Hrs | mL | ng * Hrs/mL | Hrs | Hrs |
| End. | 153 | 1 | 1912 | 2 | 9528 | 10800 | 3.05 | 3 |
| | 1.25 mg/kg | 2 | 2224 | 2 | 12112 | 13100 | 3.43 | 2.57 |
| | (Liver | Mean | 2068 | 2 | 10820 | 11950 | 3.24 | 2.79 |
| | Group) | | | | | | | |
| | 153 | 1 | NA | NA | NA | NA | NA | NA |
| | 1.25 mg/kg | 2 | NA | NA | NA | NA | NA | NA |
| | (Brian | Mean | NA | NA | NA | NA | NA | NA |
| | Group) | | | | | | | |

TABLE 6.13
Analytical data of 157 plasma concentration (ng/mL) in SD
rats following PO administration.
1.5 mg/kg Plasma concentration (ng/ml)

| Time | | | | |
|---|---|---|---|---|
| (hr) | Rat 1 | Rat 2 | Mean | SD |
| 0.5 | 5.92 | 6.35 | 6.14 | 0.3 |
| 1 | 1.48 | 1.26 | 1.37 | 0.2 |
| 2 | 0.303 | 0.194 | 0.249 | 0.1 |
| 6 | ND | ND | NA | NA |
| 10 | ND | ND | NA | NA |
| 24 | ND | ND | NA | NA |

TABLE 6.14
Analytical data of 157 plasma concentration (ng/mL) in SD
rats following iv administration.
1.5 mg/kg Plasma concentration (ng/ml)

| Time | | | | |
|---|---|---|---|---|
| (hr) | Rat 1 | Rat 2 | Mean | SD |
| 0.25 | 116 | 114 | 115 | 1.4 |
| 1 | 2.67 | 3.57 | 3.12 | 0.6 |
| 2 | 0.491 | 0.556 | 0.524 | 0 |
| 6 | ND | ND | NA | NA |
| 10 | ND | ND | NA | NA |
| 24 | ND | ND | NA | NA |

TABLE 6.15
Analytical data of 157 liver concentration (ng/g) in SD
rats following iv administration.
1.5 mg/kg Liver concentration (ng/g)

| Time | | | | |
|---|---|---|---|---|
| (hr) | Rat 1 | Rat 2 | Mean | SD |
| 0.25 | 337 | 257 | 297 | 56.6 |
| 1 | 29.4 | 17.6 | 23.5 | 8.3 |
| 2 | 6.40 | 9.72 | 8.06 | 2.3 |
| 6 | ND | ND | NA | NA |
| 10 | ND | BLQ | NA | NA |
| 24 | ND | ND | NA | NA |

TABLE 6.16
Analytical data of 157 brain concentration (ng/g) in SD
rats following iv administration.
1.5 mg/kg Brain concentration (ng/g)

| Time | | | | |
|---|---|---|---|---|
| (hr) | Rat 1 | Rat 2 | Mean | SD |
| 0.25 | 60.0 | 60.0 | 60.0 | 0.0 |
| 1 | 1.99 | 2.80 | 2.40 | 0.6 |
| 2 | BLQ | BLQ | NA | NA |
| 6 | ND | ND | NA | NA |
| 10 | ND | ND | NA | NA |
| 24 | ND | ND | NA | NA |

TABLE 6.17
Analytical data of endothal plasma concentration (ng/ml)
in SD rats following po administration of 157.
Endothal plasma concentration (ng/ml)

| Time | | | | |
|---|---|---|---|---|
| (hr) | Rat 1 | Rat 2 | Mean | SD |
| 0.25 | 93.5 | 65.4 | 79.5 | 19.9 |
| 1 | 91.8 | 150 | 121 | 41.2 |
| 2 | 142 | 68.9 | 105 | 51.7 |
| 6 | 22.7 | 31.9 | 27.3 | 6.5 |
| 10 | BLQ | BLQ | NA | NA |
| 24 | ND | ND | NA | NA |

TABLE 6.18
Analytical data of endothal plasma concentration (ng/ml)
in SD rats following iv administration of 157.
Endothal plasma concentration (ng/ml)

| Time | | | | |
|---|---|---|---|---|
| (hr) | Rat 1 | Rat 2 | Mean | SD |
| 0.25 | 76.4 | 53.4 | 64.9 | 16.3 |
| 1 | 113 | 83.2 | 98.1 | 21.1 |
| 2 | 91.5 | 45.7 | 68.6 | 32.4 |
| 6 | 47.7 | 45 | 46.4 | 1.9 |
| 10 | BLQ | BLQ | NA | NA |
| 24 | BLQ | BLQ | NA | NA |

TABLE 6.19
Analytical data of endothal liver concentration (ng/g) in
SD rats following iv administration of 157.
Endothal liver concentration (ng/g)

| Time | | | | |
|---|---|---|---|---|
| (hr) | Rat 1 | Rat 2 | Mean | SD |
| 0.25 | 3676 | 3536 | 3606 | 99.0 |
| 1 | 3124 | 3764 | 3444 | 452.5 |
| 2 | 2484 | 2272 | 2378 | 149.9 |
| 6 | 1000 | 1076 | 1038 | 53.7 |
| 10 | 218 | 344 | 281 | 89.1 |
| 24 | ND | ND | NA | NA |

TABLE 6.20
Analytical data of endothal brain concentration (ng/g) in
SD rats following iv administration of 157.
Endothal brain concentration (ng/g)

| Time | | | | |
|---|---|---|---|---|
| (hr) | Rat 1 | Rat 2 | Mean | SD |
| 0.25 | ND | ND | NA | NA |
| 1 | ND | ND | NA | NA |
| 2 | ND | ND | NA | NA |
| 6 | ND | ND | NA | NA |
| 10 | ND | ND | NA | NA |
| 24 | ND | ND | NA | NA |

TABLE 6.21
Main pharmacokinetic parameters of 157 in SD rats
following iv or po administration.

| | | Plasma | | | AUC | AUC_{0-∞} | MRT | | |
|---|---|---|---|---|---|---|---|---|---|
| | | PK | Cₘₐₓ | Tₘₐₓ | ng * Hrs/ | ng * Hrs/ | (0-t) | T_{1/2} | F |
| Dosage | Group | Parameters | ng/mL | Hrs | mL | mL | Hrs | Hrs | % |
| 1.5 mg/kg | PO | 1 | 5.92 | 0.5 | 3.4 | 3.4 | 0.988 | 0.437 | |
| | | 2 | 6.35 | 0.5 | 3 | 3 | 0.903 | 0.37 | |
| | | Mean | 6.14 | 0.5 | 3.2 | 3.2 | 0.946 | 0.404 | 6.78 |
| | IV | 1 | 116 | 0.25 | 47.1 | 47.1 | 0.333 | 0.409 | |
| | | 2 | 114 | 0.25 | 47.3 | 47.3 | 0.349 | 0.373 | |
| | | Mean | 115 | 0.25 | 47.2 | 47.2 | 0.341 | 0.391 | |

TABLE 6.22
Main pharmacokinetic parameters of 157 in SD rats liver &
brain following iv administration.

| | | | | | AUC | AUC_{0-∞} | MRT | |
|---|---|---|---|---|---|---|---|---|
| | | PK | Cₘₐₓ | Tₘₐₓ | ng * Hrs/ | ng * Hrs/ | (0-t) | T_{1/2} |
| Dosage | Tissues | Parameters | ng/mL | Hrs | mL | mL | Hrs | Hrs |
| 1.5 mg/kg | Liver | 1 | 337 | 0.25 | 168 | 168 | 0.531 | 0.455 |
| | | 2 | 257 | 0.25 | 136 | 136 | 0.647 | 1.17 |
| | | Mean | 297 | 0.25 | 152 | 152 | 0.589 | 0.813 |
| | Brain | 1 | 60 | 0.25 | 24.2 | 24.2 | 0.305 | 0.153 |
| | | 2 | 60 | 0.25 | 25 | 25 | 0.323 | 0.17 |
| | | Mean | 60 | 0.25 | 24.6 | 24.6 | 0.314 | 0.162 |

TABLE 6.23
Main pharmacokinetic parameters of Endothal in SD rats
following single iv & po administration of 157.

| | | | Plasma | | | | | MRT | |
|---|---|---|---|---|---|---|---|---|---|
| | | | PK | Cₘₐₓ | Tₘₐₓ | AUC | AUC_{0-∞} | (0-t) | T_{1/2} |
| TA | Dosage | Group | Parameters | ng/mL | Hours | ng * Hours/mL | ng * Hours/mL | Hours | Hours |
| Endothal | 157 | PO | 1 | 142 | 2 | 492.6 | 542 | 2.15 | 1.51 |
| | (1.25 mg/kg) | | 2 | 150 | 1 | 365 | 481 | 2.32 | 2.51 |
| | | | Mean | 146 | 1.5 | 429 | 512 | 2.24 | 2.01 |
| | 157 | IV | 1 | 113 | 1 | 452 | 733 | 2.52 | 4.08 |
| | (1.25 mg/kg) | | 2 | 83.2 | 1 | 297 | 803 | 2.85 | 7.8 |
| | | | Mean | 98.1 | 1 | 374 | 768 | 2.69 | 5.94 |

TABLE 6.24
Main pharmacokinetic parameters of Endothal in SD rats
liver & brain following single iv administration of 157.

| | | | | | | | | MRT | |
|---|---|---|---|---|---|---|---|---|---|
| | | | PK | Cₘₐₓ | Tₘₐₓ | AUC | AUC_{0-∞} | (0-t) | T_{1/2} |
| TA | Dosage | Tissues | Parameters | ng/mL | Hrs | ng * Hrs/mL | ng * Hrs/mL | Hrs | Hrs |
| Endothal | 157 | Liver | 1 | 3676 | 0.25 | 14759 | 15500 | 2.97 | 2.28 |
| | (1.25 mg/kg | | 2 | 3764 | 1 | 15292 | 16700 | 3.12 | 2.94 |
| | IV) | | Mean | 3720 | 0.625 | 15025 | 16100 | 3.05 | 2.61 |
| | | Brain | 1 | NA | NA | NA | NA | NA | NA |
| | | | 2 | NA | NA | NA | NA | NA | NA |
| | | | Mean | NA | NA | NA | NA | NA | NA |

### Example 9

### Pharmacokinetic Study of Compound 105

The purpose of this study was to determine the pharmacokinetics parameters of 105 and endothal in plasma and liver following single intravenous administration of 105 to male SD rats. 105 was dissolved in 4% NaHCO₃ in saline for IV administration. The detailed procedure of dosing solution preparation was presented in Appendix I.

Animal source:

| Species | Gender | Vendor | Certificate No. |
|---|---|---|---|
| SD rats | Male | SLAC | SCXK (SH) 2007-0005 |
| | | Laboratory | |
| | | Animal Co. LTD | |

Thirteen (13) animals were placed on the study. The animals in IV arm were free access to food and water. One extra animal was used for blank liver and plasma generation (5 mL per animal). The resulting blank liver and plasma was then applied to the development of bioanalytical method and sample bioanalysis for the entire study.

### In-Life Study Design

| | Body | | Route | Dose | Dose | Dose | |
|---|---|---|---|---|---|---|---|
| Treatment | Weight | No. of | of | Level* | Conc. | Volume | |
| Group | (g) | Animals | Admin. | (mg/kg) | (mg/mL) | (mL/kg) | Time points |
| 1 | 220-255 | 12 | IV | 1 | 1 | 1 | Sampling at 0.25, 1, |
| | | | | | | | 2, 6, 10 and 24 hr |
| | | | | | | | post dose. |
| | | | | | | | Terminally collect |
| | | | | | | | plasma and liver |
| | | | | | | | samples from the |
| | | | | | | | same animal. |

*Dose was expressed as free base of 105.

### Dosing, Sampling, Sample Processing and Sample Storage

The IV injection was conducted via foot dorsal vein. Animals were free access to food and water before dose.

The animal is restrained manually. Approximately 150 μL of blood/time point is collected into sodium heparin tube via cardiac puncture for terminal bleeding (anesthetized under carbon dioxide). Blood sample will be put on ice and centrifuged to obtain plasma sample (2000 g, 5 min under 4° C.) within 10 minutes.

The animal will be euthanized with carbon dioxide inhalation. Open abdominal cavity with scissor to expose internal organs. Hold the carcass in an upright position and allow the organs to fall forward. Cut the connective tissues and remove the organs. Then the organs are rinsed with cold saline, dried on filtrate paper, placed into a screw-top tube and weighed, snap frozen by placing into dry-ice immediately.

Plasma and liver samples were stored at approximately −80° C. until analysis. The backup samples will be discarded after three weeks after in-life completion unless requested. The unused dosing solutions will be discarded within three weeks after completion of the study

### LC-MS-MS Analysis Analytical Method for 105

| Instrument | UPLC/MS-MS-010 (API-4000) | |
|---|---|---|
| Matrix | SD rat plasma and liver homogenate | |
| Analyte(s) | Compound 105 | |
| Internal | Dexamethasone | |
| standard(s) | | |
| MS | ESI: Positive ion | |
| conditions | | |
| | MRM detection | |
| | LB-105: [M + H] ⁺ m/z 283.3→ 265.2 | |
| | Dexamethasone: [M + H] ⁺ m/z 393.3 ® 373.1 | |
| HPLC | Mobile Phase A: H₂O-0.1% FA-5 mM NH₄OAc | |
| conditions | Mobile Phase B: ACN | |
| | Time (min) | Mobile Phase B (%) |
| | 0.20 | 2.00 |
| | 1.00 | 95.0 |
| | 1.60 | 95.0 |
| | 1.61 | 2.00 |
| | 2.20 | stop |

| | |
|---|---|
| | Column: ACQUITY UPLC HSS T3 (2.1 × 50 mm, 1.8 |
| | μm) |
| | Flow rate: 0.60 mL/min |
| | Column temperature: 60° C. |
| | Retention time: |
| | LB-105 : 0.97 min |
| | Dexamethasone: .1.25 min |
| | For plasma samples: An aliquot of 30 μL sample |
| | was added with 100 μL IS (Dexamethasone, 100 |
| | ng/mL in ACN). The mixture was vortexed for 10 |
| | min at 750 rpm and centrifuged at 6000 rpm for |
| | 10 min. An aliquot of 3 μL supernatant was |
| | injected for LC-MS/MS analysis. |
| | For diluted samples: An aliquot of 3 μL plasma |
| | sample was diluted with 27 μL blank plasma. |
| | The following processing procedure was the |
| | same as those un-diluted plasma samples. |
| | For all the samples preparation, allow |
| | calibration, quality control, blanks, and test |
| | samples to thaw at 4° C. (nominal). And keep |
| | each step on an ice bath or at 4° C. |
| Calibration | 10.00-3000 ng/mL for LB-105 in SD rat plasma |
| Curve | and liver homogenate. |

### LC-MS-MS Analysis Analytical Method for Endothal

| Instrument | UPLC/MS-MS-015 (API-5500, Q-trap) | |
|---|---|---|
| Matrix | SD rat plasma and liver homogenate | |
| Analyte(s) | Endothal | |
| Internal | Diclofenac | |
| standard(s) | | |
| MS conditions | ESI: Negative ion | |
| | MRM detection | |
| | Endothal: [M − H] ⁻ m/z 184.9 → 141.0 | |
| | Diclofenac: [M − H] ⁻ m/z 294.2 → 249.9 | |
| HPLC | Mobile Phase A: H₂O-0.1% FA-5 mM NH₄OAc | |
| conditions | Mobile Phase B: ACN | |
| | Time (min) | Mobile Phase B (%) |
| | 0.40 | 2.00 |
| | 1.00 | 85.0 |
| | 1.50 | 85.0 |
| | 1.51 | 2.00 |
| | 2.00 | stop |

| | |
|---|---|
| | Column: ACQUITY UPLC HSS T3 (2.1 × 50 mm, 1.8 μm) |
| | Flow rate: 0.60 mL/min |
| | Column temperature: 60° C. |
| | Retention time: |
| | Endothal: 0.87 min |
| | Diclofenac: 1.28 min |
| | For plasma samples: |
| | An aliquot of 30 μL sample was added with 100 μL IS |
| | (Diclofenac, 100 ng/mL in ACN). The mixture was |
| | vortexed for 10 min at 750 rpm and centrifuged at |
| | 6000 rpm for 10 min. An aliquot of 3 μL supernatant |
| | was injected for LC-MS/MS analysis. |
| | For liver homogenate samples: |
| | The liver samples were homogenized with 3 volumes |
| | (v/w) of homogenizing solution PBS (pH7.4) for 2 |
| | mins. An aliquot of 30 μL tissue homogenate sample |
| | was added with 100 μL IS (Diclofenac, 100 ng/mL in |
| | ACN). Vortex at 750 rpm for 10 min and centrifuged |
| | at 6000 rpm for 10 min. An aliquot of 3 μL |
| | supernatant was injected for LC-MS/MS analysis. |
| | For all the samples preparation, allow calibration, |
| | quality control, blanks, and test samples to thaw at |
| | 4° C. (nominal). And keep each step on an ice bath or |
| | at 4° C. |
| Calibration | 20.00-3000 ng/mL for Endothal in SD rat plasma and |
| curve | liver homogenate.. |

### Pharmacokinetic Analysis

### Software:

The PK parameters were determined by non-compartmental model of non-compartmental analysis tool, Pharsight Phoenix WinNonlin® 6.2 software.

### “BQL” Rule:

Concentration data under 80% of LLOQ (LLOQ=10.00 ng/mL in rat plasma and liver homogenate for 105, and 20.00 ng/mL for Endothal) was replaced with “SQL” and excluded from graphing and PK parameters estimation. Concentration data within 80%-120% of LLOQ was considered within normal instrumental variation and presented in the results.

Terminal t_{1/2} Calculation:

Time points were automatic selected by “best fit” model for terminal half life estimation as the first option. Manual selection was applied when “best fit” could not well define the terminal phase.

### Clinical Observations

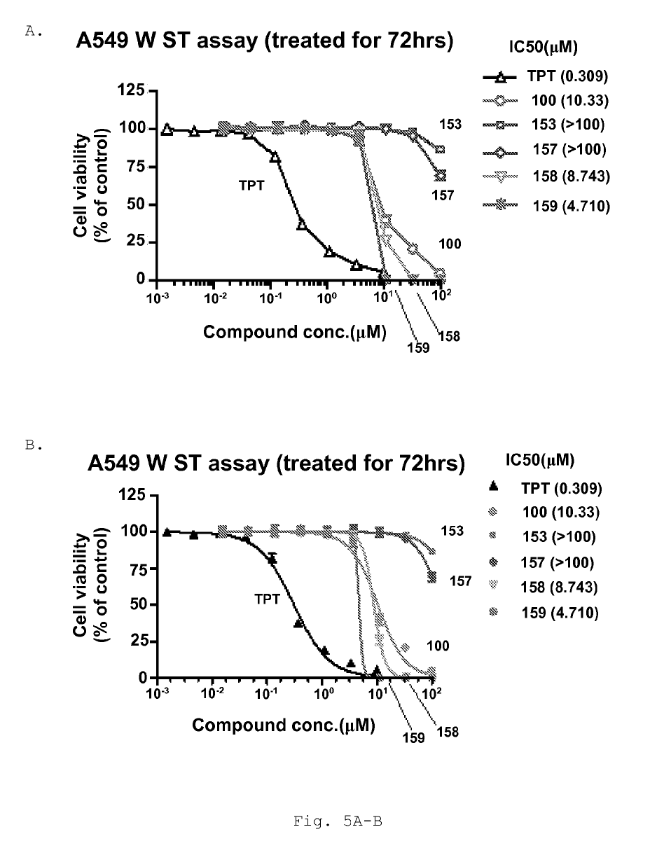

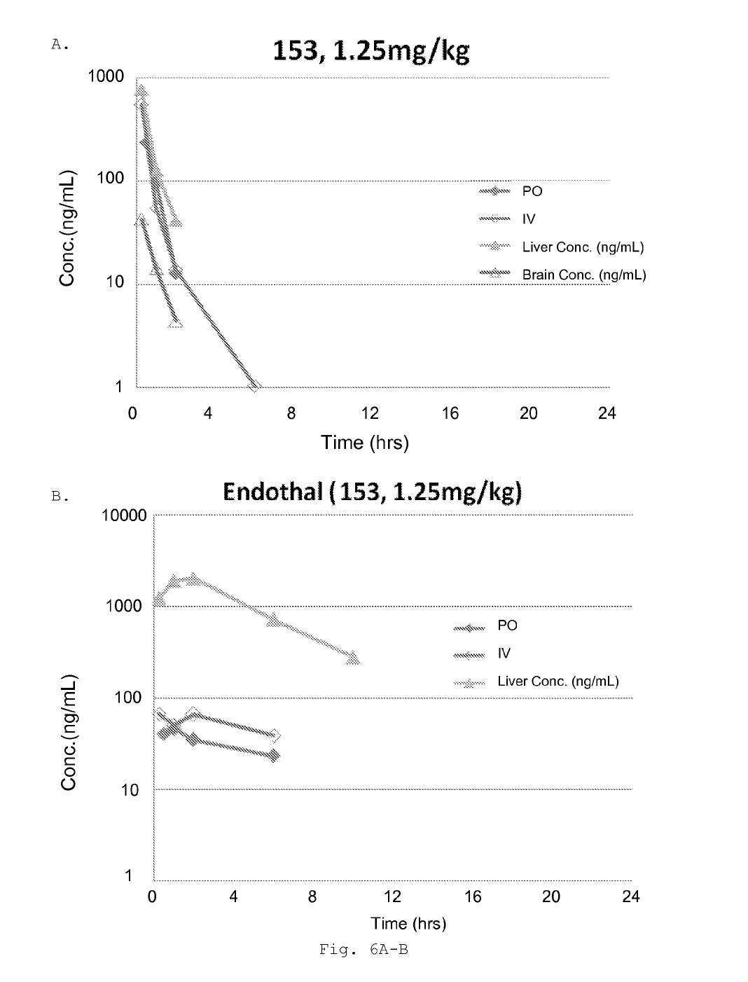

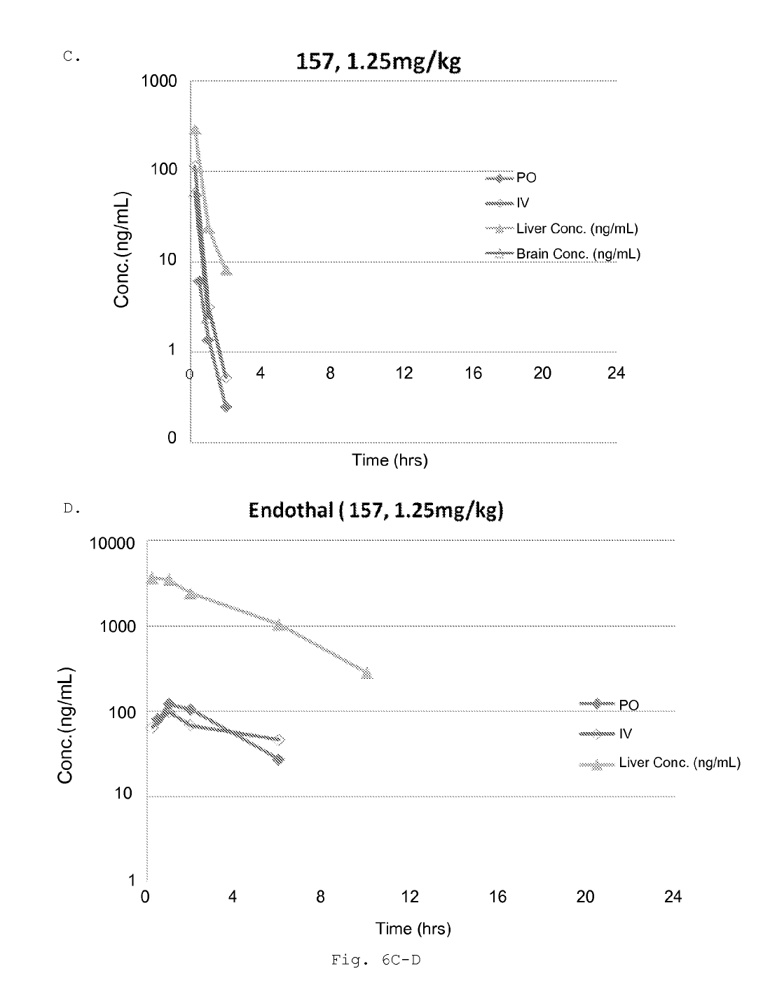

The concentration-time data and pharmacokinetic parameters of 105 and Endothal in rat plasma and liver after IV administration were listed in Tables 7.1 to 7.8, and illustrated in FIGS. 7A to 7C.

TABLE 7.1
Individual and mean plasma concentration-time data of 105
after an IV dose of 1 mg/kg in male SD rats

| Time (hr) | Individual | | Mean (ng/mL) |
|---|---|---|---|
| 0.25 | 1930 | 1530 | 1730 |
| 1 | 263 | 228 | 246 |
| 2 | 45.2 | 21.5 | 33.4 |
| 6 | BQL | BQL | BQL |
| 10 | BQL | BQL | BQL |
| 24 | BQL | BQL | BQL |

LLOQ of 105 in plasma sample is 10.0 ng/mL.
ULOQ of 105 in plasma sample is 3000 ng/mL.

### BLQ: Below Limit of Quantitation

TABLE 7.2
Individual and mean liver concentration-time data of 105
after an IV dose of 1 mg/kg in male SD rats

| Time (hr) | Individual | | Mean (ng/g) |
|---|---|---|---|
| 0.25 | 1070 | 988 | 1029 |
| 1 | 576 | 446 | 511 |
| 2 | 99.2 | 131 | 115 |
| 6 | SQL | BQL | BQL |
| 10 | SQL | SQL | SQL |
| 24 | SQL | BQL | BQL |

The liver sample is homogenized with 3 volumes (v/w) of homogenizing solution (PBS PH7.4).

Liver concentration=liver homogenate conc.×4, assuming 1 g wet liver tissue equals to 1 mL.

LLOQ of 105 in liver homogenate sample is 10.0 ng/mL.

ULOQ of 105 in liver homogenate sample is 3000 ng/mL.

BLQ: Below Limit of Quantitation

TABLE 7.3
Liver-plasma concentration ratio of 105 after an IV dose
of 1 mg/kg in male SD rats

| Time (hr) | Individual | | Mean |
|---|---|---|---|
| 0.25 | 0.554 | 0.646 | 0.600 |
| 1 | 2.19 | 1.96 | 2.07 |
| 2 | 2.19 | 6.09 | 4.14 |
| 6 | NA | NA | NA |
| 10 | NA | NA | NA |
| 24 | NA | NA | NA |

NA: Not Applicable

TABLE 7.4
Individual and mean plasma concentration-time data of
Endothal after an IV dose of 1 mg/kg 105 in SD rats

| Time (hr) | Individual | | Mean (ng/mL) |
|---|---|---|---|
| 0.25 | 263 | 188 | 226 |
| 1 | 69.7 | 45.2 | 57.5 |
| 2 | 23.2 | BQL | 23.2 |
| 6 | BQL | BQL | BQL |
| 10 | BQL | 21.9 | 21.9 |
| 24 | BQL | BQL | BQL |

LLOQ of Endothal in plasma sample is 20.0 ng/mL.

ULOQ of Endothal in plasma sample is 3000 ng/mL.

BLQ: Below Limit of Quantitation

TABLE 7.5
Individual and mean liver concentration-time data
of Endothal after an IV dose of 1 mg/kg 105 in SD rats

| Time (hr) | Individual | | Mean (ng/g) |
|---|---|---|---|
| 0.25 | 475 | 462 | 469 |
| 1 | 541 | 386 | 464 |
| 2 | 151 | 304 | 228 |
| 6 | 76.9 | 163 | 120 |
| 10 | 70.0 | 156 | 113 |
| 24 | BQL | 63.8 | 63.8 |

The liver sample is homogenized with 3 volumes (v/w) of homogenizing solution (PBS PH7.4).

Liver concentration=liver homogenate conc.×4, assuming 1 g wet liver tissue equals to 1 mL.

LLOQ of Endothal in liver homogenate sample is 20.0 ng/mL.

ULOQ of Endothal in liver homogenate sample is 3000 ng/mL.

BLQ: Below Limit of Quantitation

TABLE 7.6
Liver-plasma concentration ratio of Endothal
after an IV dose of 1 mg/kg 105 in SD rats

| Time (hr) | Individual | | Mean |
|---|---|---|---|
| 0.25 | 1.81 | 2.46 | 2.13 |
| 1 | 7.76 | 8.54 | 8.15 |
| 2 | 6.51 | NA | 6.51 |
| 6 | NA | NA | NA |
| 10 | NA | 7.12 | 7.12 |
| 24 | NA | NA | NA |

NA: Not Applicable

TABLE 7.7
Mean Pharmacokinetics Parameters of 105 after an
IV dose of 1 mg/kg in male SD rats

| | Dosing | | | | | | | | | |
|---|---|---|---|---|---|---|---|---|---|---|
| | Route | AUC₍₀₋ₜ₎ | AUC_{(0-∞)} | t_{1/2z} | Tₘₐₓ | Cₘₐₓ | CL | Vₛₛ | MRT_{INF} | AUCₗₐₛₜ₋ₗᵢᵥₑᵣ/ |
| Matrix | (Dose) | h * ng/mL | h * ng/mL | hr | hr | ng/mL | L/hr/kg | L/kg | hr | AUCₗₐₛₜ₋ₚₗₐₛₘₐ |
| Plasma | IV (1 mg/kg) | 1511 | 1526 | 0.309 | NA | NA | 0.655 | 0.215 | 0.328 | NA |
| Liver | | 1019 | NA | NA | 0.25 | 1029 | NA | NA | NA | 67.4 |

NA: Not Applicable

TABLE 7.8
Mean Pharmacokinetics Parameters of Endothal after an IV
dose of 1 mg/kg 105 in male SD rats

| | Dosing | | | | | | |
|---|---|---|---|---|---|---|---|
| | Route | AUC₍₀₋ₜ₎ | AUC_{(0-∞)} | t_{1/2} | Tₘₐₓ | Cₘₐₓ | AUCₗₐₛₜ₋ₗᵢᵥₑᵣ/ |
| Matrix | (Dose) | h * ng/mL | h * ng/mL | hr | hr | ng/mL | AUCₗₐₛₜ₋ₚₗₐₛₘₐ |
| Plasma | IV (1 mg/kg) | 355 | 673 | 10.1 | 0.250 | 226 | NA |
| Liver | | 3152 | 4896 | 19.0 | 0.250 | 469 | 888 |

NA: Not Applicable

IV-1 mg/kg 105

After an IV dose of 105 at 1 mg/kg in male SD rats, concentration of 105 in rat plasma declined with a terminal half life (T₁₁₂) of 0.309 hours. The area under curve from time 0 to last time point (AUCₗₐₛₜ) and from time 0 to infinity (AUC_{INF}) were 1511 and 1526 hr*ng/mL respectively. The total clearance CL and volume of distribution at steady state Vₛₛ were 0.655 L/hr/kg and 0.215 L/kg, respectively.

The mean values of Cₘₐₓ in liver was 1029 ng/g and corresponding Tₘₐₓ value was 0.25 hr. The mean value of AUC₍₀₋ₗₐₛₜ₎ was 1019 ng/g*hr. AUC₍₀₋ₜ₎ ratio of liver over plasma was 67.4.

### Endothal

Following intravenous administration of 1 mg/kg 105 to Male SD rats, concentration of Endothal in rat plasma declined with a terminal half-life (T_{1/2}) of 10.1 hours. The area under curve from time 0 to last time point (AUCₗₐₛₜ) and from time 0 to infinity (AUC_{INF}) were 355 and 673 hr*ng/mL respectively. The mean values of Cₘₐₓ and Tₘₐₓ in plasma were 226 ng/mL and 0.25 hr, respectively.

The mean values of Cₘₐₓ in liver was 469 ng/g and corresponding Tₘₐₓ value was 0.25 hr. The mean value of AUC₍₀₋ₗₐₛₜ₎ and AUC_{(0-∞)} were 3152 and 4896 ng/g*hr, respectively. AUC₍₀₋ₜ₎ ratio of liver over plasma was 888.

### Example 10

### Pharmacokinetic Study of Compound 113

The purpose of this study was to determine the pharmacokinetics parameters of 113, 100 and Endothal following single intravenous (IV) or oral (PO) administrations of 113 to male SD rats. 113 was dissolved in 4% NaHCO3 in saline for IV administration. The detailed procedure of dosing solution preparation was presented in Appendix I.

Animal source

| Species | Gender | Vendor | Certificate No. |
|---|---|---|---|
| SD rats | Male | SLAC Laboratory | SCXK (SH) |
| | | Animal Co. LTD | 2007-0005 |

15 animals were placed on the study. The animals in IV arm were free access to food and water. For PO dose group, the animals were fasted overnight prior to dosing and the food was resumed 4 hours postdose.

One extra animal was used for blank liver, brain and plasma generation (5 mL per animal). The resulting blank liver, brain and plasma were then applied to the development of bioanalytical method and sample bioanalysis for the entire study.

### In-Life Study Design

| | Body | | Route | Dose | Dose | Dose | |
|---|---|---|---|---|---|---|---|
| Treatment | Weight | No. of | of | Level* | Conc. | Volume | |
| Group | (g) | Animals | Admin. | (mg/kg) | (mg/mL) | (mL/kg) | Time points |
| 1 | 275-295 | 12 | IV | 1.4 | 1.4 | 1 | Sampling at 0.25, 1, |
| | | | | | | | 2, 6, 10 and 24 hr |
| | | | | | | | post dose. Terminally |
| | | | | | | | collect plasma, brain |
| | | | | | | | and liver samples from |
| | | | | | | | the same animal. |
| 2 | 275-295 | 2 | PO | 1.4 | 0.14 | 10 | Sampling at 0.25, 1, |
| | | | | | | | 2, 6, 10 and 24 hr |
| | | | | | | | post dose. Serial |
| | | | | | | | bleeding from the same |
| | | | | | | | animal for plasma |
| | | | | | | | only. |

*Dose was expressed as free base of 113.

### Dosing, Sampling, Sample Processing and Sample Storage

The IV injection was conducted via foot dorsal vein. PO via oral gavage.

Blood collection: The animal is restrained manually. Approximately 200 μL of blood/time point is collected into sodium heparin tube via cardiac puncture for terminal bleeding (anesthetized under carbon dioxide). Blood sample will be put on ice and centrifuged to obtain plasma sample (2000 g, 5 min under 4° C.) within 10 minutes.

Liver collection: The animal will be euthanized with carbon dioxide inhalation. Open abdominal cavity with scissor to expose internal organs. Hold the carcass in an upright position and allow the organs to fall forward. Cut the connective tissues and remove the organs. Then the organs are rinsed with cold saline, dried on filtrate paper, placed into a screw-top tube and weighed, snap frozen by placing into dry-ice immediately.

Brain collection: Make a mid-line incision in the animals scalp and retract the skin. Using small bone cutters and rongeurs, remove the skull overlying the brain. Remove the brain using a spatula and rinse with cold saline, dried on filtrate paper, placed into a screw-top tube and weighed, snap frozen by placing into dry-ice immediately. Brain tissue will be homogenized for 2 min with 3 volumes (v/w) of homogenizing solution (PBS pH 7.4) right before analysis. Plasma, brain and liver samples were stored at approximately −80° C. until analysis. The backup samples will be discarded after three weeks after in-life completion unless requested. The unused dosing solutions will be discarded within three weeks after completion of the study.

### LC-MS-MS Analysis Analytical Method for 113

| Instrument | UPLC/MS-MS-010 (API-4000) | |
|---|---|---|
| Matrix | SD rat plasma, brain and liver homogenate | |
| Analyte(a) | 113 | |
| Internal | Dexamethasone/Propranolol | |
| standard(s) | | |
| MS | ESI: Positive ion | |
| conditions | | |
| | MRM detection | |
| | LB-113: [M + H] ⁺ m/z 399.1-251.2 | |
| | Dexamethasone: [M + H] ⁺ m/z 393.3 ® 373.1 | |
| | Propranolol: [M + H] ⁺ m/z 260.2 → 116.1 | |
| HPLC | Mobile Phase A: H₂0-0.1% FA-5 mM NH₄OAc | |
| conditions | Mobile Phase B: ACM | |
| | Time (min) | Mobile Phase B (0) |
| | 0.20 | 2.00 |
| | 0.60 | 95.0 |
| | 1.20 | 95.0 |
| | 1.21 | 2.00 |
| | 1.80 | stop |

| | |
|---|---|
| | Column: ACQUITY UPLC HSS T3 (2.1 × 50 mm, 1.8 μm) |
| | Flow rate: 0.60 ml/min |
| | Column temperature: 60° C. |
| | Retention time: |
| | LB-113: 0.95 min |
| | Dexamethasone: .1.02 min |
| | Propranolol: 0.92 min |
| | For plasma samples: |
| | An aliquot of 30 μL sample was added with 100 μL IS |
| | (Dexamethasone, 100 ng/mL and Propranolol, 50 ng/mL |
| | in ACN). The mixture was vortexed for 10 min at 750 |
| | rpm and centrifuged at 6000 rpm for 10 min. An aliquot |
| | of 1 μL supernatant was injected for LC-MS/MS analysis. |
| | For diluted plasma samples: |
| | An aliquot of 3 μL plasma sample was diluted with 27 μL |
| | blank plasma. The following processing procedure was |
| | the same as those un-diluted plasma samples. |
| | For brain homogenate samples: |
| | The brain samples were homogenized with 3 volumes |
| | (v/w) of homogenizing solution PBS (pH 7.4) for 2 mins. |
| | An aliquot of 30 μL tissue homogenate sample was added |
| | with 100 μL IS (Dexamethasone, 100 ng/ml and |
| | Propranolol, 50 ng/mL in ACN). Vortex at 750 rpm for 10 |
| | min and centrifuged at 6000 rpm for 10 min. An aliquot of |
| | 1 μL supernatant was injected for LC-MS/MS analysis. |
| | For liver homogenate samples: |
| | The liver samples were homogenized with 3 volumes |
| | (v/w) of homogenizing solution PBS (pH 7.4) for 2 mins. |
| | An aliquot of 30 μL tissue homogenate sample was added |
| | with 100 μL IS (Dexamethasone, 100 ng/mL and |
| | Propranolol, 50 ng/mL in ACN). Vortex at 750 rpm for 10 |
| | min and centrifuged at 6000 rpm for 10 min. An aliquot of |
| | 1 μL supernatant was injected for LC-MS/MS analysis. |
| | For all the samples preparation, allow calibration, quality |
| | control, blanks, and test samples to thaw at 4° C. (nominal). |
| | And keep each step on an ice bath or at 4° C. . |
| Calibration | 1.00-3000 ng/mL for LB-113 in SD rat plasma, brain and |
| curve | liver homogenate. |

### LC-MS-MS Analysis Analytical Method for Endothal

| Instrument | UPLC/MS-MS-015 (API-5500, Q-trap) | |
|---|---|---|
| Matrix | SD rat plasma, brain and liver homogenate | |
| Analyte(s) | Endothal | |
| Internal | Diclofenac | |
| standard(s) | | |
| MS | ESI: Negative ion | |
| conditions | | |
| | MRM detection | |
| | Endothal: [M + H] ⁻ m/z 184.9 → 141.0 | |
| | Diclofenac: [M + H] ⁻ m/z 294.2 → 249.9 | |
| HPLC | Mobile Phase A: H₂0-0.1% FA-5 mM NH₄OAc | |
| conditions | Mobile Phase B: ACN | |
| | Time (min) | Mobile Phase B (%) |
| | 0.40 | 2.00 |
| | 1.00 | 85.0 |
| | 1.50 | 85.0 |
| | 1.51 | 2.00 |
| | 2.00 | stop |

| | |
|---|---|
| | Column: ACQUITY UPLC HSS T3 (2.1 × 50 mm, 1.8 μm) |
| | Flow rate: 0.60 mL/min |
| | Column temperature: 60 ° C. |
| | Retention time: |
| | Endothal: 0.87 min |
| | Diclofenac: 1.28 min |
| | For plasma samples: |
| | An aliquot of 30 μL sample was added with 100 μL IS |
| | (Diclofenac, 100 ng/mL in ACN). The mixture was |
| | vortexed for 10 min at 750 rpm and centrifuged |
| | at 6000 μm for 10 min. An aliquot of 3 μL |
| | supernatant was injected for LC-MS/MS analysis. |
| | For brain homogenate samples: |
| | The brain samples were homogenized with 3 volumes |
| | (v/w) of homogenizing solution PBS (pH 7.4) for 2 |
| | mins. An aliquot of 30 μL tissue homogenate sample |
| | was added with 100 μL IS (Diclofenac, 100 ng/mL |
| | in ACN). Vortex at 750 rpm for 10 min and centrifuged |
| | at 6000 rpm for 10 min. An aliquot of 3 μL supernatant |
| | was injected for LC-MS/MS analysis. |
| | For liver homogenate samples:. |
| | The liver samples were homogenized with 3 volumes |
| | (v/w) of homogenizing solution PBS (pH 7.4) for 2 |
| | mins. An aliquot of 30 μL tissue homogenate sample |
| | was added with 100 μL IS (Diclofenac, 100 ng/mL |
| | in ACN). Vortex at 750 rpm for 10 min and centrifuged |
| | at 6000 rpm for 10 min. An aliquot of 3 μL supernatant |
| | was injected for LC-MS/MS analysis. |
| | For all the samples preparation, allow calibration, quality |
| | control, blanks, and test samples to thaw at 4° C. |
| | (nominal). And keep each step on an ice bath or at 4° C. . |
| Calibration | 20.00-3000 ng/mL for Endothal in SD rat plasma, brain |
| curve | and liver homogenate. |

### LC-MS-MS Analysis Analytical Method for Compound 100

| Instrument | UPLC/MS-MS-010 (API-4000) | |
|---|---|---|
| Matrix | SD rat plasma, brain and liver homogenate | |
| Analyte(s) | 100 | |
| Internal | Diclofenac/Propranolol | |
| standard(s) | | |
| MS | ESI: Positive ion | |
| conditions | | |
| | MRM detection | |
| | LB-100: [M + H] ⁺ m/z 269.3 → 101.1 | |
| | Diclofenac: [M + H] ⁺ m/z 296.0 ® 250.3 | |
| | Propranolol: [M + H] ⁺ m/z 260.2 → 116.1 | |
| HPLC | Mobile Phase A: H₂0-0.1% FA-5 mM NH₄OAc | |
| conditions | Mobile Phase B: ACN | |
| | Time (min) | Mobile Phase B (9) |
| | 0.20 | 15.0 |
| | 1.60 | 98.0 |
| | 3.10 | 98.0 |
| | 3.11 | 15.0 |
| | 5.00 | stop |

| | |
|---|---|
| | Column: Agilent Eclipse XDB-C18 (4.6 × 150 mm, 5 μm) |
| | Flow rate: 0.80 mL/min |
| | Column temperature: 40 ° C. |
| | Retention time: |
| | LB-100: 1.75 min |
| | Diclofenac: 3.56 min |
| | Propranolol: 2.77 min |
| | For plasma samples: |
| | An aliquot of 30 μL sample was added with 100 μL IS |
| | (Diclofenac, 100 ng/mL and Propranolol, 50 ng/mL in ACN). |
| | The mixture was vortexed for 10 min at 750 rpm and |
| | centrifuged at 6000 rpm for 10 min. An aliquot of 5 μL |
| | supernatant was injected for LC-MS/MS analysis. |
| | For brain homogenate samples: |
| | The brain samples were homogenized with 3 volumes (v/w) of |
| | homogenizing solution PBS (pH 7.4) for 2 mins. An aliquot |
| | of 30 μL tissue homogenate sample was added with 100 μL IS |
| | (Diclofenac, 100 ng/mL and Propranolol, 50 ng/mL in ACN). |
| | Vortex at 750 rpm for 10 min and centrifuged at 6000 rpm |
| | for 10 min. An aliquot of 5 μL supernatant was injected |
| | for LC-MS/MS analysis. |
| | For liver homogenate samples: |
| | The liver samples were homogenized with 3 volumes (v/w) of |
| | homogenizing solution PBS (pH 7.4) for 2 mins. An aliquot |
| | of 30 μL tissue homogenate sample was added with 100 μL IS |
| | (Diclofenac, 100 ng/mL and Propranolol, 50 ng/mL in ACN). |
| | Vortex at 750 rpm for 10 min and centrifuged at 6000 rpm |
| | for 10 min. An aliquot of 5 μL supernatant was injected |
| | for LC-MS/MS analysis. |
| | For all the samples preparation, allow calibration, |
| | quality control, blanks, and test samples to thaw at 4° C. |
| | (nominal). And keep each step on an ice bath or at 4° C. . |
| Calibration | 3-3000 ng/mL for LB-100 in SD rat plasma; |
| curve | 6-3000 ng/mL for LB-100 in SD rat brain and liver |
| | homogenate. |

### Pharmacokinetic Analysis

Software:

The PK parameters were determined by non-compartmental model of non-compartmental analysis tool, Pharsight Phoenix WinNonlin® 6.2 software.

### “BQL” Rule:

Concentration data under 80% of LLOQ (LLOQ=1.00 ng/mL in rat plasma, brain and liver homogenate for 113. LLOQ=20.00 ng/mL in rat plasma, brain and liver homogenate for Endothal. LLOQ=3.00 ng/mL for 100 in rat plasma, 6.00 ng/mL for 100 in rat brain and liver homogenate) was replaced with “BQL” and excluded from graphing and PK parameters estimation. Concentration data within 80%-120% of LLOQ was considered within normal instrumental variation and presented in the results.

Terminal t_{1/2} calculation:

Time points were automatic selected by “best fit” model for terminal half life estimation as the first option. Manual selection was applied when “best fit” could not well define the terminal phase.

### Results

No abnormal clinical symptom was observed after IV and PO administrations.

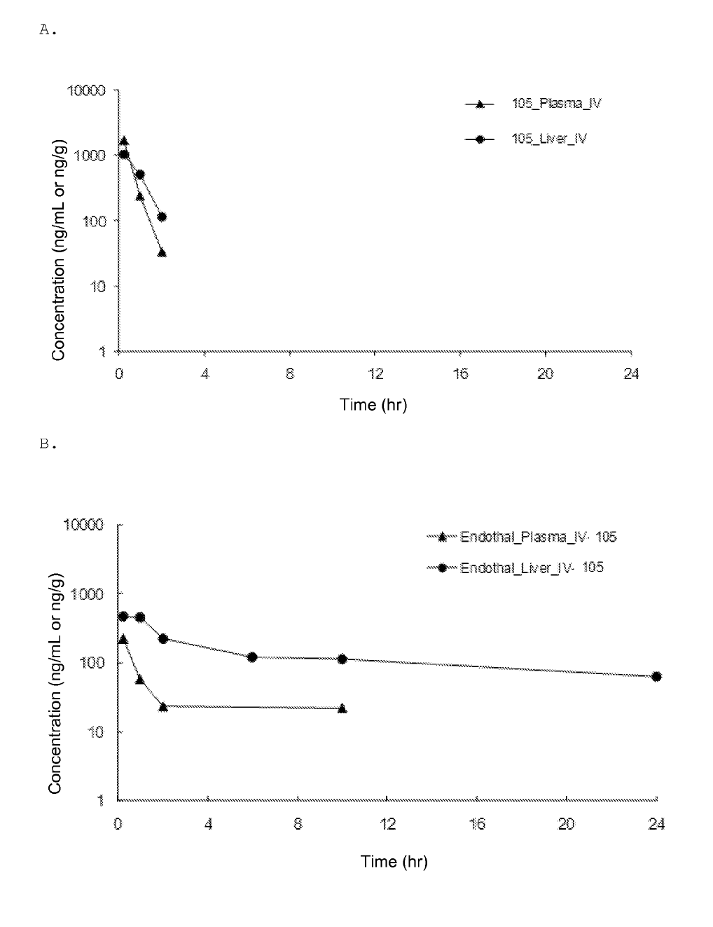

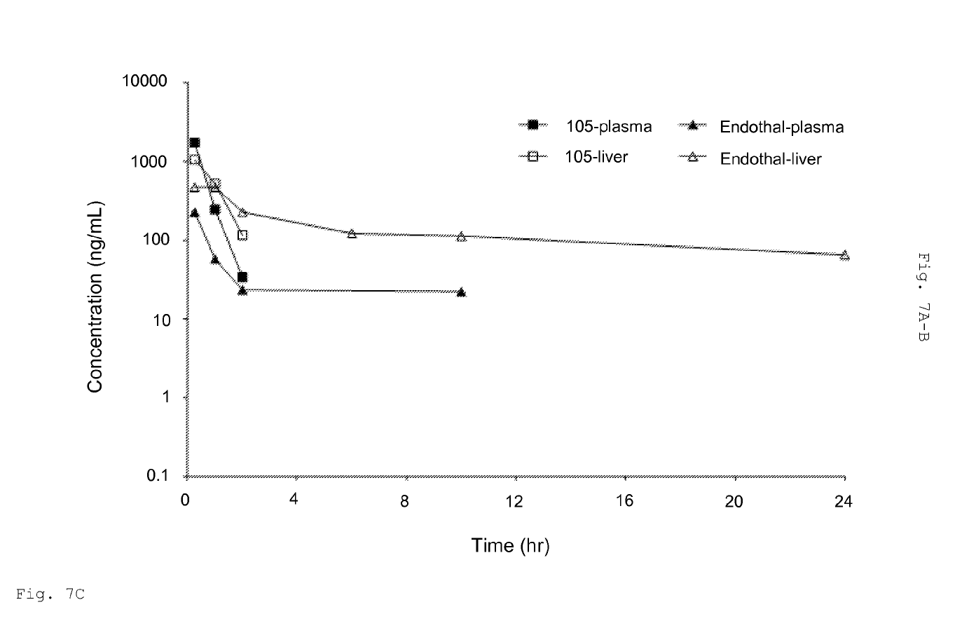

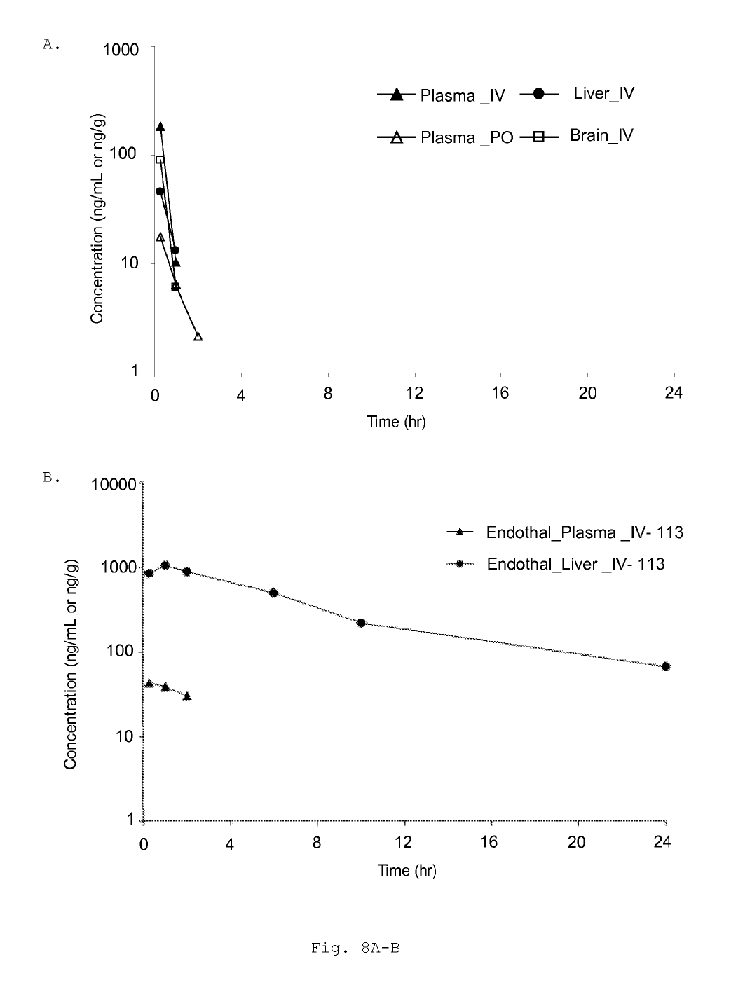

The concentration-time data and pharmacokinetic parameters of 113, 100 and Endothal in rat plasma, brain and liver after IV or PO administrations were listed in Tables 8.1 to 8.19, and illustrated in FIGS. 8A-8D.

TABLE 8.1
Individual and mean plasma concentration-time data
of 113 after an IV dose of 1.4 mg/kg in male SD rats

| Time (hr) | Individual | | Mean (ng/mL) |
|---|---|---|---|
| 0.25 | 173 | 193 | 183 |
| 1 | 10.8 | 9.96 | 10.4 |
| 2 | BQL | BQL | SQL |
| 6 | BQL | BQL | BQL |
| 10 | BQL | BQL | BQL |
| 24 | BQL | BQL | BQL |

LLOQ of 113 in plasma sample is 1.00 ng/mL.

ULOQ of 113 in plasma sample is 3000 ng/mL.

BLQ: Below Limit of Quantitation

TABLE 8.2
Individual and mean plasma concentration-time data of 113
after a PO dose of 1.4 mg/kg in male SD rats

| Time (hr) | Individual | | Mean (ng/mL) |
|---|---|---|---|
| 0.25 | 18.3 | 17.0 | 17.7 |
| 1 | 4.61 | 8.56 | 6.59 |
| 2 | BQL | 2.15 | 2.15 |
| 6 | BQL | BQL | BQL |
| 10 | BQL | BQL | BQL |
| 24 | BQL | BQL | BQL |

LLOQ of 113 in plasma sample is 1.00 ng/mL.

ULOQ of 113 in plasma sample is 3000 ng/mL.

BLQ: Below Limit of Quantitation

TABLE 8.3
Individual and mean liver concentration-time data of 113
after an IV dose of 1.4 mg/kg in male SD rats

| Time (hr) | Individual | | Mean (ng/g) |
|---|---|---|---|
| 0.25 | 55.5 | 36.9 | 46.2 |
| 1 | 14.6 | 11.8 | 13.2 |
| 2 | BQL | BQL | BQL |
| 6 | BQL | BQL | BQL |
| 10 | BQL | BQL | BQL |
| 24 | BQL | BQL | BQL |

The liver sample is homogenized with 3 volumes (v/w) of homogenizing solution (PBS PH7.4).

Liver concentration=liver homogenate conc.×4, assuming 1 g wet liver tissue equals to 1 mL.

LLOQ of 113 in liver homogenate sample is 1.00 ng/mL.

ULOQ of 113 in liver homogenate sample is 3000 ng/mi.

BLQ: Below Limit of Quantitation

TABLE 8.4
Liver-plasma concentration ratio of 113 after an
IV dose of 1.4 mg/kg in male SD rats

| Time (hr) | Individual | | Mean |
|---|---|---|---|
| 0.25 | 0.321 | 0.191 | 0.256 |
| 1 | 1.35 | 1.18 | 1.27 |
| 2 | NA | NA | NA |
| 6 | NA | NA | NA |
| 10 | NA | NA | NA |
| 24 | NA | NA | NA |

NA: Not Applicable

TABLE 8.5
Individual and mean brain concentration-time data of 113
after an IV dose of 1.4 mg/kg in male SD rats

| Time (hr) | Individual | | Mean (ng/g) |
|---|---|---|---|
| 0.25 | 86.2 | 94.5 | 90.4 |
| 1 | 5.80 | 6.42 | 6.11 |
| 2 | BQL | BQL | BQL |
| 6 | BQL | BQL | BQL |
| 10 | BQL | BQL | BQL |
| 24 | BQL | BQL | BQL |

The brain sample is homogenized with 3 volumes (v/w) of homogenizing solution (PBS PH7.4).

Brain concentration=brain homogenate conc.×4, assuming 1 g wet brain tissue equals to 1 mL.

LLOQ of 113 in brain homogenate sample is 1.00 ng/mL.

ULOQ of 113 in brain homogenate sample is 3000 ng/mL.

BLQ: Below Limit of Quantitation

TABLE 8.6
Brain-plasma concentration ratio of 113after an
IV dose of 1.4 mg/kg in male SD rats

| Time (hr) | Individual | | Mean |
|---|---|---|---|
| 0.25 | 0.498 | 0.490 | 0.494 |
| 1 | 0.537 | 0.645 | 0.591 |
| 2 | NA | NA | NA |
| 6 | NA | NA | NA |
| 10 | NA | NA | NA |
| 24 | NA | NA | NA |

NA: Not Applicable

TABLE 8.7
Individual and mean plasma concentration-time data of
Endothal after an IV dose of 1.4 mg/kg 113 in SD rats

| Time (hr) | Individual | | Mean (ng/mL) |
|---|---|---|---|
| 0.25 | 24.9 | 61.2 | 43.1 |
| 1 | 41.6 | 36.1 | 38.9 |
| 2 | 43.3 | 17.4 | 30.4 |
| 6 | BQL | BQL | BQL |
| 10 | BQL | BQL | BQL |
| 24 | BQL | BQL | BQL |

LLOQ of Endothal in plasma sample is 20.0 ng/mL.

ULOQ of Endothal in plasma sample is 3000 ng/mL.

BLQ: Below Limit of Quantitation

TABLE 8.8
Individual and mean liver concentration-time data of
Endothal after an IV dose of 1.4 mg/kg 113 in SD rats

| Time (hr) | Individual | | Mean (ng/g) |
|---|---|---|---|
| 0.25 | 727 | 988 | 858 |
| 1 | 902 | 1230 | 1066 |
| 2 | 998 | 795 | 897 |
| 6 | 526 | 477 | 502 |
| 10 | 288 | 157 | 223 |
| 24 | 66.9 | 68.8 | 67.9 |

The liver sample is homogenized with 3 volumes (v/w) of homogenizing solution (PBS PH7.4).

Liver concentration=liver homogenate conc.×4, assuming 1 g wet liver tissue equals to 1 mL.

LLOQ of Endothal in liver homogenate sample is 20.0 ng/mL.

ULOQ of Endothal in liver homogenate sample is 3000 ng/mL.

BLQ: Below Limit of Quantitation

TABLE 8.9
Liver-plasma concentration ratio of Endothal
after an IV dose of 1.4 mg/kg 113 in SD rats

| Time (hr) | Individual | | Mean |
|---|---|---|---|
| 0.25 | 29.2 | 16.1 | 22.7 |
| 1 | 21.7 | 34.1 | 27.9 |
| 2 | 23.0 | 45.7 | 34.4 |
| 6 | NA | NA | NA |
| 10 | NA | NA | NA |
| 24 | NA | NA | NA |

NA: Not Applicable

TABLE 8.10
Individual and mean brain concentration-time data of
Endothal after an IV dose of 1.4 mg/kg 113 in SD rats

| Time (hr) | Individual | | Mean (ng/g) |
|---|---|---|---|
| 0.25 | BQL | BQL | BQL |
| 1 | BQL | BQL | BQL |
| 2 | BQL | BQL | BQL |
| 6 | BQL | BQL | BQL |
| 10 | BQL | BQL | BQL |
| 24 | BQL | BQL | BQL |

The brain sample is homogenized with 3 volumes (v/w) of homogenizing solution (PBS PH7.4).

Brain concentration=brain homogenate conc.×4, assuming 1 g wet brain tissue equals to 1 mL.

LLOQ of Endothal in brain homogenate sample is 20.0 ng/mL.

ULOQ of Endothal in brain homogenate sample is 3000 ng/mL.

BLQ: Below Limit of Quantitation

TABLE 8.11
Brain-plasma concentration ratio of Endothal after
an IV dose of 1.4 mg/kg 113 in SD rats

| Time (hr) | Individual | | Mean |
|---|---|---|---|
| 0.25 | NA | NA | NA |
| 1 | NA | NA | NA |
| 2 | NA | NA | NA |
| 6 | NA | NA | NA |
| 10 | NA | NA | NA |
| 24 | NA | NA | NA |

NA: Not Applicable

TABLE 8.12
Individual and mean plasma concentration-time data
of 100 after an IV dose of 1.4 mg/kg 113 in SD rats

| Time (hr) | Individual | | Mean (ng/mL) |
|---|---|---|---|
| 0.25 | 510 | 598 | 554 |
| 1 | 273 | 170 | 222 |
| 2 | 135 | 45.3 | 90.2 |
| 6 | 3.25 | BQL | 3.25 |
| 10 | SQL | BQL | BQL |
| 24 | SQL | BQL | BQL |

LLOQ of 100 in plasma sample is 3.00 ng/mL.

ULOQ of 100 in plasma sample is 3000 ng/mL.

BLQ: Below Limit of Quantitation

TABLE 13
Individual and mean liver concentration-time data
of 100 after an IV dose of 1.4 mg/kg 113 in SD rats

| Time (hr) | Individual | | Mean (ng/g) |
|---|---|---|---|
| 0.25 | 2090 | 1700 | 1895 |
| 1 | 1360 | 690 | 1025 |
| 2 | 425 | 306 | 366 |
| 6 | 23.8 | 21.8 | 22.8 |
| 10 | BQL | BQL | BQL |
| 24 | BQL | BQL | BQL |

The liver sample is homogenized with 3 volumes (v/w) of homogenizing solution (PBS pH7.4).

Liver concentration=liver homogenate conc.×4, assuming 1 g wet liver tissue equals to 1 mL.

LLOQ of 100 in liver homogenate sample is 6.00 ng/mL.

ULOQ of 100 in liver homogenate sample is 3000 ng/mL.

BLQ: Below Limit of Quantitation

TABLE 8.14
Liver-plasma concentration ratio of 100 after
an IV dose of 1.4 mg/kg 113 in SD rats

| Time (hr) | Individual | | Mean |
|---|---|---|---|
| 0.25 | 4.10 | 2.84 | 3.47 |
| 1 | 4.98 | 4.06 | 4.52 |
| 2 | 3.15 | 6.75 | 4.95 |
| 6 | 7.32 | NA | 7.32 |
| 10 | NA | NA | NA |
| 24 | NA | NA | NA |

NA: Not Applicable

TABLE 8.15
Individual and mean brain concentration-time data
of 100 after an IV dose of 1.4 mg/kg 113 in SD rats

| Time (hr) | Individual | | Mean (ng/g) |
|---|---|---|---|
| 0.25 | BQL | BQL | BQL |
| 1 | BQL | BQL | BQL |
| 2 | BQL | BQL | BQL |
| 6 | BQL | BQL | BQL |
| 10 | BQL | BQL | BQL |
| 24 | BQL | BQL | BQL |

The brain sample is homogenized with 3 volumes (v/w) of homogenizing solution (PBS PH7.4).

Brain concentration=brain homogenate conc.×4, assuming 1 g wet brain tissue equals to 1 mL.

LLOQ of 100 in brain homogenate sample is 6.00 ng/mL.

ULOQ of 100 in brain homogenate sample is 3000 ng/mL.

BLQ: Below Limit of Quantitation

TABLE 8.16
Brain-plasma concentration ratio of 100 after
an IV dose of 1.4 mg/kg 113 in SD rats

| Time (hr) | Individual | | Mean |
|---|---|---|---|
| 0.25 | NA | NA | NA |
| 1 | NA | NA | NA |
| 2 | NA | NA | NA |
| 6 | NA | NA | NA |
| 10 | NA | NA | NA |
| 24 | NA | NA | NA |

NA: Not Applicable

TABLE 8.17
Mean Pharmacokinetics Parameters of 113 after an IV dose
of 1.4 mg/kg in male SD rats

| | Dosing | | | | | | | | | | |
|---|---|---|---|---|---|---|---|---|---|---|---|
| | Route | AUC₍₀₋ₜ₎ | AUC_{(0-∞)} | t_{1/2} | Tₘₐₓ | Cₘₐₓ | CL | Vₛₛ | MRT_{INF} | F | AUC_{last-liver(brain)}/ |
| Matrix | (Dose) | h * ng/mL | h * ng/mL | hr | hr | ng/mL | L/hr/kg | L/kg | hr | % | AUCₗₐₛₜ₋ₚₗₐₛₘₐ |
| Plasma | PO | 15.7 | NA | NA | 0.25 | 17.7 | NA | NA | NA | 10.1 | NA |
| | (1.4 mg/kg) | | | | | | | | | | |
| Plasma | IV | 155 | NA | NA | NA | NA | NA | NA | NA | NA | NA |
| Liver | (1.4 mg/kg) | 28.1 | NA | NA | 0.25 | 46.2 | NA | NA | NA | NA | 18.1 |
| Brain | | 47.5 | NA | NA | 0.25 | 90.4 | NA | NA | NA | NA | 30.6 |

TABLE 8.18
Mean Pharmacokinetics Parameters of Endothal after an IV
dose of 1.4 mg/kg 113 in male SD rats

| | Dosing | | | | | | |
|---|---|---|---|---|---|---|---|
| | Route | AUC₍₀₋ₜ₎ | AUC_{(0-∞)} | t_{1/2} | Tₘₐₓ | Cₘₐₓ | AUCₗₐₛₜ₋ₗᵢᵥₑᵣ/ |
| Matrix | (Dose) | h * ng/mL | h * ng/mL | hr | hr | ng/mL | AUCₗₐₛₜ₋ₚₗₐₛₘₐ |
| Plasma | IV | 70.7 | NA | NA | 0.25 | 43.1 | NA |
| Liver | (1.4 mg/kg) | 8086 | 8678 | 6.04 | 1 | 1066 | 11438 |
| Brain | | NA | NA | NA | NA | NA | NA |

TABLE 8.19
Mean Pharmacokinetics Parameters of 100 after an IV dose
of 1.4 mg/kg 113 in male SD rats

| | Dosing | | | | | | |
|---|---|---|---|---|---|---|---|
| | Route | AUC₍₀₋ₜ₎ | AUC_{(0-∞)} | t_{1/2z} | Tₘₐₓ | Cₘₐₓ | AUCₗₐₛₜ₋ₗᵢᵥₑᵣ/ |
| Matrix | (Dose) | h * ng/mL | h * ng/mL | hr | hr | ng/mL | AUCₗₐₛₜ₋ₚₗₐₛₘₐ |
| Plasma | IV (1 mg/kg) | 703 | 707 | 0.825 | 0.25 | 554 | NA |
| Liver | | 2804 | 2834 | 0.934 | 0.25 | 1895 | 399 |
| Brain | | NA | NA | NA | NA | NA | NA |

IV-1.4 mg/kg 113

After an IV dose of 113 at 1.4 mg/kg in male SD rats, the area under curve from time 0 to last time point (AUCₗₐₛₜ) was 155 hr*ng/mL.

The mean values of Cₘₐₓ in liver was 46.2 ng/g and corresponding Tₘₐₓ value was 0.25 hr. The mean value of AUC₍₀₋ₗₐₛₜ₎ was 28.1 ng/g*hr. AUC₍₀₋ₜ₎ ratio of liver over plasma was 18.1.

The mean values of Cₘₐₓ in brain was 90.4 ng/g and corresponding Tₘₐₓ value was 0.25 hr. The mean value of AUC₍₀₋ₗₐₛₜ₎ was 47.5 ng/g*hr. AUC₍₀₋ₜ₎ ratio of liver over plasma was 30.6.

PO-1.4 mg/kg 113

After a PO dose of 113 at 1.4 mg/kg, the Cₘₐₓ value in rat plasma was 17.7 ng/mL, and corresponding mean Tₘₐₓ value was 0.250 hr. The area under curve from time 0 to last time point AUCₗₐₛₜ was 15.7 hr*ng/mL. After the IV dose of 1.4 mg/kg and the PO dose of 1.4 mg/kg, the bioavailability of this compound in SD rat was estimated to be 10.1%.

### Endothal

Following intravenous administration of 1.4 mg/kg 113 to Male SD rats, the area under curve from time 0 to last time point (AUCₗₐₛₜ) was 70.7 hr*ng/mL. The mean values of Cₘₐₓ and Tₘₐₓ in plasma were 43.1 ng/mL and 0.25 hr, respectively.

The mean values of Cₘₐₓ in liver was 1066 ng/g and corresponding Tₘₐₓ value was 1.00 hr. The mean value of AUC₍₀₋ₗₐₛₜ₎ and AUC_{(0-∞)} were 8086 and 8678 ng/g*hr, respectively. AUC₍₀₋ₜ₎ ratio of liver over plasma was 11438.

### Compound 100

The mean values of Cₘₐₓ and Tₘₐₓ in plasma were 554 ng/mL and 0.25 hr, respectively. The mean value of AUC₍₀₋ₗₐₛₜ₎ and AUC_{(0-∞)} were 703 ng/mL*hr and 707 ng/mL*hr, respectively.

The mean values of Cₘₐₓ in liver was 1895 ng/g and corresponding Tₘₐₓ value was 0.25 hr. The mean value of AUC₍₀₋ₗₐₛₜ₎ and AUC_{(0-∞)} were 2804 ng/g*hr and 2834 ng/g*hr, respectively. AUC₍₀₋ₜ₎ ratio of liver over plasma was 399.

### Example 11

### Pharmacokinetic Study of Compound 151

A pharmacokinetic study of 151 was conducted in SD rats. The study consisted of two dose levels at 1.0 (iv) and 10 (oral) mg/kg. The blood samples were collected at predetermined times from rats and centrifuged to separate plasma. An LC/MS/MS method was developed to determine the test article in plasma samples. The pharmacokinetic parameters of 151 following iv and oral administration to SD rats were calculated. The absolute bioavailability was evaluated.

### Study Design

A total of 5 male SD rats were assigned to this study as shown in the table below:

| | Number of | | Dose | Dose |
|---|---|---|---|---|
| | rats | Route of | level | volume |
| Groups | (male) | administration | (mg/kg) | (ml/kg) |
| 1 | 3 | oral | 10 | 10 |
| 2 | 2 | iv | 1.0 | 5.0 |

### Dose Preparation and Dose Administration

151 (MW 282.34, purity 99.2%, lot no. 20110512) was prepared by dissolving the article in PBS (pH 7.4) on the day of dosing. The final concentration of the test article was 0.2 mg/mL for iv administration and 1.0 mg/mL for oral administration. The test article solutions were administered using the most recent body weight for each animal.

### Sample Collection

Blood (approximately 0.3 mL) were collected via orbital plexus into tubes containing sodium heparin at 0.25, 0.5, 1, 2, 3, 5, 7, 9, and 24 hours after oral administration; at 5 min, 15 min, 0.5, 1, 2, 3, 5, 7, 9 and 24 hours after iv administration. Samples were centrifuged for 5 min, at 4° C. with the centrifuge set at 11,000 rpm to separate plasma. The obtained plasma samples were stored frozen at a temperature of about −70° C. until analysis.

### Preparation of Plasma Samples

Frozen plasma samples were thawed at room temperature and vortexed thoroughly. With a pipette, an aliquot (30 μL) of plasma was transferred into a 1.5-mL conical polypropylene tube. To each sample, 160 μL of acetonitrile were added. The samples were then vigorously vortex-mixed for 1 min. After centrifugation at 11000 rpm for 5 min, a 15 μL aliquot of the supernatant was injected into the LC-MS/MS system for analysis.

### Preparation of Calibration Samples

Calibration standards were prepared by spiking 30 μL of the 151 standard solutions into 30 μL of heparinized blank rat plasma. The nominal standard concentrations in the standard curve were 1.00, 3.00, 10.0, 30.0, 100, 300, 1000 and 3000 ng/mL.

### LC/MS/MS System

The analysis was performed using an LC-MS/MS system consisting of the following components—HPLC system: Agilent 1200 series instrument consisting of G1312B vacuum degasser, G1322A binary pump, G1316B column oven and G1367D autosampler (Agilent, USA); MS/MS system: Agilent 6460 triple quadrupole mass spectrometer, equipped with an APCI Interface (Agilent, USA); Data system: MassHunter Software (Agilent, USA).

### Chromatographic Conditions

Chromatographic separation was carried out at room temperature—Analytical column: C₈ column (4.6 mm×150 mm I.D., 5 μm, Agilent, USA); Mobile phase: Acetonitrile:10 mM ammonium acetate (75:25, v/v); Flow rate: 0.80 mL/min; Injection volume: 15 μL.

### Mass Spectrometric Conditions

The mass spectrometer was operated in the positive mode. Ionization was performed applying the following parameters: gas temperature, 325° C.; vaporizer temperature, 350° C.; gas flow, 4 L/min; nebulizer, 20 psi; capillary voltage, 4500 V; corona current, 4 μA. 151 was detected using MRM of the transitions m/z 283→m/z 123 and m/z 283→m/z 251, simultaneously. The optimized collision energies of 25 eV and 10 eV were used for m/z 123 and m/z 251, respectively.

### Quantification

Quantification was achieved by the external standard method. Concentrations of the test article were calculated using a weighted least-squares linear regression (W=1/x²).

### Pharmacokinetic Interpretation

The pharmacokinetic parameters were evaluated using WinNonlin version 5.3 (Pharsight Corp., Mountain View, Calif., USA), assuming a non-compartmental model for drug absorption and distribution.
AUC₀₋ₜ is the area under the plasma concentration-time curve from time zero to last sampling time, calculated by the linear trapezoidal rule.
AUC_{0-∞} is the area under the plasma concentration-time curve from time zero extrapolating to infinity.
T_{1/2} is the elimination half-life associated with the terminal (log-linear) elimination phase, which is estimated via linear regression of time vs. log concentrations.
CL is the total body clearance.
Vₛₛ is the volume of distribution at steady-state.

### Calibration Curve for Plasma Samples

The calibration curve for L151 in rat plasma was linear throughout the study in the range of 1.00-3000 ng/mL. The linear regression equation of the calibration curve was y=885.6448 x+791.9622, r²=0.9927, where y represents the peak area of 151 and x represents the plasma concentrations of 151.

### Plasma Concentrations of 151 in SD Rats

Following iv (1.0 mg/kg) and oral (10 mg/kg) administration of 151 to SD rats, plasma concentrations of the test articles were determined by the LC/MS/MS method described above. The plasma concentrations at each sampling time are listed in Tables 9.1 and 9.2.

### Interpretation of Pharmacokinetics

The major pharmacokinetic parameters of 151 in plasma are summarized in Tables 9.3 and 9.4. Following oral administration of 10 mg/kg to SD rats (n=3), 151 was rapidly absorbed with peak plasma concentration occurring at 0.5 h after dose. The elimination of 151 was fast with mean half-life of 1.26 h. Following iv administration of 1.0 mg/kg (n=2), the elimination half-life of 151 was 0.89 h. The mean clearance of 151 from rat plasma and the volume of distribution at steady state were 859 ml/h/kg and 736 ml/kg. Based on the exposure (AUC_{0-∞}), the absolute bioavailability (F) of 151 was 54.6% following oral administration at 10 mg/kg to SD rats.

TABLE 9.1
Analytical data of 151 plasma concentration (ng/mL)
in SD rats following PO administration at 10 mg/kg.

| | Time (h) | | | | | | | | |
|---|---|---|---|---|---|---|---|---|---|
| Rat No. | 0.25 | 0.50 | 1.0 | 2.0 | 3.0 | 5.0 | 7.0 | 9.0 | 24 |
| 1 | 2231 | 2451 | 2204 | 1100 | 521 | 125 | 42.6 | 52.1 | BLQ |
| 2 | 2029 | 3934 | 2581 | 1237 | 660 | 99.4 | 20.7 | 38.2 | BLQ |
| 3 | 2731 | 3343 | 2538 | 1582 | 794 | 192 | 68.0 | 66.1 | BLQ |
| Mean | 2330 | 3243 | 2441 | 1306 | 658 | 139 | 43.8 | 52.1 | |
| SD | 361 | 747 | 206 | 248 | 136 | 48 | 23.6 | 13.9 | |

BLQ: Below the lower limit of quantification 1.00 ng/mL.

TABLE 9.2
Analytical data of 151 plasma concentration (ng/mL)
in SD rats following IV administration at 1.0 mg/kg.

| | Time (h) | | | | | | | | | |
|---|---|---|---|---|---|---|---|---|---|---|
| Rat No. | 0.083 | 0.250 | 0.50 | 1.0 | 2.0 | 3.0 | 5.0 | 7.0 | 9.0 | 24 |
| 4 | 1677 | 1160 | 760 | 381 | 95.8 | 39.6 | 9.75 | 12.2 | BLQ | BLQ |
| 5 | 1301 | 949 | 807 | 314 | 103 | 28.1 | 3.63 | 1.83 | 2.01 | BLQ |
| Mean | 1489 | 1055 | 683 | 348 | 99.6 | 33.8 | 6.69 | 7.02 | 1.00 | |

TABLE 9.3
The main pharmacokinetic parameters of 151 in SD
rats following PO administration at 10 mg/kg.

| Rat | Tmax | Cmax | AUC₀₋ₜ | AUC_{0-∞} | T_{1/2} | MRT | F |
|---|---|---|---|---|---|---|---|
| No. | (ng/ml) | (ng/ml) | (ng · h/ml) | (ng · h/ml) | (h) | (h) | (%) |
| 1 | 0.50 | 2451 | 5399 | 5499 | 1.33 | 1.86 | |
| 2 | 0.50 | 3934 | 6423 | 6484 | 1.10 | 1.62 | |
| 3 | 0.50 | 3343 | 7199 | 7328 | 1.35 | 1.95 | |
| Mean | 0.50 | 3243 | 6340 | 6437 | 1.26 | 1.81 | 54.6 |
| SD | 0.00 | 747 | 903 | 916 | 0.14 | 0.17 | |
| CV (%) | 0.0 | 23.0 | 14.2 | 14.2 | 11.0 | 9.4 | |

TABLE 9.4
The main pharmacokinetic parameters of 151
in SD rats following IV administration at 1.0 mg/kg.

| Rat | AUC₀₋ₜ | AUC_{0-∞} | T_{1/2} | MRT | Vₛₛ | CL |
|---|---|---|---|---|---|---|
| No. | (ng · h/ml) | (ng · h/ml) | (h) | (h) | (ml/kg) | (ml/h/kg) |
| 4 | 1293 | 1309 | 0.91 | 0.91 | 696 | 764 |
| 5 | 1045 | 1047 | 0.87 | 0.81 | 775 | 955 |
| Mean | 1169 | 1178 | 0.89 | 0.86 | 736 | 859 |

100 concentrations of the 151 plasma samples were also measured and pharmacokinetic parameters were calculated. 151 was converted to LB100 (see Tables 9.5-9.8).

TABLE 9.5
Plasma Concentrations of 100 after PO administration
of 10 mg/kg 151 to SD rat (ng/mL)

| | Rat | Time (h) | | | | | | | | |
|---|---|---|---|---|---|---|---|---|---|---|
| Group | No. | 0.25 | 0.50 | 1.0 | 2.0 | 3.0 | 5.0 | 7.0 | 9.0 | 24 |
| PO-10 mg/kg | 1 | 966 | 1426 | 882 | 734 | 236 | 81.1 | 37.9 | 31.6 | BLQ |
| | 2 | 522 | 1489 | 1141 | 645 | 396 | 79.4 | 20.3 | 22.5 | BLQ |
| | 3 | 1056 | 1439 | 1447 | 963 | 624 | 185 | 56.0 | 39.6 | BLQ |
| | Mean | 848 | 1451 | 1156 | 781 | 419 | 115 | 38.1 | 31.3 | |
| | SD | 286 | 33 | 283 | 164 | 195 | 61 | 17.9 | 8.6 | |

BLQ: Below the lower limit of quantification 10.0 ng/mL

TABLE 9.6
Plasma Concentrations of 100 after iv administration
of 1.0 mg/kg 151 to SD rat (ng/mL)

| | Rat | Time (h) | | | | | | | | | |
|---|---|---|---|---|---|---|---|---|---|---|---|
| Group | No. | 0.083 | 0.25 | 0.5 | 1.0 | 2.0 | 3.0 | 5.0 | 7.0 | 9.0 | 24 |
| IV-1 mg/kg | 4 | 646 | 345 | 308 | 257 | 125 | 32.2 | 10.2 | BLQ | BLQ | BLQ |
| | 5 | 430 | 239 | 231 | 182 | 114 | 33.3 | BLQ | BLQ | BLQ | BLQ |
| | Mean | 538 | 292 | 270 | 219 | 120 | 32.7 | 5.10 | | | |

BLQ: Below the lower limit of quantification 10.0 ng/ml.

TABLE 9.7
PK parameters of 100 after PO administration of 10 mg/kg
151 to SD rat

| | Rat | Tₘₐₓ | Cₘₐₓ | AUC₀₋ₜ | AUC_{0-∞} | T_{1/2} | MRT |
|---|---|---|---|---|---|---|---|
| Group | No. | (h) | (ng/ml) | (ng · h/ml) | (ng · h/ml) | (h) | (h) |
| PO-10 | 1 | 0.50 | 1426 | 2795 | 2862 | 1.45 | 2.06 |
| mg/kg | 2 | 0.50 | 1489 | 3006 | 3046 | 1.25 | 1.96 |
| | 3 | 1.00 | 1447 | 4309 | 4391 | 1.43 | 2.29 |
| | Mean | 0.67 | 1454 | 3370 | 3433 | 1.38 | 2.10 |
| | SD | 0.29 | 32 | 820 | 835 | 0.11 | 0.17 |
| | CV | 43.3 | 2.2 | 24.3 | 24.3 | 8.1 | 8.1 |
| | (%) | | | | | | |

TABLE 9.8
PK parameters of 100 after iv administration of 1.0 mg/kg
151 to SD rat

| | Rat | Tₘₐₓ | Cₘₐₓ | AUC₀₋ₜ | AUC_{0-∞} | T_{1/2} | MRT |
|---|---|---|---|---|---|---|---|
| Group | No. | (h) | (ng/ml) | (ng · h/ml) | (ng · h/ml) | (h) | (h) |
| IV-1 | 4 | 0.083 | 646 | 681 | 694 | 0.88 | 1.16 |
| mg/kg | 5 | 0.083 | 430 | 481 | 526 | 0.93 | 1.27 |
| | Mean | 0.083 | 538 | 581 | 610 | 0.91 | 1.21 |

### Example 13

### Pharmacokinetic Study of Compound 100

The pharmacokinetic studies on 100 and its metabolite endothal were conducted in SD rats. 100 was administrated via iv route at 0.5, 1.0 and 1.5 mg/kg into SD rats. The blood, liver and brain tissue samples were collected at predetermined times from rats. The LC/MS/MS methods were developed to determine 100 and endothal in plasma, liver and brain samples. In the report, the concentrations of 100 and endothal in plasma, liver and brain samples were presented.

### Sample Collection

Twelve (12) female SD rats per group were dosed by iv with 100. The rats were fasted overnight prior to dosing, with free access to water. Foods were withheld for 2 hours post-dose. Blood, liver and brain tissue samples in two animals each group were collected at each time point, within 10% of the scheduled time for each time point. Two extra animals were used for analytic method development. Blood (>0.3 mL) were collected via aorta abdominalis in anaesthetic animalsinto tubes containing heparin at 15 min, 1, 2, 6, 10 and 24 hours after iv administration. Liver and brain tissues were collected immediately after animal death. The liver and brain tissues were excised and rinsed with cold saline to avoid blood residual. Upon collection, each sample was placed on ice and the blood samples were subsequently centrifuged (4° C., 11000 rpm, 5 min) to separate plasma. The obtained plasma, liver and brain tissue samples were stored at −70° C. until LC-MS/MS analysis.

### Pharmacokinetic Interpretation

The pharmacokinetic parameters were evaluated using WinNonlin version 5.3 (Pharsight Corp., Mountain View, Calif., USA), assuming a non-compartmental model for drug absorption and distribution. AUC₀₋ₜ (AUCₗₐₛₜ) is the area under the plasma concentration-time curve from time zero to last sampling time, calculated by the linear trapezoidal rule. AUC_{0-∞} (AUC/NF) is the area under the plasma concentration-time curve with last concentration extrapolated based on the elimination rate constant.

### Plasma, Liver and Brain Tissue Concentrations of Test Articles in SD Rats

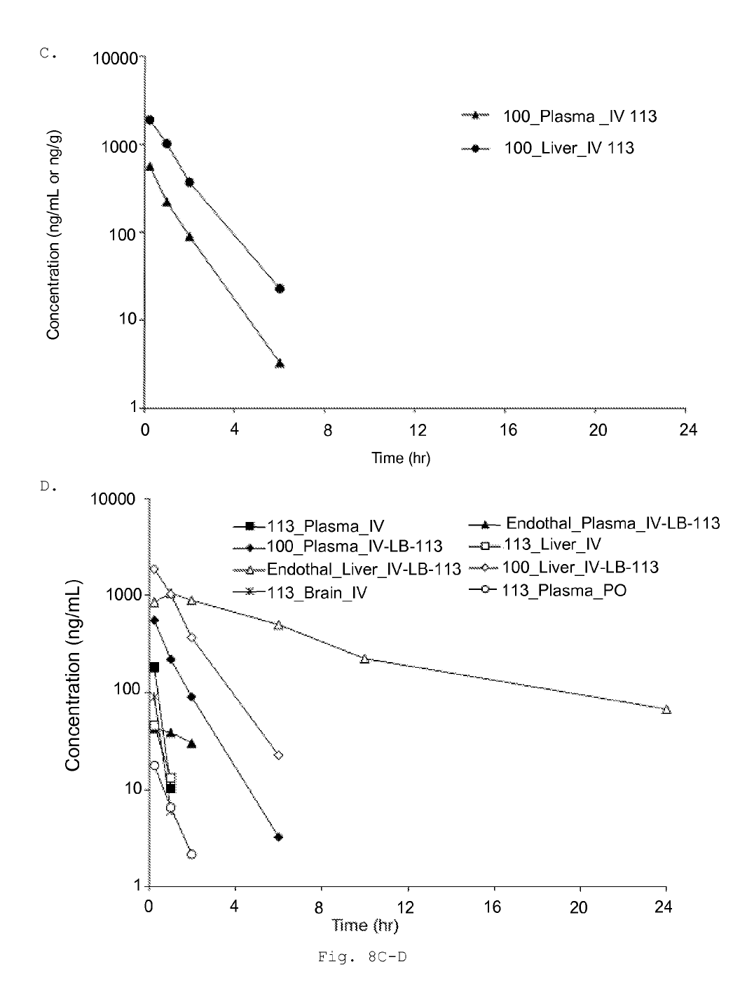

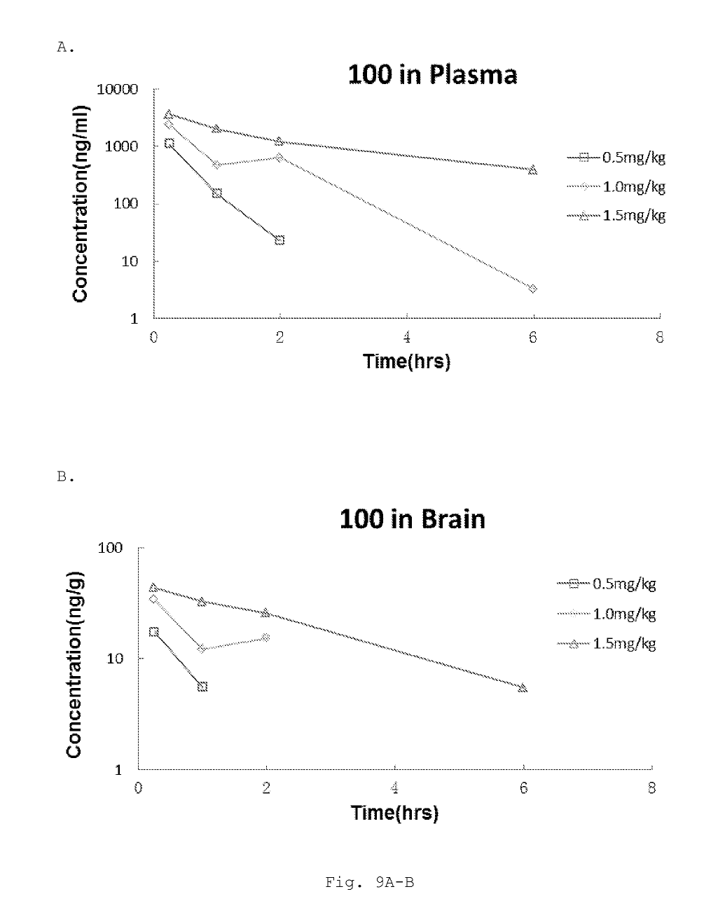

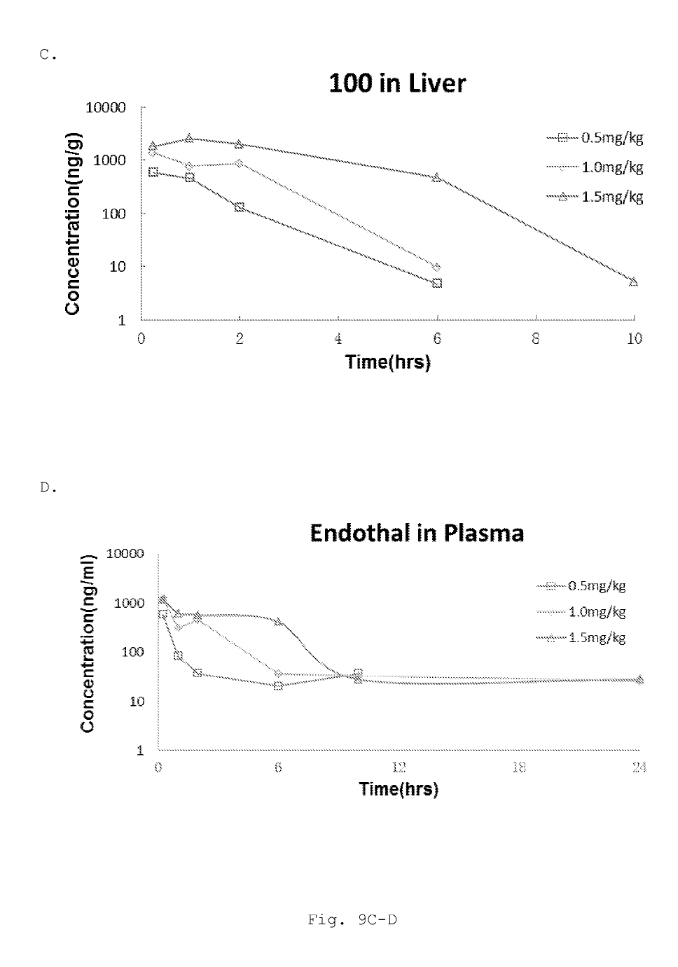

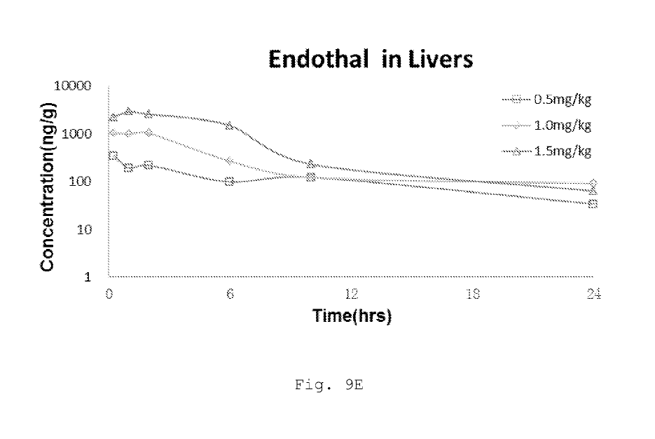

Following single iv administration of 100 to SD rats, plasma, liver and brain tissue concentrations of both 100 and endothal were determined by the LC/MS/MS method described above. The plasma, liver and brain tissue concentrations at each sampling time are listed in Tables 10.1-10.6 and FIG. 9A-9D. The calculated pharmacokinetic parameters are listed in Table 10.7-10.8. 100 could pass through blood-brain barrior (BBB) following iv administration at 0.5, 1.0 and 1.5 mg/kg to SD rats. The mean Cₘₐₓ in plasma was 11103664 ng/ml. The mean Cₘₐₓ in liver and brain were 586˜2548 ng/kg and 17.4˜43.5 ng/kg, respectively. AUCₗₐₛₜ in plasma was 695.8˜7399.6 ng·h/ml, with 758.6˜9081.0 ng·h/g in liver and 10.8˜125.5 ng·h/g in brain, respectively. TV2 in plasma, liver and brain were 0.31˜2.20 h, 0.78˜2.01 h and 1.67˜1.93 h, respectively.

As shown in table 10.4-10.6 and FIG. 9D-9E, endothal was detectable in plasma and liver samples following single iv administration of 100 at 0.5, 1.0 and 1.5 mg/kg, and the concentrations in plasma and liver increased with dose level of 100, whereas endothal was not detectable in brain samples. The mean Cₘₐₓ in plasma and liver were 577-1230 ng/ml and 349-2964 ng/ml, respectively. AUCₗₐₛₜ in plasma and liver were 546-4476 ng·h/ml and 2598-18434 ng·h/g, respectively. T_{1/2} in plasma and liver were 6.25-7.06 h and 4.57-10.1 h, respectively.

Following single iv administration, the mean Cₘₐₓ of 100 in plasma was 1110˜3664 ng/ml and T_{1/2} in plasma was 0.31˜2.20 h. AUCₗₐₛₜ in plasma was 695.8˜7399.6 ng·h/ml, and AUC increased proportionally with the dose level of 100. Following single iv administration, 100 was both detectable in liver and brain tissue samples. The concentration of 100 in liver samples was much higher than that in brain samples at same sampling time point, but 100 in liver and brain tissues was both below limit of quantification 24 hours after iv administration. Following single iv administration of 100, endothal was detectable and stay a long time in plasma and liver tissue. The mean Cₘₐₓ in plasma and liver were 577-1230 ng/ml and 349-2964 ng/ml, respectively. AUCₗₐₛₜ in plasma and liver were 546-4476 ng·h/ml and 2598-18434 ng·h/g, respectively. T_{1/2} in plasma and liver were 6.25-7.06 h and 4.57-10.1 h, respectively. However, endothal was undetectable in brain tissue.

TABLE 10.1
Analytical data of 100 plasma concentration (ng/mL)
in SD rats following iv administration.

| | | | | |
|---|---|---|---|---|
| Time (hr) | Rat 1 | Rat 2 | Mean | SD |

0.5 mg/kg Plasma concentration (ng/ml)

| | | | | |
|---|---|---|---|---|
| 0.25 | 1000 | 1219 | 1110 | 154.68 |
| 1 | 192 | 103 | 148 | 62.78 |
| 2 | 25.8 | 19.4 | 22.6 | 4.58 |
| 6 | BLQ | BLQ | BLQ | N/A |
| 10 | BLQ | BLQ | BLQ | N/A |
| 24 | BLQ | BLQ | BLQ | N/A |

1.0 mg/kg Plasma concentration (ng/ml)

| | | | | |
|---|---|---|---|---|
| 0.25 | 2118 | 2648 | 2383 | 374.46 |
| 1 | 354 | 595 | 474 | 170.92 |
| 2 | 1030 | 239 | 634.4 | 55912 |
| 6 | 3.27 | BLQ | BLQ | N/A |
| 10 | BLQ | BLQ | BLQ | N/A |
| 24 | BLQ | BLQ | BLQ | N/A |

1.5 mg/kg Plasma concentration (ng/ml)

| | | | | |
|---|---|---|---|---|
| 0.25 | 3779 | 3548 | 3664 | 162.94 |
| 1 | 1758 | 2273 | 2015 | 364.20 |
| 2 | 1314 | 1104 | 1209 | 148.70 |
| 6 | 263 | 519 | 391 | 180.40 |
| 10 | BLQ | BLQ | BLQ | N/A |
| 24 | BLQ | BLQ | BLQ | N/A |

TABLE 10.2
Analytical data of 100 liver concentration (ng/g)
in SD rats following iv administration.

| | | | | |
|---|---|---|---|---|
| Time (hr) | Rat 1 | Rat 2 | Mean | SD |

0.5 mg/kg Liver concentration (ng/g)

| | | | | |
|---|---|---|---|---|
| 0.25 | 520 | 651 | 586 | 92.76 |
| 1 | 695 | 223 | 459 | 333.91 |
| 2 | 109 | 148 | 128 | 27.06 |
| 6 | BLQ | 4.80 | BLQ | N/A |
| 10 | BLQ | BLQ | BLQ | N/A |
| 24 | BLQ | BLQ | BLQ | N/A |

1.0 mg/kg Liver concentration (ng/g)

| | | | | |
|---|---|---|---|---|
| 0.25 | 1299 | 1442 | 1371 | 101.47 |
| 1 | 865 | 682 | 773 | 129.61 |
| 2 | 1318 | 398 | 858 | 650.73 |
| 6 | 13.9 | 5.73 | 9.83 | 5.81 |
| 10 | BLQ | BLQ | BLQ | N/A |
| 24 | BLQ | BLQ | BLQ | N/A |

1.5 mg/kg Liver concentration (ng/g)

| | | | | |
|---|---|---|---|---|
| 0.25 | 1980 | 1709 | 1844 | 191.66 |
| 1 | 2144 | 2953 | 2548 | 571.97 |
| 2 | 2404 | 1585 | 1995 | 579.17 |
| 6 | 407 | 536 | 471 | 91.77 |
| 10 | BLQ | 5.25 | BLQ | N/A |
| 24 | BLQ | BLQ | BLQ | N/A |

TABLE 10.3
Analytical data of 100 brain concentration (ng/g)
in SD rats following iv administration.

| | | | | |
|---|---|---|---|---|
| Time (hr) | Rat 1 | Rat 2 | Mean | SD |

0.5 mg/kg Brain concentration (ng/g)

| | | | | |
|---|---|---|---|---|
| 0.25 | 15.3 | 19.5 | 17.42 | 3.02 |
| 1 | 6.31 | 4.77 | 5.54 | 1.09 |
| 2 | BLQ | BLQ | BLQ | N/A |
| 6 | BLQ | BLQ | BLQ | N/A |
| 10 | BLQ | BLQ | BLQ | N/A |
| 24 | BLQ | BLQ | BLQ | N/A |

1.0 mg/kg Brain concentration (ng/g)

| | | | | |
|---|---|---|---|---|
| 0.25 | 21.9 | 45.8 | 33.90 | 16.90 |
| 1 | 16.3 | 8.05 | 12.20 | 5.84 |
| 2 | 24.3 | 6.60 | 15.40 | 12.49 |
| 6 | BLQ | BLQ | BLQ | N/A |
| 10 | BLQ | BLQ | BLQ | N/A |
| 24 | BLQ | BLQ | BLQ | N/A |

1.5 mg/kg Brain concentration (ng/g)

| | | | | |
|---|---|---|---|---|
| 0.25 | 46.9 | 40.1 | 43.49 | 4.82 |
| 1 | 28.2 | 36.9 | 32.56 | 6.18 |
| 2 | 27.2 | 24.1 | 25.66 | 2.16 |
| 6 | 4.23 | 6.77 | 5.50 | 1.79 |
| 10 | BLQ | BLQ | BLQ | N/A |
| 24 | BLQ | BLQ | BLQ | N/A |

TABLE 10.4
Analytical data of endothal plasma concentration (ng/g)
in SD rats following iv administration.

| | | | | |
|---|---|---|---|---|
| Time (hr) | Rat 1 | Rat 2 | Mean | SD |

0.5 mg/kg Endothal plasma concentration (ng/ml)

| | | | | |
|---|---|---|---|---|
| 0.25 | 355 | 798 | 576 | 313.25 |
| 1 | 104 | 59.5 | 81.75 | 31.47 |
| 2 | 44.6 | 28.1 | 36.35 | 11.67 |
| 6 | 20.3 | BLQ | 20.3 | N/A |
| 10 | 48.1 | 25.3 | 36.70 | 16.12 |
| 24 | BLQ | BLQ | BLQ | N/A |

1.0 mg/kg Endothal plasma concentration (ng/ml)

| | | | | |
|---|---|---|---|---|
| 0.25 | 1310 | 1150 | 1230 | 113.14 |
| 1 | 164 | 456 | 310 | 206.48 |
| 2 | 699 | 213 | 456 | 343.65 |
| 6 | 33.6 | 38.2 | 35.90 | 3.25 |
| 10 | 32.9 | 31.8 | 32.35 | 0.78 |
| 24 | 29.4 | 22.0 | 25.70 | 5.23 |

1.5 mg/kg Endothal plasma concentration (ng/ml)

| | | | | |
|---|---|---|---|---|
| 0.25 | 1610 | 745 | 1177 | 611.65 |
| 1 | 760 | 458 | 609 | 213.55 |
| 2 | 539 | 600 | 569.50 | 43.13 |
| 6 | 373 | 444 | 408.50 | 50.20 |
| 10 | 22.3 | 33.1 | 27.70 | 7.64 |
| 24 | 21.5 | 34.1 | 27.80 | 8.91 |

TABLE 10.5
Analytical data of endothal liver concentration (ng/g)
in SD rats following iv administration of 100.

| | | | | |
|---|---|---|---|---|
| Time (hr) | Rat 1 | Rat 2 | Mean | SD |

0.5 mg/kg Endothal liver concentration (ng/g)

| | | | | |
|---|---|---|---|---|
| 0.25 | 316 | 382 | 349 | 46.67 |
| 1 | 256 | 131 | 193.50 | 88.39 |
| 2 | 168 | 273 | 220.50 | 74.25 |
| 6 | 85.8 | 112 | 98.90 | 18.53 |
| 10 | 129 | 118 | 123.50 | 7.78 |
| 24 | 32.0 | 36.4 | 34.20 | 3.11 |

1.0 mg/kg Endothal liver concentration (ng/g)

| | | | | |
|---|---|---|---|---|
| 0.25 | 768 | 1320 | 1044 | 390.32 |
| 1 | 1380 | 618 | 999 | 538.82 |
| 2 | 1530 | 542 | 1036 | 698.62 |
| 6 | 298 | 241 | 269.50 | 40.31 |
| 10 | 151 | 94.2 | 122.60 | 40.16 |
| 24 | 66.6 | 115 | 90.80 | 34.22 |

1.5 mg/kg Endothal liver concentration (ng/g)

| | | | | |
|---|---|---|---|---|
| 0.25 | 2298 | 2160 | 2229 | 97.58 |
| 1 | 2874 | 2976 | 2925 | 72.12 |
| 2 | 2952 | 2226 | 2589 | 513.36 |
| 6 | 1686 | 1326 | 1506 | 254.56 |
| 10 | 137 | 329 | 233 | 135.76 |
| 24 | 75.0 | 52.1 | 63.55 | 16.19 |

TABLE 10.6
Analytical data of endothal brain concentration (ng/g) in
SD rats following iv administration of 100.

| | | | | |
|---|---|---|---|---|
| Time (hr) | Rat 1 | Rat 2 | Mean | SD |

0.5 mg/kg Endothal brain concentration (ng/g)

| | | | | |
|---|---|---|---|---|
| 0.25 | BLQ | BLQ | BLQ | N/A |
| 1 | BLQ | BLQ | BLQ | N/A |
| 2 | BLQ | BLQ | BLQ | N/A |
| 6 | BLQ | BLQ | BLQ | N/A |
| 10 | BLQ | BLQ | BLQ | N/A |
| 24 | BLQ | BLQ | BLQ | N/A |

1.0 mg/kg Endothal brain concentration (ng/g)

| | | | | |
|---|---|---|---|---|
| 0.25 | BLQ | BLQ | BLQ | N/A |
| 1 | BLQ | BLQ | BLQ | N/A |
| 2 | BLQ | BLQ | BLQ | N/A |
| 6 | BLQ | BLQ | BLQ | N/A |
| 10 | BLQ | BLQ | BLQ | N/A |
| 24 | BLQ | BLQ | BLQ | N/A |

1.5 mg/kg Endothal brain concentration (ng/g)

| | | | | |
|---|---|---|---|---|
| 0.25 | BLQ | BLQ | BLQ | N/A |
| 1 | BLQ | BLQ | BLQ | N/A |
| 2 | BLQ | BLQ | BLQ | N/A |
| 6 | BLQ | BLQ | BLQ | N/A |
| 10 | BLQ | BLQ | BLQ | N/A |
| 24 | BLQ | BLQ | BLQ | N/A |

TABLE 10.7
Main pharmacokinetic parameters of 100 in SD rats following
iv administration.

| | | | | | Cmax | AUClast | AUCINF | |
|---|---|---|---|---|---|---|---|---|
| | Dose of | | | | ng/ml | ng · h/ml | ng · h/ml | |
| Analyte | LB-100 mg/kg | Tissue | T½ h | Tmax h | or ng/g | or ng · h/g | or ng · h/g | MRT h |
| 100 | 0.5 | Brain | / | 0.25 | 17.4 | 10.8 | / | / |
| | | Liver | 0.78 | 0.25 | 586 | 758.6 | 902.2 | 1.17 |
| | | Plasma | 0.31 | 0.25 | 1110 | 695.8 | 706.0 | 0.45 |
| | 1.0 | Brain | 1.67 | 0.25 | 33.9 | 35.3 | 72.5 | 2.68 |
| | | Liver | 0.79 | 0.25 | 1371 | 3526.5 | 3537.7 | 1.51 |
| | | Plasma | 0.99 | 0.25 | 2383 | 1923.5 | 2830.2 | 1.57 |
| | 1.5 | Brain | 1.93 | 0.25 | 43.5 | 125.5 | 140.8 | 2.57 |
| | | Liver | 2.01 | 1.0 | 2548 | 9081.0 | 10449.1 | 2.90 |
| | | Plasm | 2.20 | 0.25 | 3664 | 7399.6 | 8641.4 | 2.82 |

TABLE 10.8
Main pharmacokinetic parameters of Endothal in SD rats
following single iv administration of 100.

| | | | | | Cmax | AUClast | AUCINF | |
|---|---|---|---|---|---|---|---|---|
| | Dose of | | | | ng/ml | ng · h/ml | ng · h/ml | |
| Analyte | LB-100 mg/kg | Tissue | T½ h | Tmax h | or ng/g | or ng · h/g | or ng · h/g | MRT h |
| Endothal | 0.5 | Brain | / | / | / | / | / | / |
| | | Liver | 10.1 | 0.25 | 349 | 2598 | 3095 | 7.90 |
| | | Plasma | 6.65 | 0.25 | 577 | 546 | 828 | 2.96 |
| | 1.0 | Brain | / | / | / | / | / | / |
| | | Liver | 6.10 | 0.25 | 1425 | 6673 | 7370 | 6.14 |
| | | Plasma | 7.06 | 0.25 | 1230 | 2487 | 2750 | 4.38 |
| | 1.5 | Brain | / | / | / | / | / | / |
| | | Liver | 4.57 | 0.25 | 2964 | 18434 | 18850 | 4.54 |
| | | Plasma | 6.25 | 0.25 | 1178 | 4476 | 4730 | 4.57 |

Endothal concentrations of the 100 plasma samples were measured and pharmacokinetic parameters were calculated. LB100 was converted to endothal.

### Example 14

### Administration of an Endothal Prodrug

An amount of compound 105, 113, 151, 153 or 157 is administered to a subject afflicted with cancer. The amount of the compound is effective to deliver endothal to cancers cells in the subject.

An amount of compound 105, 113, 151, 153 or 157 is administered to a subject afflicted with brain cancer. The amount of the compound is effective to deliver endothal to brain cancers cells in the subject.

An amount of compound 105, 113, 151, 153 or 157 is administered to a subject afflicted with diffuse intrinsic pontine glioma or glioblastoma multiforme. The amount of the compound is effective to deliver endothal to diffuse intrinsic pontine glioma cells or glioblastoma multiforme cells in the subject.

An amount of compound 105, 113, 151, 153 or 157 is administered to a subject afflicted with brain cancer. The amount of the compound is effective to deliver endothal across the blood brain barrier of the subject.

### Discussion

Inhibition of PP2A interferes with multiple aspects of the DNA damage repair (DDR) mechanisms and with exit from mitosis. These mechanisms sensitize cancer cells to cancer treatments that cause acute DNA injury. Compound 100 (see U.S. Pat. No. 7,998,957 B2) has anti-cancer activity when used alone (Lu et al. 2009a) and significantly potentiates in vivo, without observable increase in toxicity, the anti-tumor activity of standard cytotoxic anti-cancer drugs including temozolomide (Lu et al. 2009b, Martiniova et al. 2010), doxorubicin (Zhang et al. 2010), and docetaxel. 100 was recently approved for Phase I clinical evaluation alone and in combination with docetaxel and is in clinical trial.

Diffuse Intrinsic Pontine Glioma (DIPG) is a uniformly fatal brain tumor of children for which no standard treatment other that radiation is available. Pediatric neurooncologists believe it is appropriate to treat even previously untreated patients on an investigational protocol that offers a new approach. There has been no advance in overall survival in Glioblastoma Multiforme (GSM) patients since the definite but marginal improvement shown years ago by the addition of temozolomide to radiation after surgery. Recurrent GBM is often treated with Avastin as second line therapy but following relapse after Avastin, experimental treatment is the standard. Of interest concerning inhibition of PP2A in brain tumors is the recent report that increased levels of PP2A are present in GBM and that patients with the highest levels of PP2A in their gliomas have the worst prognosis (Hoffstetter et al., 2012).

Compound 1D0 is a serine-threonine phosphatase inhibitor that potentiates the activity of standard chemotherapeutic drugs and radiation. The mechanism of potentiation is impairment of multiple steps in a DNA-damage repair process and inhibition of exit from mitosis. Compound 100 has been shown to potentiate the activity of temozolomide, doxorubicin, taxotere, and radiation against a variety of human cancer cell lines growing as subcutaneous xenografts. Compound 100 treatment yields a radiation dose enhancement factor of 1.45. Mice bearing subcutaneous (sc) xenografts of U251 human GBM cells were treated with compound 100 intraperitoneally together with radiation, each given daily for 5 days×3 courses. The drug/radiation combination was no more toxic that radiation alone and eliminated 60% of the xenografts (6 months plus follow-up). The remaining 40% of xenografts treated with the combination recurred two months later than xenografts treated with radiation alone. Wei et al. (2013) showed that inhibition of PP2A by compound 100 enhanced the effectiveness of targeted radiation in inhibiting the growth of human pancreatic cancer xenografts in an animal model. Thus, 100 would seem to be an ideal agent to combine with radiation to treat localized cancers such as brain tumors.

Compound 100 is highly effective against xenografts of human gliomas in combination with temozolomide and/or radiation. Compound 100, which has an IC₅₀ of 1-3 μM for a broad spectrum of human cancer cell lines, is a highly water soluble zwitterion that does not readily pass the blood brain barrier (BBB) as determined in rats and non-human primates. GLP toxokinetic studies of compound 100 given intravenously daily×5 days were performed in the rat and dog. The major expected toxicities at clinically tolerable doses expected to inhibit the target enzyme, PP2A, in vivo (3-5 mg/m²) are reversible microscopic renal proximal tubule changes and microscopic alterations in epicardial cells. It is of interest that fostriecin, a natural-product selective inhibitor of PP2A, was evaluated given iv daily for 5 days in phase I trials several years ago. Dose limiting toxicity was not achieved before the studies were terminated for lack of a reliable drug supply. In those studies, the major toxicities were reversible non-cumulative increases in serum creatinine and hepatic enzymes.

Compound 100 is considered stable relative to verapamil in the presence of mouse, rat, dog, monkey, and human microsomes. Compound 100 is poorly absorbed from or broken down in the gut so that little is present in plasma after oral administration. In glp studies in the male and female Sprague Dawley rat, the PK parameters for compound 100 given by slow iv bolus daily×5 days were also dose dependent and comparable on day 1 and day 4. The values for female rats after drug at 0.5, 0.75, and 1.25 mg/kg on day 4 were respectively: Cₒ (ng/ml) 1497, 2347, and 3849; AUCₗₐₛₜ (ng·h/ml) 452, 691, and 2359; SC AUCₗₐₛₜ (ng·h/ml) 17.7, 54.0, and 747; DN AUCₗₐₛₜ 904, 921, and 1887; AUC* (ng·h/ml) 479, 949, and 2853; % AUC* Extrapolated 5.6, 27, and 17; T_{1/2} (h) 0.25, 0.59, and 1.8; Cl (mL/h/kg) 1045, 790, 438 (MALE 1071, 1339, 945); V_{z} (ml/kg) 378, 677, and 1138. In glp studies in the male and female dog, the toxicokinetic parameters for compound 100 given iv over 15 minutes daily for 5 days were dose dependent and comparable on day 1 and day 4. The values for the female dogs on after drug at 0.15, 0.30, and 0.50 mg/kg on day 4 were respectively: Cₒ (ng/ml) 566, 857, and 1930; AUCₗₐₛₜ (ng·h/ml) 335, 1020, and 2120; Cmax (ng/ml) 370, 731, 1260; Tₘₐₓ (hr) 0.25, 0.35, and 0.25; and, T_{1/2} (h) 0.47, 0.81, and 1.2 (IND No. 109,777: compound 100 for Injection). Inhibition of the abundant PP2A in circulating white blood cells (isolated by Ficoll-Hypaque) has been shown to be dose dependent in the rat following slow iv administration of 100 at 0.375, 0.75, and 1.5 mg/kg resulting 9, 15 and 25% inhibition, respectively.

The methyl ester of 100, compound 151, which has an oral bioavailability of about 60% versus 1% for compound 100, was given by mouth to rats. Compound 151 treatment resulted in substantial levels of compound 100 in the plasma with an apparently much greater half life compared with 100 given intravenously. However, compound 151 was barely detectable in brain tissue.

A series of analogs of compound 100 have been developed and tested. Without wishing to be bound by theory, it is believed that C₂-C₂₀ alkyl, C₂-C₂₀ alkenyl, and C₂-C₂₀ alkynyl esters of compound 100 cross the BBB to release sufficient amounts of compound 100 thereby inhibiting PP2A sufficiently to treat brain cancer or to enhance the effectiveness of standard radiation treatment with or without adjuvant chemotherapy against brain cancer. Brain cancer includes, but is not limited to, pediatric DIPGs and adult GBMs. Enhancement of the efficacy of radiation treatment for these diseases leads to a greater reduction in tumor mass, to a more rapid and profound reduction in symptoms, and an increased life-span. Also, the number of treatment days required is reduced.

Based on the data contained herein and without wishing to be bound by theory, it is believed that further increasing lipophilicity, i.e., increasing the length of the alkyl chain of compound 151, allows the compound (given orally or parenterally) to penetrate the BBB and release amounts of compound 100 sufficient to treat intracerebral (brain) cancers or sensitize intracerebral (brain) cancers to radiation and cytotoxic drugs.

The C₂-C₂₀ alkyl, C₂-C₂₀ alkenyl, and C₂-C₂₀ alkynyl esters of compound 100 cross the BBB to release sufficient amounts of endothal thereby inhibiting PP2A sufficiently to treat brain cancer or to enhance the effectiveness of standard radiation treatment with or without adjuvant chemotherapy against brain cancer. Brain cancer includes, but is not limited to, pediatric DIPGs and adult GBMs. Enhancement of the efficacy of radiation treatment for these diseases leads to a greater reduction in tumor mass, to a more rapid and profound reduction in symptoms, and an increased life-span. Also, the number of treatment days required is reduced.

Based on the data contained herein and without wishing to be bound by theory, it is believed that further increasing lipophilicity, i.e., increasing the length of the alkyl chain of compound 151, allows the compound (given orally or parenterally) to penetrate the BBB and release amounts of endothal sufficient to treat intracerebral (brain) cancers or sensitize intracerebral (brain) cancers to radiation and cytotoxic drugs.

The analogs of compound 100 disclosed herein cross the BBB to release sufficient amounts of endothal thereby inhibiting PP2A sufficiently to treat brain cancer or to enhance the effectiveness of standard radiation treatment with or without adjuvant chemotherapy against brain cancer. Brain cancer includes, but is not limited to, pediatric DIPGs and adult GBMs. Enhancement of the efficacy of radiation treatment for these diseases leads to a greater reduction in tumor mass, to a more rapid and profound reduction in symptoms, and an increased life-span. Also, the number of treatment days required is reduced.

Based on the data contained herein and without wishing to be bound by theory, it is believed that further increasing lipophilicity, i.e., replcing the OH with O-alkyl or other amide or ester derivative, allows the analog of compound 100 (given orally or parenterally) to penetrate the BBB and release amounts of endothal sufficient to treat intracerebral (brain) cancers or sensitize intracerebral (brain) cancers to radiation and cytotoxic drugs.

Based on the data contained in Examples 8-11, compounds 105, 113, 151, 153 and 157 are converted to endothal in the plasma when administered to rats. Accordingly, compounds 105, 113, 151, 153 and 157 and derivative thereof are useful as prodrugs of endothal.

Pre-clinical data suggests that if PP2A can be inhibited in brain tumors, current standard and minimally effective modalities of treatment, particularly radiation, will produce greater regression of tumor mass with improvement in symptoms and, as the major goal, improvement in productive life-span. Animal models of intracranial human glioma are available and were used to demonstrate that parenteral doses of compound 100 combined with radiation can eradicate a majority of subcutaneous xenografts.

### REFERENCES

Bastian et al. (2004), Gene, Vol. 328, pp. 1-16.
Giannini, R. and Cvallini, A. (2005) Anticancer Research Vol. 36, No. 6B, pp. 4287-4292.
Graziano, M. J. and Casida, J. E. (1987) Toxicol Lett. 37, 143-148.
Havrilesky, L J et al. (2001) J. Soc. Gynecology. Investig. Vol. 8, pp. 104-113.
Hawkins C E et al (2011) Journal of Clinical Oncology, Vol 29, No. 30, 3954-3956.
Hermanson et al. (2002) Nature, Vol. 419, pp. 934-939.
Hofstetter C P et al (2012) PLoS ONE 7(1):1-11.
Honkanan, R. E. et al. (1993) FESS Lett. 330, 283-286.
Li, Y. M. et al. (1992) Proc. Natl. Acad. Sci. USA, 89, 11867-11870.
Li, Y. M. et al. (1993) Biochem. Pharmacol. 46, 1435-1443.
Lu J et al (2009a) J Neurosurgery Vol. 113, No. 2, Pages 225-233.
Lu J et al (2009b) PNAS 106(28), 11697-11702.
Martiniova L et al (2011) PLoS ONE 6(2):1-8.
Myers, E. et al. (2005) Clin. Cancer Res. Vol. 11, pp. 2111-2122.
Park D M et al (2007) Cell Cycle 6(4):467-470.
Stupp R et al (2009) Lancet Oncol 10:459-466.
Thiery J P, et al. (1999) Hepatology, 29, 1406-17.
Tsauer, W. et al. (1997) Anticancer Research 17, 2095-2098.
Wang, D. S. (1989) Journal of Ethnopharmacology 26, 147-162.
Warren K et al (2012) Cancer 118:3607-3613.
Waters, C E et al. (2004) J. Endocrinol. Vol. 183, pp. 375-383.
Wei et al (2013) Clin. Cancer Res. 19, 4422-4432.
Ynag, Y. et al. (2011) Acta Pharmaceutica Sinica B, 1(3), 143-159.
Zhang C et al (2010) Biomaterials 31, 9535-9543.
Zhuang Z et al (2009) Cell Cycle 8(20):3303-3306.

1. A method for in vivo delivery of endothal to a target cell in a subject, the method comprising administering to the subject a compound having the structure: 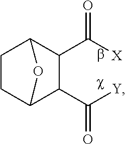
wherein
X is OR₃ or NR₄R₅,
wherein each of R₃, R₄ and R₅ is H or an organic moiety, or R₄ and R₅ combine to form an organic moiety:
Y is OR₆ or NR₇R₈;
wherein each of R₆, R₇ and R₈ is H or an organic moiety, or R₇ and R₈ combine to form an organic moiety:
wherein when one of X or Y is OH, then the other of X or Y is other than OH, NR₄R₅ or NR₇R₈ where R₄ and R₅ or R₇ and R₈ combine to form an N-methyl piperazine,
or a pharmaceutically acceptable salt or ester of the compound,
wherein if X is OH, bond χ is subject to in vivo hydrolytic cleavage in the subject; if Y is OH, bond β is subject to in vivo hydrolytic cleavage in the subject; and if neither X nor Y is OH, bond X and bond β are subject to in vivo hydrolytic cleavage in the subject,
so as to thereby deliver endothal to the target cell in the subject.

2. The method of claim 1,
wherein when one of X or Y is OH, then the other of X or Y is other than NR₄R₅ or NR₇R₈ where R₄ and R₅ or R₇ and R₈ combine to form an N-tert-butylcarboxylate piperazine.

3. The method of claim 1,
wherein when one of X or Y is OH, then the other of X or Y is other NR₄R₅ or NR₇R₈ where R₄ and R₅ or R₇ and R₈ combine to form an unsubstituted or substituted piperazine, morpholine or thiomorpholine.

4. The method of claim 1, wherein
X is OR₃ or NR₄R₅,
wherein
R₃ is H, alkyl, alkenyl, alkynyl, aryl, heteroaryl, alkylaryl, alkylheteroaryl, alkenylaryl, alkenylheteroaryl, alkynylaryl, alkynylheteroaryl, heteroalkyl, heteroalkenyl, heteroalkynyl, hydroxyalkyl, hydroxyalkenyl, hydroxyalkynyl, alkyl —P(O)(OR₉)₂, alkyl-OP(O)(OR₉)₂, alkyl-O(CO)—OR₁₀, alkyl-P(O)(O-alkyl-O(CO)—OR₁₀)₂, or alkyl-OP(O)(O-alkyl-O(CO)—OR₁₀)₂;
R₄ and R₅ are each independently H, alkyl, alkenyl, alkynyl, aryl, heteroaryl, alkylaryl, alkylheteroaryl, alkenylaryl, alkenylheteroaryl, alkynylaryl, alkynylheteroaryl, heteroalkyl, heteroalkenyl, heteroalkynyl, hydroxyalkyl, hydroxyalkenyl, hydroxyalkynyl, alkyl-P(O)(OR₉)₂, alkyl-OP(O)(OR₉)₂, alkyl-O(CO)—OR₁₀, alkyl-P(O)(O-alkyl-O(CO)—OR₁₀)₂, or alkyl-OP(O)(O-alkyl-O(CO)—OR₁₀)₂,
or R₄ and R₅ combine to form an unsubstituted or substituted cycloalkyl, cycloalkenyl, cycloalkynyl or heterocycloalkyl,
wherein, R₉ and R₁₀ are each independently H, alkyl, alkenyl, alkynyl, or aryl; and
Y is OR₆ or NR₇R₈,
wherein
R₆ is H, alkyl, alkenyl, alkynyl, aryl, heteroaryl, alkylaryl, alkylheteroaryl, alkenylaryl, alkenyiheteroaryl, alkynylaryl, alkynylheteroaryl, heteroalkyl, heteroalkenyl, heteroalkynyl, hydroxyalkyl, hydroxyalkenyl, hydroxyalkynyl, alkyl-P(O)(OR₉)₂, alkyl-OP(O)(OR₉)₂, alkyl-O(CO)—OR₁₀, alkyl-P(O)(O-alkyl-O(CO)—OR₁₀)₂, or alkyl-OP(O)(O-alkyl-O(CO)—OR₁₀)₂;
R₇ and R₈ are each independently H, alkyl, alkenyl, alkynyl, aryl, heteroaryl, alkylaryl, alkylheteroaryl, alkenylaryl, alkenylheteroaryl, alkynylaryl, alkynylheteroaryl, heteroalkyl, heteroalkenyl, heteroalkynyl, hydroxyalkyl, hydroxyalkenyl hydroxyalkynyl, alkyl-P(O)(OR₉)₂, alkyl-OP(O)(OR₉)₂, alkyl-O(CO)—OR₁₀, alkyl-P(O)(O-alkyl-O(CO)—OR₁₀)₂, or alkyl-OP(O)(O-alkyl-O(CO)—OR₁₀)₂,
or R₇ and R₈ combine to form an unsubstituted or substituted cycloalkyl, cycloalkenyl, cycloalkynyl or heterocycloalkyl,
wherein R₉ and R₁₀ are each independently H, alkyl, alkenyl, alkynyl, or aryl,
or a pharmaceutically acceptable salt or ester of the compound.

5. The method of claim 4,
wherein
X is OR₃ or NR₄R₅,
wherein
R₃ is H, alkyl, alkenyl, hydroxyalkyl, alkylaryl, alkyl-P(O)(OR₉)₂, alkyl-OP(O)(OR₉)₂, alkyl-O(CO)—OR₁₀, alkyl-P(O)(O-alkyl-O(CO)—OR₁₀)₂, or alkyl-OP(O)(O-alkyl-O(CO)—OR₁₀)₂;
R₄ and R₅ are each independently H, alkyl, alkenyl, hydroxyalkyl, alkyl-P(O)(OR₉)₂, alkyl-OP(O)(OR₉)₂, alkyl-O(CO)—OR₁₀, alkyl-P(O)(O-alkyl-O(CO)—OR₁₀)₂, or alkyl-OP(O)(O-alkyl-O(CO)—OR₁₀)₂,
or R₄ and R₅ combine to form an unsubstituted or substituted heterocycloalkyl,
wherein R₉ and R₁₀ are each independently H, alkyl, alkenyl, alkynyl, or aryl; and
Y is OR₆ or NR₇R₈,
wherein
R₆ is H, alkyl, alkenyl, hydroxyalkyl, alkylaryl, alkyl-P(O) (OR₉)₂, alkyl-OP(O)(OR₉)₂, alkyl-O(CO)—OR₁₀, alkyl-P(O)(O-alkyl-O(CO)—OR₁₀)₂, or alkyl-OP(O)(O-alkyl-O(CO)—OR₁₀)₂;
R₇ and R₈ are each independently H, alkyl, alkenyl, hydroxyalkyl, alkyl-P(O)(OR₉)₂, alkyl-OP(O)(OR₉)₂, alkyl-O(CO)—OR₁₀, alkyl-P(O)(O-alkyl-O(CO)—OR₁₀)₂, or alkyl-OP(O)(O-alkyl-O(CO)—OR₁₀)₂,
or R₇ and R₈ combine to form an unsubstituted or substituted heterocycloalkyl,
wherein R₉ and R₁₀ are each independently H, alkyl, alkenyl, alkynyl, or aryl,
or a pharmaceutically acceptable salt or ester of the compound.

6. The method of claim 5,
wherein
X is OR₃, 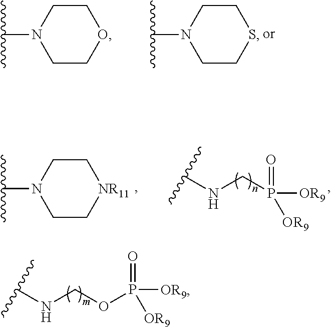
wherein R₃ is H, alkyl, alkenyl, hydroxyalkyl, alkylaryl, alkyl P(O)(OR₉)₂, alkyl-OP(O)(OR₉)₂, alkyl-O(CO)—OR₁₀, alkyl-P(O) (O-alkyl-O(CO)—OR₁₀)₂, or alkyl-OP(O)(O-alkyl-O(CO)—OR₁₀)₂,
wherein R₉ and R₁₀ are each independently H, alkyl, alkenyl, or alkynyl,
wherein R₁₁ is H, alkyl, hydroxyalkyl, alkenyl, alkenyl, alkynyl, aryl, alkylaryl, heteroaryl, alkylheteroaryl, 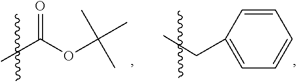
—CH₂CN, —CH₂CO₂R₁₂, —CH₂COR₁₂, —NHR₁₂, or —NH′(R₁₂)₂,
where each R₁₂ is independently H, alkyl, alkenyl or alkynyl; and
Y is OR₆, 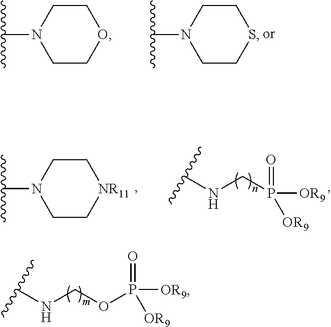
wherein R₆ is H, alkyl, alkenyl, hydroxyalkyl, alkylaryl, alkyl-P(O)(OR₉)₂, alkyl-OP(O)(OR₉)₂, alkyl-O(CO)—OR₁₀, alkyl-P(O)(O-alkyl-O(CO)—OR₁₀)₂, or alkyl-OP(O)(O-alkyl-O(CO)—OR₁₀)₂,
wherein R₉ and R₁₀ are each independently H, alkyl, alkenyl, or alkynyl,
wherein R₁₁ is H, alkyl, hydroxyalkyl, alkenyl, alkenyl, alkynyl, aryl, alkylaryl, heteroaryl, alkylheteroaryl, 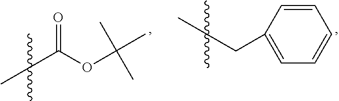
—CH₂CN, —CH₂CO₂R₁₂, —CH₂COR₁₂, —NHR₁₂, or —NH⁺(R₁₂)₂,
wherein R₉ and R₁₀ are each independently H, alkyl, alkenyl, or alkynyl,
where each R₁₂ is independently H, alkyl, alkenyl or alkynyl.

7.-13. (canceled)

14. The method of claim 5, wherein the compound has the structure: 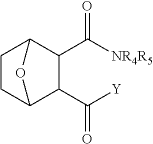
wherein
R₄ and R₅ are each H, alkyl, alkenyl, alkynyl, alkyl-P(O)(OR₉)₂, alkyl-OP(O)(OR₉)₂, alkyl-O(CO)—OR₁₀, alkyl-P(O)(O-alkyl-O(CO)—OR₁₀)₂, or alkyl-OP(O)(O-alkyl-O(CO)—OR₁₀)₂,
wherein R₉ and R₁₀ are each independently H, alkyl, alkenyl, or alkynyl; and
Y is OR₆, 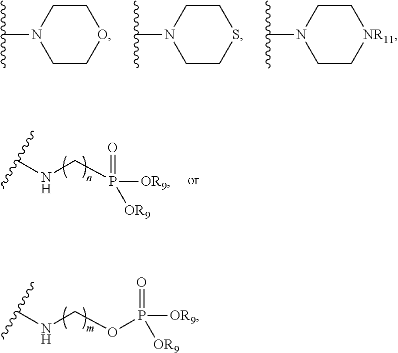
wherein R₆ is H, alkyl, alkenyl, hydroxyalkyl, alkylaryl, alkyl-P(O)(OR₉)₂, alkyl-OP(O)(OR₉)₂, alkyl-O(CO)—OR₁₀, alkyl-P(O)(O-alkyl-O(CO)—OR₁₀)₂, or alkyl-OP(O)(O-alkyl-O(CO)—OR₁₀)₂,
wherein R₉ and R₁₀ are each independently H, alkyl, alkenyl, or alkynyl,
wherein R₁₁ is H, alkyl, hydroxyalkyl, alkenyl, alkenyl, alkynyl, aryl, alkylaryl, heteroaryl, alkylheteroaryl, 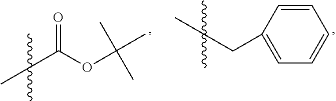
—CH₂CN, —CH₂CO₂R₁₂, —CH₂COR₁₂, —NHR₁₂, or —NH⁺(R₁₂)₂,
where each R₁₂ is independently H, alkyl, alkenyl or alkynyl.

15.-21. (canceled)

22. The method of claim 4, wherein the compound has the structure: 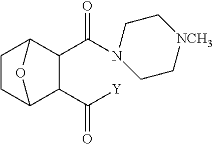
wherein
Y is OR₆, 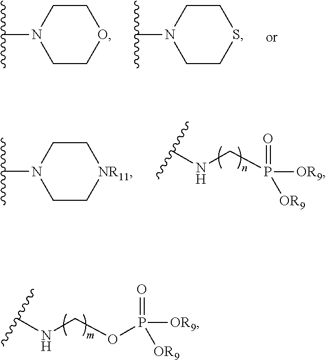
wherein R₆ is H, alkyl, alkenyl, hydroxyalkyl, alkylaryl, alkyl-P(O)(OR₉)₂, alkyl-OP(O)(OR₉)₂, alkyl-O(CO)—OR₁₀, alkyl-P(O)(O-alkyl-O(CO)—OR₁₀)₂, or alkyl-OP(O)(O-alkyl-O(CO)—OR₁₀)₂,
wherein R₉ and R₁₀ are each independently H, alkyl, alkenyl, or alkynyl,
wherein R₁₁ is H, alkyl, hydroxyalkyl, alkenyl, alkenyl, alkynyl, aryl, alkylaryl, heteroaryl, alkylheteroaryl, 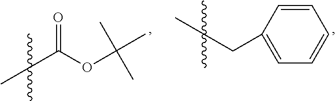
—CH₂CN, —CH₂CO—R₁₂, —CH₂COR₁₂, —NHR₁₂, or —NH⁺(R₁₂)₂,
where each R₁₂ is independently H, alkyl, alkenyl or alkynyl.

23.-28. (canceled)

29. The method of claim 1, wherein
wherein
X is OH, O⁻, OR₁₃, O(CH₂)₁₋₆R₁₃, SH, S⁺, or SR₁₃,
wherein R₁₃ is H, alkyl, alkenyl, alkynyl or aryl;
Y is 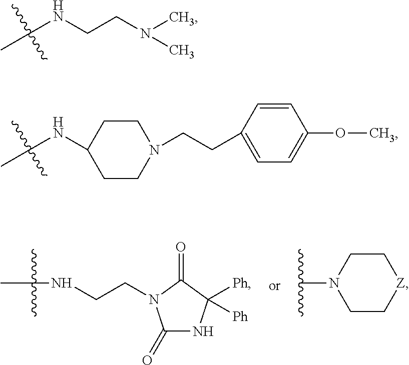
where Z is O, S, NR₁₄, N⁺HR₁₄ or N⁺R₁₄R₁₄,
where each Rje is independently H, alkyl, alkenyl, alkynyl, aryl, 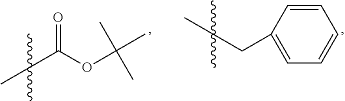
—CH₂CN, —CH₂COR₁₅, or —CH₂COR₁₅,
wherein each R₁₅ is independently H, alkyl, alkenyl or alkynyl.

30.-39. (canceled)

40. The method of claim 1, wherein
X is O(CH₂)₁₋₆R₁₆ or OR₁₆
where each R₁₆ is H, alkyl, C₂-C₁₂ alkyl, substituted alkyl, alkenyl, alkynyl, aryl, (C₆H₅)(CH₂)₁₋₆(CHNHBOC)CO₂H, (C₆H₅)(CH₂)₁₋₆(CHNH₂)CO₃H, (CH₂)₁₋₆(CHNHBOC)CO₂H, (CH₂)₁₋₆(CHNH₂)CO₂H or (CH₂)₁₋₆CCl₃; and
Y is 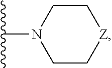
where Z is O, S, NR₁₄, N⁺HR₁₄ or N⁺R₁₄R₁₄,
where each R₁₄ is independently H, alkyl, hydroxyalkyl, C₂-C₁₂ alkyl, alkenyl, C₄-C₁₂ alkenyl, alkynyl, aryl, 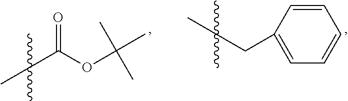
—CH₂CN, —CH₂CO₂R₁₅, or —CH₂COR₁₅,
wherein each R₁₅ is independently H, alkyl, alkenyl or alkynyl.

41.-49. (canceled)

50. The method of claim 1, wherein the compound has the structure: 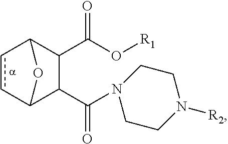
wherein
bond α is absent;
R₁ is C₂-C₂₀ alkyl, C₂-C₂₀ alkenyl, or C₂-C₂₀ alkynyl;
R₂ is H, C₁-C₁₂ alkyl, C₁-C₁₂ alkenyl, C₁-C₁₂ alkynyl, C₁-C₁₂ alkyl-(phenyl), C₁-C₁₂ alkyl-(OH), or C(O)C(CH₃)₃,
or a pharmaceutically acceptable salt of the compound.

51.-60. (canceled)

61. The method of claim 1 wherein the compound has the structure: 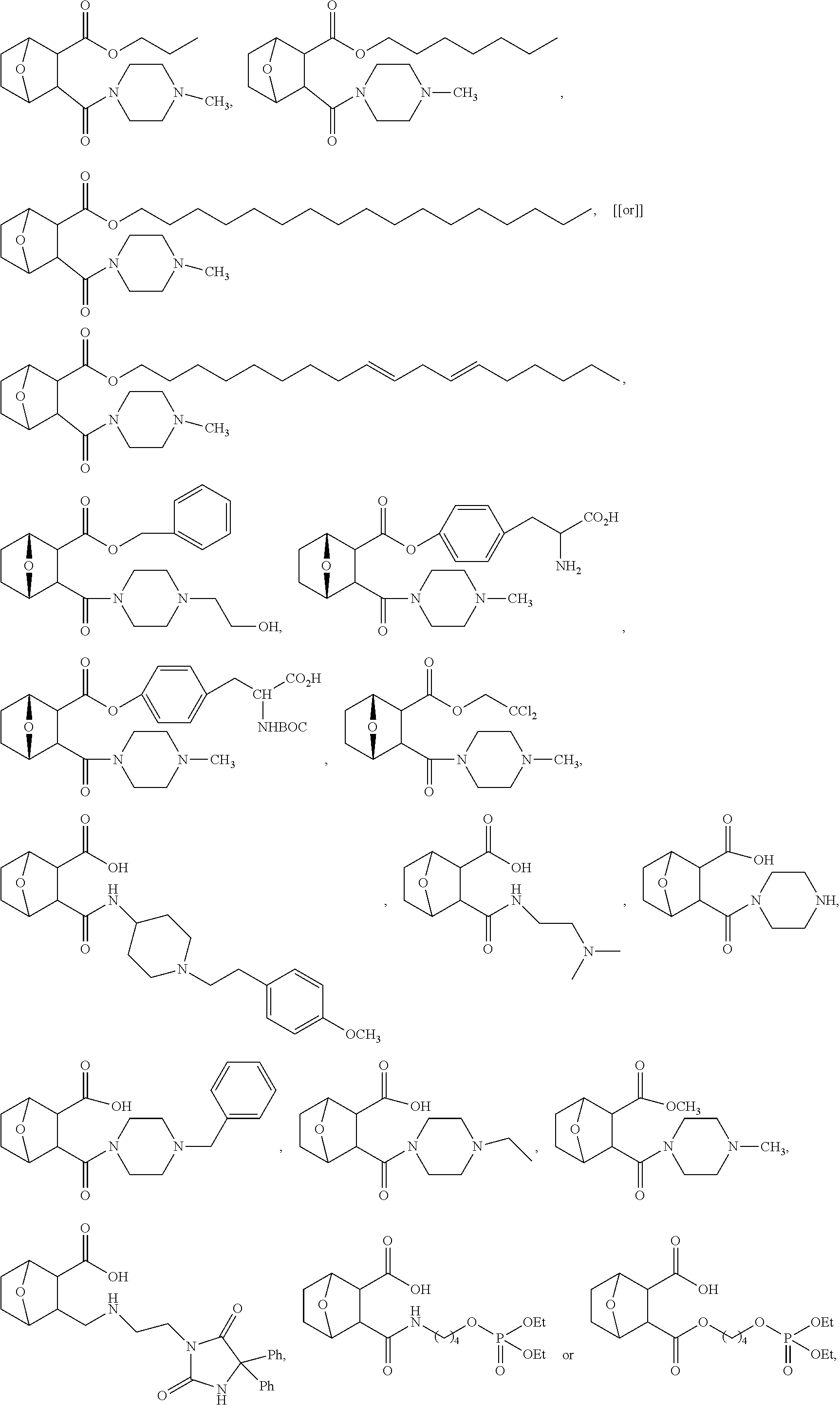
or a pharmaceutically acceptable salt or ester of the compound.

62. The method of claim 1, wherein the delivery of the endothal to the target cell in the subject is effective to treat a disease in the subject afflicted with the disease.

63. The method of claim 62, wherein the disease is cancer.

64.-66. (canceled)

67. The method of claim 63, further comprising administering to the subject an anti-cancer agent.

68.-76. (canceled)

77. A compound having the structure: 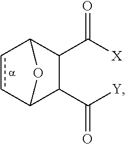
wherein
bond α is absent or present;
X is OR₁, OR₃ or NR₄R₅,
wherein
R₁ is C₁-C₂₀ alkyl, C₂-C₂₀ alkenyl, or C₂-C₂₀ alkynyl;
R₃ is H, alkyl, alkylaryl, alkyl-P(O)(OR₉)₂, alkyl-OP(O)(OR₉)₂, alkyl-O(CO)—OR₁₀, alkyl-P(O)(O-alkyl-O(CO)—OR₁₀)₂, or alkyl-OP(O)(O-alkyl-O(CO)—OR₁₀)₂;
R₄ and R₅ are each independently H, alkyl-P(O)(OR₉)₂, alkyl-OP(O)—(OR₉)₂, alkyl-O(CO)—OR₁₀, alkyl-P(O)(O-alkyl-O(CO)—OR₁₀)₂, or alkyl-OP(O)(O-alkyl-O(CO)—OR₁₀)₂,
or R₄ and R₅ combine to form an unsubstituted or substituted cycloalkyl, cycloalkenyl, cycloalkynyl or heterocycloalkyl,
wherein R₉ and R₁₀ are each independently H, alkyl, alkenyl, or alkynyl; and
Y is OR₁, OR₆ or NR₇R₈,
wherein
R₁ is C₁-C₂₀ alkyl, C₂-C₂₀ alkenyl, or C₂-C₂₀ alkynyl;
R₆ is alkyl-P(O)(OR₉)₂, alkyl-OP(O)(OR₉)₂, alkyl-O(CO)—OR₁₀, alkyl-P(O)(O-alkyl-O(CO)—OR₁₀)₂, or alkyl-OP(O)(O-alkyl-O(CO)—OR₁₀)₂; and
R₇ and R₈ are each independently H, alkyl-P(O)(OR₉)₂, alkyl-OP(O)(OR₉)₂, alkyl-O(CO)—OR₁₀, alkyl-P(O)(O-alkyl-O(CO)—OR₁₀)₂, or alkyl-OP(O)(O-alkyl-O(CO)—OR₁₀)₂,
or R₇ and R₈ combine to form an unsubstituted or substituted cycloalkyl, cycloalkenyl, cycloalkynyl or heterocycloalkyl,
wherein R₉ and R₁₀ are each independently H, alkyl, alkenyl, or alkynyl,
wherein one of X is OH, OCH₃ or O-alkylaryl, then Y is other than NR₇R₈ where R₇ and R₈ combine to form an unsubstituted or substituted piperazine, morpholine or thiomorpholine,
or a pharmaceutically acceptable salt or ester of the compound.

78-91. (canceled)

92. The compound of claim 77 having the structure: 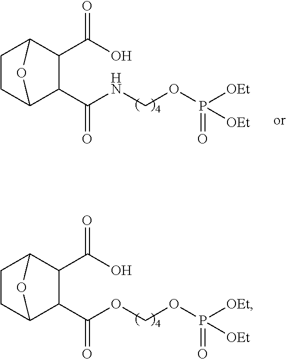
or a pharmaceutically acceptable salt or ester of the compound.

93-98. (canceled)

99. A compound of claim 77 having the structure: 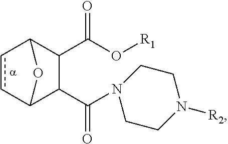
wherein
bond α is absent or present;
R₁ is C₂-C₂₀ alkyl, C₂-C₂₀ alkenyl, or C₂-C₂₀ alkynyl;
R₂ is H, C₁-C₁₂ alkyl, C₁-C₁₂ alkenyl, C₁-C₁₂ alkynyl, C₁-C₁₂ alkyl-(phenyl), C₁-C₁₂ alkyl-(OH), or C(O)C(CH₃)₃,
or a pharmaceutically acceptable salt of the compound.

100.-111. (canceled)

112. The compound of claim 99, having the structure: 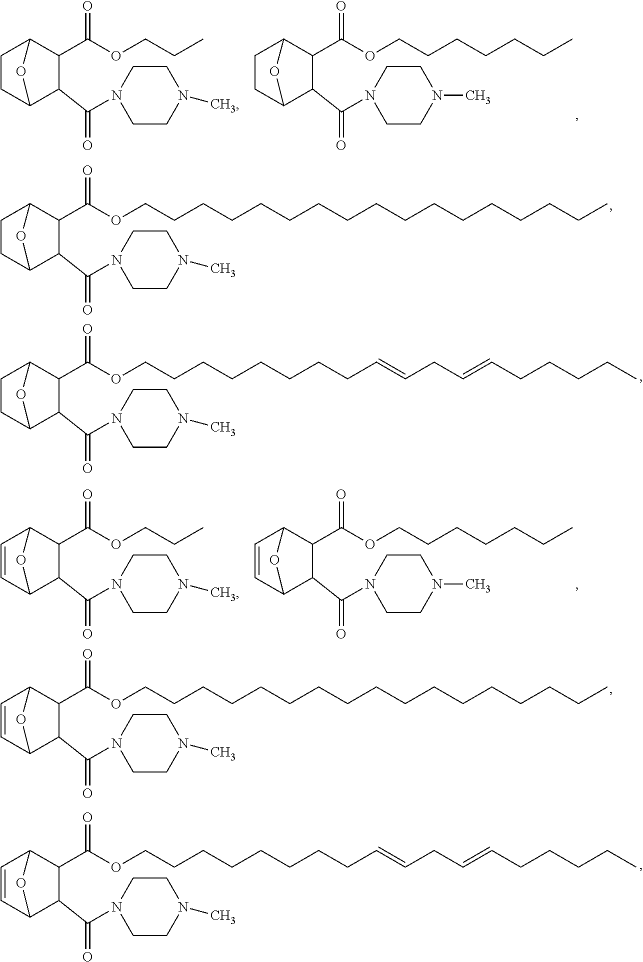
or a pharmaceutically acceptable salt of the compound.

113.-120. (canceled)

121. A method of treating a subject afflicted with cancer comprising administering to the subject a therapeutically effective amount of the compound of claim 77.

122.-159. (canceled)
